(19) United States
(12) Patent Application Publication
(10) Pub. No.: US 2026/0306669 A1
(43) Pub. Date: Oct. 8, 2026

(54) METHODS OF TREATING NEURODEGENERATIVE DISORDERS

(71) Applicant: Seelos Therapeutics, Inc., New York, NY (US)

(72) Inventors: Raj Mehra, New York, NY (US); Krishnamurthy Ganapathy Subramanian, Mechanicsville, VA (US)

(21) Appl. No.: 19/481,357

(22) PCT Filed: May 9, 2024
(86) PCT No.: PCT/US2024/028599

§ 371 Date: Nov. 4, 2025

Related U.S. Application Data
(60) Provisional application No. 63/465,688, filed on May 11, 2023.

(51) Int. Cl. A61K 48/00; A61K 9/00; A61K 38/00; A61P 25/28; C12N 9/10; C12N 9/16; C12N 15/62; C12N 15/86

(52) U.S. Cl. CPC A61K 48/005; A61K 9/0085; A61K 48/0075; A61P 25/28; C12N 9/1051; C12N 9/16; C12N 15/62; C12N 15/86; A61K 38/00; C07K 2319/00; C12N 2750/14143; C12N 2750/14151; C12N 2830/15; C12Y 204/01015; C12Y 301/03012

(57) ABSTRACT

The present disclosure relates to compositions comprising a vector (e.g., an adeno-associated viral vector) encoding trehalose biosynthesis polypeptides (e.g., OtsA and OtsB, or fusion polypeptides thereof), and methods of using same for the treatment of neurodegenerative disorders. For example, disclosed are methods of treating a neurodegenerative disorder comprising administering a vector comprising a nucleic acid that encodes trehalose-6-phosphate synthase (OtsA), a nucleic acid that encodes trehalose 6-phosphate phosphatase (OtsB), a nucleic acid that encodes OtsA and OtsB, or a nucleic acid that encodes a trehalose biosynthesis fusion polypeptide.

### CLAIM OF PRIORITY

This application claims benefit of U.S. Provisional Patent Application Ser. No. 63/465,688, filed May 11, 2023. The entire contents of this application are hereby incorporated by reference.

### SEQUENCE LISTING

This application contains a Sequence Listing that has been submitted electronically as an XML file named 47542-0044WO1_ST26_SL.xml. The XML file, created on Apr. 22, 2024, is 494,502 bytes in size. The material in the XML file is hereby incorporated by reference in its entirety.

### TECHNICAL FIELD

The present disclosure relates to compositions comprising nucleic acids encoding trehalose-6-phosphate synthase (OtsA) and/or trehalose 6-phosphate phosphatase (OtsB), or a trehalose biosynthesis fusion polypeptide in viral expression vectors and methods of using same for the treatment of neurodegenerative disorders.

### BACKGROUND

Neurodegenerative disorders are a class or brain disorder in which the subject slowly loses, for example, their memory, thinking ability, and social skills. It is characterized by changes in the brain that lead to deposits of certain proteins, or plaques. Well-known neurodegenerative diseases include Alzheimer's disease and Parkinson's disease. About 6.5 million people in the United States age 65 and older live with Alzheimer's disease. Current therapies focus on limiting symptoms and can be time-consuming. For example, monthly infusions may be necessary. There is a clear need for more effective treatments of neurodegenerative disorders.

### SUMMARY

Described herein are methods of treating a subject having a neurodegenerative disorder that include administering to the subject a therapeutically effective amount of a vector comprising a nucleic acid that encodes trehalose-6-phosphate synthase (OtsA), a nucleic acid that encodes trehalose 6-phosphate phosphatase (OtsB), a nucleic acid that encodes OtsA and OtsB, or a nucleic acid that encodes a trehalose biosynthesis fusion polypeptide.

Also described herein are methods of preventing a neurodegenerative disorder that include administering to the subject a therapeutically effective amount of a vector comprising a nucleic acid that encodes trehalose-6-phosphate synthase (OtsA), a nucleic acid that encodes trehalose 6-phosphate phosphatase (OtsB), a nucleic acid that encodes OtsA and OtsB, or a nucleic acid that encodes a trehalose biosynthesis fusion polypeptide.

Also described herein are methods of treating a subject having a neurodegenerative disorder that include administering to the subject a therapeutically effective amount of trehalose and a therapeutically effective amount of a vector comprising a nucleic acid that encodes trehalose-6-phosphate synthase (OtsA), a nucleic acid that encodes trehalose 6-phosphate phosphatase (OtsB), a nucleic acid that encodes OtsA and OtsB, or nucleic acid that encodes a trehalose biosynthesis fusion polypeptide.

Also described herein are methods of treating a subject in need thereof that includes (a) identifying the subject as having a neurodegenerative disorder; and (b) administering to the subject a therapeutically effective amount of a vector comprising a nucleic acid that encodes trehalose-6-phosphate synthase (OtsA), a nucleic acid that encodes trehalose 6-phosphate phosphatase (OtsB), a nucleic acid that encodes OtsA and OtsB, or a nucleic acid that encodes a trehalose biosynthesis fusion polypeptide.

Also described herein are methods of treating a subject previously determined to have a neurodegenerative disorder, the method includes administering to the subject a therapeutically effective amount of a vector comprising a nucleic acid that encodes trehalose-6-phosphate synthase (OtsA), a nucleic acid that encodes trehalose 6-phosphate phosphatase (OtsB), a nucleic acid that encodes OtsA and OtsB, or a nucleic acid that encodes a trehalose biosynthesis fusion polypeptide.

Also described herein are methods of preventing, ameliorating, or preventing and ameliorating muscle atrophy, cramps and/or fasciculations in a subject at risk of developing a neurodegenerative disorder, that method includes administering to the subject a therapeutically effective amount of a vector comprising a nucleic acid that encodes trehalose-6-phosphate synthase (OtsA), a nucleic acid that encodes trehalose 6-phosphate phosphatase (OtsB), a nucleic acid that encodes OtsA and OtsB, or a nucleic acid that encodes a trehalose biosynthesis fusion polypeptide.

Also described herein are methods of preventing, ameliorating, or preventing and ameliorating paralysis of voluntary muscles in a subject at risk of developing a neurodegenerative disorder, that method includes administering to the subject a therapeutically effective amount of a vector comprising a nucleic acid that encodes trehalose-6-phosphate synthase (OtsA), a nucleic acid that encodes trehalose 6-phosphate phosphatase (OtsB), a nucleic acid that encodes OtsA and OtsB, or a nucleic acid that encodes a trehalose biosynthesis fusion polypeptide.

Also described herein are methods of preventing, ameliorating, or preventing and ameliorating dementia in a subject at risk of developing a neurodegenerative disorder, that method includes administering to the subject a therapeutically effective amount of a vector comprising a nucleic acid that encodes trehalose-6-phosphate synthase (OtsA), a nucleic acid that encodes trehalose 6-phosphate phosphatase (OtsB), a nucleic acid that encodes OtsA and OtsB, or a nucleic acid that encodes a trehalose biosynthesis fusion polypeptide.

Also described herein are methods of preventing, ameliorating, or preventing and ameliorating decline in motor and cognitive function (e.g., a sudden or progressive decline) in a subject at risk of developing a neurodegenerative disorder, that method includes administering to the subject a therapeutically effective amount of a vector comprising a nucleic acid that encodes trehalose-6-phosphate synthase (OtsA), a nucleic acid that encodes trehalose 6-phosphate phosphatase (OtsB), a nucleic acid that encodes OtsA and OtsB, or a nucleic acid that encodes a trehalose biosynthesis fusion polypeptide.

Also described herein are methods of preventing, ameliorating, or preventing and ameliorating protein aggregation in the brain of a subject at risk of developing a neurodegenerative disorder, that method includes administering to the subject a therapeutically effective amount of a vector comprising a nucleic acid that encodes trehalose-6-phosphate synthase (OtsA), a nucleic acid that encodes trehalose 6-phosphate phosphatase (OtsB), a nucleic acid that encodes OtsA and OtsB, or a nucleic acid that encodes a trehalose biosynthesis fusion polypeptide.

Also described herein are methods of preventing, ameliorating, or preventing and ameliorating ataxia in a subject at risk of developing a neurodegenerative disorder, that method includes administering to the subject a therapeutically effective amount of a vector comprising a nucleic acid that encodes trehalose-6-phosphate synthase (OtsA), a nucleic acid that encodes trehalose 6-phosphate phosphatase (OtsB), a nucleic acid that encodes OtsA and OtsB, or a nucleic acid that encodes a trehalose biosynthesis fusion polypeptide.

Also described herein are methods of preventing, ameliorating, or preventing and ameliorating the formation of amyloid fibers in a subject at risk of developing a neurodegenerative disorder, that method includes administering to the subject a therapeutically effective amount of a vector comprising a nucleic acid that encodes trehalose-6-phosphate synthase (OtsA), a nucleic acid that encodes trehalose 6-phosphate phosphatase (OtsB), a nucleic acid that encodes OtsA and OtsB, or a nucleic acid that encodes a trehalose biosynthesis fusion polypeptide.

In any of the methods described herein, the neurodegenerative disorder is associated with the formation of protein plaques. In any of the methods described herein, the neurodegenerative disorder is selected from the group consisting of Alzheimer's disease (AD), Huntington's disease (HD), amyotrophic lateral sclerosis (ALS), Parkinson's disease (PD), ataxia (e.g., Friedreich's ataxia), progressive supranuclear palsy (PSLP), spinocerebellar ataxia (SCA) (e.g., SCA3), and amyloidosis.

In any of the methods described herein, the administering is intrathecal, intraparenchymal, intracisterna (ICM), or intracerebroventricular injection (ICV).

In any of the methods described herein, the subject is a mammal. In some cases, the mammal is a human.

Also disclosed herein are methods of treating a subject having a disorder associated with excessive inflammation comprising administering to the subject a therapeutically effective amount of a vector comprising a nucleic acid that encodes trehalose-6-phosphate synthase (OtsA), a nucleic acid that encodes trehalose 6-phosphate phosphatase (OtsB), a nucleic acid that encodes OtsA and OtsB, or a nucleic acid encoding a trehalose biosynthesis fusion polypeptide.

Also disclosed herein are methods of preventing symptoms of a subject having a disorder associated with excessive inflammation comprising administering to the subject a therapeutically effective amount of a vector comprising a nucleic acid that encodes trehalose-6-phosphate synthase (OtsA), a nucleic acid that encodes trehalose 6-phosphate phosphatase (OtsB), a nucleic acid that encodes OtsA and OtsB, or a nucleic acid encoding a trehalose biosynthesis fusion polypeptide. In some cases, the administering comprises intravenous administration. In some cases, the disorder associated with excessive inflammation is atopic dermatitis. In some cases, the subject is a mammal. In some cases the mammal is a human.

Also described herein are vectors that include a nucleic acid that encodes trehalose-6-phosphate synthase (OtsA), a nucleic acid that encodes trehalose 6-phosphate phosphatase (OtsB), a nucleic acid that encodes OtsA and OtsB, or a nucleic acid that encodes a trehalose biosynthesis fusion polypeptide.

In any of the methods or compositions described herein, the vector includes a nucleic acid that encodes OtsA and OtsB. In some cases, the vector also includes a peptide linker sequence located between a nucleic acid encoding OtsA and a nucleic acid encoding OtsB. In some cases, the vector also includes an enhancer.

In any of the methods or compositions described herein, the vector is an adeno-associated viral (AAV) vector. In some cases, the AAV vector is any one of AAV1, AAV2, AAV3, AAV4, AAV5, AAV6, AAV7, AAV8, or AAV9. In some cases, the AAV vector is a recombinant adeno-associated virus (rAAV). In some cases, the AAV vector is a rAAV9 vector.

In any of the methods or compositions described herein, the vector includes a nucleic acid that encodes a trehalose biosynthesis fusion polypeptide. In some cases, the trehalose biosynthesis fusion polypeptide includes OtsA and OtsB, or functional fragments thereof. In some cases, the nucleic acid encoding the trehalose biosynthesis fusion polypeptide includes a nucleic acid encoding OtsA, or a functional fragment thereof and a nucleic acid encoding OtsB, or a functional fragment thereof.

In any of the methods or compositions described herein, the vector includes a nucleic acid encoding the trehalose biosynthesis fusion polypeptide, which also includes one or more self-cleaving peptides. In some cases, the self-cleaving peptides are selected from the group consisting of P2A and T2A. In some cases, the self-cleaving peptides are individually selected from the group consisting of P2A and T2A.

In any of the methods or compositions described herein, the nucleic acid that encodes trehalose-6-phosphate synthase (OtsA), the nucleic acid that encodes trehalose 6-phosphate phosphatase (OtsB), the nucleic acid that encodes OtsA and OtsB, or the nucleic acid encoding a trehalose biosynthesis fusion polypeptide is operably linked to a promoter.

Also disclosed herein are methods of producing any of the vector described herein from a host cell, the method including culturing the host cell containing any of the vectors described herein in a medium in which the host cell can express the vector.

### DESCRIPTION OF DRAWINGS

FIG. 1A is a representative AAV vector map that results in production of OtsA and OtsB

FIG. 1B is a representative AAV vector map that results in production of OtsB and OtsA.

FIG. 1C is a representative AAV vector map that results in production of OtsA, OtsB, and the mCherry fluorescent protein.

FIG. 1D is a representative image of fluorescence microscopy of cells transfected with the OtsA, OtsB, and mCherry producing AAV vector of FIG. 1E.

FIG. 1E is a representative AAV vector map that results in production of OtsA and OtsB.

FIG. 1F is a representative AAV vector map that results in production of OtsA and OtsB.

FIG. 1G is a representative AAV vector map that results in the production of an OtsA/OtsB fusion protein.

FIG. 2 is a representative image of a mouse spine and the cervical thoracic and lumbar regions.

FIG. 3 is a representative image of glass slides with SC sections. Each slide had 3 serial sections 1100 μm apart from 2 animals (#1, #2). Cervical region had total of 2 slides, thoracic region had a total of 4 slides, and lumbar region had a total of 2 slides. C=Cervical, T=Thoracic, L=Lumbar.

FIG. 4 is a graph of the quantification (cells per mm²) of mCherry fluorescence distribution along various ROIs of whole mouse brains after the indicated treatment as determined by immunohistochemistry. Results expressed as Mean±SEM.

FIG. 5 is a graph showing quantification of total mCherry biodistribution in the brain ROIs 4-9 for animal groups 1-6 as cell counts/mm². Results expressed as Mean±SEM. Statistically significant difference were observed in between low titer (group 1, 4) and high titer groups (3, 6). Results expressed as Mean±SEM. Groups 1-3: One-Way ANOVA with Tukey's multiple comparisons test, **p<0.01. Groups 4-6: One-Way ANOVA with Tukey's multiple comparisons test, *p<0.05.

FIG. 6 is a graph showing quantification of total mCherry biodistribution in the brain ROIs 4-9 for animal groups 7 and 9 as cell counts/mm². Results expressed as Mean±SEM. Statistically significant difference were observed between the different titer groups. Results expressed as Mean±SEM. One-Way ANOVA with Tukey's multiple comparisons test, **p<0.01, ***p<0.001.

FIG. 7 is a graph showing quantification of total mCherry biodistribution in the hemi-brain ROIs 4-9 for animal groups 7 and 9 as cell counts/mm². Results expressed as Mean±SEM.

FIG. 8 is a graph showing quantification of mCherry biodistribution in the individual spinal cord (SC) cervical, thoracic & lumbar ROIs for animal groups 1-6 as cell counts/mm². Results expressed as Mean±SEM. MN=motor neurons.

FIG. 9 is a graph showing an analysis of total mCherry biodistribution in the spinal cord (SC) for animal groups 1-6 as cell counts/mm². Results expressed as Mean±SEM. MN=motor neurons.

FIG. 10 is a graph showing quantification of mCherry biodistribution in the spinal cord (SC) cervical, thoracic & lumbar ROIs for animal groups 7 and 9 along all 24 ROIs expressed as cell counts/mm². Results expressed as Mean±SEM. MN=motor neurons.

FIG. 11 is a graph showing an analysis of total mCherry biodistribution in the spinal cord (SC) cervical, thoracic & lumbar ROIs for animal groups 7 and 9 as cell counts/mm². Results expressed as Mean±SEM. MN=motor neurons.

FIG. 12A is a graph showing quantification and comparison of total mCherry biodistribution in the group 1-6 brain and spinal cord (SC) tissues. Statistically significant differences between mCherry were observed between group 3, 5, and 6. Results expressed as Mean±SEM. Two-Way Repeated Measures ANOVA with Šídák's multiple comparisons test, *p<0.05, **p<0.01, ***p<0.001.

FIGS. 12B-12C show increased cytoplasmic TDP-43 mislocalisation (FIG. 12B), and altered mitochondria in ALS iPSC motor neurons (FIG. 12B; Scale bar=100 μm), and motor neuron death (FIG. 12C).

FIG. 12D is a representative image of cells treated with and without trehalose at 20 mM. Cells were stained for ChAT, β3-tubulin, and DAPI.

FIG. 12E is a representative schematic of an experimental protocol.

FIG. 13 is a representative schematic of the Phase I main study arm.

FIG. 14 is a representative schematic of the Phase I survival study arm.

FIG. 15 is a representative schematic of the Phase II study arm.

FIG. 16 is a schematic of Hamilton needle tip styles.

FIG. 17 is a graph of mouse body weight over time in the Phase I Main study arm. Data are presented as mean±SEM. Group 1, n=11; Group 2, n=13; Group 3, n=12→9; Group 4, n=12→6. Statistical significances: *p<0.05, Group 2 vs. Group 1 (Mixed-effects model (REML), Uncorrected Fisher's LSD).

FIGS. 18A-18B are graphs of mouse body weight over time in the Phase I survival study arm. Data are presented as mean±SEM. Group 1, n=12; Group 2, n=11→0; Group 3, n=12→0; Group 4, n=12→0. Statistical significances: *p<0.05, Group 2 vs. Group 1 (Mixed-effects model (REML), Uncorrected Fisher's LSD).

FIGS. 19A-19B are graphs of mouse body weight over time in Phase II, all animals included. A portion of animals were euthanized at the scheduled end point. Data are presented as mean±SEM. Group 5, n=4→0; Group 6, n=5→0; Group 7, n=18→0; Group 9, n=1. No statistically significant differences between the groups were observed (p>0.05, Mixed-effects model (REML)).

FIGS. 20A-20B are graph of mouse body weight over time in Phase I and Phase II, only survival study arm animals included from both phases. A portion of animals were euthanized at the scheduled end point. Data are presented as mean±SEM. Group 1, n=12; Group 2, n=12→0; Group 3, n=12→0; Group 4, n=13→0; Group 5, n=7→0; Group 6, n=9→0; Group 7, n=9→0. Statistical tests were performed between Phase II groups only. No statistically significant differences between groups were observed (p>0.05, Mixed-effects model (REML)).

FIG. 21 is a graph of a Kaplan-Meier survival analysis of Phase I survival study arm. Group 1, n=12; Group 2, n=11; Group 3, n=12; Group 4, n=12. Statistical significances: p<0.0001 Group 1 vs. Group 2 (Log-rank (Mantel-Cox) test).

FIG. 22 is a graph of a Kaplan-Meier survival analysis of Phase II survival study arm. Group 5, n=7; Group 6, n=9; Group 7, n=9. Statistical significances: Group 5 vs 6: p=0.0070; Group 5 vs. 7: p<0.0001; Group 6 vs 7: p<0.0001. Log-rank (Mantel-Cox) test.

FIG. 23 is a graph of a Kaplan-Meier survival analysis of Phase II survival study arm. Group 1, n=12; Group 2, n=11; Group 3, n=12; Group 4, n=12; Group 5, n=7; Group 6, n=9; Group 7, n=9. No statistical comparisons between the groups were performed.

FIG. 24 is a graph of the percentage of positive Iba-1 staining by IHC in a region area in the striatum in Phase I and Phase II groups. Data are presented as mean±SEM. Group 1, n=11, Group 2, n=11, Group 3, n=12, Group 4, n=5, Group 5, n=2, Group 6, n=1, Group 7, n=7, Group 9, n=1.

FIG. 25 is a graph of the percentage of positive GFAP staining by IHC in the striatum in Phase I and Phase II groups. Data are presented as mean±SEM. Group 1, n=11, Group 2, n=11, Group 3, n=12, Group 4, n=5, Group 5, n=2, Group 6, n=1, Group 7, n=7, Group 9, n=1.

FIG. 26 is a graph of the percentage of cells per mm² that showed positive EM48 immunostaining in the striatum in Phase I and Phase II groups. Data are presented as mean±SEM. Group 1, n=11, Group 2, n=13, Group 3, n=8, Group 4, n=6, Group 5, n=2, Group 6, n=1, Group 7, n=7, Group 9, n=1.

FIG. 27 is a graph of the percentage of cells per mm² that showed positive EM48 immunostaining in the cortex in Phase I and II groups. Data are presented as mean±SEM. Group 1, n=11, Group 2, n=13, Group 3, n=8, Group 4, n=6, Group 5, n=2, Group 6, n=1, Group 7, n=7, Group 9, n=1.

FIG. 28 is a graph of the percentage of cells per mm² that showed positive DARPP-32 immunostaining in the striatum in Phase 1 and 2. Data are presented as mean±SEM. Group 1, n=12, Group 2, n=11, Group 3, n=8, Group 4, n=6, Group 5, n=2, Group 6, n=1, Group 7, n=7, Group 9, n=1a.

FIG. 29 is a graph of the percentage of cells per mm² that showed positive AT8 (a neuronal marker NeuN with p-tau marker; i.e., a marker for phosphorylated tau protein) immunostaining in the entorhinal cortex (ERC). Data are presented as mean±SEM.

### DETAILED DESCRIPTION

### Background

Previously, neurodegenerative disorders have been proposed to be treated with anti-sense oligonucleotides (ASOs). However, ASOs are limited in the size of DNA included, are limited in biodistribution of the subject, and require multiple and often regular (e.g., monthly) administration. The presently discloses nucleic acids and/or vectors, such as AAV vectors containing nucleic acids encoding OtsA and/or OtsB for trehalose production, or a fusion protein thereof, contain several advantages. First, the treatment is specific to mutant protein found in neurodegenerative disorders, such a mutant proteins forming protein plaques. Furthermore, trehalose is not endogenously made by vertebrates, and the disclosed constructs provide continuous in vivo production of trehalose, meaning that a single, or very few administrations are required.

The present disclosure provides methods for treating neurodegenerative disorders (e.g., AD, ALS, HD, PD, or SCA3) in a subject by delivering a composition including a nucleic acid(s) and/or vector(s), the nucleic acid(s), and/or vector(s) encoding trehalose-6-phosphate synthase (OtsA) and/or trehalose 6-phosphate phosphatase (OtsB), or a fusion protein thereof to the subject in need thereof.

Before the present composition, methods, and treatment methodology are described, it is to be understood that this disclosure is not limited to particular compositions, methods, and experimental conditions described, as such compositions, methods, and conditions may vary. It is also to be understood that the terminology used herein is for purposes of describing particular embodiments only, and is not intended to be limiting, since the scope of the present disclosure will be limited only in the appended claims.

Trehalose and its Biosynthesis with OtsA and OtsB

Trehalose is a naturally occurring sugar consisting of two molecules of glucose. It is also known as mycose or tremalose. Trehalose is a disaccharide formed by a 1,1-glycosidic bond between two α-glucose units. It is found in nature as a disaccharide and also as a monomer in some polymers. Two other isomers exist, α,β-trehalose, otherwise known as neotrehalose, and β,β-trehalose (also referred to as isotrehalose). Trehalose is an approved ingredient in all major markets designated as GRAS food ingredient by the FDA

Five biosynthesis pathways have been reported for trehalose metabolism, but none in vertebrates, including humans. The most common pathway is TPS/TPP pathway. The TSP/TPP pathway uses three enzymes: trehalose-6-phosphate synthase (TPS, or OtsA), which converts uridine diphosphate (UDP)-glucose and glucose-6-phosphate to trehalose-6-phosphate (T6P), trehalose-6-phosphatase (TPP, or OtsB), which dephosphorylates T6P to produce trehalose, and trehalase (TRE), which is responsible for trehalose catabolismecond, trehalose synthase (TS) in certain types of bacteria could produce trehalose by using maltose and another disaccharide with two glucose units as substrates. Third, the TreY-TreZ pathway in some bacteria converts starch that contain maltooligosaccharide or glycogen directly into trehalose. Fourth, in primitive bacteria, trehalose glycisyltransferring synthase (TreT) produces trehalose from ADP-glucose and glucose. Fifth, trehalose phosphorylase (TreP) either hydrolyses trehalose into glucose-1-phosphate and glucose or may act reversibly in certain species.

Vertebrates do not have the ability to synthesize or store trehalose. Trehalase, an enzyme to break down trehalose, in humans is found only in specific location such as the intestinal mucosa, renal brush-border, liver and blood.

As used herein “substantially purified trehalose” refers to trehalose that contains less than 1% contaminants.

As used herein the “active ingredient” in a formulation means that the referenced compound is the ingredient in a formulation that produces the intended therapeutic effect. For example, in a formulation comprising trehalose or vector including nucleic acids encoding OtsA and/or OtsB, or a fusion protein thereof, as the active ingredient and a trehalase inhibitor as described herein, trehalose or the vectors is the active ingredient—the trehalase inhibitor does not produce a therapeutic effect, but rather may enhance the effect of the trehalose or the vector. Moreover, when a formulation comprises trehalose or a vector including nucleic acids encoding OtsA and/or OtsB, or a fusion protein thereof as the active ingredient, this phrase limits the additional components to non-active ingredients.

### Trehalose Biosynthesis Polynucleotides and Polypeptides

The trehalose-6-phosphate synthase gene (tps or otsA) encodes a trehalose-6-phosphate synthase, which converts uridine diphosphate (UDP)-glucose and glucose-6-phosphate to trehalose-6-phosphate (T6P). The trehalose-6-phosphatase gene (tpp or otsB) encodes a trehalose-6-phosphatase, which dephosphorylates T6P to produce trehalose.

### Trehalose Biosynthesis Polypeptides

Polynucleotides (e.g., viral vectors) described herein encode for a polypeptide involved in trehalose biosynthesis, for example, OtsA or OtsB, isomers thereof, or fusion proteins thereof, such as a trehalose biosynthesis fusion polypeptide. “Polypeptide” is used in its conventional meaning, i.e., as a sequence of amino acids. The polypeptides are not limited to a specific length of the product; thus, peptides, oligopeptides, and proteins are included within the definition of polypeptide, and such terms may be used interchangeably herein unless specifically indicated otherwise. This term also does not refer to or exclude post-translational modifications of the polypeptide, for example, glycosylations, acetylations, phosphorylations and the like, as well as other modifications known in the art, both naturally occurring and non-naturally occurring. A polypeptide may be an entire protein, or a subsequence thereof.

As disclosed herein, OtsA polypeptides include, but are not limited to, the otsA gene encoding the OtsA polypeptide of GenBank sequences such as NC_000913.3, which is publicly available and incorporated herein by reference. An exemplary OtsA polypeptide is shown in Table 1a.

As disclosed herein, OtsB polypeptides include, but are not limited to, the otsB gene encoding the OtsB polypeptide of GenBank sequences such as NC_000913.3, which publicly available and incorporated herein by reference. An exemplary OtsB polypeptide is shown in Table 1a.

As disclosed herein, a trehalose biosynthesis fusion polypeptide is a functional combination of an OtsA polypeptide and an OtsB polypeptide. For example, a trehalose biosynthesis fusion polypeptide can include a functional OtsA polypeptide fragment and a functional OtsB polypeptide fragment fused together that can synthesize trehalose from UDP-glucose and glucose-6-phosphate when expressed in a cell. An exemplary trehalose biosynthesis fusion polypeptide is shown in in Table 1a.

In some cases, additional elements can be included. For example, self-cleaving peptides can separate the OtsA polypeptide, OtsA polypeptide variant, or OtsA polypeptide fragment from the OtsB polypeptide, OtsA polypeptide variant, or OtsB polypeptide variant. Examples of self-cleaving peptides include, but are not limited to, 2A self-cleaving peptides such as a P2A self-cleaving peptide.

TABLE 1a
Exemplary Polynucleotides and Polypeptides.

| SEQ | | | |
|---|---|---|---|
| ID NO | Description | Species | Sequence (5′ -> 3′)ª |
| 1 | Polynucleotide | Escherichia | ATGAGTCGTTTAGTCGTAGTATCTAACCGGATTGCACCACCAGACGAGCAC |
| | encoding OtsA | coli K-12 | GCCGCCAGTGCCGGTGGCCTTGCCGTTGGCATACTGGGGGCACTGAAAGC |
| | polypeptide | subspec. | CGCAGGCGGACTGTGGTTTGGCTGGAGTGGTGAAACAGGGAATGAGGAT |
| | | MG1655 | CAGCCGCTAAAAAAGGTGAAAAAAGGTAACATTACGTGGGCCTCTTTTAA |
| | | | CCTCAGCGAACAGGACCTTGACGAATACTACAACCAATTCTCCAATGCCGT |
| | | | TCTCTGGCCCGCTTTTCATTATCGGCTCGATCTGGTGCAATTTCAGCGTCCT |
| | | | GCCTGGGACGGCTATCTACGCGTAAATGCGTTGCTGGCAGATAAATTACT |
| | | | GCCGCTGTTGCAAGACGATGACATTATCTGGATCCACGATTATCACCTGTT |
| | | | GCCATTTGCGCATGAATTACGCAAACGGGGAGTGAATAATCGCATTGGTTT |
| | | | CTTTCTGCATATTCCTTTCCCGACACCGGAAATCTTCAACGCGCTGCCGACA |
| | | | TATGACACCTTGCTTGAACAGCTTTGTGATTATGATTTGCTGGGTTTCCAGA |
| | | | CAGAAAACGATCGTCTGGCGTTCCTGGATTGTCTTTCTAACCTGACCCGCG |
| | | | TCACGACACGTAGCGCAAAAAGCCATACAGCCTGGGGCAAAGCATTTCGA |
| | | | ACAGAAGTCTACCCGATCGGCATTGAACCGAAAGAAATAGCCAAACAGGC |
| | | | TGCCGGGCCACTGCCGCCAAAACTGGCGCAACTTAAAGCGGAACTGAAAA |
| | | | ACGTACAAAATATCTTTTCTGTCGAACGGCTGGATTATTCCAAAGGTTTGCC |
| | | | AGAGCGTTTTCTCGCCTATGAAGCGTTGCTGGAAAAATATCCGCAGCATCA |
| | | | TGGTAAAATTCGTTATACCCAGATTGCACCAACGTCGCGTGGTGATGTGCA |
| | | | AGCCTATCAGGATATTCGTCATCAGCTCGAAAATGAAGCTGGACGAATTAA |
| | | | TGGTAAATACGGGCAATTAGGCTGGACGCCGCTTTATTATTTGAATCAGCA |
| | | | TTTTGACCGTAAATTACTGATGAAAATATTCCGCTACTCTGACGTGGGCTTA |
| | | | GTGACGCCACTGCGTGACGGGATGAACCTGGTAGCAAAAGAGTATGTTGC |
| | | | TGCTCAGGACCCAGCCAATCCGGGCGTTCTTGTTCTTTCGCAATTTGCGGG |
| | | | AGCGGCAAACGAGTTAACGTCGGCGTTAATTGTTAACCCCTACGATCGTGA |
| | | | CGAAGTTGCAGCTGCGCTGGATCGTGCATTGACTATGTCGCTGGCGGAAC |
| | | | GTATTTCCCGTCATGCAGAAATGCTGGACGTTATCGTGAAAAACGATATTA |
| | | | ACCACTGGCAGGAGTGCTTCATTAGCGACCTAAAGCAGATAGTTCCGCGA |
| | | | AGCGCGGAAAGCCAGCAGCGCGATAAAGTTGCTACCTTTCCAAAGCTTGC |
| | | | GTAG |
| 2 | OtsA | Escherichia | MSRLVVVSNRIAPPDEHAASAGGLAVGILGALKAAGGLWFGWSGETGNEDQ |
| | polypeptide | coli K-12 | PLKKVKKGNITWASFNLSEQDLDEYYNQFSNAVLWPAFHYRLDLVQFQRPA |
| | | subspec. | WDGYLRVNALLADKLLPLLQDDDIIWIHDYHLLPFAHELRKRGVNNRIGFFLHI |
| | | MG1655 | PFPTPEIFNALPTYDTLLEQLCDYDLLGFQTENDRLAFLDCLSNLTRVTTRSAKS |
| | | | HTAWGKAFRTEVYPIGIEPKEIAKQAAGPLPPKLAQLKAELKNVQNIFSVERLD |
| | | | YSKGLPERFLAYEALLEKYPQHHGKIRYTQIAPTSRGDVQAYQDIRHQLENEAG |
| | | | RINGKYGQLGWTPLYYLNQHFDRKLLMKIFRYSDVGLVTPLRDGMNLVAKEY |
| | | | VAAQDPANPGVLVLSQFAGAANELTSALIVNPYDRDEVAAALDRALTMSLAE |
| | | | RISRHAEMLDVIVKNDINHWQECFISDLKQIVPRSAESQQRDKVATFPKLA |
| 3 | Polynucleotide | Escherichia | GTGACAGAACCGTTAACCGAAACCCCTGAACTATCCGCGAAATATGCCTG |
| | encoding OtsB | coli K-12 | GTTTTTTGATCTTGATGGAACGCTGGCGGAAATCAAACCGCATCCCGATCA |
| | polypeptide | subspec. | GGTCGTCGTGCCTGACAATATTCTGCAAGGACTACAGCTACTGGCAACCGC |
| | | MG1655 | AAGTGATGGTGCATTGGCATTGATATCAGGGCGCTCAATGGTGGAGCTTG |
| | | | ACGCACTGGCAAAACCTTATCGCTTCCCGTTAGCGGGCGTGCATGGGGCG |
| | | | GAGCGCCGTGACATCAATGGTAAAACACATATCGTTCATCTGCCGGATGCG |
| | | | ATTGCGCGTGATATTAGCGTGCAACTGCATACAGTCATCGCTCAGTATCCC |
| | | | GGCGCGGAGCTGGAGGCGAAAGGGATGGCTTTTGCGCTGCATTATCGTCA |
| | | | GGCTCCGCAGCATGAAGACGCATTAATGACATTAGCGCAACGTATTACTCA |
| | | | GATCTGGCCACAAATGGCGTTACAGCAGGGAAAGTGTGTTGTCGAGATCA |
| | | | AACCGAGAGGTACCAGTAAAGGTGAGGCAATTGCAGCTTTTATGCAGGAA |
| | | | GCTCCCTTTATCGGGCGAACGCCCGTATTTCTGGGCGATGATTTAACCGAT |
| | | | GAATCTGGCTTCGCAGTCGTTAACCGACTGGGCGGAATGTCAGTAAAAAT |
| | | | TGGCACAGGTGCAACTCAGGCATCATGGCGACTGGCGGGTGTGCCGGAT |
| | | | GTCTGGAGCTGGCTTGAAATGATAACCACCGCATTACAACAAAAAAGAGA |
| | | | AAATAACAGGAGTGATGACTATGAGTCGTTTAGTCGTAGTATC |
| 4 | OtsB | Escherichia | MTEPLTETPELSAKYAWFFDLDGTLAEIKPHPDQVVVPDNILQGLQLLATASD |
| | polypeptide | coli K-12 | GALALISGRSMVELDALAKPYRFPLAGVHGAERRDINGKTHIVHLPDAIARDIS |
| | | subspec. | VQLHTVIAQYPGAELEAKGMAFALHYRQAPQHEDALMTLAQRITQIWPQM |
| | | MG1655 | ALQQGKCVVEIKPRGTSKGEAIAAFMQEAPFIGRTPVFLGDDLTDESGFAVVN |
| | | | RLGGMSVKIGTGATQASWRLAGVPDVWSWLEMITTALQQKRENNRSDDYE |
| | | | SFSRSI |
| 5 | Polynucleotide | OtsA/OtsB | ATGAGTCGTTTAGTCGTAGTATCTAACCGGATTGCACCACCAGACGAGCAC |
| | encoding a | Fusion | GCCGCCAGTGCCGGTGGCCTTGCCGTTGGCATACTGGGGGCACTGAAAGC |
| | trehalose | Protein | CGCAGGCGGACTGTGGTTTGGCTGGAGTGGTGAAACAGGGAATGAGGAT |
| | biosynthesis | | CAGCCGCTAAAAAAGGTGAAAAAAGGTAACATTACGTGGGCCTCTTTTAA |
| | fusion | | CCTCAGCGAACAGGACCTTGACGAATACTACAACCAATTCTCCAATGCCGT |
| | polypeptide | | TCTCTGGCCCGCTTTTCATTATCGGCTCGATCTGGTGCAATTTCAGCGTCCT |
| | | | GCCTGGGACGGCTATCTACGCGTAAATGCGTTGCTGGCAGATAAATTACT |
| | | | GCCGCTGTTGCAAGACGATGACATTATCTGGATCCACGATTATCACCTGTT |
| | | | GCCATTTGCGCATGAATTACGCAAACGGGGAGTGAATAATCGCATTGGTTT |
| | | | CTTTCTGCATATTCCTTTCCCGACACCGGAAATCTTCAACGCGCTGCCGACA |
| | | | TATGACACCTTGCTTGAACAGCTTTGTGATTATGATTTGCTGGGTTTCCAGA |
| | | | CAGAAAACGATCGTCTGGCGTTCCTGGATTGTCTTTCTAACCTGACCCGCG |
| | | | TCACGACACGTAGCGCAAAAAGCCATACAGCCTGGGGCAAAGCATTTCGA |
| | | | ACAGAAGTCTACCCGATCGGCATTGAACCGAAAGAAATAGCCAAACAGGC |
| | | | TGCCGGGCCACTGCCGCCAAAACTGGCGCAACTTAAAGCGGAACTGAAAA |
| | | | ACGTACAAAATATCTTTTCTGTCGAACGGCTGGATTATTCCAAAGGTTTGCC |
| | | | AGAGCGTTTTCTCGCCTATGAAGCGTTGCTGGAAAAATATCCGCAGCATCA |
| | | | TGGTAAAATTCGTTATACCCAGATTGCACCAACGTCGCGTGGTGATGTGCA |
| | | | AGCCTATCAGGATATTCGTCATCAGCTCGAAAATGAAGCTGGACGAATTAA |
| | | | TGGTAAATACGGGCAATTAGGCTGGACGCCGCTTTATTATTTGAATCAGCA |
| | | | TTTTGACCGTAAATTACTGATGAAAATATTCCGCTACTCTGACGTGGGCTTA |
| | | | GTGACGCCACTGCGTGACGGGATGAACCTGGTAGCAAAAGAGTATGTTGC |
| | | | TGCTCAGGACCCAGCCAATCCGGGCGTTCTTGTTCTTTCGCAATTTGCGGG |
| | | | AGCGGCAAACGAGTTAACGTCGGCGTTAATTGTTAACCCCTACGATCGTGA |
| | | | CGAAGTTGCAGCTGCGCTGGATCGTGCATTGACTATGTCGCTGGCGGAAC |
| | | | GTATTTCCCGTCATGCAGAAATGCTGGACGTTATCGTGAAAAACGATATTA |
| | | | ACCACTGGCAGGAGTGCTTCATTAGCGACCTAAAGCAGATAGTTCCGCGA |
| | | | AGCGCGGAAAGCCAGCAGCGCGATAAAGTTGCTACCTTTCCAAAGCTTGC |
| | | | GGCTACTAACTTCAGCCTGCTGAAGCAGGCTGGCGACGTGGAGGAGAACC |
| | | | CTGGACCTGTGACAGAACCGTTAACCGAAACCCCTGAACTATCCGCGAAAT |
| | | | ATGCCTGGTTTTTTGATCTTGATGGAACGCTGGCGGAAATCAAACCGCATC |
| | | | CCGATCAGGTCGTCGTGCCTGACAATATTCTGCAAGGACTACAGCTACTGG |
| | | | CAACCGCAAGTGATGGTGCATTGGCATTGATATCAGGGCGCTCAATGGTG |
| | | | GAGCTTGACGCACTGGCAAAACCTTATCGCTTCCCGTTAGCGGGCGTGCAT |
| | | | GGGGCGGAGCGCCGTGACATCAATGGTAAAACACATATCGTTCATCTGCC |
| | | | GGATGCGATTGCGCGTGATATTAGCGTGCAACTGCATACAGTCATCGCTCA |
| | | | GTATCCCGGCGCGGAGCTGGAGGCGAAAGGGATGGCTTTTGCGCTGCATT |
| | | | ATCGTCAGGCTCCGCAGCATGAAGACGCATTAATGACATTAGCGCAACGT |
| | | | ATTACTCAGATCTGGCCACAAATGGCGTTACAGCAGGGAAAGTGTGTTGT |
| | | | CGAGATCAAACCGAGAGGTACCAGTAAAGGTGAGGCAATTGCAGCTTTTA |
| | | | TGCAGGAAGCTCCCTTTATCGGGCGAACGCCCGTATTTCTGGGCGATGATT |
| | | | TAACCGATGAATCTGGCTTCGCAGTCGTTAACCGACTGGGCGGAATGTCA |
| | | | GTAAAAATTGGCACAGGTGCAACTCAGGCATCATGGCGACTGGCGGGTGT |
| | | | GCCGGATGTCTGGAGCTGGCTTGAAATGATAACCACCGCATTACAACAAA |
| | | | AAAGAGAAAATAACAGGAGTGATGACTATGAGTCGTTTAGTCGTAGTATC |
| | | | GAGGGCAGAGGAAGTCTTCTAACATGCGGTGACGTGGAGGAGAATCCCG |
| | | | GCCCT |
| 6 | Trehalose | OtsA/OtsB | MSRLVVVSNRIAPPDEHAASAGGLAVGILGALKAAGGLWFGWSGETGNEDQ |
| | biosynthesis | Fusion | PLKKVKKGNITWASFNLSEQDLDEYYNQFSNAVLWPAFHYRLDLVQFQRPA |
| | fusion | Protein | WDGYLRVNALLADKLLPLLQDDDIIWIHDYHLLPFAHELRKRGVNNRIGFFLHI |
| | polypeptide | | PFPTPEIFNALPTYDTLLEQLCDYDLLGFQTENDRLAFLDCLSNLTRVTTRSAKS |
| | | | HTAWGKAFRTEVYPIGIEPKEIAKQAAGPLPPKLAQLKAELKNVQNIFSVERLD |
| | | | YSKGLPERFLAYEALLEKYPQHHGKIRYTQIAPTSRGDVQAYQDIRHQLENEAG |
| | | | RINGKYGQLGWTPLYYLNQHFDRKLLMKIFRYSDVGLVTPLRDGMNLVAKEY |
| | | | VAAQDPANPGVLVLSQFAGAANELTSALIVNPYDRDEVAAALDRALTMSLAE |
| | | | RISRHAEMLDVIVKNDINHWQECFISDLKQIVPRSAESQQRDKVATFPKLAAT |
| | | | NFSLLKQAGDVEENPGPMTEPLTETPELSAKYAWFFDLDGTLAEIKPHPDQVV |
| | | | VPDNILQGLQLLATASDGALALISGRSMVELDALAKPYRFPLAGVHGAERRDI |
| | | | NGKTHIVHLPDAIARDISVQLHTVIAQYPGAELEAKGMAFALHYRQAPQHED |
| | | | ALMTLAQRITQIWPQMALQQGKCVVEIKPRGTSKGEAIAAFMQEAPFIGRTP |
| | | | VFLGDDLTDESGFAVVNRLGGMSVKIGTGATQASWRLAGVPDVWSWLEMI |
| | | | TTALQQKRENNRSDDYESFSRSIEGRGSLLTCGDVEENPGP |
| 7 | Polynucleotide | Porcine | GCTACTAACTTCAGCCTGCTGAAGCAGGCTGGCGACGTGGAGGAGAACCC |
| | encoding P2A | Teschovirus- | TGGACCT |
| | self-cleaving | 1 | |
| | peptide | | |
| 8 | P2A self- | Porcine | ATNFSLLKQAGDVEENPGP |
| | cleaving | Teschovirus- | |
| | peptide | 1 | |
| 9 | Polynucleotide | Thosea | GAGGGCAGAGGAAGTCTTCTAACATGCGGTGACGTGGAGGAGAATCCCG |
| | encoding T2A | asigna virus | GCCCT |
| | self-cleaving | | |
| | peptide | | |
| 10 | T2A self- | Thosea | EGRGSLLTCGDVEENPGP |
| | cleaving | asigna virus | |
| | peptide | | |

^{a}Bold underlined sequences indicate start codons or alternate start codons. The underlined italizized M in SEQ ID NO: 6 can also be a V.

Disclosed herein are OtsA polypeptides having, for example, the sequence recited in SEQ ID NO: 2. In some cases, the OtsA polypeptide has the sequence recited in SEQ ID NO: 2. In some cases, a variant of the OtsA polypeptide described herein can be used. In some cases, an OtsA polypeptide variant can have at least about 80%, 85%, 90%, 91%, 92%, 93%, 94%, 95%, 96%, 97%, 98%, or 99% or greater sequence identity, along its length, to the polypeptide sequence set forth as SEQ ID NO: 2. In some cases, OtsA polypeptide variants can have 1-10 (e.g., 1, 2, 3, 4, 5, 6, 7, 8, 9, or 10) amino acid substitutions, additions, or deletions compared to any of SEQ ID NO: 2.

Also disclosed herein are OtsB polypeptides having, for example, the sequence recited in SEQ ID NO: 4. In some cases, the OtsB polypeptide has the sequence recited in SEQ ID NO: 4. In some cases, a variant of the OtsB polypeptide described herein can be used. In some cases, an OtsB polypeptide variant can have at least about 80%, 85%, 90%, 91%, 92%, 93%, 94%, 95%, 96%, 97%, 98%, or 99% or greater sequence identity, along its length, to the polypeptide sequences set forth as SEQ ID NO: 4. In some cases, OtsB polypeptide variants can have 1-10 (e.g., 1, 2, 3, 4, 5, 6, 7, 8, 9, or 10) amino acid substitutions, additions, or deletions compared to any of SEQ ID NO: 4.

In some cases, a trehalose biosynthesis polypeptide is a fusion polypeptide, referred to as a trehalose biosynthesis fusion polypeptide, that includes 1) an OtsA polypeptide, functional fragment, or variant thereof, and 2) an OtsB polypeptide, functional fragment thereof, or variant thereof. For example, an OtsA polypeptide can be fused to an OtsB polypeptide to produce a trehalose biosynthesis fusion polypeptide, and can produce trehalose when provided UDP-glucose and glucose-6-phosphate. In some cases, a trehalose biosynthesis fusion polypeptide includes at least one of 1) the OtsA polypeptide, functional fragment thereof, or variant thereof, of SEQ ID NO: 2, as described herein, and 2) the OtsB polypeptide, functional fragment thereof, or variant thereof, of SEQ ID NOs: 4, as described herein. In some cases, the vector encoding the trehalose biosynthesis fusion polypeptide can include an unrelated sequence, such as a known viral protein. A fusion partner can, for example, assist in expressing the protein (an expression enhancer) at higher yields than the native recombinant protein.

Fusion polypeptides may generally be prepared using standard techniques, including chemical conjugation. In some embodiments, a trehalose biosynthesis fusion polypeptide is expressed as a recombinant polypeptide, allowing the production of trehalose when inserted in a subject. Briefly, DNA sequences encoding the polypeptide components may be assembled separately, and ligated into an appropriate expression vector. The 3′ end of the DNA sequence encoding one polypeptide component is ligated, with or without a peptide linker, to the 5′ end of a DNA sequence encoding the second polypeptide component so that the reading frames of the sequences are in phase. This permits translation into a single fusion polypeptide that retains the biological activity of both component polypeptides.

The present disclosure also encompasses “peptide” or “peptide portion” or “fragment” or “peptide fragment” and equivalents thereof, which is used broadly herein to mean two or more amino acids linked by a peptide bond. Further, the term “functional fragment” or “functional portion” or equivalents thereof means that OtsA polypeptide fragment or OtsB polypeptide fragment has functional OtsA or OtsB activity, for example, a functional fragment of OtsA or OtsB has functional OtsA activity or OtsB activity.

It should be recognized that the term “peptide” is not used herein to suggest a particular size or number of amino acids comprising the molecule, and that a peptide of the disclosure can contain several amino acid residues or more.

Also, as used herein, the “translation product” or “polypeptide” refers to peptides, polypeptides, oligopeptides and proteins or protein fragments which have a desired biological effect in vivo or in vitro. In contrast, an “analog” of a molecule refers to a non-natural molecule substantially similar to either the entire molecule or a fragment thereof. All these are embodied in the disclosure so long as in vivo delivery of the therapeutic composition containing nucleic acids encoding a OtsA polypeptide and/or OtsB polypeptide, fragment, or variant thereof, or a trehalose biosynthesis fusion polypeptide to a subject suffering from any of the neurodegenerative diseases described herein ameliorates or effectively treats the subject in need thereof.

A peptide linker sequence may be employed to separate the first and second polypeptide components (e.g., to separate the OtsA polypeptide and the OtsB polypeptide) by a distance sufficient to ensure that each polypeptide folds into its secondary and tertiary structures. Such a peptide linker sequence can be incorporated into the fusion polypeptide using standard techniques well known in the art. Suitable peptide linker sequences may be chosen based on the following factors: (1) their ability to adopt a flexible extended conformation; (2) their inability to adopt a secondary structure that could interact with functional epitopes on the first and second polypeptides; and (3) the lack of hydrophobic or charged residues that might react with the polypeptide functional epitopes. In some embodiments, the peptide linker sequences contain Gly, Asn, and Ser residues. Other near neutral amino acids, such as Thr and Ala may also be used in the linker sequence. Amino acid sequences which may be usefully employed as linkers are generally known in the art. The linker sequence may generally be from 1 to about 50 amino acids in length. In some instances, the linker sequence is from 1 to 30 (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30) amino acids in length. Linker sequences are not required when the first and second polypeptides have non-essential N-terminal amino acid regions that can be used to separate the functional domains and prevent steric interference.

The skilled artisan can perform an optimal alignment of OtsA polypeptide or OtsB polypeptides, or nucleic acid sequences for comparison using, for example, the Megalign program in the Lasergene suite of bioinformatics software (DNASTAR, Inc., Madison, Wis.), using default parameters. This program embodies several alignment schemes described in the following references: Dayhoff, M. O. (1978) A model of evolutionary change in proteins-Matrices for detecting distant relationships. In Dayhoff, M. O. (ed.) Atlas of Protein Sequence and Structure, National Biomedical Research Foundation, Washington D.C. Vol. 5, Suppl. 3, pp. 345-358; Hein J. (1990) Unified Approach to Alignment and Phylogenes pp. 626-645 Methods in Enzymology vol. 183, Academic Press, Inc., San Diego, Calif.; Higgins, D. G. and Sharp, P. M. (1989) CABIOS 5:151-153; Myers, E. W. and Muller W. (1988) CABIOS 4:11-17; Robinson, E. D. (1971) Comb. Theor 11:105; Saitou, N. Nei, M. (1987) Mol. Biol. Evol. 4:406-425; Sneath, P. H. A. and Sokal, R. R. (1973) Numerical Taxonomy—the Principles and Practice of Numerical Taxonomy, Freeman Press, San Francisco, Calif.; Wilbur, W. J. and Lipman, D. J. (1983) Proc. Natl. Acad., Sci. USA 80:726-730, each of which is incorporated herein by reference.

Alternatively, optimal alignment of sequences for comparison may be conducted, for example, by the local identity algorithm of Smith and Waterman (1981) Add. APL. Math 2:482, by the identity alignment algorithm of Needleman and Wunsch (1970) J. Mol. Biol. 48:443, by the search for similarity methods of Pearson and Lipman (1988) Proc. Natl. Acad. Sci. USA 85:2444, by computerized implementations of these algorithms (GAP, BESTFIT, BLAST, FASTA, and TFASTA in the Wisconsin Genetics Software Package, Genetics Computer Group (GCG), 575 Science Dr., Madison, Wis.), or by inspection.

Non-limiting examples of algorithms that are suitable for determining percent sequence identity and sequence similarity include the BLAST and BLAST 2.0 algorithms, which are described in Altschul et al. (1977) Nucl. Acids Res. 25:3389-3402 and Altschul et al. (1990) J. Mol. Biol. 215:403-410, respectively. BLAST and BLAST 2.0 can be used, for example, with the parameters described herein, to determine percent sequence identity for the polynucleotides and polypeptides of the disclosure. Software for performing BLAST analyses is publicly available through the National Center for Biotechnology Information. For amino acid sequences, a scoring matrix can be used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment.

### Trehalose Biosynthesis Polynucleotides

Also disclosed herein are nucleic acids that encode for OtsA and/or OtsB, or a trehalose biosynthesis fusion polypeptide. Accordingly, the present disclosure provides polynucleotides encoding a polypeptide called trehalose-6-phosphate synthase (TSP or OtsA) and/or trehalose-6-phosphatase gene (TPP or OtsB).

OtsA and OtsB are found in bacteria, fungi, and plants, and synthesize trehalose from UDP-glucose and glucose-6-phosphate. Trehalose can serve as a carbon source, as a storage carbohydrate, and as a stress protectant. For example, trehalose can stabilize cells during osmotic stress, and help increase cell viability during exposure to environmental stresses, including heat shock, dehydration, and hypoxia. See, for example, Arguelles. Arch. Microbiol. 2000; 174:217-224; Alvarez-Peral et al. Microbiology (Read.). 2002; 148:2599-2606; or Chen et al. J. Exp. Biol. 2004; 207:3125-3129. Furthermore, trehalose has been shown to be effective in preventing protein misfolding in rodent models of disease (Khalifeh et al. Bioessays. 2020 August, 42(8):e1900195). It can prevent misfolding by activating autophagy pathways. It is currently in clinical trials for indications other than neurodegenerative diseases.

In some embodiments, disclosed herein are nucleic acids that encode for any one of SEQ ID NOs: 2, 4, 6, 8, or 10. In some embodiments, the present disclosure provides variants of nucleotides that encode for any one of SEQ ID NOs: 2, 4, 6, 8, or 10. In some embodiments, disclosed herein are nucleic acid variants have at least about 70%, 75%, 80%, 85%, 90%, 91%, 92%, 93%, 94%, 95%, 96%, 97%, 98%, or 99% or greater sequence identity, along its length, to a nucleotide sequence that encodes for any one of SEQ ID NOs: 2, 4, 6, 8, or 10. In some cases, the nucleic acids have any one of the SEQ IDs: 1, 3, 5, 7, or 9. In some embodiments, disclosed herein are nucleic acid variants have at least about 70%, 75%, 80%, 85%, 90%, 91%, 92%, 93%, 94%, 95%, 96%, 97%, 98%, or 99% or greater sequence identity, along its length, to a nucleic acid having the sequence of SEQ ID NOs: 1, 3, 5, 7, or 9.

In some embodiments, any one of the nucleic acids described herein (e.g., any nucleotide encoding for any one of SEQ ID NOs: 2, 4, 6, 8, or 10, or functional fragment or a variant thereof) can be included as part of a vector that produces an OtsA polypeptide and/or an OtsB polypeptide, or a trehalose biosynthesis fusion polypeptide. In some embodiments, any one of the nucleic acids described herein (e.g., any nucleotide encoding for any one of SEQ ID NOs: 2, 4, 6, 8, or 10, or a functional fragment or a variant thereof) can be included as part of an adeno-associated viral vector. In some embodiments, any one of the nucleic acids described herein (e.g., any nucleic acid encoding for any one of SEQ ID NOs: 2, 4, 6, 8, or 10, or a functional fragment or a variant thereof) can be included as part of any serotype of the adeno-associated viral vector (e.g., AAV1, AAV2, AAV3, AAV4, AAV5, AAV6, AAV7, AAV8, AAV9). In some cases, any of the nucleic acids described herein (e.g., any nucleic acid encoding for any one of SEQ ID NOs: 2, 4, 6, 8, or 10, or a functional fragment or a variant thereof) can be included as part of an AAV1 vector, an AAV5 vector, or an AAV9 vector. In some cases, any of the nucleic acids described herein can be included as part of an AAV9 vector.

Various nucleic acid sequences are embodied in the present disclosure. As used herein, “nucleic acid” sequence or equivalents thereof refer to a DNA or RNA sequence. The term captures sequences that include any of the known base analogues of DNA and RNA such as, but not limited to 4-acetylcytosine, 8-hydroxy-N-6-methyladenosine, aziridinylcytosine, pseudoisocytosine, 5-(carboxyhydroxylmethyl) uracil, 5-fluorouracil, 5-bromouracil, 5-carboxymethylaminomethyl-2-thiouracil, 5-carboxymethyl-aminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxyamino-methyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, -uracil-5-oxyacetic acid methylester, uracil-5-oxyacetic acid, pseudouracil, queosine, 2-thiocytosine, and 2,6-diaminopurine.

In some embodiments, the nucleic acids disclosed herein include other nucleic acid sequences, including but not limited to control sequences or regulatory sequences. Control sequences refer collectively to promoter sequences, polyadenylation signals, transcription termination sequences, upstream regulatory domains, origins of replication, internal ribosome entry sites (“IRES”), enhancers, and the like, which collectively provide for the replication, transcription and translation of a coding sequence in a recipient cell. Not all of these control sequences need always be present so long as the selected coding sequence is capable of being replicated, transcribed and translated in an appropriate host cell.

In some cases, disclosed herein are nucleic acids that include one or more “promoter” sequences, which are used herein in its ordinary sense to refer to a nucleotide region comprising a DNA regulatory sequence, wherein the regulatory sequence is derived from a gene which is capable of binding RNA polymerase and initiating transcription of a downstream (3′-direction) coding sequence. Non-limiting examples of promoters includes a human synapsin promoter, a chicken beta-actin promoter, SV40 early promoter, mouse mammary tumor virus terminal repeat (LTR) promoter, an adenovirus major late promoter (AdMLP), a herpes simplex virus (HSV) promoter, a cytomegalovirus (CMV) promoter, or an EM7 promoter.

In some cases, the promoter is inducible. Examples of inducible promoters can include, but are not limited to T7 RNA polymerase promoter, T3 RNA polymerase promoter, Isopropyl-beta-D-thiogalactopyranoside (IPTG)-regulated promoter, lactose induced promoter, heat shock promoter, tetracycline-regulated promoter, steroid-regulated promoter, metal-regulated promoter, estrogen receptor-regulated promoter, etc. Inducible promoters can therefore be regulated by molecules including, but not limited to, doxycycline; RNA polymerase, e.g., T7 RNA polymerase; an estrogen receptor; etc.

In some cases, the promoter is a spatially restricted promoter (i.e., cell type specific promoter, tissue specific promoter, etc.) such that in a multi-cellular organism, the promoter is active (i.e., “ON”) in a subset of specific cells. Spatially restricted promoters may also be referred to as enhancers, transcriptional control elements, control sequences, etc. Any convenient spatially restricted promoter may be used and the choice of suitable promoter (e.g., a brain specific promoter, a promoter that drives expression in a subset of neurons, etc.) will depend on the organism. For example, various spatially restricted promoters are known for mammals. Some spatially restricted promoters are also temporally restricted such that the promoter is in the “ON” state or “OFF” state during specific stages of embryonic development or during specific stages of a biological process (e.g., hair follicle cycle in mice). Examples of spatially restricted promoters include, but are not limited to, neuron-specific promoters, glial-specific promoters, synapse-specific promoters, astrocyte-specific promoters, etc. Neuron-specific spatially restricted promoters include, but are not limited to, a neuron-specific enolase (NSE) promoter (see, e.g., EMBL HSEN02, X51956); an aromatic amino acid decarboxylase (AADC) promoter; a neurofilament promoter (see, e.g., GenBank HUMNFL, L04147); a synapsin promoter (see, e.g., GenBank HUMSYNIB, M55301); a thy-1 promoter (see, e.g., Chen et al. (1987) Cell 51:7-19; and Llewellyn, et al. (2010) Nat. Med. 16(10): 1161-1166); a serotonin receptor promoter (see, e.g., GenBank S62283); a tyrosine hydroxylase promoter (TH) (see, e.g., Oh et al. (2009) Gene Ther 16:437; Sasaoka et al. (1992) Mol. Brain Res. 16:274; Boundy et al. (1998) J. Neurosci. 18:9989; and Kaneda et al. (1991) Neuron 6:583-594); a GnRH promoter (see, e.g., Radovick et al. (1991) Proc. Natl. Acad. Sci. USA 88:3402-3406); an L7 promoter (see, e.g., Oberdick et al. (1990) Science 248:223-226); a DNMT promoter (see, e.g., Bartge et al. (1988) Proc. Natl. Acad. Sci. USA 85:3648-3652); an enkephalin promoter (see, e.g., Comb et al. (1988) EMBO J. 17:3793-3805); a myelin basic protein (MBP) promoter; a Ca2+-calmodulin-dependent protein kinase II-alpha (CamKIIa) promoter (see, e.g., Mayford et al. (1996) Proc. Natl. Acad. Sci. USA 93:13250; and Casanova et al. (2001) Genesis 31:37); a CMV enhancer/platelet-derived growth factor-β promoter (see, e.g., Liu et al. (2004) Gene Therapy 11:52-60); and the like.

The nucleic acids embodied in the present disclosure are operably linked to each other or linked to a protein or peptide. As used herein, “operatively linked” refers to an arrangement of elements wherein the described components are configured to perform their usual function. Thus, control sequences operably linked to a coding sequence are capable of expressing of the coding sequence. The control sequences need not be contiguous with the coding sequence, so long as they function to direct the expression thereof. Thus, for example, intervening untranslated yet transcribed sequences can be present between a promoter sequence and the coding sequence and the promoter sequence can still be considered “operably linked” to the coding sequence.

The ligated DNA sequences are operably linked to suitable transcriptional or translational regulatory elements. The regulatory elements responsible for expression of DNA are located only 5′ to the DNA sequence encoding the first polypeptides. Similarly, stop codons required to end translation and transcription termination signals are only present 3′ to the DNA sequence encoding the secondary, tertiary, or quaternary, etc., polypeptide (i.e., a stop codon will be present on the ultimate polypeptide depending on the number of distinct polypeptides making up a chimeric protein molecule).

In addition to OtsA polypeptides and/or OtsB polypeptides, and trehalose biosynthesis fusion polypeptides, the present disclosure provides compositions including polynucleotides encoding an OtsA polypeptide and/or an OtsB polypeptide, or a trehalose biosynthesis fusion polynucleotide. The terms “DNA” and “polynucleotide” are used interchangeably herein to refer to a DNA molecule that has been isolated free of total genomic DNA of a particular species. “Isolated,” as used herein, means that a polynucleotide is substantially separated from other coding sequences, and that the DNA molecule does not contain large portions of unrelated coding DNA, such as large chromosomal fragments or other functional genes or polypeptide coding regions. Of course, this refers to the DNA molecule as originally isolated, and does not exclude genes or coding regions later added to the segment through genetic engineering or laboratory manipulation.

The phrase “OtsA gene” or “OtsA transgene”, for example, or “OtsB gene” or “OtsA transgene”, or other OtsA or OtsB isomers thereof, refers to DNA or RNA and can include sense and antisense strands. Also, as used herein, “polynucleotide” refers to a polymer of deoxyribonucleotides or ribonucleotides, in the form of a separate fragment or as a component of a larger construct. Polynucleotides of the disclosure include functional derivatives of known polynucleotides which operatively encode an OtsA polypeptide and/or an OtsB polypeptide, or a trehalose biosynthesis fusion polypeptide.

The polynucleotide sequence can be deduced from the genetic code, however, the degeneracy of the code must be taken into account. Polynucleotides of the disclosure include sequences which are degenerate as a result of the genetic code, which sequences may be readily determined by those of ordinary skill in the art.

Further, the term “heterologous” as it relates to nucleic acid sequences such as coding sequences and control sequences, denotes sequences that are not normally joined together, and/or are not normally associated with a particular cell. Thus, a “heterologous” region of a nucleic acid construct or a vector is a segment of nucleic acid within or attached to another nucleic acid molecule that is not found in association with the other molecule in the nucleic acid's native context. For example, a heterologous region of a nucleic acid construct could include a coding sequence flanked by sequences not found in association with the coding sequence in the coding sequence's native context. Another example of a heterologous coding sequence is a construct where the coding sequence itself is not found in nature (e.g., synthetic sequences having codons different from the native gene). Similarly, a cell transformed with a construct which is not normally present in the cell would be considered heterologous for purposes of this disclosure. Allelic variation or naturally occurring mutational events do not give rise to heterologous DNA, as used herein.

Accordingly, a “coding sequence” or a sequence which “encodes” a particular polypeptide, is a nucleic acid sequence which is transcribed (in the case of DNA) and translated (in the case of mRNA) into a polypeptide in vitro or in vivo when placed under the control of appropriate regulatory sequences. The boundaries of the coding sequence are determined by a start codon at the 5′ (amino) terminus and a translation stop codon at the 3′ (carboxy) terminus. A coding sequence can include, but is not limited to, cDNA from prokaryotic or eukaryotic mRNA, genomic DNA sequences from prokaryotic or eukaryotic DNA, and even synthetic DNA sequences. A transcription termination sequence will usually be located 3′ to the coding sequence.

It will be obvious to one skilled in the art, that to obtain and use OtsA and/or OtsB sequences, or nucleic acids encoding trehalose biosynthesis fusion polypeptide sequences included methods in this disclosure and those known in the art. In addition, DNA and/or RNA may also be synthesized using automated nucleic acid synthesis by any method known now known or later discovered (e.g., PCR, cDNA synthesis (see, for example, Sambrook et al., Molecular Cloning: A Laboratory Manual, 3rd Ed., Cold Spring Harbor Laboratory, Cold Spring Harbor, N.Y., 2001, and Current Protocols in Molecular Biology, M. Ausubel et al., eds., (Current Protocols, a joint venture between Greene Publishing Associates, Inc. and John Wiley & Sons, Inc., most recent Supplement).

As will be also recognized by the skilled artisan, polynucleotides of the disclosure may be single-stranded or double-stranded, and may be DNA (genomic, cDNA or synthetic) or RNA molecules. RNA molecules may include HnRNA molecules, which contain introns and correspond to a DNA molecule in a one-to-one manner, and mRNA molecules, which do not contain introns. Additional coding or noncoding sequences may, but need not, be present within a polynucleotide of the present disclosure, and a polynucleotide may, but need not, be linked to other molecules and/or support materials.

Polynucleotides can encode a combination of different sequences, e.g., a transgene, a viral polynucleotide, a nucleic acid encoding a selectable marker and the like.

In the compositions of the present disclosure, the polynucleotide or nucleic acid can be either a DNA molecule or an RNA molecule. The sequences in question can be of natural or artificial origin, and in particular genomic DNA, cDNA, mRNA, tRNA, rRNA, hybrid sequences or synthetic or semi-synthetic sequences. In addition, the nucleic acid can be very variable in size, ranging from oligonucleotide to chromosome. These nucleic acids may be of human, animal, vegetable, bacterial, viral, and the like, origin. They may be obtained by any technique known to a person skilled in the art, and in particular by the screening of libraries, by chemical synthesis or alternatively by mixed methods including the chemical or enzymatic modification of sequences obtained by the screening of libraries. They can, moreover, be incorporated into vectors, such as plasmid vectors.

In some cases, the nucleic acids of the present disclosure have undergone a chemical or biological modification to render them more stable. Exemplary modifications to a nucleic acid include the depletion of a base (e.g., by deletion or by the substitution of one nucleotide for another) or modification of a base, for example, the chemical modification of a base. The phrase “chemical modifications” as used herein, includes modifications which introduce chemistries which differ from those seen in naturally occurring nucleic acids, for example, covalent modifications such as the introduction of modified nucleotides, (e.g., nucleotide analogs, or the inclusion of pendant groups which are not naturally found in such nucleic acid molecules).

In addition, suitable modifications include alterations in one or more nucleotides of a codon such that the codon encodes the same amino acid but is more stable than the codon found in the wild-type version of the nucleic acid. For example, an inverse relationship between the stability of RNA and a higher number cytidines (C's) and/or uridines (U's) residues has been demonstrated, and RNA devoid of C and U residues have been found to be stable to most RNases (Heidenreich, et al. J Biol Chem 269, 2131-8 (1994)). In some cases, the number of C and/or U residues in an mRNA sequence is reduced. In another case, the number of C and/or U residues is reduced by substitution of one codon encoding a particular amino acid for another codon encoding the same or a related amino acid. Contemplated modifications to the mRNA nucleic acids of the present disclosure also include the incorporation of pseudouridines. The incorporation of pseudouridines into the mRNA nucleic acids of the present disclosure may enhance stability and translational capacity, as well as diminishing immunogenicity in vivo. (See, e.g., Kariko, K., et al., Molecular Therapy 16 (11): 1833-1840 (2008)). Substitutions and modifications to the nucleic acids of the present disclosure may be performed by methods readily known to one or ordinary skill in the art.

The constraints on reducing the number of C and U residues in a sequence will likely be greater within the coding region of an mRNA, compared to an untranslated region, (i.e., it will likely not be possible to eliminate all of the C and U residues present in the message while still retaining the ability of the message to encode the desired amino acid sequence). The degeneracy of the genetic code, however presents an opportunity to allow the number of C and/or U residues that are present in the sequence to be reduced, while maintaining the same coding capacity (i.e., depending on which amino acid is encoded by a codon, several different possibilities for modification of RNA sequences may be possible). For example, the codons for Gly can be altered to GGA or GGG instead of GGU or GGC.

The term modification also includes, for example, the incorporation of non-nucleotide linkages or modified nucleotides into the nucleic acid sequences of the present disclosure (e.g., modifications to one or both the 3′ and 5′ ends of an mRNA molecule encoding a functional protein or enzyme). Such modifications include the addition of bases to a nucleic acid sequence (e.g., the inclusion of a poly A tail or a longer poly A tail), the alteration of the 3′ UTR, or the 5′ UTR, complexing the nucleic acid with an agent (e.g., a protein or a complementary nucleic acid molecule), and inclusion of elements which change the structure of a nucleic acid molecule (e.g., which form secondary structures).

The poly A tail is thought to stabilize natural messengers and synthetic sense RNA. Therefore, in one embodiment a long poly A tail can be added to an mRNA molecule thus rendering the RNA more stable. Poly A tails can be added using a variety of art-recognized techniques. For example, long poly A tails can be added to synthetic or in vitro transcribed RNA using poly A polymerase (Yokoe, et al. Nature Biotechnology. 1996; 14:1252-1256). A transcription vector can also encode long poly A tails. In addition, poly A tails can be added by transcription directly from PCR products. Poly A may also be ligated to the 3′ end of a sense RNA with RNA ligase (see, e.g., Molecular Cloning A Laboratory Manual, 2nd Ed., ed. by Sambrook, Fritsch and Maniatis (Cold Spring Harbor Laboratory Press: 1991 edition)). In one embodiment, the length of the poly A tail is at least about 90, 200, 300, 400 at least 500 nucleotides. In one embodiment, the length of the poly A tail is adjusted to control the stability of a modified sense mRNA molecule of the disclosure and, thus, the transcription of protein. For example, since the length of the poly A tail can influence the half-life of a sense mRNA molecule, the length of the poly A tail can be adjusted to modify the level of resistance of the mRNA to nucleases and thereby control the time course of protein expression in a cell. In one embodiment, the stabilized nucleic acid molecules are sufficiently resistant to in vivo degradation (e.g., by nucleases), such that they may be delivered to the target cell without a transfer vehicle.

Accordingly, in order to express a desired polypeptide, the nucleotide sequences encoding any of the polypeptides described herein, e.g., OtsA polypeptide and/or OtsB polypeptide, or a trehalose biosynthesis fusion polypeptide, or functional equivalents, may be inserted into appropriate expression vector, i.e., a vector which contains the necessary elements for the transcription and translation of the inserted coding sequence. Methods that are well known to those skilled in the art may be used to construct expression vectors containing sequences encoding a polypeptide of interest and appropriate transcriptional and translational control elements. These methods include in vitro recombinant DNA techniques, synthetic techniques, and in vivo genetic recombination. Such techniques are described, for example, in Sambrook, J. et al. (1989) Molecular Cloning: A Laboratory Manual, Cold Spring Harbor Press, Plainview, N.Y., and Ausubel, F. M. et al. (1989) Current Protocols in Molecular Biology, John Wiley & Sons, New York. N.Y, incorporated herein by reference.

Specific initiation signals may also be used to achieve more efficient translation of sequences encoding a polypeptide of interest. Such signals include the ATG initiation codon and adjacent sequences. In cases where sequences encoding the polypeptide, its initiation codon, and upstream sequences are inserted into the appropriate expression vector, no additional transcriptional or translational control signals may be needed. However, in cases where only coding sequence, or a portion thereof, is inserted, exogenous translational control signals including the ATG initiation codon should be provided. Furthermore, the initiation codon should be in the correct reading frame to ensure translation of the entire insert. Exogenous translational elements and initiation codons may be of various origins, both natural and synthetic. The efficiency of expression may be enhanced by the inclusion of enhancers which are appropriate for the particular cell system which is used, such as those described in the literature (Scharf, D. et al. (1994) Results Probl. Cell Differ. 20:125-162, incorporated herein by reference).

Moreover, gene delivery involves polymers which form complexes, nanoparticles (defined as less than 1 micron in diameter), or even microparticles (defined as 1 micron in diameter or greater) with DNA plasmids and other nucleic acids. Many kinds of polymers have been described that enhance the expression of genes encoded by nucleic acids in cells.

For example, cationic polymers such as poly-L-lysine, poly-L-glutamate, or block co-polymers may also be delivery agents for nucleic acids. Further, the use of poly [alpha-(4-aminobutyl)-1-glycolic acid] (PAGA) has been used to deliver plasmid DNAs to tumor-bearing mice. Still, the use of water-soluble lipopolymer (WSLP), using branched polyethylenimine and cholesteryl chloroformate has also been described. Polyethylenimine-based vesicle-polymer hybrid gene delivery as another way to deliver plasmid DNA expression vectors, including the use of poly(propylenimine) dendrimers as delivery agents Also, polyethylene glycol (PEG) copolymers were found to improve plasmid DNA delivery, including various kinds of polymers that can be used for the controlled release of plasmid DNA and other nucleic acids. Such molecules include poly(lactic acid) and its derivatives, PEGylated poly(lactic acid), poly(lactic-co-glycolic acid) and its derivatives, poly(ortho esters) and their derivatives, PEGylated poly(ortho esters), poly(caprolactone) and its derivatives, PEGylated poly(caprolactone), polylysine and its derivatives, PEGylated polylysine, poly(ethylene imine) and its derivatives, PEGylated poly(ethylene imine), poly(acrylic acid) and its derivatives, PEGylated poly(acrylic acid), poly(urethane) and its derivatives, PEGylated poly(urethane), and combinations of all of these. One object of the present disclosure is the use of polymeric lipid-protein-sugar microparticles for the delivery of nucleic acids. These and other polymers are well known in the art.

For efficient production of trehalose in vivo, the ratio of the OtsA polypeptide and the OtsB polypeptide can be altered. For example, in some cases, it may be beneficial to produce an uneven ratio or altered ratio of OtsA polypeptide, or fragment thereof to the OtsB polypeptide, or fragment thereof. In some cases, more of the OtsB polypeptide, or fragment thereof, may be produced than of the OtsA polypeptide, or fragment thereof. Altered ratios can be achieved in a variety of way. For example, the OtsA polypeptide, or fragment thereof, and the OtsB polypeptide, or fragment thereof, can be operably linked to two different promoters of different strengths. The OtsB polypeptide can be operably linked to a promoter that is stronger than a second promoter operably linked to the OtsA polypeptide. If two different promoters have different strengths, the stronger promoter results in the production of a higher level or activity of the polypeptide to which the stronger promoter is operably linked, compared to the second promoter operably linked to a different polypeptide. Inducible promoters can have varying strengths depending on the concentration of the substance that induces expression. Another way to change the ratio of the polypeptides (e.g., the ratio of the OtsB polypeptide to the OtsA polypeptide), is to have the polypeptides expressed on different vectors with different copy numbers. For example, the nucleic acid encoding the OtsB polypeptide can be expressed from a vector with a higher copy number than the vector that includes the nucleic acid encoding the OtsA polypeptide.

### Adeno-Associated Virus (AAV)

Adeno-associated virus (AAV) has shown promise for delivering genes for gene therapy in clinical trials in humans. See, for example, Naso et al, BioDrugs. 2017; 31(4): 317-334. As the only viral vector system based on a nonpathogenic and replication-defective virus, recombinant AAV virions have been successfully used to establish efficient and sustained gene transfer of both proliferating and terminally differentiated cells in a variety of tissues.

The AAV genome is a linear, single-stranded DNA molecule containing about 4681 nucleotides. The AAV genome generally comprises an internal nonrepeating genome flanked on each end by inverted terminal repeats (ITRs). The ITRs are approximately 145 base pairs (bp) in length. The ITRs have multiple functions, including as origins of DNA replication, and as packaging signals for the viral genome. The internal nonrepeated portion of the genome includes two large open reading frames, known as the AAV replication (rep) and capsid (cap) genes. The rep and cap genes code for viral proteins that allow the virus to replicate and package into a virion. In particular, a family of at least four viral proteins is expressed from the AAV rep region, Rep 78, Rep 68, Rep 52, and Rep 40, named according to their apparent molecular weight. The AAV cap region encodes at least three proteins, VP1, VP2, and VP3.

As used herein, the term “vector” means any genetic element, such as a plasmid, phage, transposon, cosmid, chromosome, virus, virion, etc., which is capable of replication when associated with the proper control elements and which can transfer gene sequences between cells. Thus, the term includes cloning and expression vehicles, as well as viral vectors previously discussed. Similarly, “recombinant expression vector” refers to systems of polynucleotide(s) which operatively encode polypeptides expressible in eukaryotes or prokaryotes. Methods of expressing DNA sequences having eukaryotic or viral sequences in prokaryotes are well known in the art. Biologically functional viral and plasmid DNA vectors capable of expression and replication in a host are also well known in the art. Hosts can include microbial, yeast, insect and mammalian organisms.

The vectors or recombinant expression vectors provided herein are easily manufactured, and combine the advantages of adenovirus (high titer, high infectivity, large capacity, lack of association with human malignancy) but with the integration capability of AAV, making them particularly suitable for stable gene transfer which is useful in, for example, gene therapy approaches such as that described herein. A further advantage of the described AAV vectors is that, by virtue of containing AAV TR or ITR and D sequences that flank the gene of interest, it is expected that they integrate into cellular chromosomal DNA. Integration is important for stable gene transfer into cells. Another advantage of the AAV vectors provided herein is that they are packaged efficiently into stable virus particles whether small or large polynucleotides are used. Still, another advantage of the AAV vectors provided herein is that they are less cytotoxic than first generation adenovirus vectors since no adenovirus genes are expressed within transduced cells.

The AAV-vector and/or AAV-OtsA polypeptide and/or OtsB polypeptide, or AAV-trehalose biosynthesis fusion polypeptide containing virions described herein are “transfected”, which refers to the uptake of foreign DNA by a cell. That is, a cell “transfected” with exogenous DNA and the DNA is introduced inside the cell membrane. A number of transfection techniques are generally known in the art. See, e.g., Graham et al. (1973) Virology, 52:456, Sambrook et al. (1989) Molecular Cloning, a laboratory manual, Cold Spring Harbor Laboratories, New York, Davis et al. (1986) Basic Methods in Molecular Biology, Elsevier, and Chu et al. (1981) Gene 13:197, incorporated herein by reference. Such techniques can be used to introduce one or more exogenous DNA moieties, such as a nucleotide integration vector and other nucleic acid molecules, into suitable host cells.

The term “host cell” or “host” denotes, for example, mammalian cells, that can be, or have been, used as recipients of an AAV helper construct, an AAV vector plasmid, an accessory function vector, or other transfer DNA. Similarly, the terms “subject”, “individual” or “patient” are used interchangeably herein and refer to a vertebrate, and in some cases, a mammal. Mammals include, but are not limited to, murines, simians, humans, farm animals, sport animals and pets. The term includes the progeny of the original cell which has been transfected. Thus, a “host cell” or “host” as used herein generally refers to a cell which has been transfected with an exogenous DNA sequence. It is understood that the progeny of a single parental cell may not necessarily be completely identical in morphology or in genomic or total DNA complement as the original parent, due to natural, accidental, or deliberate mutation.

As used herein, the term “cell line” refers to a population of cells capable of continuous or prolonged growth and division in vitro. Often, cell lines are clonal populations derived from a single progenitor cell. It is further known in the art that spontaneous or induced changes can occur in karyotype during storage or transfer of such clonal populations. Therefore, cells derived from the cell line referred to may not be precisely identical to the ancestral cells or cultures, and the cell line referred to includes such variants.

Additional viral vectors useful for delivering the polynucleotides encoding polypeptides of the present disclosure by gene transfer include those derived from the pox family of viruses, such as vaccinia virus and avian poxvirus. By way of example, vaccinia virus recombinants expressing the novel molecules can be constructed as follows. The DNA encoding a polypeptide is first inserted into an appropriate vector so that it is adjacent to a vaccinia promoter and flanking vaccinia DNA sequences, such as the sequence encoding thymidine kinase (TK). This vector is then used to transfect cells which are simultaneously infected with vaccinia. Homologous recombination serves to insert the vaccinia promoter plus the gene encoding the polypeptide of interest into the viral genome. The resulting TK(−) recombinant can be selected by culturing the cells in the presence of 5-bromodeoxyuridine and picking viral plaques resistant thereto.

A vaccinia-based infection/transfection system can be conveniently used to provide for inducible, transient expression or coexpression of one or more polypeptides described herein in host cells of an organism. In this particular system, cells are first infected in vitro with a vaccinia virus recombinant that encodes the bacteriophage T7 RNA polymerase. This polymerase displays exquisite specificity in that it only transcribes templates bearing T7 promoters. Following infection, cells are transfected with the polynucleotide or polynucleotides of interest, driven by a T7 promoter. The polymerase expressed in the cytoplasm from the vaccinia virus recombinant transcribes the transfected DNA into RNA which is then translated into polypeptide by the host translational machinery. The method provides for high level, transient, cytoplasmic production of large quantities of RNA and its translation products. See, e.g., Elroy-Stein and Moss, Proc. Natl. Acad. Sci. USA (1990) 87:6743-6747; Fuerst et al. Proc. Natl. Acad. Sci. USA (1986) 83:8122-8126, incorporated herein by reference.

Alternatively, avipoxviruses, such as the fowlpox and canarypox viruses, can also be used to deliver the coding sequences of interest. Recombinant avipox viruses, expressing immunogens from mammalian pathogens, are known to confer protective immunity when administered to non-avian species. The use of an Avipox vector is particularly desirable in human and other mammalian species since members of the Avipox genus can only productively replicate in susceptible avian species and therefore are not infective in mammalian cells. Methods for producing recombinant Avipoxviruses are known in the art and employ genetic recombination, as described above with respect to the production of vaccinia viruses. See, e.g., WO 91/12882; WO 89/03429; and WO 92/03545, incorporated herein by reference.

Any of a number of alphavirus vectors can also be used for delivery of polynucleotide compositions of the present disclosure, such as those vectors described in U.S. Pat. Nos. 5,843,723; 6,015,686; 6,008,035 and 6,015,694, incorporated herein by reference. Certain vectors based on Venezuelan Equine Encephalitis (VEE) can also be used, illustrative examples of which can be found in U.S. Pat. Nos. 5,505,947 and 5,643,576, both of which are incorporated herein by reference.

Moreover, molecular conjugate vectors, such as the adenovirus chimeric vectors described in Michael et al. J. Biol. Chem. (1993) 268:6866-6869 and Wagner et al. Proc. Natl. Acad. Sci. USA (1992) 89:6099-6103, incorporated herein by reference, can also be used for gene delivery under the disclosure.

AAV has been engineered to deliver genes of interest by deleting the internal nonrepeating portion of the AAV genome (i.e., the rep and cap genes) and inserting a heterologous gene between the ITRs. The heterologous gene is typically functionally or operatively linked to a heterologous promoter (constitutive, cell-specific, or inducible) capable of driving gene expression in the patient's target cells under appropriate conditions. Termination signals, such as polyadenylation sites, can also be included.

As used herein, the term “AAV vector” means a vector derived from an adeno-associated virus serotype, including without limitation, AAV1, AAV2, AAV3, AAV4, AAV5, AAV6, AAV7, AAV8, AAV9, and mutated forms thereof. AAV vectors can have one or more of the AAV wild-type genes deleted in whole or part, the rep and/or cap genes, but retain functional flanking ITR sequences. Despite the high degree of homology, the different serotypes have tropisms for different tissues. For example, adeno-associated virus serotypes AAV1, AAV2, AAV4, AAV5, AAV8, and AAV9 exhibit central nervous system tropism. See, for example, Kwon et al., Pharm Res. 2008 Mar. 25 (3): 489-99; Wu et al., Mol. Ther. 2006 Sep. 14 (3): 316-27; Burger et al., Mol. Ther. 2004 Aug. 10 (2): 302-17; or Grimm et al., Curr Gene Ther. 2003 Aug. 3 (4): 281-304. The AAV serotype 9 (AAV9) has been shown to cross the blood brain barrier and preferentially transduce neurons in neonates and astrocytes in adults (Foust et al. 2009; Bourdenx et al. 2014). AAV9 effectively eliminates the need for direct injection into the CNS as systemic delivery should reach CNS targets. In some cases, the AAV vector is derived from an adeno-associated virus serotype AAV1, an adeno-associated virus serotype AAV5, or an adeno-associated virus serotype AAV9. In some cases, the AAV vector is derived from an adeno-associated virus serotype AAV1. In some cases, the AAV vector is derived from an adeno-associated virus serotype AAV5. In some cases, the AAV vector is derived from an adeno-associated virus serotype AAV9.

Functional ITR sequences are necessary for the rescue, replication and packaging of the AAV virion. Thus, an AAV vector is defined herein to include at least those sequences required in cis for replication and packaging (e.g., functional ITRs) of the virus. The ITRs need not be the wild-type nucleotide sequences, and may be altered, for example, by the insertion, deletion or substitution of nucleotides, as long as the sequences provide for functional rescue, replication and packaging.

AAV vectors must have one copy of the AAV inverted terminal repeat sequences (ITRs) at each end of the genome in order to be replicated, packaged into AAV particles and integrated efficiently into cell chromosomes. However, the nucleic acid promoted by ITR can be any desired sequence. In one embodiment, the nucleic acid encodes an OtsA polypeptide and/or an OtsB polypeptide, or a trehalose biosynthesis fusion polypeptide, which has a desired function in the cell in which the vector is expressed. For example, the OtsA polypeptide and/or OtsB polypeptide, or a trehalose biosynthesis fusion polypeptide can produce trehalose.

The ITR consists of nucleotides 1 to 145 at the left end of the AAV DNA genome and the corresponding nucleotides 4681 to 4536 (i.e., the same sequence) at the right hand end of the AAV DNA genome. Thus, AAV vectors must have a total of at least 300 nucleotides of the terminal sequence. So, for packaging large coding regions into AAV vector particles, it is important to develop the smallest possible regulatory sequences, such as transcription promoters and polyA addition signal. In this system, the adeno-associated viral vector comprising the inverted terminal repeat (ITR) sequences of adeno-associated virus and a nucleic acid encoding an OtsA polypeptide and/or an OtsB polypeptide, or a trehalose biosynthesis fusion polypeptide, fragments and/or variants, wherein the inverted terminal repeat sequences promote expression of the nucleic acid in the absence of another promoter.

Accordingly, as used herein, AAV refers to all serotypes of AAV (i.e., 1-9) and mutated forms thereof. Thus, it is routine in the art to use the ITR sequences from other serotypes of AAV since the ITRs of all AAV serotypes are expected to have similar structures and functions with regard to replication, integration, excision and transcriptional mechanisms.

AAV is also a helper-dependent virus. That is, it requires coinfection with a helper virus (e.g., adenovirus, herpesvirus or vaccinia), in order to form AAV virions. In the absence of coinfection with a helper virus, some AAVs establish a latent state in which the viral genome inserts into a host cell chromosome, but infectious virions are not produced. In some AAVs do not establish a latent state (i.e., the viral genome is not inserted into the host cell chromosome, but instead remains as an episome). Subsequent infection by a helper virus “rescues” the integrated genome, allowing it to replicate and package its genome into an infectious AAV virion. While AAV can infect cells from different species, the helper virus must be of the same species as the host cell. Thus, for example, human AAV will replicate in canine cells coinfected with a canine adenovirus.

The term “AAV helper functions” refer to AAV-derived coding sequences which can be expressed to provide AAV gene products that, in turn, function in trans for productive AAV replication. Thus, AAV helper functions include both of the major AAV open reading frames (ORFs), rep and cap. The Rep expression products have been shown to possess many functions, including, among others: recognition, binding and nicking of the AAV origin of DNA replication; DNA helicase activity; and modulation of transcription from AAV (or other heterologous) promoters. The Cap expression products supply necessary packaging functions. AAV helper functions are used herein to complement AAV functions in trans that are missing from AAV vectors.

Accordingly, the term “AAV helper construct” refers generally to a nucleic acid molecule that includes nucleotide sequences providing AAV functions deleted from an AAV vector which is to be used to produce a transducing vector for delivery of a nucleotide sequence of interest. AAV helper constructs are commonly used to provide transient expression of AAV rep and/or cap genes to complement missing AAV functions that are necessary for lytic AAV replication; however, helper constructs lack AAV ITRs and can neither replicate nor package themselves. AAV helper constructs can be in the form of a plasmid, phage, transposon, cosmid, virus, or virion. A number of AAV helper constructs and vectors that encode Rep and/or Cap expression products have been described.

Typically, recombinant AAV (rAAV) virus is made by cotransfecting a plasmid containing the gene of interest flanked by the two AAV terminal repeats and/or an expression plasmid containing the wild-type AAV coding sequences without the terminal repeats, for example pIM45. The cells are also infected and/or transfected with adenovirus and/or plasmids carrying the adenovirus genes required for AAV helper function. rAAV virus stocks made in such fashion are contaminated with adenovirus which must be physically separated from the rAAV particles (for example, by cesium chloride density centrifugation or column chromatography). Alternatively, adenovirus vectors containing the AAV coding regions and/or cell lines containing the AAV coding regions and/or some or all of the adenovirus helper genes could be used. Cell lines carrying the rAAV DNA as an integrated provirus can also be used. In some cases, recombinant AAVs (rAAVs) have tissue-specific targeting capabilities, such that a transgene of the rAAV will be delivered specifically to one or more predetermined tissue(s).

Methods for obtaining recombinant AAVs having a desired capsid protein are well known in the art. (See, for example, US 2003/0138772), the contents of which are incorporated herein by reference in their entirety). Typically the methods involve culturing a host cell which contains a nucleic acid sequence encoding an AAV capsid protein or fragment thereof, a functional rep gene; a recombinant AAV vector composed of, AAV inverted terminal repeats (ITRs) and a transgene; and sufficient helper functions to permit packaging of the recombinant AAV vector into the AAV capsid proteins.

The recombinant AAV vector, rep sequences, cap sequences, and helper functions required for producing the rAAV of the disclosure may be delivered to the packaging host cell using any appropriate genetic element (vector). The selected genetic element may be delivered by any suitable method, including those described herein. The methods used to construct any embodiment of this disclosure are known to those with skill in nucleic acid manipulation and include genetic engineering, recombinant engineering, and synthetic techniques. See, e.g., Sambrook et al, Molecular Cloning: A Laboratory Manual, Cold Spring Harbor Press, Cold Spring Harbor, N.Y. Similarly, methods of generating rAAV virions are well known and the selection of a suitable method is not a limitation on the present disclosure. See, e.g., K. Fisher et al, J. Virol., 70:520-532 (1993) and U.S. Pat. No. 5,478,745.

In some cases, recombinant AAVs may be produced using the triple transfection method (described in detail in U.S. Pat. No. 6,001,650). Typically, the recombinant AAVs are produced by transfecting a host cell with a recombinant AAV vector (comprising a transgene, such as a nucleic acid encoding an OtsA polypeptide and/or an OtsB polypeptide, or a trehalose biosynthesis fusion polypeptide) to be packaged into AAV particles, an AAV helper function vector, and an accessory function vector. An AAV helper function vector encodes the “AAV helper function” sequences (i.e., rep and cap), which function in trans for productive AAV replication and encapsidation. In some cases, the AAV helper function vector supports efficient AAV vector production without generating any detectable wild-type AAV virions (i.e., AAV virions containing functional rep and cap genes). Non-limiting examples of vectors suitable for use with the present disclosure include pHLP19, described in U.S. Pat. No. 6,001,650 and pRep6cap6 vector, described in U.S. Pat. No. 6,156,303, the entirety of both incorporated by reference herein. The accessory function vector encodes nucleotide sequences for non-AAV derived viral and/or cellular functions upon which AAV is dependent for replication (i.e., “accessory functions”). The accessory functions include those functions required for AAV replication, including, without limitation, those moieties involved in activation of AAV gene transcription, stage specific AAV mRNA splicing, AAV DNA replication, synthesis of cap expression products, and AAV capsid assembly. Viral-based accessory functions can be derived from any of the known helper viruses such as adenovirus, herpesvirus (other than herpes simplex virus type-1), and vaccinia virus.

As used herein, the term “accessory functions” refers to non-AAV derived viral and/or cellular functions upon which AAV is dependent for its replication. Thus, the term captures proteins and RNAs that are required in AAV replication, including those moieties involved in activation of AAV gene transcription, stage specific AAV mRNA splicing, AAV DNA replication, synthesis of Cap expression products and AAV capsid assembly. Viral-based accessory functions can be derived from any of the known helper viruses such as adenovirus, herpesvirus (other than herpes simplex virus type-1) and vaccinia virus.

Accordingly, “accessory function vector” refers generally to a nucleic acid molecule that includes nucleotide sequences providing accessory functions. An accessory function vector can be transfected into a suitable host cell, wherein the vector is then capable of supporting AAV virion production in the host cell. Expressly excluded from the term are infectious viral particles as they exist in nature, such as adenovirus, herpesvirus or vaccinia virus particles. Thus, accessory function vectors can be in the form of a plasmid, phage, transposon or cosmid.

In particular, it has been demonstrated that the full-complement of adenovirus genes is not required for accessory helper functions. In particular, adenovirus mutants incapable of DNA replication and late gene synthesis have been shown to be permissive for AAV replication. Similarly, mutants within the E2B and E3 regions have been shown to support AAV replication, indicating that the E2B and E3 regions are probably not involved in providing accessory functions. However, adenoviruses defective in the E1 region, or having a deleted E4 region, are unable to support AAV replication. Thus, ELA and E4 regions are likely required for AAV replication, either directly or indirectly. Other characterized Ad mutants include: E1B; E2A; E2B; E3; and E4. Although studies of the accessory functions provided by adenoviruses having mutations in the E1B coding region have produced conflicting results, recently it has been reported that E1B is required for AAV virion production, while E1B19k is not.

Exemplary accessory function vectors include an adenovirus VA RNA coding region, an adenovirus E4 ORF6 coding region, an adenovirus E2A 72 kD coding region, an adenovirus E1A coding region, and an adenovirus E1B region lacking an intact E1B55k coding region.

By “capable of supporting efficient rAAV virion production” is meant the ability of an accessory function vector or system to provide accessory functions that are sufficient to complement rAAV virion production in a particular host cell at a level substantially equivalent to or greater than that which could be obtained upon infection of the host cell with an adenovirus helper virus. Thus, the ability of an accessory function vector or system to support efficient rAAV virion production can be determined by comparing rAAV virion titers obtained using the accessory vector or system with titers obtained using infection with an infectious adenovirus. In some cases, an accessory function vector or system supports efficient rAAV virion production substantially equivalent to, or greater than, that obtained using an infectious adenovirus when the amount of virions obtained from an equivalent number of host cells is not more than about 200 fold less than the amount obtained using adenovirus infection; in some cases, not more than about 100 fold less; and in some cases, equal to, or greater than, the amount obtained using adenovirus infection.

Hence, by “AAV virion” is meant a complete virus particle, such as a wild-type (wt) AAV virus particle (comprising a linear, single-stranded AAV nucleic acid genome associated with an AAV capsid protein coat). In this regard, single-stranded AAV nucleic acid molecules of either complementary sense, e.g., “sense” or “antisense” strands, can be packaged into any one AAV virion and both strands are equally infectious.

Similarly, a “recombinant AAV virion,” or “rAAV virion” is defined herein as an infectious, replication-defective virus including an AAV protein shell, encapsidating a heterologous nucleotide sequence of interest which is flanked on both sides by AAV ITRs. A rAAV virion is produced in a suitable host cell which has had an AAV vector, AAV helper functions and accessory functions introduced therein. In this manner, the host cell is rendered capable of encoding AAV polypeptides that are required for packaging the AAV vector (containing a recombinant nucleotide sequence of interest) into infectious recombinant virion particles for subsequent gene delivery.

The AAV system of the disclosure, may also include a sequence encoding a selectable marker. The phrase, “selectable marker” or “selectable gene product” as used herein, refers to the use of a gene which may include but is not limited to: bacterial aminoglycoside 3′ phosphotransferase gene (also referred to as the neo gene) which confers resistance to the drug G418 in mammalian cells; bacterial hygromycin G phosphotransferase (hyg) gene which confers resistance to the antibiotic hygromycin; and the bacterial xanthine-guanine phosphoribosyl transferase gene (also referred to as the gpt gene) which confers the ability to grow in the presence of mycophenolic acid. In addition, the AAV system of the disclosure may also include sequences encoding a visual detectable marker, e.g., green fluorescent protein (GFP) or any other detectable marker standard in the art and can be identified and utilized by one skilled in the art without undue experimentation.

Those of skill in the art have circumvented some of the limitations of adenovirus-based vectors by using adenovirus “hybrid” viruses, which incorporate desirable features from adenovirus as well as from other types of viruses as a means of generating unique vectors with highly specialized properties. For example, viral vector chimeras were generated between adenovirus and adeno-associated virus (AAV). These aspects of the disclosure do not deviate from the scope of the disclosure described herein.

Another method for delivery of the polynucleotide for gene therapy involves the use of an adenovirus expression vector. As used herein, the term “adenovirus expression vector” is meant to include those constructs containing adenovirus sequences sufficient (a) to support packaging of the construct and/or (b) to ultimately express a tissue and/or cell-specific construct that has been cloned therein.

In one case, the expression vector comprises a genetically engineered form of adenovirus. Knowledge of the genetic organization of adenovirus, a 36 kb, linear, double-stranded DNA virus, allows substitution of large pieces of adenoviral DNA with foreign sequences up to 7 kb (Grunhaus and Horwitz, Seminar in Virology, 1992; 3:237-252). In contrast to retrovirus, the adenoviral infection of host cells does not result in chromosomal integration because adenoviral DNA can replicate in an episomal manner without potential genotoxicity. Also, adenoviruses are structurally stable, and no genome rearrangement has been detected after extensive amplification.

Adenovirus growth and manipulation is known to those of skill in the art, and exhibits broad host range in vitro and in vivo. This group of viruses can be obtained in high titers, e.g., 10⁹ to 10¹¹ plaque-forming units per mL, and they are highly infective. The life cycle of adenovirus does not require integration into the host cell genome. The foreign genes delivered by adenovirus vectors are episomal and, therefore, have low genotoxicity to host cells. No side effects have been reported in studies of vaccination with wild-type adenovirus, demonstrating their safety and/or therapeutic potential as in vivo gene transfer vectors.

Adenovirus vectors have been used in eukaryotic gene expression and vaccine development. Recently, animal studies suggested that recombinant adenovirus could be used for gene therapy (see, e.g., Stratford-Perricaudet et al., Hum. Gene. Ther., 1991; 1:242-256; Rich et al., 1993). Studies in administering recombinant adenovirus to different tissues include muscle injection, peripheral intravenous injections and stereotactic inoculation into the brain. Recombinant adenovirus and adeno-associated virus can both infect and transduce non-dividing human primary cells.

### Methods of Treating Neurodegenerative Disorders

“Neurodegenerative diseases” or “neurodegenerative disorders” refers to hereditary and sporadic conditions which are characterized by progressive nervous system dysfunction. These disorders are often associated with atrophy of the affected central or peripheral nervous system structures. Alzheimer's disease, Parkinson's disease, Creutzfeldt-Jakob, as well as multiple sclerosis, are due to neuronal degeneration in the central nervous system, disclosed herein.

Human subjects suitable to be selected for treatment include those suffering from neurodegenerative diseases or neurodegenerative disorders selected from dementia absent other prominent neurologic signs, Alzheimer's disease, Senile dementia of the Alzheimer type, or Pick's disease (lobar atrophy), neurodegenerative diseases that include syndromes combining progressive dementia with other prominent neurologic abnormalities, progressive neurodegenerative disease mainly afflicting adults and including progressive neurodegenerative forms of Huntington's disease, multiple system atrophy combining dementia with ataxia and/or manifestation of Parkinson's disease, progressive supranuclear palsy (Steele-Richardson-Olszewski), diffuse Lewy body disease, or corticodentatinigral degeneration. Additional subjects can be suffering from progressive neurodegenerative disease that mainly afflicts young adults and children and include Hallervorden-Spatz disease and progressive familial myoclonic epilepsy, progressive neurodegenerative disease that includes syndromes of gradually developing abnormalities of posture and movement, or disease that includes paralysis agitans (Parkinson's disease), striatonigral degeneration, progressive supranuclear palsy, torsion dystonia (torsion spasm; dystonia musculorum deformans), spasmodic torticollis and other restricted dyskinesias, Familial tremor, or Gilles de la Tourette syndrome, syndromes of progressive ataxia, cerebellar degenerations or spinocerebellar degenerations, cerebellar cortical degeneration or olivopontocerebellar atrophy (OPCA), spinocerebellar degenerations including spinocerebellar degenerations (Friedreich's ataxia and related disorders). Other indications include central autonomic nervous system failure (Shy-Drager syndrome), syndromes of muscular weakness and wasting without sensory changes (motor neuron disease), amyotrophic lateral sclerosis (ALS), spinal muscular atrophy, infantile spinal muscular atrophy (Werdnig-Hoffmann), juvenile spinal muscular atrophy (Wohlfart-Kugelberg-Welander), or other forms of familial spinal muscular atrophy, primary lateral sclerosis or hereditary spastic paraplegia, syndromes combining muscular weakness and wasting with sensory changes (progressive neural muscular atrophy; chronic familial polyneuropathies), peroneal muscular atrophy (Charcot-Marie-Tooth), hypertrophic interstitial polyneuropathy (Deferine-Sottas), or miscellaneous forms of chronic progressive neuropathy, progressive neurodegenerative diseases that include syndromes of progressive visual loss. Other indications treatable with the present invention are pigmentary degeneration of the retina (retinitis pigmentosa), or hereditary optic atrophy (Leber's disease), motor neuron disease and the progressive ataxias; sporadic progressive neurodegenerative diseases, multifocal motor neuropathy with conduction block, motor neuropathy with paraproeinemia, motor-predominant peripheral neuropathies, olivopontocerebellar atrophy, Azorean (Machado-Joseph) disease, familial progressive neurodegenerative diseases such as familial amyotrophic lateral sclerosis, spinal muscular atrophies, familial spastic paraparesis, hereditary biochemical disorders, arthrogryposis multiplex congenital, or progressive juvenile bulbar palsy (Fazio-Londe)—Examples of hereditary biochemical disorders are superoxide dismutase deficiency, hexosaminidase A and B deficiency, or androgen receptor mutation (Kennedy's syndrome). Furthermore, progressive neurodegenerative diseases can include viral and prion diseases, such as HTLV-1 associated myelopathy, progressive multifocal leukoencephalopathy, Creutzfeldt-Jakob disease, Gerstmann-Straussler-Scheinker disease, kuru, fatal familial insomnia, or Alper's disease.

Any of the compositions or pharmaceutical compositions described herein can be administered in combination with any standard treatment of any of the neurodegenerative diseases described herein. For example, treating or preventing symptoms of any of the neurodegenerative diseases can include administering a therapeutic composition comprising a vector comprising a nucleic acid that encodes trehalose-6-phosphate synthase (OtsA), a nucleic acid that encodes trehalose 6-phosphate phosphatase (OtsB), a nucleic acid that encodes OtsA and OtsB, or a nucleic acid that encodes a trehalose biosynthesis fusion polypeptide, or fragments of any of the above, or variants of any of the above to a subject in need.

### Methods of Treating ALS

Amyotrophic lateral sclerosis (ALS), also known as Lou Gehrig's disease, is the most frequent adult motor neuron disease. It was first described in 1869 by the French neurologist Jean-Martin Charcot. This disease is characterized by degeneration and death of motor neurons, which leads to generalized weakness and muscle atrophy. The course of the disorder is inexorably progressive, with 50% of patients dying within 3 years of onset. ALS appears as a rare disease with a prevalence of 4-6 per 100 000 each year and an incidence of 1-2 per 100,000 each year.

Most cases (90%) are classified as sporadic ALS (SALS), and the remainder 10% are inherited and referred to as familial ALS (FALS), with a Mendelian pattern of inheritance. From a clinical standpoint, familial (FALS) and sporadic (SALS) cases cannot be distinguished from one another, apart from a mean age at onset for SALS that is 10 years later than for FALS (56 years versus 46 years). The causes for most cases of ALS are unknown and the clinical course is highly variable, suggesting that multiple factors underlie the disease mechanism. Few treatments are available.

The hallmark of this disease is the selective death of motor neurons located in the brainstem, motor cortex and spinal cord leading to paralysis of voluntary muscles. The paralysis begins focally and disseminates in a pattern that suggests that degeneration is spreading among contiguous pools of motor neurons. Mortality normally results when control of the diaphragm is impaired and the ability to breathe is lost.

ALS is characterized by progressive manifestations of dysfunction of both lower and upper motor neurons. Lower motor neurons connect the brainstem and spinal cord to muscle fibers. Their dysfunction leads to muscle atrophy, cramps and fasciculations (small, local, involuntary muscle contraction). Upper motor neurons originate in motor region of the cerebral cortex or the brainstem and carry motor information down to motor neurons that are directly responsible for stimulating the target muscle. Their dysfunction leads to spasticity (continuous muscle contraction that interfere with gait, movement, and speech) and pathological reflexes, The other related motor neuron diseases are usually distinguished by the type of nerve cells impaired, i.e. upper or lower motor neurons: they are known as Primary Lateral Sclerosis (PLS), Progressive Muscular Atrophy (PMA), Pseudobulbar Palsy and Progressive Bulbar Palsy (PBP).

Diagnosis of ALS is based on clinical signs, established by neurologist on the basis of history, topographic distribution of the neuronal loss and the finding of some characteristic cytological changes. However, there is no clear-cut diagnostic test of ALS available. Clinical features are classified in accordance with affected neurological regions that are bulbar, cervical, and lumbar.

Several mutated genes or genomic regions have been reported to cause or predispose to ALS as well as to ALS with frontotemporal dementia (FTD). For instance, about 20-25% of all FALS cases and around 1% of SALS cases arise because of mutations in superoxide dismutase SOD1. Multiple clinical presentations within the same family are obtained with the same SOD1 mutation which does not necessarily cause a homogenous phenotype. There is no clear correlation between enzyme activity, clinical progression and disease phenotype. However, the period of the disease is similar whatever the mutation is. Historically, the discovery of SOD1 mutations led to the generation of the first animal models of ALS. They develop a motor neuron disease closely resembling human ALS, Among other genes implicated in development of ALS or related motor neuron diseases, alsin, an exchange factor for Rab5A, senataxin, potentially involved in RNA processing, VAPB protein regulating vesicle transport, the major axonal retrograde motor protein dynactin, mitochondrial genes for cytochrome c oxidase and isoleucine tRNA synthetase, angiogenic modulators VEGF and angiogenin, can be mentioned.

ALS is a complex disease with multiple causes and the precise mechanisms involved in the pathogenesis of this disease are not yet resolved. This challenges the discovery of effective pharmacologic therapies. Clinical trials had shown that survival, but not function, is modestly prolonged by riluzole in ALS. Nevertheless, riluzole is currently the only drug approved and the only known therapy for ALS. Regarding to the severity of disease, it is consequently administered as a disease-modifying compound to all the patients suffering from ALS.

Disclosed herein are methods of treating ALS. In one aspect, provided herein is a method of treating ALS, comprising administering a vector comprising a nucleic acid that encodes trehalose-6-phosphate synthase (OtsA), a nucleic acid that encodes trehalose 6-phosphate phosphatase (OtsB), a nucleic acid that encodes OtsA and OtsB, or a nucleic acid that encodes a trehalose biosynthesis fusion polypeptide, or fragments of any of the above, or variants of any of the above to a subject in need thereof. In another aspect, provided herein is a method of treating ALS, comprising administering a therapeutically effective amount of trehalose and a vector comprising a nucleic acid that encodes trehalose-6-phosphate synthase (OtsA), a nucleic acid that encodes trehalose 6-phosphate phosphatase (OtsB), a nucleic acid that encodes OtsA and OtsB, or a nucleic acid that encodes a trehalose biosynthesis fusion polypeptide, or fragments of any of the above, or variants of any of the above to a subject in need thereof.

In some cases, the subject has not been previously diagnosed with ALS.

In some cases, the subject is not being treated for ALS.

In one aspect of the disclosure, the therapeutic composition to treat ALS includes a vector comprising a nucleic acid that encodes trehalose-6-phosphate synthase (OtsA), a nucleic acid that encodes trehalose 6-phosphate phosphatase (OtsB), a nucleic acid that encodes OtsA and OtsB, or a nucleic acid that encodes a trehalose biosynthesis fusion polypeptide, or fragments of any of the above, or variants of any of the above. In another aspect of the disclosure, the therapeutic composition to treat or reduce symptoms associated with ALS includes a polynucleotide encoding a protein capable of indirectly modulating a symptom associated with ALS

In a related aspect, the therapeutic compositions of the present disclosure are administered to a subject as a prophylactic or ameliorative modality. As used herein, “ameliorative,” means to improve or relieve a subject of symptoms associated with a disorder, and includes curing such a disorder.

The therapeutic agents, including polynucleotides, polynucleotides in combination with a vector, both viral and non-viral, can be used in the preparation of a medicament for the treatment of ALS, where the medicament is administered via intravenous, intramuscular, subcutaneous or intraperitoneal injection.

The therapeutic compositions of the present disclosure are delivered to a “target cell” or “host cell” or “target tissue” and equivalents thereof, which refers to the cell, tissue of the host in which expression of the operatively encoding polynucleotide is sought for treatment of ALS.

As disclosed herein, vectors useful in the present disclosure have varying transduction efficiencies. As a result, the viral or non-viral vector transduces more than, equal to, or at least about 10%, 20%, 30%, 40%, 50%, 55%, 60%, 65%, 70%, 75%, 80%, 85%, 90%, 95%, 99%, 100% of the cells of the targeted vascular territory. In some cases, more than one vector (viral or non-viral, or combination thereof) can be used simultaneously, or in sequence. This can be used to transfer more than one polynucleotide, and/or target more than one type of cell. Where multiple vectors or multiple agents are used, more than one transduction/transfection efficiency can result.

Any of the compositions or pharmaceutical compositions described herein can be administered in combination with any standard treatment of ALS. For example, any of the compositions or pharmaceutical compositions described herein can be administered in combination with riluzole. In some cases, a subject was previously administered a standard treatment of ALS. For example, in some cases, a subject was previously administered riluzole.

### Methods of Treating Alzheimer's Disease

Alzheimer's disease (AD) is a neurodegenerative disease and the most common cause of dementia. This disease manifests as a gradual but progressive decline in memory, thinking skills and behavior that is accelerated relative to normal aging (Reitz et al. 2011. Nat Rev Neurol 7:137-152). Eventually, patients are unable to recognize familiar people or carry out the simplest task. Alzheimer's disease is, at this time, the sixth leading cause of death in the United States.

Signs and symptoms of Alzheimer's disease include a reduced ability to understand and remember information; impacted ability to make decisions, perform simple tasks, or follow a conversation; memory loss of recent events, and eventually for long-term events and memories; confusion and inability to remember where one is; loss of interest in favorite hobbies; less expressive, apathetic, and withdrawn from activities; repetition of words, sentences, and thoughts; unordinary behavior such as hiding possessions, physical outbursts, and restlessness; feeling paranoid or threatened; placing items in strange places; and reduced fine motor skills that affects the individual's coordination and mobility to the point where performing day-to-day tasks such as eating, bathing, and getting dressed become difficult.

There are two predominant forms of the disease: Familial Alzheimer's disease can be caused by dominant mutations in one of three genes (APP, PSEN1, or PSEN2). This form of the disease is a rare and devastating illness with onset occurring in mid-life. The second and far more common form of the disease is Sporadic or Late onset Alzheimer's disease (hereinafter “Alzheimer's disease” or “AD”). Onset of Alzheimer's disease typically occurs after the age of 62 years.

Based on the intensity of the typical Alzheimer's symptoms, it can be classified into the following subtypes: mild Alzheimer's disease; moderate Alzheimer's disease; and severe Alzheimer's disease. Mild AD includes the beginning of cognitive impairment that causes difficulties in remembering daily routine such as tasks at work, paying bills, and others. Because these symptoms are not very serious, the patients at this stage manage to remain functional with a certain amount of difficulty. They take longer to perform the same task which they used to do quicker before, and this becomes a pattern. For moderate AD, because of a significant amount of neuronal damage, the symptoms of moderate Alzheimer's are more intense. The confusion becomes worse and due to the amount of memory loss, they become increasingly dependent on others. These individuals, even though physically agile, are not able to perform routine tasks as the delusions take over the sensory processing of their thoughts. In severe AD, as the plaques and tangles spread, the brain cells start dying. This results in shrinkage of brain tissue. The patients with this condition are typically bedridden and are hardly able to communicate.

As the world population and human longevity increase, so do the numbers of people affected by Alzheimer's disease globally. The estimated worldwide costs of dementia, of which Alzheimer's disease accounts for up to 80% of cases, was US$604 billion in 2010, which was greater than 1% of US GDP (Wimo and Prince 2010 World Alzheimer Report 2010: The Global Economic Impact of Dementia 1-93). The cost of caring for Alzheimer patients in the US is expected to increase from US$172 million in 2010, to US$1.07 trillion in 2050 (Alzheimer's Association. “Changing the Trajectory of Alzheimer's Disease: A National Imperative (2010)”).

At this time, the few drugs that are approved for treatment of this disease provide some symptomatic relief, but this is typically of relatively short duration, and the therapies do not alter the course of disease progression (Alzheimer's Association. “Changing the Trajectory of Alzheimer's Disease: A National Imperative (2010)”). Therapies that delay the onset of the disease, reduce the rate of disease progression, or that can do both are urgently needed. Therapies that can achieve either of these goals will reduce the number of individuals with disease, or reduce the number of individuals with the more advanced and debilitating stages of disease (Brookmeyer et al. 2007 Alzheimer's Dement 3:186-191). It is projected that if the onset of Alzheimer's disease is delayed by 5 years due to availability of a breakthrough therapy in 2015, 43% of the 13.5 million Americans expected to have the condition in 2050 would not have the disease, and there will be fewer people with advanced disease.

The principal risk factor for Alzheimer's disease is age, and prevalence of the disease increases with age (approximately 10% of individuals over 65 and approximately 50% of individuals over 85). The incidence of the disease doubles every 5 years after 65 years of age, with the diagnosis of about 1275 new cases per year per 100,000 persons older than 65 years of age (Querfurth et al., 2010 NEJM 362:4). Both men and women are affected by Alzheimer's disease, but women generally represent a higher percentage of cases overall (roughly 60% to 40%), possibly due to greater longevity. People suffering from Alzheimer's disease tend to live approximately 3 to 9 years after diagnosis, on average.

Disclosed herein are methods of treating Alzheimer's disease (AD). In one aspect, provided herein is a method of treating AD, comprising administering a therapeutically effective amount of a vector comprising a nucleic acid that encodes trehalose-6-phosphate synthase (OtsA), a nucleic acid that encodes trehalose 6-phosphate phosphatase (OtsB), a nucleic acid that encodes OtsA and OtsB, or a nucleic acid that encodes a trehalose biosynthesis fusion polypeptide, or fragments of any of the above, or variants of any of the above to a subject in need thereof. In another aspect, provided herein is a method of treating AD, comprising administering a therapeutically effective amount of trehalose and a therapeutically effective amount a vector comprising a nucleic acid that encodes trehalose-6-phosphate synthase (OtsA), a nucleic acid that encodes trehalose 6-phosphate phosphatase (OtsB), a nucleic acid that encodes OtsA and OtsB, or a nucleic acid that encodes a trehalose biosynthesis fusion polypeptide, or fragments of any of the above, or variants of any of the above to a subject in need thereof.

In some cases, the subject has not been previously diagnosed with AD.

In some cases, the subject is not being treated for AD. In some cases, the subject has mild Alzheimer's disease. In some cases, the subject has moderate Alzheimer's disease. In some cases, the subject has severe Alzheimer's disease.

In one aspect of the disclosure, the therapeutic composition to treat AD includes a vector comprising a nucleic acid that encodes trehalose-6-phosphate synthase (OtsA), a nucleic acid that encodes trehalose 6-phosphate phosphatase (OtsB), a nucleic acid that encodes OtsA and OtsB, or a nucleic acid that encodes a trehalose biosynthesis fusion polypeptide, or fragments of any of the above, or variants of any of the above. In another aspect of the disclosure, the therapeutic composition to treat or reduce symptoms associated with AD includes a polynucleotide encoding a protein capable of indirectly modulating a symptom associated with AD.

In a related aspect, the therapeutic compositions of the present disclosure are administered to a subject as a prophylactic or ameliorative modality. As used herein, “ameliorative,” means to improve or relieve a subject of symptoms associated with a disorder, and includes curing such a disorder.

The therapeutic agents, including polynucleotides, polynucleotides in combination with a vector, both viral and non-viral, can be used in the preparation of a medicament for the treatment of AD, where the medicament is administered via intravenous, intramuscular, subcutaneous or intraperitoneal injection.

The therapeutic compositions of the present disclosure are delivered to a “target cell” or “host cell” or “target tissue” and equivalents thereof, which refers to the cell, tissue of the host in which expression of the operatively encoding polynucleotide is sought for treatment of AD.

As disclosed herein, vectors useful in the present disclosure have varying transduction efficiencies. As a result, the viral or non-viral vector transduces more than, equal to, or at least about 10%, 20%, 30%, 40%, 50%, 55%, 60%, 65%, 70%, 75%, 80%, 85%, 90%, 95%, 99%, 100% of the cells of the targeted vascular territory. In some cases, more than one vector (viral or non-viral, or combination thereof) can be used simultaneously, or in sequence. This can be used to transfer more than one polynucleotide, and/or target more than one type of cell. Where multiple vectors or multiple agents are used, more than one transduction/transfection efficiency can result.

Any of the compositions or pharmaceutical compositions described herein can be administered in combination with any standard treatment of AD.

### Methods of Treating Huntington's Disease

Huntington's disease (HD) is a neurodegenerative disease characterized by accumulations of abnormal protein aggregates that results in a progressive decline in motor and cognitive function. It is caused by CAG repeat expansions in the huntingtin gene (HTT) that is translated into a mutant huntingtin protein (mHTT), which is prone to aggregation and is responsible for the progressive loss of neurological function.

The number of CAG repeats in the HTT gene typically ranges from 6 to 35 in healthy individuals. Individuals with 36 to 39 CAG repeats experience few to no symptoms of HD. However, individuals with 40 or more CAG repeats are nearly certain to develop HD and experience symptoms of HD. Current therapeutic strategies are confined to symptomatic treatment of the disease such as spasticity, dopaminergic agents for parkinsonian symptoms, antidepressants, and general supportive care.

Clinical diagnosis of HD is typically based on confirmed family history or positive genetic test (i.e. confirmation of CAG repeat expansion_>36); and onset of motor disturbance as defined by the Unified Huntington's Disease Rating Scale (UHDRS) total motor score (TMS) diagnostic confidence score (DCS), which ranges from 0 (no motor abnormalities suggestive of HD) to 4 (motor abnormalities>99% likely to be due to HD), wherein a score of 4 defines “motor onset” or “manifest” HD. The usual age of onset (i.e. DCS of 4) ranges between 30 to 50 years and the average duration of survival post-diagnosis is 15 to 20 years. This can also be referred to as adult onset Huntington's disease. However, juvenile onset Huntington's disease (JHD) is a form of Huntington's disease that affects children and teenagers. As used herein, “Huntington's disease” refers to both adult onset and juvenile onset Huntington's disease.

Currently, after onset, it is “function” (i.e. assessment of functional capacities), rather than motor signs, which determines disease stage of a subject (e.g. in Neurology, 1979, 29, 1-3 or in Neurology, 1981, 31, 1333-1335). The Total Functional Capacity (TFC) scale (e.g. in Movement Disorders, 1996, 11, 136-142) is a component of the UHDRS and ranges from 0 (fully dependent for all care) to 13 (fully independent) the level of independence of a person with HD (referred to herein as “UHDRS-TFC” score). This scale assesses functional status of a HD patient in terms of ability to work, handle household finances, manage domestic chores, perform activities of daily living, and level of care needed. Based on the UHDRS total functional capacity (TFC), HD is divided into stages 1 to 5 of disease progression. The categorization of HD, based on TFC score (also referred to as Shoulson and Fahn stages), are also described as early stage HD (corresponding to stages 1 or 2, based on TFC score), moderate stage or mid stage HD (corresponding to stage 3, based on TFC score) and advanced stage or late stage HD (corresponding to stage 4 or 5, based on TFC score).

Disclosed herein are methods of treating Huntington's Disease (HD). In one aspect, provided herein is a method of treating HD, comprising administering a therapeutically effective amount of a vector comprising a nucleic acid that encodes trehalose-6-phosphate synthase (OtsA), a nucleic acid that encodes trehalose 6-phosphate phosphatase (OtsB), a nucleic acid that encodes OtsA and OtsB, or a nucleic acid that encodes a trehalose biosynthesis fusion polypeptide, or fragments of any of the above, or variants of any of the above to a subject in need thereof. In another aspect, provided herein is a method of treating HD, comprising administering a therapeutically effective amount of trehalose and a therapeutically effective amount a vector comprising a nucleic acid that encodes trehalose-6-phosphate synthase (OtsA), a nucleic acid that encodes trehalose 6-phosphate phosphatase (OtsB), a nucleic acid that encodes OtsA and OtsB, or a nucleic acid that encodes a trehalose biosynthesis fusion polypeptide, or fragments of any of the above, or variants of any of the above to a subject in need thereof.

In some cases, the subject has not been previously diagnosed with HD.

In some cases, the subject is not being treated for HD. In some embodiments, the subject has early stage HD. In some embodiments, the subject has moderate stage HD. In some embodiments, the subject has late stage HD. In some embodiments, the subject has juvenile HD.

In one aspect of the disclosure, the therapeutic composition to treat HD includes a vector comprising a nucleic acid that encodes trehalose-6-phosphate synthase (OtsA), a nucleic acid that encodes trehalose 6-phosphate phosphatase (OtsB), a nucleic acid that encodes OtsA and OtsB, or a nucleic acid that encodes a trehalose biosynthesis fusion polypeptide, or fragments of any of the above, or variants of any of the above. In another aspect of the disclosure, the therapeutic composition to treat or reduce symptoms associated with HD includes a polynucleotide encoding a protein capable of indirectly modulating a symptom associated with AD.

Some embodiments provide a method of decreasing soluble mutant huntingtin protein (mHTT) levels in a subject having HD, comprising administering a therapeutically effective amount a vector comprising a nucleic acid that encodes trehalose-6-phosphate synthase (OtsA), a nucleic acid that encodes trehalose 6-phosphate phosphatase (OtsB), a nucleic acid that encodes OtsA and OtsB, or a nucleic acid that encodes a trehalose biosynthesis fusion polypeptide, or fragments of any of the above, or variants of any of the above to a subject in need thereof.

In some embodiments, the soluble mHTT level is decreased by about 25% to about 90% (e.g., 25% to 90%, 25% to 80%, 25% to 70%, 25% to 60%, 25% to 50%, 25% 5o 40%, 25% to 30%, 30% to 90%, 40% to 90%, 50% to 90%, 60% to 90%, 70% to 90%, 80% to 90%) after administering the therapeutically effective amount of a vector comprising a nucleic acid that encodes trehalose-6-phosphate synthase (OtsA), a nucleic acid that encodes trehalose 6-phosphate phosphatase (OtsB), a nucleic acid that encodes OtsA and OtsB, or a nucleic acid that encodes a trehalose biosynthesis fusion polypeptide, or fragments of any of the above, or variants of any of the above, relative to the mHTT levels prior to administration of the vector. In some embodiments, soluble mHTT levels in blood plasma of a subject having HD is decreased by about 25% to about 90% following administration of any of the compositions described herein. In some embodiments, soluble mHTT in CSF of a subject having HD is decreased by about 25% to about 90% following administration of any of the compositions described herein.

In some embodiments, wild-type Huntington protein (HTT) levels are about the same before and after administration of any of the compositions described herein.

In a related aspect, the therapeutic compositions of the present disclosure are administered to a subject as a prophylactic or ameliorative modality. As used herein, “ameliorative,” means to improve or relieve a subject of symptoms associated with a disorder, and includes curing such a disorder. Non-limiting symptoms associated with HD include behavioral disturbances, clumsiness, moodiness, irritability, paranoia, apathy anxiety, hallucinations, abnormal eye movements, depression, impaired ability to detect odors, dystonia, involuntary movements, trouble with balance and walking, chorea with twisting and writhing motions, unsteady gait (style of walking), slow reaction time, general weakness, weight loss, dysarthria (speech difficulties), stubbornness, rigidity (continual tension of the muscles), bradykinesia (difficulty initiating and continuing movements), severe chorea, serious weight loss, inability to speak, inability to walk, swallowing problems, inability to care for oneself, and combinations thereof.

Some embodiments provide a method of reducing the rate of cognitive decline in a subject having HD, comprising administering a therapeutically effective amount of a vector comprising a nucleic acid that encodes trehalose-6-phosphate synthase (OtsA), a nucleic acid that encodes trehalose 6-phosphate phosphatase (OtsB), a nucleic acid that encodes OtsA and OtsB, or a nucleic acid that encodes a trehalose biosynthesis fusion polypeptide, or fragments of any of the above, or variants of any of the above to a subject in need thereof.

In some embodiments, the cognitive decline of a subject is determined by one or more scores from the Huntington's Disease Cognitive Assessment Battery (HD-CAB) as described herein.

### Huntington's Disease Cognitive Assessment Battery (HD-CAB) Test

1. Color Shape Cued Set Shifting (COSH). This computerized test assesses cued set shifting. Subjects are presented with a cue, indicating what stimulus dimension they should respond to (either “Color” or “C” for color, and either “Shape” or “S” for shape), followed by a stimulus, for example, a circle or a triangle that is either red or blue. Trials are presented in a fixed order (up to, for example, 96 trials). The outcome measure is switch cost (msec), computed using the response times for correct letter-cued trials that are preceded by correct trials. To determine the switch cost for each subject (i.e., set shifting cost), the difference between mean response times when cues changed across consecutive trials (i.e., color to shape, or shape to color) and mean response times when cues stayed the same on consecutive trials is calculated. Larger switch costs indicate poorer performance. This version is modified from a 192 trial version, which intermixed 96 trials using a 200 msec cue to stimulus delay, and 96 trials using a 1200 msec delay. To ease subject burden and make the administration time feasible in the context of a short, multi-test battery, the shortened version included only the 200 msec delay trials, which are previously found to be more sensitive.
2. Emotion Recognition (EMO). This computerized test assesses the ability to recognize facial expressions of emotion. For the test, subjects are shown a series of photographs of faces from a commonly used stimulus set. They respond by selecting the word that best describes the expression from among seven response options (anger, fear, sadness, disgust, happiness, surprise, or neutral). The test required the subject to press and hold a purple circle called the “Home key”. Pressing the home key triggers the onset of a face stimulus displaying either an emotion or a neutral expression. The face stimulus is displayed for up to four seconds. Response options, which appeared simultaneously with the face stimuli, are available for up to eight seconds. Trials end and the face and response options are replaced by a cross-hair when a subject responds or eight seconds have elapsed, whichever comes first. There can be up to 70 trials in total, with 10 of each of the six emotions, and 10 additional trials depicting neutral expressions. The outcome measure is the number of correctly identified negative emotions (anger, fear, sadness, disgust), out of 40 possible.
3. The Hopkins Verbal Learning Test-Revised (HVLT-R). This is a commercially-available paper and pencil test assessing verbal learning and memory. For the test, subjects try to learn and recall a set of 12 words across three ‘learning trials’. Specifically, examiners read aloud a series of 12 words, at a pace of one every two seconds, after which subjects recalled aloud the words they could remember, in any order. Examiners then repeat the list again, in the same order, and subjects are asked again to recall all of the words they could remember, including the ones they recalled before. This process is repeated three times in total, making up the three learning trials.
4. Judgment of Line Orientation (JLOT). This computerized test assesses visuospatial perception and processing. For the JLOT, subjects view two stimulus lines at angles to each other that appeared at the top of the screen. Simultaneously, at the bottom of the screen, they view a set of response options consisting of 17 lines that are fanned out from 0 to 180 degrees in 11.25 degree increments. The 17 lines are numbered for easy identification. To respond, subjects indicate the numbers of the two lines at the bottom of the screen that exactly match the angles of the two stimulus lines at the top of the screen. The difficulty of the modified test is also enhanced by a) using shorter stimulus lines, b) using stimulus lines that are offset relative to each other rather than exactly matching the relative positioning of the response lines, c) avoiding vertical or horizontal stimulus lines and nearest neighbors of vertical or horizontal lines, and d) avoiding stimulus line pairs that are mirror reflections about the vertical. There are 20 trials. The outcome measure is the number of correct responses out of 40 (2 lines per trial).
5. The Map Search (MAP) is a subtest of the Test of Everyday Attention. MAP is a paper and pencil test assessing visuospatial attention and processing speed. Subjects are presented with a map of Philadelphia on A3 sized paper, on which are printed numerous small symbols indicating points of interest such as places to eat, petrol/gas stations, and auto repair garages. Subjects are given 60 seconds to circle (with a pen) a specified target symbol. There are three alternate forms: the Form A target is a fork and knife, the form B target is a screwdriver and spanner/wrench, and the form C target is a petrol/gas pump. Subjects completed each form only once, and form order for the three visits is counterbalanced across subjects. The outcome measure is the number of correctly circled symbols (maximum 80).
6. The Montreal Cognitive Assessment (MOCA). This is a test used to screen for mild cognitive impairment. The MOCA includes 11 subtests assessing visuospatial functions, executive function, naming, memory, attention, language, abstraction, delayed recall, and orientation. Each subtest is allotted between 1 and 5 points. CAB Beta subjects completed all subtests of the MOCA. The MOCA is scored as a total of the points earned for correct responses across all subtest scores. One point is added to the total score for individuals with education levels below 12 years, for a maximum score of 31.
7. The modified CANTAB One Touch Stockings of Cambridge (OTS). This is a computerized test assessing planning and working memory (Cambridge Cognition, Bottisham, Cambridge, UK.) For OTS, subjects are shown an image of colored balls stacked inside three ‘stockings’. A second image appears at the same time as the first, showing the same balls stacked in a different order inside the stockings. By imagining moving the balls one at a time (not actually moving them), the subject has to determine the minimum number of moves required to move the balls in the second image to match the first. Subjects respond, indicating the number of moves required, by selecting from response options 1 through 5 that are displayed at the bottom of the computer screen. If an incorrect response is selected, the subject continues attempting to solve the problem, until they determine the correct answer. The 10 trials in this test vary in difficulty, ranging from one to five moves required. The outcome measure is the mean time to reach a correct response (sec), averaged across all 10 trials. Thus, higher values indicated worse performance (longer time to a correct response).
8. Paced Tapping (PTAP). This is a computerized test used to assess timing and psychomotor coordination. Specifically, PTAP assesses the degree to which subjects can tap, using their thumbs in alternation, in a consistent, regular rhythm. Paced Tapping is administered at two rates, a faster 3 Hz rate and a slower 1.8 Hz rate, in two separate blocks of trials, with the order of the two blocks counterbalanced. A trial begins by presenting a series of tones, paced at 3 Hz or 1.8 Hz. Subjects are instructed to listen to the tone, and when ready, to begin tapping in time with the tone. Once the subject has tapped 11 times, the tone is discontinued, and subjects are to attempt to continue tapping at that same pace for 32 additional taps. Four trials are administered at each tapping speed. The main outcome is the consistency of the tapping rate, computed as the reciprocal of the standard deviation of the intertap intervals, using all taps from one or both frequency trials that occur following cessation of the pacing tones. This outcome is selected based on prior studies that demonstrated the reciprocal standard deviation of the intertap intervals is more influenced by HD status than other outcomes, such as the mean intertap interval.
9. Prospective Memory (PROM). This is a brief paper and pencil test assessing the ability to remember to perform an action, without prompting, following a delay. At the beginning of the testing session, subjects are instructed that once they came to a test that used a map, they should select a pen of a particular color from a set of three different colored pens. At each visit, a different colored pen is requested, for example, red at Visit 1, blue at Visit 2, and green at Visit 3. The outcome measure is a binomial variable ‘yes’ or ‘no’ which indicated whether they picked up the correct colored pen at the specified time, without prompting.
10. Speeded Tapping (STAP). This is a computerized test of psychomotor speed and coordination. Using a computer mouse placed in a small stabilizing platform, subjects are asked to place their non-dominant hand on the platform, in a flat position, with their index finger placed over the mouse. After the beginning of the trial is signaled by the sound of a tone, subjects are to tap as quickly as possible for 10 seconds. The end of the trial is also signaled by a tone. Up to four tapping trials are administered, all using the index finger of the non-dominant hand. The outcome measure is the mean of the intertap intervals across taps from all four trials, with higher values indicating worse performance (i.e., slower tapping).
11. Spot the Change (SPOT). This is a computerized test of visuospatial working memory. On each trial of this test, subjects view a display of five randomly placed colored squares for 250 msec (target display). After a 1000 msec delay (blank display), the target display is replaced with a display of five squares in the same locations, but one of the squares is encircled (response display). The non-encircled squares in the response display are the same colors as those in the target display. However, on half of the trials, the encircled square changed color. The subject is asked to respond ‘same’ if the encircled square is the same color, or ‘different’ if the color of the encircled square had changed. Subjects are instructed to respond both as quickly and as accurately as possible. There are up to 32 trials. The main outcome measure is Cowan's k²², the number of correct trials, adjusted for guessing (k=5 ([number correct hits/32]+[number correct rejections/32]−1).
12. Stroop Word Reading. This is a paper and pencil test that assesses processing and psychomotor speed. The Word Reading test is one of three components of the traditional Stroop Color Word Interference test. Subjects are shown a printed page on which the words ‘red’, ‘green’, and ‘blue’ are printed in black ink in a series of rows. They are asked to read the words in order across the rows as quickly as possible. The outcome measure from the test is the number of words correctly read in 45 seconds.
13. Symbol Digit Modalities Test (SDMT). This is a paper and pencil test assessing visuospatial attention, processing speed, and working memory. Subjects viewed a piece of paper that displayed, at the top, a ‘key’ matching a series of 9 symbols with the digits 1 through 9. The remainder of the paper shows a series of rows containing boxes in which the symbols are displayed, but the boxes below the symbols are empty. Subjects are asked to refer to the key to fill in the missing digits across the rows, as quickly and accurately as possible. The outcome measure is the number of items correctly completed in 90 seconds (maximum 110).
14. The Trail Making Test. This is a paper and pencil test assessing visuospatial attention, psychomotor speed, and, flexibility. There are two conditions. For Trail Making Test A, subjects are given a sheet of paper on which the numbers 1 through 25 are displayed across the page. They are asked to connect the numbers, in ascending order, as quickly as possible. For Trail Making Test B, subjects are given a page on which both numbers (1-13) and letters (A-L) are displayed. They are instructed to connect the numbers and letters in alternation, in ascending order (i.e., 1 to A, A to 2, 2 to B, etc.). For each condition, the outcome measure is time to completion, in seconds, with longer times indicating worse performance. Each condition is limited to 300 seconds whether or not the subject had reached the final number. The value 300 is used as the data point for cases in which the subject had failed to complete.
15. The University of Pennsylvania Smell Identification Test (UPSIT). This is a paper and pencil test that assesses olfactory recognition. Test stimuli consisted of cardboard booklets with each page impregnated with a different odor. Each page also included four words that served as response options. To perform the test, the subject has to scratch the odor patch and then select the word that best described the odor. The outcome variable is the number of correctly identified odors out of, for example, 20 to 40 possible odors.

The therapeutic agents, including polynucleotides, polynucleotides in combination with a vector, both viral and non-viral, can be used in the preparation of a medicament for the treatment of HD, where the medicament is administered via intravenous, intramuscular, subcutaneous or intraperitoneal injection.

The therapeutic compositions of the present disclosure are delivered to a “target cell” or “host cell” or “target tissue” and equivalents thereof, which refers to the cell, tissue of the host in which expression of the operatively encoding polynucleotide is sought for treatment of HD.

As disclosed herein, vectors useful in the present disclosure have varying transduction efficiencies. As a result, the viral or non-viral vector transduces more than, equal to, or at least about 10%, 20%, 30%, 40%, 50%, 55%, 60%, 65%, 70%, 75%, 80%, 85%, 90%, 95%, 99%, 100% of the cells of the targeted vascular territory. In some embodiments, more than one vector (viral or non-viral, or combination thereof) can be used simultaneously, or in sequence. This can be used to transfer more than one polynucleotide, and/or target more than one type of cell. Where multiple vectors or multiple agents are used, more than one transduction/transfection efficiency can result.

Any of the compositions or pharmaceutical compositions described herein can be administered in combination with any standard treatment of HD.

Methods of Treating Spinocerebellar ataxia type 3 (SCA3) Spinocerebellar ataxia-type 3 (SCA3), which is also known as Machado-Joseph disease, is an autosomal dominant, progressive neurodegenerative disorder with variable age of onset and severity. SCA3 was originally described in people of Portuguese descent, and in particular from the Azores islands where SCA3 is most prevalent (e.g., the incidence of SCA3 is 1/140 in the small island of Flores) (Sudarsky L., et al., Clin. Neurosci. 1995; 3:17-22). SCA3 was subsequently identified in several other countries and is now considered to be the most common dominantly inherited hereditary ataxia.

Clinically, patients with SCA3 present with progressive gait and limb ataxia, dysarthria and a variable combination of other symptoms including pyramidal signs, dystonia, oculomotor disorders, faciolingual weakness, neuropathy, progressive sensory loss and parkinsonian features. In its more severe forms, SCA3 is characterized by defects in both pyramidal (e.g., motor, somatosensory) and extrapyramidal (e.g., muscle tone) neural functions. Within affected families, this form of ataxia also demonstrates an anticipation effect, which is characterized by an earlier disease onset and more severe symptoms with each new affected generation.

SCA3 are attributable to an unstable and iterative genetic expansion of a (CAG) n tract in the coding region of ATXN3 on chromosome 14q32.1 that encodes a pathogenic poly-glutamine region or tract in the translated ATXN3 protein (Kawaguchi Y., et al., Nature Genet. 1994; 8:221-228). The unstable and iterative expansion of the (CAG) n tract in the coding region of ATXN3 (and the pathogenic poly-glutamine tract encoded thereby) causes an increase in protein misfolding, which results in aggregation and formation of nuclear and cytoplasmic inclusions (Paulson H L, et al., 1997, Neuron 19, 333-344). Misfolded protein aggregates are not only a characteristic of SCA3 and Machado-Joseph disease, but are also a common feature of many other neurodegenerative diseases, including Alzheimer's and Parkinson's diseases.

Disclosed herein are methods of treating SCA3. In one aspect, provided herein is a method of treating SCA3, comprising administering a vector comprising a nucleic acid that encodes trehalose-6-phosphate synthase (OtsA), a nucleic acid that encodes trehalose 6-phosphate phosphatase (OtsB), a nucleic acid that encodes OtsA and OtsB, or a nucleic acid that encodes a trehalose biosynthesis fusion polypeptide, or fragments of any of the above, or variants of any of the above. In another aspect, provided herein is a method of treating SCA3, comprising administering a therapeutically effective amount of trehalose and a vector comprising a nucleic acid that encodes trehalose-6-phosphate synthase (OtsA), a nucleic acid that encodes trehalose 6-phosphate phosphatase (OtsB), a nucleic acid that encodes OtsA and OtsB, or a nucleic acid that encodes a trehalose biosynthesis fusion polypeptide, or fragments of any of the above, or variants of any of the above to a subject in need thereof.

In some cases, the subject has not been previously diagnosed with SCA3.

In some cases, the subject is not being treated for SCA3.

In one aspect of the disclosure, the therapeutic composition to treat a vector comprising a nucleic acid that encodes trehalose-6-phosphate synthase (OtsA), a nucleic acid that encodes trehalose 6-phosphate phosphatase (OtsB), a nucleic acid that encodes OtsA and OtsB, or a nucleic acid that encodes a trehalose biosynthesis fusion polypeptide, or fragments of any of the above, or variants of any of the above. In another aspect of the disclosure, the therapeutic composition to treat or reduce symptoms associated with SCA3 includes a polynucleotide encoding a protein capable of indirectly modulating a symptom associated with SCA3.

In a related aspect, the therapeutic compositions of the present disclosure are administered to a subject as a prophylactic or ameliorative modality. As used herein, “ameliorative,” means to improve or relieve a subject of symptoms associated with a disorder, and includes curing such a disorder.

The therapeutic agents, including polynucleotides, polynucleotides in combination with a vector, both viral and non-viral, can be used in the preparation of a medicament for the treatment of SCA3, where the medicament is administered via intravenous, intramuscular, subcutaneous or intraperitoneal injection.

The therapeutic compositions of the present disclosure are delivered to a “target cell” or “host cell” or “target tissue” and equivalents thereof, which refers to the cell, tissue of the host in which expression of the operatively encoding polynucleotide is sought for treatment of SCA3.

As disclosed herein, vectors useful in the present disclosure have varying transduction efficiencies. As a result, the viral or non-viral vector transduces more than, equal to, or at least about 10%, 20%, 30%, 40%, 50%, 55%, 60%, 65%, 70%, 75%, 80%, 85%, 90%, 95%, 99%, 100% of the cells of the targeted vascular territory. In some cases, more than one vector (viral or non-viral, or combination thereof) can be used simultaneously, or in sequence. This can be used to transfer more than one polynucleotide, and/or target more than one type of cell. Where multiple vectors or multiple agents are used, more than one transduction/transfection efficiency can result.

Any of the compositions or pharmaceutical compositions described herein can be administered in combination with any standard treatment of SCA (e.g., SCA3).

### Methods of Treating Amyloidosis

“Amyloidosis” or “amyloid disease” refers to a pathological condition characterized by the presence of amyloid fibers. Amyloid is a generic term referring to a group of diverse but specific protein deposits (intracellular or extracellular) which are seen in a number of different diseases. Though diverse in their occurrence, all amyloid deposits have common morphologic properties, stain with specific dyes (e.g., Congo red), and have a characteristic red-green birefringent appearance in polarized light after staining. They also share common ultrastructural features and common X-ray diffraction and infrared spectra. The term “amyloid-β diseases” includes those diseases, conditions, pathologies, and other abnormalities of the structure or function of the brain, including components thereof, in which the causative agent is amyloid. The area of the brain affected in an amyloid-β disease can be the stroma including the vasculature or the parenchyma including functional or anatomical regions, or neurons themselves. A subject need not have received a definitive diagnosis of a specifically recognized amyloid-β disease.

Local deposition of amyloid is common in the brain, particularly in elderly individuals. The most frequent type of amyloid in the brain is composed primarily of Aβ peptide fibrils, resulting in dementia associated with sporadic (non-hereditary) Alzheimer's disease. In fact, the incidence of sporadic Alzheimer's disease greatly exceeds forms shown to be hereditary. Nevertheless, fibril peptides forming plaques are very similar in both types.

Disclosed herein are methods of treating amyloidosis. In one aspect, provided herein is a method of treating amyloidosis, comprising administering a vector comprising a nucleic acid that encodes trehalose-6-phosphate synthase (OtsA), a nucleic acid that encodes trehalose 6-phosphate phosphatase (OtsB), a nucleic acid that encodes OtsA and OtsB, or a nucleic acid that encodes a trehalose biosynthesis fusion polypeptide, or fragments of any of the above, or variants of any of the above to a subject in need thereof. In another aspect, provided herein is a method of treating amyloidosis, comprising administering a therapeutically effective amount of trehalose and a vector comprising a nucleic acid that encodes trehalose-6-phosphate synthase (OtsA), a nucleic acid that encodes trehalose 6-phosphate phosphatase (OtsB), a nucleic acid that encodes OtsA and OtsB, or a nucleic acid that encodes a trehalose biosynthesis fusion polypeptide, or fragments of any of the above, or variants of any of the above to a subject in need thereof.

In some cases, the subject has not been previously diagnosed with amyloidosis.

In some cases, the subject is not being treated for amyloidosis.

In one aspect of the disclosure, the therapeutic composition to treat amyloidosis includes a vector comprising a nucleic acid that encodes trehalose-6-phosphate synthase (OtsA), a nucleic acid that encodes trehalose 6-phosphate phosphatase (OtsB), a nucleic acid that encodes OtsA and OtsB, or a nucleic acid that encodes a trehalose biosynthesis fusion polypeptide, or fragments of any of the above, or variants of any of the above. In another aspect of the disclosure, the therapeutic composition to treat or reduce symptoms associated with amyloidosis includes a polynucleotide encoding a protein capable of indirectly modulating a symptom associated with amyloidosis.

In a related aspect, the therapeutic compositions of the present disclosure are administered to a subject as a prophylactic or ameliorative modality. As used herein, “ameliorative,” means to improve or relieve a subject of symptoms associated with a disorder, and includes curing such a disorder.

The therapeutic agents, including polynucleotides, polynucleotides in combination with a vector, both viral and non-viral, can be used in the preparation of a medicament for the treatment of amyloidosis, where the medicament is administered via intravenous, intramuscular, subcutaneous or intraperitoneal injection.

The therapeutic compositions of the present disclosure are delivered to a “target cell” or “host cell” or “target tissue” and equivalents thereof, which refers to the cell, tissue of the host in which expression of the operatively encoding polynucleotide is sought for treatment of amyloidosis.

As disclosed herein, vectors useful in the present disclosure have varying transduction efficiencies. As a result, the viral or non-viral vector transduces more than, equal to, or at least about 10%, 20%, 30%, 40%, 50%, 55%, 60%, 65%, 70%, 75%, 80%, 85%, 90%, 95%, 99%, 100% of the cells of the targeted vascular territory. In some cases, more than one vector (viral or non-viral, or combination thereof) can be used simultaneously, or in sequence. This can be used to transfer more than one polynucleotide, and/or target more than one type of cell. Where multiple vectors or multiple agents are used, more than one transduction/transfection efficiency can result.

Any of the compositions or pharmaceutical compositions described herein can be administered in combination with any standard treatment of amyloidosis.

### Methods of Treating Autophagy-Sensitive Symptoms

Also disclosed herein are methods of treating autophagy-sensitive symptoms. For example, autophagy can have an anti-infection effect and can negatively regulate the inflammatory response, which can prevent excessive inflammation from causing tissue damage. Atopic dermatitis, also known as eczema, is associated with excessive inflammation. Administration of any of the compositions or pharmaceutical compositions described herein can limit excessive inflammation. In some cases, administration in intravenous.

Disclosed herein are methods of treating a subject having a disease associated with excessive inflammation comprising administering to the subject a therapeutically effective amount of a vector comprising a nucleic acid that encodes trehalose-6-phosphate synthase (OtsA), a nucleic acid that encodes trehalose 6-phosphate phosphatase (OtsB), a nucleic acid that encodes OtsA and OtsB, or a nucleic acid encoding a trehalose biosynthesis fusion polypeptide. In some cases, the disease associated with excessive inflammation is atopic dermatitis.

Atopic dermatitis is a chronic, systemic disease associated with excessive and/or persistent inflammation. For some patients, eczema can flare up unexpectedly and can include dry skin, cracked skin, itchiness (pruritus), rash on swollen skin, oozing and crusting on the skin, thickened skin, darkening of the skin around the eyes, and raw and/or sensitive skin from scratching or itching.

### Pharmaceutical Compositions

Any of the compositions described herein can be administered as an aqueous or other solvent-based solution. For any of the compositions described herein to be administered as aqueous or other solvent-based dosage forms (e.g., for interstatial administration), a variety of liquid carriers may be used. Aqueous solutions may include salts, buffers, and the like.

Salts are compounds that ionize in aqueous solutions and may be employed, for example, to adjust the tonicity of the solution. If the active agent is present in the form of a salt, additional salts may be added to the composition in order, for example, to effect ion exchange with the active agent. Salts suitable for use with the compositions described herein are known in the art and include, for example, lithium, sodium, potassium, calcium, and magnesium salts having appropriate counterions that may be selected from chloride, bromide, iodide, carbonate, phosphate, nitrate, silicate, sulfate, phosphite, nitrite, sulfite, and the like.

Buffers are compounds or solutions that are employed to aid in maintaining the concentration of an analyte within a desired range. For example, pharmaceutically acceptable pH buffers are used to maintain the acidity or basicity of a solution within a pharmaceutically acceptable range. Buffers for use in the compositions disclosed herein may be any known or hereafter discovered buffer.

Excipients are inactive ingredients that may be employed in the compositions described herein for a variety of reasons. A wide range of excipients are described in the literature (e.g., Rowe et al., Handbook of Pharmaceutical Excipients, McGraw Hill, 2006).

Another aspect of the present disclosure administers an “adjuvant” or equivalents thereof, with any of the compositions disclosed herein. An “adjuvant” to a compound or mixture that enhances the immune response to an antigen. An adjuvant can serve as a tissue depot that slowly releases the antigen and also as a lymphoid system activator that non-specifically enhances the immune response. Adjuvants include, but are not limited to, complete Freund's adjuvant, incomplete Freund's adjuvant, saponin, mineral gels such as aluminum hydroxide, surface active substances such as lysolecithin, pluronic polyols, polyanions, peptides, oil or hydrocarbon emulsions, keyhole limpet hemocyanins, dinitrophenol, and potentially useful human adjuvants such as BCG (bacille Calmette-Guerin) and Corynebacterium parvum. In some embodiments, the adjuvant is pharmaceutically acceptable. In some cases, the adjuvant enhances the immune response without causing life-threatening symptoms.

In a related aspect, the term “molecular adjuvant” is defined as a protein, lipid, nucleic acid, carbohydrate, or chemical compound for which dendritic cells (DCs), macrophages, B cells, T cells, and/or NK cells have a known receptor whose occupancy leads to a defined sequence of intracellular signal transduction and a change in the phenotype resulting in an improvement in the quantity or quality of the ensuing immune response. In a related aspect, the cells as described above are collectively referred to as “immune cells.”

Illustrative pharmaceutical forms suitable for injectable use include sterile aqueous solutions or dispersions and sterile powders for the extemporaneous preparation of sterile injectable solutions or dispersions (for example, see U.S. Pat. No. 5,466,468, incorporated herein by reference). In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and/or by the use of surfactants. The prevention of the action of microorganisms can be facilitated by various antibacterial and antifungal agents, for example, parabens, chlorobutanol, phenol, sorbic acid, thimerosal, and the like. In some embodiments, isotonic agents, for example, sugars or sodium chloride, will be included. Prolonged absorption of the injectable compositions can be brought about by the use in the compositions of agents delaying absorption, for example, aluminum monostearate and gelatin.

The carriers can further comprise any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions. The phrase “pharmaceutically-acceptable” refers to molecular entities and compositions that do not produce an allergic or similar untoward reaction when administered to a human. As is apparent to those skilled in the art in view of the teachings of this specification, an effective amount of viral vector which must be added can be empirically determined. Administration can be effected in one dose, continuously or intermittently throughout the course of treatment. Methods of determining the most effective means and dosages of administration are well known to those of skill in the art and will vary with the viral vector, the composition of the therapy, the target cells, and the subject being treated. Single and multiple administrations can be carried out with the dose level and pattern being selected by the treating physician.

It should be understood that more than one transgene can be expressed by the delivered recombinant virion. Alternatively, separate vectors, each expressing one or more different transgenes, can also be delivered as described herein. Furthermore, it is also intended that the viral vectors delivered by the methods of the present disclosure be combined with other suitable compositions and therapies. For example, where the transgene is under the control of an inducible promoter, certain systemically delivered compounds such as muristerone, ponasteron, tetracyline or aufin may be administered in order to regulate expression of the transgene.

### Administration

Administration is accomplished such that that the maximum endotoxin level is less than 5 EU per kilogram of body weight per hour. In particular aspects, the endotoxin level is less than about 1, 2, 3, or less than about 4 endotoxin units per kilogram of body weight per hour.

Administration can be daily, weekly, biweekly, monthly, yearly, or one in a life time. In some cases, the administration is once in a life time or yearly. The disclosure also encompasses discontinuous dosing schedules. The exact parameters of discontinuous administration schedules will vary according the formulation, method of delivery and the clinical needs of the subject.

Administration of the compositions described herein may be carried out as part of a treatment regimen that may include a single or multiple instances of administration of trehalose-containing compositions as well as administration of other pharmaceutically active compositions. Such a regimen may be designed as a method of treatment for a neurological disorder as described herein, and/or as a method of long-term maintenance of the health of a patient after having been treated for a neurological disorder (e.g., preventing recurrences). The treatment regimen may be designed as a method of treating a subject that is asymptomatic for a neurological disorder, in which the subject that has been genetically diagnosed with a neurological disorder but does not have any symptoms. Such treatment regimen will delay the onset of symptoms associated with the neurological disorder in a subject. It will be appreciated that determination of appropriate treatment regimens is within the skill of practitioners in the art. Administration of the compositions described herein may be carried out using any appropriate mode of administration and dosage form. Administration methods can include any appropriate central nervous system administration method. Exemplary central nervous system administration methods include, but are not limited to, direct injection (e.g., direct injection into the striata or cortex), intrathecal, intraparenchymal, intracisterna (also known as inter cisterna magna or ICM), intracerebroventricular injection (ICV). Administration into the brain can include one or both sides of the brain. In some cases, any of the compositions described herein can be administered intrathecally. In some cases, any of the compositions described herein can be administered intraparenchymlally. In some cases, any of the compositions described herein can be administered intracisternally. In some cases, any of the compositions described herein can be administered through intracerebroventricular injection (ICV). In some cases, for example, when treating a subject having a disorder associated with excessive inflammation, intravenous administration is used.

Any of the compositions or pharmaceutical formulations described herein can be administered in combination with trehalase inhibitors. Trehalase is an enzyme that catabolized trehalose into its monomeric sugars. An exemplary sequence of a trehalase enzyme can be found in UniProt Entry No. 043290. Trehalase inhibitor prevent, or lessen, trehalase activity, resulting in slower or no degradation of trehalose. Non-limiting examples of trehalase inhibitors include alidimycin A, trehazolin, amygdalin; or a combination thereof; or a pharmaceutically acceptable salt thereof.

Some embodiments further comprise administering a trehalase inhibitor as a non-fixed combination with any of the compositions or formulations described herein. In some embodiments, the trehalase inhibitor is administered simultaneously or sequentially in either order with the composition or pharmaceutical formulation described herein.

### Method of Making

### Exemplary Host Cells

AAVs can be made using a host cell, also known as a packaging cell. Any appropriate host cells for AAV production can be used, such as mammalian or insect production cell lines. See, for example, Naso et al. BioDrugs. 2017; 31 (4): 317-334. Host cells can include, but are not limited to, HEK293 human cells, NHK human cells, HeLa human cells, and Sf9 insect cells. Host cells can be cultured in any appropriate medium that results in production of any of the AAVs described herein (e.g., an AAV comprising a nucleic acid that encodes trehalose-6-phosphate synthase (OtsA), a nucleic acid that encodes trehalose 6-phosphate phosphatase (OtsB), a nucleic acid that encodes OtsA and OtsB, or a nucleic acid that encodes a trehalose biosynthesis fusion polypeptide).

The host cell must also be rendered capable of providing nonAAV-derived functions, or “accessory functions”, in order to produce rAAV virions. Accessory functions are nonAAV-derived viral and/or cellular functions upon which AAV is dependent for its replication, including at least those nonAAV proteins and RNAs that are required in AAV replication, including those involved in activation of AAV gene transcription, stage specific AAV mRNA splicing, AAV DNA replication, synthesis of Cap expression products and AAV capsid assembly. Viral-based accessory functions can be derived from any of the known helper viruses.

In particular, accessory functions can be introduced into and then expressed in host cells using methods known to those of skill in the art. Typically, accessory functions are provided by infection of the host cells with an unrelated helper virus. A number of suitable helper viruses are known, including adenoviruses; herpesviruses, e.g., herpes simplex virus types 1 and 2; and vaccinia viruses. Nonviral accessory functions will also find use herein, such as those provided by cell synchronization using any of various known agents. See, e.g., Buller et al. (1981) J. Virol. 40:241 247; McPherson et al. (1985) Virology 147:217 222; Schlehofer et al. (1986) Virology 152:110 117.

Alternatively, accessory functions can be provided using an accessory function vector as defined above. See, e.g., U.S. Pat. No. 6,004,797 and International Publication No. WO 01/83797, each of which is incorporated herein by reference in its entirety. Nucleic acid sequences providing the accessory functions can be obtained from natural sources, such as from the genome of an adenovirus particle, or constructed using recombinant or synthetic methods known in the art. Also, the full-complement of adenovirus genes is not required for accessory helper functions. In fact, adenovirus mutants incapable of DNA replication and late gene synthesis have been shown to be permissive for AAV replication. Ito et al., (1970) J. Gen. Virol. 9:243; Ishibashi et al, (1971) Virology 45:317; and Carter et al., (1983) Virology 126:505. For example, reports show that E1A and E4 regions are likely required for AAV replication, either directly or indirectly. Laughlin et al., (1982) J. Virol 41:868; Janik et al., (1981) Proc. Natl. Acad. Sci. USA 78:1925; Carter et al., (1983) Virology 126:505. In addition, International Publication WO 97/17458 and Matshushita et al., (1998) Gene Therapy 5:938 945, describe accessory function vectors encoding various adenoviral genes.

Infection of the host cell with a helper virus, or transfection of the host cell with an accessory function vector, allows expression of the accessory functions which transactivate the AAV helper construct to produce AAV Rep and/or Cap proteins. The Rep expression products in turn excise the recombinant DNA (including the DNA of interest, e.g., a nucleic acid that encodes trehalose-6-phosphate synthase (OtsA), a nucleic acid that encodes trehalose 6-phosphate phosphatase (OtsB), a nucleic acid that encodes OtsA and OtsB, or a nucleic acid that encodes a trehalose biosynthesis fusion polypeptide) from the AAV expression vector. The Rep proteins also serve to duplicate the AAV genome. The expressed Cap proteins assemble into capsids, and the recombinant AAV genome is packaged into the capsids, AAV replication proceeds, and the DNA is packaged into rAAV virions.

Following recombinant AAV replication, rAAV virions can be purified from the host cell using a variety of conventional purification methods, such as column chromatography, CsCl gradients, and the like. For example, a plurality of column purification steps can be used, such as purification over an anion exchange column, an affinity column and/or a cation exchange column. See, for example, International Publication No. WO 02/12455. Further, if infection is employed to express the accessory functions, residual helper virus can be inactivated, using known methods. For example, adenovirus can be inactivated by heating to temperatures of approximately 60° C. for, e.g., 20 minutes or more. This treatment effectively inactivates only the helper virus since AAV is extremely heat stable while the helper adenovirus is heat labile. The resulting rAAV virions containing a nucleic acid that encodes trehalose-6-phosphate synthase (OtsA), a nucleic acid that encodes trehalose 6-phosphate phosphatase (OtsB), a nucleic acid that encodes OtsA and OtsB, or a nucleic acid that encodes a trehalose biosynthesis fusion polypeptide, or fragment, or functional fragment, or portion thereof can then be used for gene delivery using the techniques described below.

### Exemplary Methods of Purification

Methods to separate produced AAVs from the cellular debris of the host cell. See, for example, Naso et al. BioDrugs. 2017; 31 (4): 317-334. For example, various affinity resins and/or ion exchange chromatography can be used to separate produced AA Vs from cellular debris. An exemplary method can include using a columns with carbohydrates, such as a heparin column, because AAVs use cell membrane-associated carbohydrates as the primary cell receptor for transduction. Methods are also known to separate loaded AAVs (AAVs that contain the transgene) from empty AAVs (AAVs that lack the transgene).

### Definitions

In order that the present disclosure can be more readily understood, certain terms are first defined. As used in this application, except as otherwise expressly provided herein, each of the following terms shall have the meaning set forth below. Additional definitions are set forth throughout the application.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure. For purposes of the present disclosure, the following terms are defined.

The practice of the present disclosure will employ, unless indicated specifically to the contrary, conventional methods of virology, immunology, microbiology, molecular biology and recombinant DNA techniques within the skill of the art, many of which are described below for the purpose of illustration. Such techniques are explained fully in the literature. See, e.g., Sambrook, et al. Molecular Cloning: A Laboratory Manual (2nd Edition, 1989); Maniatis et al. Molecular Cloning: A Laboratory Manual (1982); DNA Cloning: A Practical Approach, vol. I & II (D. Glover, ed.); Oligonucleotide Synthesis (N. Gait, ed., 1984); Nucleic Acid Hybridization (B. Hames & S. Higgins, eds., 1985); Transcription and Translation (B. Hames & S. Higgins, eds., 1984); Animal Cell Culture (R. Freshney, ed., 1986); Perbal, A Practical Guide to Molecular Cloning (1984).

Units, prefixes, and symbols are denoted in their Système International de Unites (SI) accepted form. Numeric ranges are inclusive of the numbers defining the range. The headings provided herein are not limitations of the various aspects of the disclosure, which can be had by reference to the specification as a whole. Accordingly, the terms defined immediately below are more fully defined by reference to the specification in its entirety.

The terms “a,” “an,” or “the” as used herein not only include aspects with one member, but also include aspects with more than one member. For instance, the singular forms “a,” “an,” and “the” include plural referents unless the context clearly dictates otherwise. Thus, for example, reference to “a cell” includes a plurality of such cells and reference to “the agent” includes reference to one or more agents known to those skilled in the art, and so forth.

The term “or” as used herein should in general be construed non-exclusively. For example, a claim to “a composition comprising A or B” would typically present an aspect with a composition comprising both A and B. “Or” should, however, be construed to exclude those aspects presented that cannot be combined without contradiction (e.g., a composition pH that is between 9 and 10 or between 7 and 8).

The group “A or B” is typically equivalent to the group “selected from the group consisting of A and B.”

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

The terms “about” and “approximately” as used herein shall generally mean an acceptable degree of error for the quantity measured given the nature or precision of the measurements. Typical, exemplary degrees of error are within 20 percent (%), within 10%, or within 5% of a given value or range of values. Any reference to “about X” specifically indicates at least the values X, 0.95X, 0.96X, 0.97X, 0.98X, 0.99X, 1.01X, 1.02X, 1.03X, 1.04X, and 1.05X. Thus, “about X” is intended to provide written description support for a claim limitation of, e.g., “0.98X.” The terms “about” and “approximately,” particularly in reference to a given quantity, encompass and describe the given quantity itself.

“Treatment” or “therapy” of a subject refers to any type of intervention or process performed on, or the administration of an active agent to, the subject with the objective of reversing, alleviating, ameliorating, inhibiting, or slowing down, the onset, progression, development, severity, or recurrence of a symptom, complication, condition, or biochemical indicia associated with a disease.

As used herein, “alleviate” or “alleviation,” refer to reduction, in whole or in part, of symptoms associated with any of the neurodegenerative disorders described herein.

The term “prophylactic” or “prophylactically” refers to any type of intervention or process performed on, or the administration of an active agent to, the subject with the objective of protecting or preventing a disease or condition from developing or at least not developing fully (e.g., to reduce the symptoms or severity of the disease or condition) such as in the development of a side effect.

“Administering” or “administration” refer to the physical introduction of a therapeutic agent to a subject, using any of the various methods and delivery systems known to those skilled in the art. Routes of administration can include oral, intravenous, intramuscular, subcutaneous, intraperitoneal, spinal or other parenteral routes of administration, for example by injection or infusion (e.g., intravenous infusion). Administration can also be performed, for example, once, a plurality of times, and/or over one or more extended periods.

A “subject” includes any human or non-human animal. Non-human animals include, but is not limited to, vertebrates such as non-human primates, sheep, dogs, and rodents such as mice, rats, and guinea pigs. In some embodiments, the subject is a human. In some embodiments, the subject has experienced and/or exhibited at least one symptom of the neurodegenerative disorder to be treated. The terms “subject” and “patient” and “individual” are used interchangeably herein.

An “effective amount” or “therapeutically effective amount” or “therapeutically effective dosage” of a drug or therapeutic agent is any amount of the drug that, when used alone or in combination with another therapeutic agent, slowing down the onset of a disease or promotes disease regression evidenced by a decrease in severity of disease symptoms, an increase in frequency and duration of disease symptom-free periods, or a ameliorating an impairment or disability due to the disease affliction. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

The phrase “pharmaceutically acceptable” indicates that the substance or composition must be compatible chemically and/or toxicologically, with the other ingredients comprising a formulation, and/or the mammal being treated therewith.

### EXAMPLES

### Example 1: Exemplary AAV Constructs for Treating Neurodegenerative Diseases

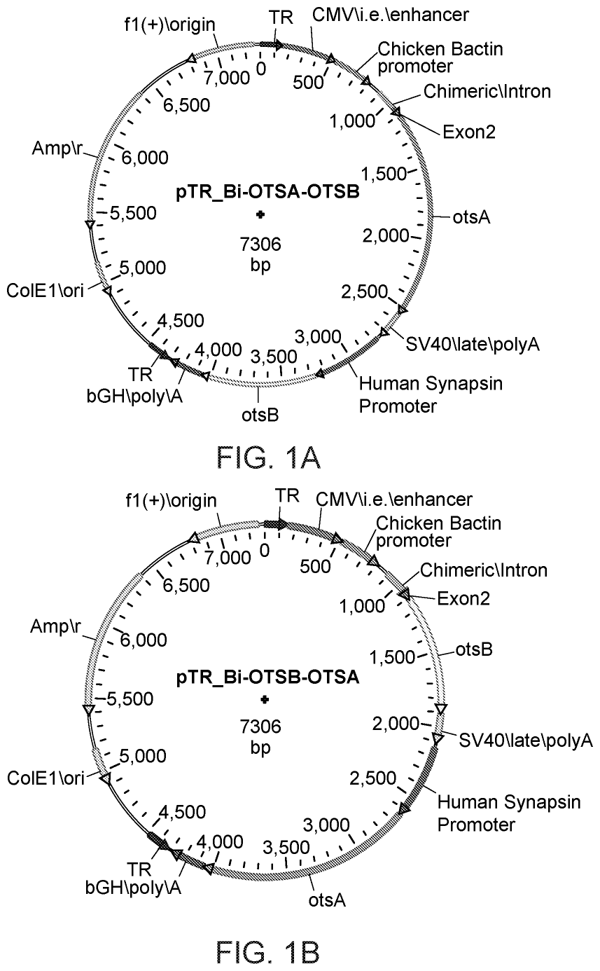

Exemplary constructs that produced OtsA polypeptide, an OtsB polypeptide, a combination thereof, or a trehalose biosynthesis fusion polypeptide (FIG. 1I) in AAV vectors are shown in FIGS. 1A-1G and described in Table 1B. Vectors are designed to express nucleic acids encoding OtsA and/or OtsB, either as separate proteins or as fusion proteins that can be cleaved during translation processing.

TABLE 1b
Exemplary constructs.

| Figure | Plasmid | AAV Vector |
|---|---|---|
| FIG. 1A | pTR_BiOTSA-OTSB or AAV1-otsA-otsB-T2A- | AAV1 |
| | mCherry | |
| FIG. 1B | pTR_BiOTSB-OTSA | AAV1 |
| | or AAV1-otsB-otsA-T2A-mCherry | |
| FIG. 1C | pBK1802-AAV9-CAG-otsA-P2A-otsB-T2A- | AAV9 |
| | mCherry-WPRE | |
| FIG. 1E | pBK1928-AAV9-otsA-P2A-otsB | AAV9 |
| FIG. 1F | pBK19360otsB-IRES-OtsA | AAV9 |
| FIG. 1G | AAV5-otsAB-t2A-mCherry | AAV5 |

### Example 2: Safety and Biodistribution of AAV1-otsA-otsB-T2A-mCherry in the Brain and Spinal Cord of C57BL/6 Mice

This example examined the biodistribution of AAV1-otsA-otsB-T2A-mCherry in the brain and spinal cord of C57BL/6 mice. For this purpose, mice were bilaterally injected with different titers of AAV1-otsA-otsB-T2A-mCherry into the striatum (STR). Two, four, or twelve weeks post-injection, brains and spinal cords were collected and shipped to CRL-Kuopio to measure mCherry distribution. Whole blood, plasma, serum, brain, liver, kidney, quadricep muscles, and small intestines were also collected for further analyses.

### Materials and Methods

Animals—Thirty-six adult C57BL/6 mice (Vendor: CRL, CRL-provided) were used in the study. Animals were individually-housed (males) or group-housed (females) and had access to food and water ad libitum. Animals were kept on a 12/12 hour light/dark cycle.
Formulation and Dosing—Test articles were stored at −80° C. until use. On the day of usage, test compounds were kept on ice when not loading into the infusion syringes. Formulations are described in Table 2. Animals were divided into treatment groups as described below in Table 3.

TABLE 2
Test Articles

| Test Article | Stock titer | Dosing Titer | Lot/Batch | Formulation |
|---|---|---|---|---|
| AAV1-otsA-otsB- | 0.5E+12 | 0.5E+12 | 68121 | As supplied |
| T2A-mCherry | GC/mL | GC/mL | | |
| AAV1-otsA-otsB- | 1.0E+12 | 1.0E+12 | 68121 | As supplied |
| T2A-mCherry | GC/mL | GC/mL | | |
| AAV1-otsA-otsB- | 2.0E+12 | 2.0E+12 | 68121 | As supplied |
| T2A-mCherry | GC/mL | GC/mL | | |

TABLE 3
Treatment Groups and Infusion Setting.

| | | | | | Injec- | |
|---|---|---|---|---|---|---|
| | Group | Test | Dosing | Route/ | tion | End- |
| | size | Article | titer | brain | volume | point |
| Group | (n) | (TA) | (GC/mL) | area | (μL) | (weeks) |
| 1 | 4 | AAV1-otsA- | 0.5E12 | Bilat- | 3/STR | 2 |
| | | otsB-T2A- | | eral | | |
| | | mCherry | | STR | | |
| 2 | 4 | AAV1-otsA- | 1.0E12 | Bilat- | 3/STR | 2 |
| | | otsB-T2A- | | eral | | |
| | | mCherry | | STR | | |
| 3 | 4 | AAV1-otsA- | 2.0E12 | Bilat- | 3/STR | 2 |
| | | otsB-T2A- | | eral | | |
| | | mCherry | | STR | | |
| 4 | 4 | AAV1-otsA- | 0.5E12 | Bilat- | 3/STR | 4 |
| | | otsB-T2A- | | eral | | |
| | | mCherry | | STR | | |
| 5 | 4 | AAV1-otsA- | 1.0E12 | Bilat- | 3/STR | 4 |
| | | otsB-T2A- | | eral | | |
| | | mCherry | | STR | | |
| 6 | 4 | AAV1-otsA- | 2.0E12 | Bilat- | 3/STR | 4 |
| | | otsB-T2A- | | eral | | |
| | | mCherry | | STR | | |
| 7 | 4 | AAV1-otsA- | 0.5E12 | Bilat- | 3/STR | 12 |
| | | otsB-T2A- | | eral | | |
| | | mCherry | | STR | | |
| 9 | 4 | AAV1-otsA- | 2.0E12 | Bilat- | 3/STR | 12 |
| | | otsB-T2A- | | eral | | |
| | | mCherry | | STR | | |

Surgery—Mice were infused bilaterally with 3 μL/injection of test compound into the STR (1 injection/site) at a rate of 0.2 μL/min (injection over 15 min/site), with a 5 min rest period following injection.

Pre and post-operative care: Mice were anesthetized using Isoflurane (2%, 800 mL/min O2). Lidocaine/bupivacaine was used for local analgesia and carprofen was used for peri-/post-operative analgesia. Carprofen (5 mg/kg s.c) was administered before the surgery and Carprofen (0.068 mg/mL) was provided in the drinking water to animals for 3 days after surgery. Animals were carefully monitored by laboratory personnel on the day of surgery and once daily for additional 3 days after surgery.

The animals were placed in a stereotaxic frame (Kopf instruments, USA). The following coordinates were used for the STR (AP+0.8 mm, lateral+/−1.8 mm, DV −3.0 mm from dura).

Utilizing a Hamilton syringe (model #80308; 10 μL syringe with corresponding 30 gage blunt tip needle) and the stereotactic micromanipulator, the location of the burr holes were designated and drilled (0.9 mm burr, Fine Science Tools, Item #19007-09). The drill was only used to penetrate the bone (~1 mm). The dura was punctured with a 26 ga bevel needle, and the infusion needle was lowered into the brain to the depth of the desired location. The injection settings for the ultra-micropump III with micro4 controller (World Precision Instruments) were: injection volume: 3 μL; injection rate: 0.2 μL/min. Saline was put on the top of the skull then the needle was slowly lowered (over approximately 1 minute) to DV-0.1 mm below the infusion coordinates (−3.1 mm and then raise it to DV-3.0 mm). The needle was allowed to equilibrate for 1 min. The volume in the syringe was noted and injection started by pressing RUN button. Volume in the syringe was checked after completion of the run. When delivery was completed, there was a 5 min wait before withdrawal of the needle such that it took approximately 1 min to be fully withdrawn. It was verified that the liquid was still able to flow from the syringe needle. Once the infusion was complete, the burr holes were covered with bone wax (Medline DYNJBW25) and the incision was closed with skin glue. Subjects underwent health checks and had body weights taken on study days 1, 2, 3, and then weekly until the end of the study (See Table 4).

TABLE 4
Body weights over the study period.

| | Injection | 1 Day | 2 Day | 3 Day | 1 Wk | 2 Wk | 3 Wk | 4 Wk |
|---|---|---|---|---|---|---|---|---|
| TX | Weight | Weight | Weight | Weight | Weight | Weight | Weight | Weight |
| Grp | (g) | (g) | (g) | (g) | (g) | (g) | (g) | (g) |
| 1 | 22.8 | 22.5 | 22.3 | 22.4 | 22.9 | 23.3 | N/A | N/A |
| 2 | 20.2 | 19.3 | 19.8 | 20.3 | 20.5 | 21.2 | N/A | N/A |
| 3 | 23.2 | 23.3 | 23.6 | 23.8 | 24 | 24.4 | N/A | N/A |
| 4 | 21.5 | 21.5 | 21.5 | 21.9 | 22.3 | 21.8 | 22.3 | 22.3 |
| 5 | 21.8 | 22.2 | 22.4 | 22.6 | 22.6 | 23.3 | 24.6 | 25.6 |
| 6 | 23.2 | 21.5 | 21.3 | 21.8 | 22.7 | 22.7 | 23.7 | 24 |
| 7 | 21.1 | 21.8 | 21.5 | 21.6 | 22 | 21.4 | 21.4 | 23.6 |
| 9 | 24.4 | 24.6 | 25.1 | 25.2 | 25.7 | 26.2 | 27 | 26.7 |
| 1 | 21.6 | 22.4 | 22.7 | 22.8 | 22.2 | 22.1 | N/A | N/A |
| 2 | 22.3 | 22.6 | 23.3 | 23.1 | 23.1 | 23.5 | N/A | N/A |
| 3 | 21.8 | 22 | 22.1 | 22 | 22.3 | 22.8 | N/A | N/A |
| 4 | 23.3 | 23.8 | 23.8 | 24.1 | 24.1 | 24.4 | 25.5 | 26.7 |
| 5 | 23 | 22.8 | 23.5 | 23.3 | 23.8 | 24.2 | 24.6 | 24.96 |
| 6 | 21.1 | 21 | 21.5 | 21.5 | 22.3 | 22.2 | 23.4 | 23.7 |
| 7 | 23.2 | 23.9 | 24 | 23.8 | 23.9 | 25 | 26.5 | 27.4 |
| 9 | 24.2 | 24.5 | 25.1 | 25.2 | 26 | 26.8 | 28.5 | 28.9 |
| 1 | 23.5 | 23 | 23.2 | 23.6 | 23.9 | 22.1 | N/A | N/A |
| 2 | 22.4 | 21.4 | 21.6 | 21.8 | 22.4 | 23.5 | N/A | N/A |
| 3 | 20.9 | 21.4 | 21.5 | 21.1 | 21.3 | 22.8 | N/A | N/A |
| 4 | 22.5 | 22.4 | 22.5 | 22.8 | 22.7 | 22.7 | 23.9 | 24.8 |
| 5 | 22 | 21.8 | 21.6 | 21.4 | 22.1 | 22.8 | 23 | 23.9 |
| 6 | 23.4 | 24.1 | 24.3 | 24 | 25.2 | 25.3 | 26.1 | 26.4 |
| 7 | 21.2 | 20.6 | 21.5 | 21.5 | 22.2 | 22.6 | 23.4 | 24.4 |
| 9 | 20.9 | 20.5 | 20.4 | 20.5 | 21.6 | 22.2 | 22.3 | 22.8 |
| 1 | 24.8 | 24.4 | 25 | 24.7 | 26.3 | 24.2 | N/A | N/A |
| 2 | 20.5 | 20.8 | 20.8 | 21.1 | 21.8 | 23.7 | N/A | N/A |
| 3 | 22.2 | 22.1 | 22.7 | 22.3 | 22.7 | 22.3 | N/A | N/A |
| 4 | 22 | 21.7 | 22.2 | 22.1 | 22.4 | 22.4 | 23.6 | 24.3 |
| 5 | 21.4 | 20 | 21.6 | 21.9 | 22.4 | 22.5 | 23.6 | 24.4 |
| 6 | 21 | 21.4 | 21.4 | 20.8 | 21.6 | 22.1 | 23 | 23.7 |
| 9 | 22.1 | 22.2 | 22.4 | 22.3 | 22.7 | 23.3 | 24.4 | 25.5 |
| 7 | 23.4 | 23 | 23.6 | 23.8 | 24.9 | 24.7 | 26.1 | 28.1 |

| | 5 Wk | 6 Wk | 7 Wk | 8 Wk | 9 Wk | 10 Wk | 11 Wk | 12 Wk |
|---|---|---|---|---|---|---|---|---|
| TX | Weight | Weight | Weight | Weight | Weight | Weight | Weight | Weight |
| Grp | (g) | (g) | (g) | (g) | (g) | (g) | (g) | (g) |
| 1 | N/A | N/A | N/A | N/A | N/A | N/A | N/A | N/A |
| 2 | N/A | N/A | N/A | N/A | N/A | N/A | N/A | N/A |
| 3 | N/A | N/A | N/A | N/A | N/A | N/A | N/A | N/A |
| 4 | N/A | N/A | N/A | N/A | N/A | N/A | N/A | N/A |
| 5 | N/A | N/A | N/A | N/A | N/A | N/A | N/A | N/A |
| 6 | N/A | N/A | N/A | N/A | N/A | N/A | N/A | N/A |
| 7 | 23.6 | 25 | 24.9 | 25.2 | 24.5 | 25.7 | 26.9 | 26.4 |
| 9 | 27 | 28.3 | 28.2 | 29.2 | 28.6 | 29 | 29.36 | 30.1 |
| 1 | N/A | N/A | N/A | N/A | N/A | N/A | N/A | N/A |
| 2 | N/A | N/A | N/A | N/A | N/A | N/A | N/A | N/A |
| 3 | N/A | N/A | N/A | N/A | N/A | N/A | N/A | N/A |
| 4 | N/A | N/A | N/A | N/A | N/A | N/A | N/A | N/A |
| 5 | N/A | N/A | N/A | N/A | N/A | N/A | N/A | N/A |
| 6 | N/A | N/A | N/A | N/A | N/A | N/A | N/A | N/A |
| 7 | 28.2 | 29.7 | 30.6 | 33.2 | 33 | 33.8 | 35 | 34.5 |
| 9 | 28.7 | 29.9 | 30.9 | 32.2 | 31.5 | 32.8 | 33.4 | 34.2 |
| 1 | N/A | N/A | N/A | N/A | N/A | N/A | N/A | N/A |
| 2 | N/A | N/A | N/A | N/A | N/A | N/A | N/A | N/A |
| 3 | N/A | N/A | N/A | N/A | N/A | N/A | N/A | N/A |
| 4 | N/A | N/A | N/A | N/A | N/A | N/A | N/A | N/A |
| 5 | N/A | N/A | N/A | N/A | N/A | N/A | N/A | N/A |
| 6 | N/A | N/A | N/A | N/A | N/A | N/A | N/A | N/A |
| 7 | 24 | 25.1 | 26.5 | 27.7 | 27 | 28.2 | 28.7 | 30.1 |
| 9 | 22.5 | 23.3 | 23.3 | 24.4 | 23.2 | 24 | 23.8 | 23.3 |
| 1 | N/A | N/A | N/A | N/A | N/A | N/A | N/A | N/A |
| 2 | N/A | N/A | N/A | N/A | N/A | N/A | N/A | N/A |
| 3 | N/A | N/A | N/A | N/A | N/A | N/A | N/A | N/A |
| 4 | N/A | N/A | N/A | N/A | N/A | N/A | N/A | N/A |
| 5 | N/A | N/A | N/A | N/A | N/A | N/A | N/A | N/A |
| 6 | N/A | N/A | N/A | N/A | N/A | N/A | N/A | N/A |
| 9 | 25.3 | 25.7 | 26.9 | 28.4 | 29.5 | 29.2 | 30 | 29.3 |
| 7 | 28.2 | 29.8 | 31.4 | 32.6 | 33.2 | 34.1 | 35.3 | 34.5 |

Prior to injections, Hamilton syringes were pre-conditioned with the associated infusion needles for 3 min with solution before infusing compounds. Prior to penetrating brain with injection needle, a minimal amount of solution was ejected from the syringe to visually confirm that injection needle was not obstructed.

Plasma Serial Sampling—Animals from groups 7-9 underwent serial blood sampling according to Table 5.

TABLE 5
Plasma Serial Blood Sampling.

| Time post | | |
|---|---|---|
| (weeks) | Procedure | Storage |
| 11 weeks | Collect blood (150 μL/timepoint) from the lateral | −80° C. |
| and 12 | tail vein into K2 EDTA tubes | |
| weeks | Keep on ice | |
| | Process within 30-min of collection for plasma | |
| | (centrifugation at 4° C., 2,000 g for 10 min) | |
| | Transfer into 2 mL polypropylene screw top vials | |
| | Immediately add 2.5% formic acid (in UP) for 8:2 | |
| | plasma:FA ratio | |
| | Record sample volume, freeze and store at −80° C. | |

Tissue Collection—Approximately two, four, and twelve weeks after injection, animals were deeply anesthetized with isoflurane. Blood was collected via cardiac puncture. Then animals underwent a cold heparinized saline (heparin 2.5 IU/mL) transcardial perfusion followed immediately by liver collection. Then animals (groups 1-6 only) were perfused with cold 4% PFA (made fresh daily). Groups 9-12 were perfused with cold heparinized saline only, with no 4% PFA perfusion. Tissues were collected in the following order and shipped to CRL-Kuopio or third party for analysis. Post-mortem tissue collection is summarized in Table 6.

TABLE 6
Post-mortem Tissue Collection.

| | | # | | | | |
|---|---|---|---|---|---|---|
| Tissue | Group | Samples | Procedure | Storage | | Assay |
| Terminal | 1-9 | 36 | After anesthesia with isoflurane: | −80° | C. | q-PCR for |
| Whole | | | Collected as much blood as possible | | | WPRE2 |
| Blood | | | via cardiac puncture | | | |
| | | | Place ~300 μL of whole blood into K2 | | | |
| | | | EDTA tubes (prechilled on ice) | | | |
| | | | Aliquoted 290 μL of whole blood into | | | |
| | | | 1.5 mL tube filled with 800.4 μL | | | |
| | | | PAXgene Blood RNA tubes | | | |
| | | | (cat#762165) and gently inverted several | | | |
| | | | times | | | |
| | | | Froze and stored at −80° C. until | | | |
| | | | shipment for further processing | | | |
| Terminal | 1-9 | 36 | After aliquoting out whole blood: | −80° | C. | 1.100 μL |
| Serum | | | Placed rest of blood into scrum | | | for AAV1 |
| | | | separator vials and gently invert several | | | Nab |
| | | | times | | | 2.50 μL |
| | | | Kept at room temperature for 30 min | | | for |
| | | | Processed for serum (centrifugation at | | | Trehalose |
| | | | 4° C., 10,000 g for 5 min) | | | |
| | | | Aliquoted 100 μL of serum into 1.4 | | | |
| | | | mL tubes in 96 tube rack (USA | | | |
| | | | Scientific cat#17742003) | | | |
| | | | Aliquoted 50 μL of serum into 1.4 mL | | | |
| | | | tubes in 96 tube rack (USA Scientific | | | |
| | | | cat#17742003) | | | |
| | | | Aliquoted rest of serum into 1.4 mL | | | |
| | | | tubes in 96 tube rack (USA Scientific | | | |
| | | | cat#17742003) | | | |
| | | | Recorded estimated serum volume, | | | |
| | | | froze and stored in at −80° C. until | | | |
| | | | shipment for further processing | | | |
| Median | 1-9 | 36 | After heparinized saline perfusion: | 4° C./−80° | | q-PCR for |
| Liver Lobe | | | Dissected out ~50 mg of tissue (max | C. | | WPRE2 |
| | | | 50 mg) from median liver lobe into pre- | | | |
| | | | weighed 2 mL round bottom safe-locked | | | |
| | | | tubes | | | |
| | | | Recorded tissue weight and store at | | | |
| | | | 4° C. | | | |
| | | | Add 0.5 mL RNAlater | | | |
| | | | 24 hrs later, removed RNAlater | | | |
| | | | supernatant | | | |
| | | | Froze and stored at −80° C. until | | | |
| | | | shipment for further processing | | | |
| Right | 1-9 | 36 | After heparinized saline perfusion: | 4° C./−80° | | q-PCR for |
| Liver Lobe | | | Dissected out ~50 mg of tissue (max | C. | | WPRE2 |
| | | | 50 mg) from right liver lobe into pre- | | | |
| | | | weighed 2 mL round bottom safe-locked | | | |
| | | | tubes | | | |
| | | | Recorded tissue weight and store at | | | |
| | | | 4° C. | | | |
| | | | Add 0.5 mL RNAlater | | | |
| | | | 24 hrs later, removed RNAlater | | | |
| | | | supernatant | | | |
| | | | Froze and stored at −80° C. until | | | |
| | | | shipment for further processing | | | |
| Left Liver | 1-9 | 36 | After heparinized saline perfusion: | 4° C./−80° | | q-PCR for |
| Lobc | | | Dissected out ~50 mg of tissue (max | C. | | WPRE2 |
| | | | 50 mg) from left liver lobe into pre- | | | |
| | | | weighed 2 mL round bottom safe-locked | | | |
| | | | tubes | | | |
| | | | Recorded tissue weight and store at | | | |
| | | | 4° C. | | | |
| | | | Add 0.5 mL RNAlater | | | |
| | | | 24 hrs later, removed RNAlater | | | |
| | | | supernatant | | | |
| | | | Froze and stored at −80° C. until | | | |
| | | | shipment for further processing | | | |
| Caudate | 1-9 | 36 | After heparinized saline perfusion: | 4° C./−80° | | q-PCR for |
| Liver Lobe | | | Dissected out ~50 mg of tissue (max | C. | | WPRE2 |
| | | | 50 mg) from caudate liver lobe into pre- | | | |
| | | | weighed 2 mL round bottom safe-locked | | | |
| | | | tubes | | | |
| | | | Recorded tissue weight and store at | | | |
| | | | 4° C. | | | |
| | | | Add 0.5 mL RNAlater | | | |
| | | | 24 hrs later, removed RNAlater | | | |
| | | | supernatant | | | |
| | | | Froze and stored at −80° C. until | | | |
| | | | shipment for further processing | | | |
| Right | 7-9 | 12 | After heparinized saline perfusion: | −80° | C. | Trehalose |
| Kidney | | | Dissected out right kidney into 2 mL | | | |
| | | | vial | | | |
| | | | Recorded tissue weight, froze and | | | |
| | | | stored at −80° C. until shipment to third | | | |
| | | | party | | | |
| Left | 7-9 | 12 | After heparinized saline perfusion: | −80° | C. | Trehalose |
| Kidney | | | Dissected out left kidney into 2 mL | | | |
| | | | vial | | | |
| | | | Recorded tissue weight, froze and | | | |
| | | | stored at −80° C. until shipment to third | | | |
| | | | party | | | |
| Right | 7-9 | 12 | After heparinized saline perfusion: | −80° | C. | Trehalose |
| Quadriceps | | | Dissected out right quadricep muscles | | | |
| | | | into 2 mL vial | | | |
| | | | Recorded tissue weight, froze and | | | |
| | | | stored at −80° C. until shipment to third | | | |
| | | | party | | | |
| Left | 7-9 | 12 | After heparinized saline perfusion: | −80° | C. | Trehalose |
| Quadriceps | | | Dissected out left quadricep muscles | | | |
| | | | into 2 mL vial | | | |
| | | | Recorded tissue weight, froze and | | | |
| | | | stored at −80° C. until shipment to third | | | |
| | | | party | | | |
| Small | 7-9 | 12 | After heparinized saline perfusion: | −80° | C. | Trehalose |
| Intestines | | | Dissected out small intestine (no | | | |
| (2 | | | flushing) and divided into two 5 mL | | | |
| aliquots) | | | vials | | | |
| | | | Record tissue weights, froze and stored | | | |
| | | | at −80° C. until shipment to third party | | | |
| Left | 7-9 | 12 | After heparinized saline perfusion: | −80° | C. | Trehalose |
| Striatum | | | Dissected left striatum into 2 mL vial | | | |
| | | | Record tissue weights, froze and stored | | | |
| | | | at −80° C. until shipment to third party | | | |
| Rest of | 7-9 | 12 | After heparinized saline perfusion: | −80° | C. | Trehalose |
| Left | | | Divided remaining left hemi brain into | | | |
| Hemibrain | | | two 2 mL vials | | | |
| (2 | | | Recorded tissue weights, froze and | | | |
| aliquots) | | | stored at −80° C. until shipment to third | | | |
| | | | party | | | |
| Right | 7-9 | 12 | After heparinized saline perfusion: | 4° | C. | IHC |
| Hemibrain | | | Extracted right hemibrain and place in | | | |
| (drop-fix) | | | a 50 mL tube with 25 mL 4% PFA at 4° C. | | | |
| | | | 24 hrs later, washed twice with PBS | | | |
| | | | and once with 70% EtOH | | | |
| | | | Placed in 70% EtOH in 5 mL vials | | | |
| | | | Stored at 4° C. until shipment for further | | | |
| | | | processing | | | |
| Cervical | 7-9 | 12 | After heparinized saline perfusion: | 4° | C. | IHC |
| Spinal | | | Dissected out cervical portion of spinal | | | |
| cord | | | cord (10 mm) | | | |
| | | | Positioned spinal cord into cassette | | | |
| | | | between precut foams) | | | |
| | | | Placed cassettes in container with | | | |
| | | | 4% PFA at 4° C. | | | |
| | | | 24 hrs later, washed twice with PBS | | | |
| | | | and once with 70% EtOH | | | |
| | | | Placed in 70% EtOH | | | |
| | | | Stored at 4° C. until shipment for further | | | |
| | | | processing | | | |
| Thoracic | 7-9 | 12 | After heparinized saline: | 4° | C. | IHC |
| Spinal | | | Dissect out thoracic portion of spinal | | | |
| cord | | | cord (20 mm) | | | |
| | | | Position spinal cord into cassette | | | |
| | | | between precut foams) | | | |
| | | | Placed cassettes in container with | | | |
| | | | 4% PFA at 4° C. | | | |
| | | | 24 hrs later, washed twice with PBS | | | |
| | | | and once with 70% EtOH | | | |
| | | | Placed in 70% EtOH | | | |
| | | | Stored at 4° C. until shipment for further | | | |
| | | | processing | | | |
| Lumbar | 7-9 | 12 | After heparinized saline perfusion: | 4° | C. | IHC |
| Spinal | | | Dissected out lumbar portion of spinal | | | |
| cord | | | cord (10 mm) | | | |
| | | | Positioned spinal cord into cassette | | | |
| | | | between precut foams) | | | |
| | | | Placed cassettes in container with | | | |
| | | | 4% PFA at 4° C. | | | |
| | | | 24 hrs later, washed twice with PBS | | | |
| | | | and once with 70% EtOH | | | |
| | | | Placed in 70% EtOH | | | |
| | | | Stored at 4° C. until shipment for further | | | |
| | | | processing | | | |
| Brain | 1-6 | 24 | After PFA perfusion (~1 mL/g): | 4° | C. | IHC |
| | | | Extracted brain and place in a 50 mL | | | |
| | | | tube with 25 mL 4% PFA at 4° C. | | | |
| | | | 24 hrs later, washed twice with PBS | | | |
| | | | and once with 70% EtOH | | | |
| | | | Placed in 70% EtOH in 5 mL vials | | | |
| | | | Stored at 4° C. until shipment for further | | | |
| | | | processing | | | |
| Cervical | 1-6 | 24 | After PFA perfusion (~1 mL/g): | 4° | C. | IHC |
| Spinal | | | Dissected out cervical portion of spinal | | | |
| cord | | | cord (10 mm) | | | |
| | | | Positioned spinal cord into cassette | | | |
| | | | between precut foams) | | | |
| | | | Placed cassettes in container with | | | |
| | | | 4% PFA at 4° C. | | | |
| | | | 24 hrs later, washed twice with PBS | | | |
| | | | and once with 70% EtOH | | | |
| Thoracic | 1-6 | 24 | After PFA perfusion (~1 mL/g): | 4° | C. | IHC |
| Spinal | | | Dissected out thoracic portion of spinal | | | |
| cord | | | cord (20 mm) | | | |
| | | | Positioned spinal cord into cassette | | | |
| | | | between precut foams) | | | |
| | | | Placed cassettes in container with | | | |
| | | | 4% PFA at 4° C. | | | |
| | | | 24 hrs later, washed twice with PBS | | | |
| | | | and once with 70% EtOH | | | |
| | | | Placed in 70% EtOH | | | |
| | | | Stored at 4° C. until shipment for further | | | |
| | | | processing | | | |
| Lumbar | 1-6 | 24 | After PFA perfusion (~1 mL/g): | 4° | C. | IHC |
| Spinal | | | Dissected out lumbar portion of spinal | | | |
| cord | | | cord (10 mm) | | | |
| | | | Positioned spinal cord into cassette | | | |
| | | | between precut foams) | | | |
| | | | Placed cassettes in container with | | | |
| | | | 4% PFA at 4° C. | | | |
| | | | 24 hrs later, washed twice with PBS | | | |
| | | | and once with 70% EtOH | | | |
| | | | Placed in 70% EtOH | | | |
| | | | Stored at 4° C. until shipment for further | | | |
| | | | processing | | | |

Paraffin Processing Protocol: Brain—The fixed brains stored in 70% EtOH were paraffin processed according to the protocol of Table 7.

TABLE 7
Paraffin Processing Protocols for Mouse Brain

| Reagent | Duration | Temperature | P/V | Drain |
|---|---|---|---|---|
| Ethanol 70% | 0:45 | 37° C. | P/V | 140 |
| Ethanol 96% | 0:30 | 37° C. | P/V | 140 |
| Ethanol 96% | 0:30 | 37° C. | P/V | 140 |
| Ethanol Abs. | 0:30 | 37° C. | P/V | 140 |
| Ethanol Abs. | 0:30 | 37° C. | P/V | 140 |
| Ethanol Abs. | 0:30 | 37° C. | P/V | 140 |
| Xylene | 0:20 | 37° C. | P/V | 140 |
| Xylene | 0:20 | 37° C. | P/V | 140 |
| Histowax | 0:15 | 63° C. | P/V | 140 |
| Histowax | 0:15 | 63° C. | P/V | 140 |
| Histowax | 4:00 | 63° C. | P/V | 140 |

Brain Sectioning—The PFA fixed and paraffin embedded brains were sectioned into 3 series (Table 8). Each series had 20 serial coronal paraffin sections (=ROIs) 470 μm apart and 5 μm in thickness between AP co-ordinates ~2.93 to ~−6.0 (Paxinos & Franklins, The Mouse Brain Atlas 5th edition). Series 1 was used for actual immunostaining (mCherry), series 2 as a spare while series 3 was utilized for piloting/negative controls. See Table 8. A total of 3 series with 20 coronal brain paraffin-sections (=ROIs) were collected between lateral co-ordinates ~2.93 to ~−6.0. (Paxinos & Franklins, The Mouse Brain Atlas 5th edition). In the current sectioning plan ROI=entire brain section. A total of 20 coronal brain paraffin-sections were collected between AP co-ordinates ~2.93 to ~−6.0 on 4 glass slides with 5 sections per glass slide. Each serial section was 5 μm thick and 470 μm apart. Group 1-6 were full brain sections while group 7-9 were hemibrains.

TABLE 8
Brain Coordinates for Sectioning.

| ROI | Coordinates (Relative to Series 1) | Series 1 | Series 2 | Series 3 |
|---|---|---|---|---|
| ROI 1 | 2.9 | 0 | 5 | 10 |
| ROI 2 | 2.46 | 470 | 475 | 480 |
| ROI 3 | 1.99 | 940 | 945 | 950 |
| ROI 4 | 1.52 | 1410 | 1415 | 1520 |
| ROI 5 | 1.05 | 1880 | 1885 | 1890 |
| ROI 6 | 0.58 | 2350 | 2355 | 2360 |
| ROI 7 | 0.11 | 2820 | 2825 | 2830 |
| ROI 8 | −0.36 | 3290 | 3295 | 3300 |
| ROI 9 | −0.83 | 3760 | 3765 | 3770 |
| ROI 10 | −1.3 | 4230 | 4235 | 4240 |
| ROI 11 | −1.77 | 4700 | 4705 | 4710 |
| ROI 12 | −2.24 | 5170 | 5175 | 5180 |
| ROI 13 | −2.71 | 5640 | 5645 | 5650 |
| ROI 14 | −3.18 | 6110 | 6115 | 6120 |
| ROI 15 | −3.65 | 6580 | 6585 | 6590 |
| ROI 16 | −4.12 | 7050 | 7055 | 7060 |
| ROI 17 | −4.59 | 7520 | 7525 | 7230 |
| ROI 18 | −5.06 | 7990 | 7995 | 8000 |
| ROI 19 | −5.53 | 8430 | 8465 | 8470 |
| ROI 20 | −6.0 | 8930 | 8935 | 8940 |

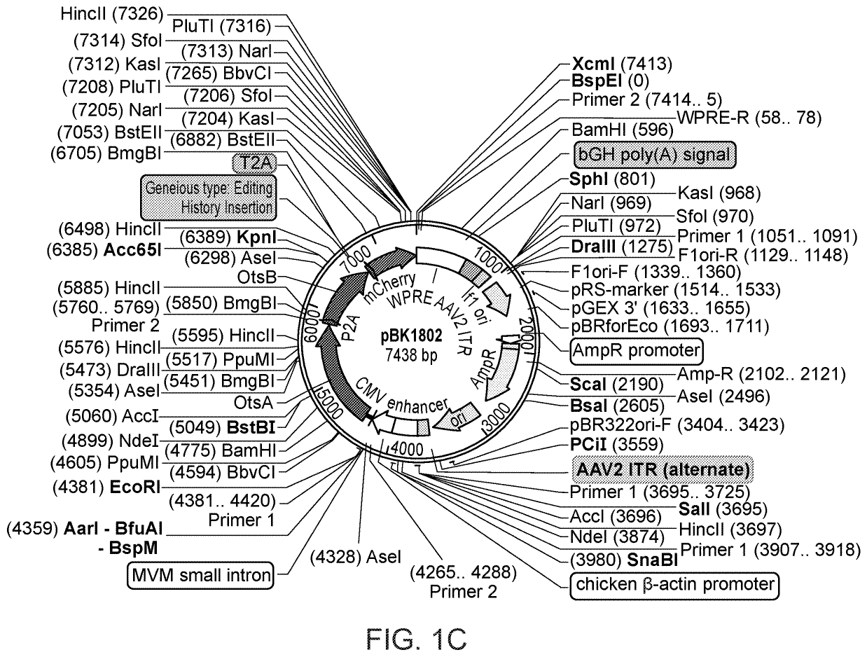

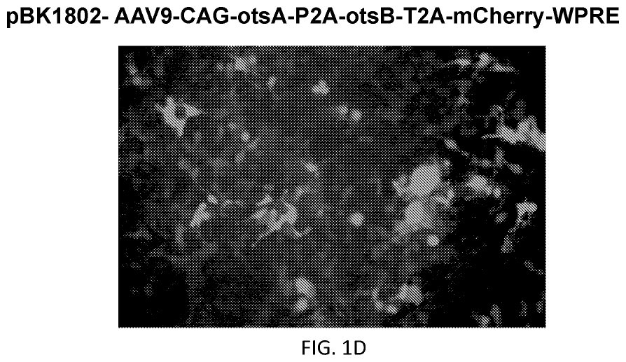

Spinal Cord (SC) Sample Collection—SC tissues samples were collected from cervical (about 10 mm section), thoracic (about 20 mm section) and lumbar regions (about 10 mm section) as illustrated in the representative figure (FIG. 2) below:
Paraffin Processing Protocol: Spinal Cord—The fixed SC sections stored in 70% EtOH were paraffin processed according to the protocol in Table 9:

TABLE 9
Paraffin Processing Protocols for Mouse Spinal Cord

| Reagent | Duration | Temperature | P/V | Drain |
|---|---|---|---|---|
| Ethanol 70% | 1:00 | | P/V | 140 |
| Ethanol 80% | 1:00 | | P/V | 140 |
| Ethanol 96% | 1:00 | | P/V | 140 |
| Ethanol 96% | 2:00 | | P/V | 140 |
| Ethanol 96% | 2:00 | | P/V | 140 |
| Ethanol Abs. | 1:00 | | P/V | 140 |
| Ethanol Abs. | 1:00 | | P/V | 140 |
| Ethanol Abs. | 1:00 | | P/V | 140 |
| Xylene | 0:30 | | P/V | 140 |
| Xylene | 0:30 | | P/V | 140 |
| Histowax | 1:00 | 62° C. | P/V | 140 |
| Histowax | 1:00 | 62° C. | P/V | 140 |
| Histowax | 2:00 | 62° C. | P/V | 140 |

Sectioning Plan—A total of 8 slides with 24 ROIs (6 cervical, 12 thoracic and 6 lumbar) were collected per animal where ROI=entire SC section. Each slide had 3 serial sections 1100 μm apart from 2 animals (#1, #2). Cervical region had total of 2 slides, thoracic region 4 slides and lumbar 2 slides. C=Cervical, T=Thoracic, L=Lumbar
Spinal cord (SC)—The SC samples were paraffin processed and cervical, thoracic and lumbar regions were separately embedded into blocks with n=2 per animal.
Cervical section—A total of 6 ROIs as serial sections 1100 μm apart were collected in 3 series on 2 glass slides. Series 1 were utilized for mCherry IHC, series 2 as an extra and series 3 as pilots (ref to Table 10.)

TABLE 10
Section Plan for Cervical Section

| | ROI 1 | ROI 2 | ROI 3 | ROI 4 | ROI 5 | ROI 6 |
|---|---|---|---|---|---|---|
| | Slide 1 | | | Slide 2 | | |
| Series 1 | 0 | 1100 | 2200 | 3300 | 4400 | 5500 |
| Series 2 | 5 | 1105 | 2205 | 3305 | 4405 | 5505 |
| Series 3 | 10 | 1110 | 2210 | 3310 | 4410 | 5510 |

Thoracic section—A total of up to 12 ROIs as serial sections 1100 μm apart were collected in 3 series on 4 glass slides. Series 1 were utilized for mCherry IHC, series 2 as an extra, and series 3 as pilots (ref to Table 11.)

TABLE 11
Section Plan for Thoracic Section

| | ROI 1 | ROI 2 | ROI 3 | ROI 4 | ROI 5 | ROI 6 |
|---|---|---|---|---|---|---|
| | Slide 1 | | | Slide 2 | | |
| Series 1 | 0 | 1100 | 2200 | 3300 | 4400 | 5500 |
| Series 2 | 5 | 1105 | 2205 | 3305 | 4405 | 5505 |
| Series 3 | 10 | 1110 | 2210 | 3310 | 4410 | 5510 |
| | ROI 7 | ROI 8 | ROI 9 | ROI 10 | ROI 11 | ROI 12 |
| | Slide 3 | | | Slide 4 | | |
| Series 1 | 6600 | 7700 | 8800 | 9900 | 11000 | 12100 |
| Series 2 | 6605 | 7705 | 8805 | 9905 | 11005 | 121005 |
| Series 3 | 6610 | 7710 | 8810 | 9910 | 11010 | 12110 |

Lumbar section—A total of 6 ROIs as serial sections 1100 μm apart were collected in 3 series on 2 glass slides. Series 1 were utilized for mCherry IHC, series 2 as an extra and series 3 as pilots (ref to Table 12.)

TABLE 12
Section Plan for Lumbar Section

| | ROI 1 | ROI 2 | ROI 3 | ROI 4 | ROI 5 | ROI 6 |
|---|---|---|---|---|---|---|
| | Slide 1 | | | Slide 2 | | |
| Series 1 | 0 | 1100 | 2200 | 3300 | 4400 | 5500 |
| Series 2 | 5 | 1105 | 2205 | 3305 | 4405 | 5505 |
| Series 3 | 10 | 1110 | 2210 | 3310 | 4410 | 5510 |

### Immunohistochemistry

Immunohistochemistry was piloted with 2 methods: ABC-DAB and immunofluorescence (IF) and finally IF method was optimized for all tissue sections.

### Immunofluorescence

The tissue sections were deparaffinized followed by permeabilization in 0.3% Triton X-100. Antigen retrieval was done in 0.1M citrate buffer pH 6.0 for 5 min at 80° C. in a microwave oven. This was followed by blocking for nonspecific background in 10% NGS. Finally sections were incubated with primary antibody overnight O/N (refer to Table 13). Following day after washing in PBST the tissue sections were stained by appropriate fluorescent secondary antibody (refer to table 3) followed by washing and mounting in DAPI.

TABLE 13
Details of primary and secondary antibodies
used in immunofluorescence IHC.

| | Dilution | | Dilution | |
|---|---|---|---|---|
| Primary Antibody | Used | Secondary Antibody | Used | Method |
| anti-mCherry antibody | 1:500 | Goat anti-rabbit | 1:500 | IF |
| ab167453 (Rabbit | (brain) | AF568 | | |
| polyclonal, Abcam) | 1:200 | (Invitrogen A11036) | | |
| | (SC) | | | |

### Results

Animals were successfully injected with AAV1-otsA-otsB-T2A-mCherry, bilaterally. No adversive side effects were seen during post-injection observations. Tissues were collected according to protocol. See Table 14 and Table 15 for details.

TABLE 14
Whole blood, serum, and plasma details.

| | Whole | Serum | Serum | Serum | Week 11 Tail | | Week 12 Tail | |
|---|---|---|---|---|---|---|---|---|
| Tx | Blood | Alq 1 | Alq 2 | Alq 3 | Vein Plasma | | Vein Plasma | |
| Group | (μL) | (μL) | (μL) | (μL) | (μL) | | (μL) | |
| 1 | 290 | 100 | 50 | 50 | N/A | | N/A | |
| 2 | 290 | 100 | 50 | 50 | N/A | | N/A | |
| 3 | 29 | 100 | 50 | 70 | N/A | | N/A | |
| 4 | 290 | 75 | not collected | not collected | N/A | | N/A | |
| 5 | 290 | 100 | 50 | 100 | N/A | | N/A | |
| 6 | 290 | 100 | 50 | 125 | N/A | | N/A | |
| 7 | 290 | 100 | 30 | not collected | 70 + 17.5 | FA | 95 + 23.8 | FA |
| 8 | 290 | 100 | 50 | 90 | 60 + 15 | FA | 70 + 17.5 | FA |
| 9 | 290 | 100 | 50 | 40 | 60 + 15 | FA | 60 + 15 | FA |
| 1 | 240 | not collected | not collected | not collected | N/A | | N/A | |
| 2 | 290 | 100 | 50 | 150 | N/A | | N/A | |
| 3 | 290 | 100 | 50 | 60 | N/A | | N/A | |
| 4 | 290 | 100 | 50 | 35 | N/A | | N/A | |
| 5 | 290 | 100 | 50 | 150 | N/A | | N/A | |
| 6 | 290 | 100 | 50 | 25 | N/A | | N/A | |
| 7 | 290 | 100 | 40 | not collected | 80 + 20 | FA | 50 12.5 | FA |
| 8 | 290 | 100 | 50 | 80 | 70 + 17.5 | FA | 60 + 15 | FA |
| 9 | 290 | 100 | 50 | 140 | 80 + 20 | FA | 60 + 15 | FA |
| 1 | 290 | 100 | 50 | 100 | N/A | | N/A | |
| 2 | 290 | 100 | 50 | 80 | N/A | | N/A | |
| 3 | 290 | 100 | 50 | 30 | N/A | | N/A | |
| 4 | 290 | 100 | 50 | 150 | N/A | | N/A | |
| 5 | 290 | 100 | 50 | 10 | N/A | | N/A | |
| 6 | 290 | 100 | 50 | 100 | N/A | | N/A | |
| 7 | 290 | 100 | 25 | not collected | 60 + 15 | FA | 30 + 7.5 | FA |
| 8 | 290 | 100 | 50 | 25 | 60 + 15 | FA | 75 + 18.75 | FA |
| 9 | 290 | 100 | 50 | 21 | 70 + 17.5 | FA | 60 + 15 | FA |
| 1 | 290 | 100 | 50 | 100 | N/A | | N/A | |
| 2 | 290 | 100 | 50 | 50 | N/A | | N/A | |
| 3 | 290 | 100 | 50 | 100 | N/A | | N/A | |
| 4 | 290 | 100 | 50 | 35 | N/A | | N/A | |
| 5 | 290 | 100 | 50 | 105 | N/A | | N/A | |
| 6 | 290 | 100 | 50 | 125 | N/A | | N/A | |
| 8 | ~114 | 100 | 50 | not collected | 90 + 22.5 | FA | 100 + 25 | FA |
| 9 | 290 | 100 | 50 | 25 | 60 + 15 | FA | 80 + 20 | FA |
| 7 | 290 | 100 | 50 | 40 | 80 + 20 | FA | 50 + 12.5 | FA |

TABLE 15
Terminal tissue details.

| | | | | | | L. Rest | L. Rest | | | | | Small | Small |
|---|---|---|---|---|---|---|---|---|---|---|---|---|---|
| | Median | Left | Right | Caudate | L. | of brain | of brain | L. | R. | L. | R. | Intestine | Intestine |
| Tx | liver | liver | liver | liver | STR | Alq 1 | Alq 2 | Kidney | Kidney | Quadriceps | Quadriceps | Alq 1 | Alq 2 |
| Grp | (mg) | (mg) | (mg) | (mg) | (mg) | (mg) | (mg) | (mg) | (mg) | (mg) | (mg) | (mg) | (mg) |
| 1 | 25.9 | 49.6 | 32.5 | 31.3 | N/A | N/A | N/A | N/A | N/A | N/A | N/A | N/A | N/A |
| 2 | 24.3 | 19.4 | 42.2 | 47.7 | N/A | N/A | N/A | N/A | N/A | N/A | N/A | N/A | N/A |
| 3 | 30.2 | 49.5 | 47.5 | 22.1 | N/A | N/A | N/A | N/A | N/A | N/A | N/A | N/A | N/A |
| 4 | 26.1 | 39 | 46.3 | 19.9 | N/A | N/A | N/A | N/A | N/A | N/A | N/A | N/A | N/A |
| 5 | 49.8 | 47.9 | 49.1 | 24.2 | N/A | N/A | N/A | N/A | N/A | N/A | N/A | N/A | N/A |
| 6 | 46.2 | 40.8 | 49.1 | 24.7 | N/A | N/A | N/A | N/A | N/A | N/A | N/A | N/A | N/A |
| 7 | 43.1 | 37.5 | 42.1 | 30.9 | 5.2 | 125.8 | 96.5 | 195.3 | 195.9 | 98.2 | 97.7 | 331.1 | 337.1 |
| 8 | 37.7 | 36.1 | 32.3 | 47.9 | 8.5 | 142 | 88.8 | 209.4 | 261 | 200.7 | 132.5 | 534.4 | 642.8 |
| 9 | 43.4 | 47.6 | 45.4 | 26.3 | 5.5 | 96.4 | 101.5 | 263.2 | 251.3 | 205.9 | 172.7 | 1012.9 | 741.9 |
| 1 | 46.2 | 28 | 39.9 | 24 | N/A | N/A | N/A | N/A | N/A | N/A | N/A | N/A | N/A |
| 2 | 48.2 | 49 | 41.4 | 39 | N/A | N/A | N/A | N/A | N/A | N/A | N/A | N/A | N/A |
| 3 | 35.6 | 49.2 | 44.7 | 34.4 | N/A | N/A | N/A | N/A | N/A | N/A | N/A | N/A | N/A |
| 4 | 42.3 | 31.8 | 45.1 | 40.1 | N/A | N/A | N/A | N/A | N/A | N/A | N/A | N/A | N/A |
| 5 | 40.7 | 38 | 47.8 | 37.2 | N/A | N/A | N/A | N/A | N/A | N/A | N/A | N/A | N/A |
| 6 | 47.2 | 49.5 | 49.4 | 33.7 | N/A | N/A | N/A | N/A | N/A | N/A | N/A | N/A | N/A |
| 7 | 30.7 | 39.1 | 48.1 | 31.4 | 5.8 | 90.1 | 118.1 | 199 | 225.3 | 151.2 | 139.1 | 574.5 | 898.3 |
| 8 | 47.2 | 39.7 | 41.8 | 25.4 | 7.7 | 127 | 121 | 219.6 | 230.9 | 233.8 | 133.2 | 918.6 | 608.5 |
| 9 | 40.4 | 43.8 | 49.5 | 35.6 | 5.1 | 126 | 87.4 | 187 | 229.1 | 213.5 | 151.5 | 779.2 | 872.4 |
| 1 | 31 | 41.3 | 44.3 | 35.8 | N/A | N/A | N/A | N/A | N/A | N/A | N/A | N/A | N/A |
| 2 | 40.3 | 36.2 | 49.8 | 40.1 | N/A | N/A | N/A | N/A | N/A | N/A | N/A | N/A | N/A |
| 3 | 33.9 | 44.5 | 46.0 | 34.2 | N/A | N/A | N/A | N/A | N/A | N/A | N/A | N/A | N/A |
| 4 | 42.7 | 35.3 | 37.4 | 39.2 | N/A | N/A | N/A | N/A | N/A | N/A | N/A | N/A | N/A |
| 5 | 45.8 | 48.4 | 48.9 | 32.1 | N/A | N/A | N/A | N/A | N/A | N/A | N/A | N/A | N/A |
| 6 | 47.5 | 47.5 | 44.2 | 34.8 | N/A | N/A | N/A | N/A | N/A | N/A | N/A | N/A | N/A |
| 7 | 42.1 | 41.6 | *58.4 | 33.8 | 3.9 | 76.6 | 98.7 | 223.9 | 221.7 | 128.5 | 202.4 | 782.3 | 526 |
| 8 | 39.3 | 41.6 | 48.9 | 29.1 | 3.4 | 92.3 | 101.3 | 260.1 | 270.7 | 215.2 | 216.9 | 777.5 | 893.8 |
| 9 | 37.3 | 46.6 | 43.9 | 32 | 1.5 | 96.4 | 105.5 | 189 | 182.9 | 216.4 | 188.2 | 562.2 | 490.4 |
| 1 | 37.1 | 39.5 | 40.2 | 45 | N/A | N/A | N/A | N/A | N/A | N/A | N/A | N/A | N/A |
| 2 | 36.4 | 45.9 | 41.9 | 27.2 | N/A | N/A | N/A | N/A | N/A | N/A | N/A | N/A | N/A |
| 3 | 48.1 | 46.2 | 47.9 | 40.1 | N/A | N/A | N/A | N/A | N/A | N/A | N/A | N/A | N/A |
| 4 | 39.5 | 43.5 | 48.7 | 39.1 | N/A | N/A | N/A | N/A | N/A | N/A | N/A | N/A | N/A |
| 5 | 36.3 | 40.9 | 43.5 | 36.6 | N/A | N/A | N/A | N/A | N/A | N/A | N/A | N/A | N/A |
| 6 | 49.7 | 38 | 49.0 | 31 | N/A | N/A | N/A | N/A | N/A | N/A | N/A | N/A | N/A |
| 8 | 47.8 | 48.1 | 39.7 | 44.5 | 9.2 | 100 | 105.2 | 249.5 | 240.1 | 258.6 | 249.1 | 844.8 | 900.2 |
| 9 | 42.5 | 49.3 | 46.1 | 23.7 | 6 | 92.8 | 105.4 | 201.5 | 181.7 | 90.6 | 223.2 | 643.6 | 555.7 |
| 7 | 38.7 | 45.1 | 47.2 | 39.2 | 4 | 97.5 | 105 | 232.6 | 226.6 | 215.3 | 151.4 | 652.7 | 1127.8 |

*weight includes some RNA later

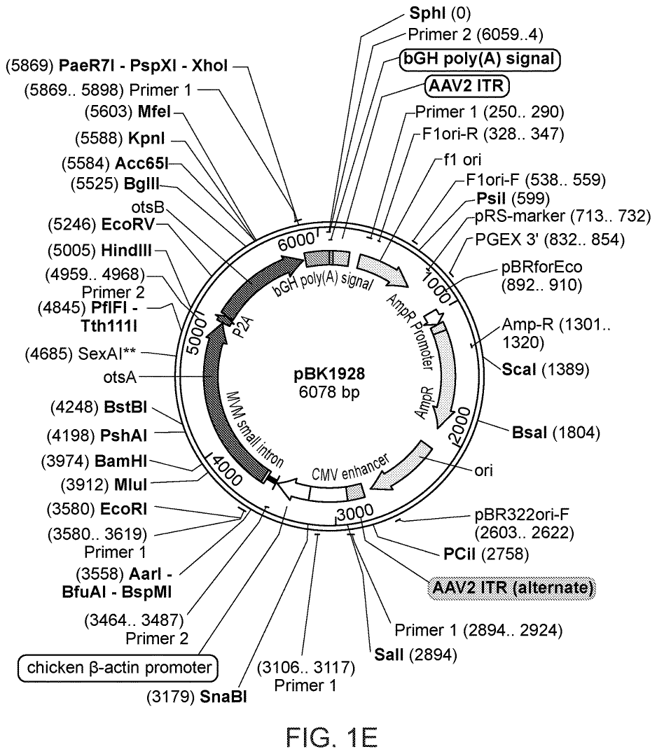

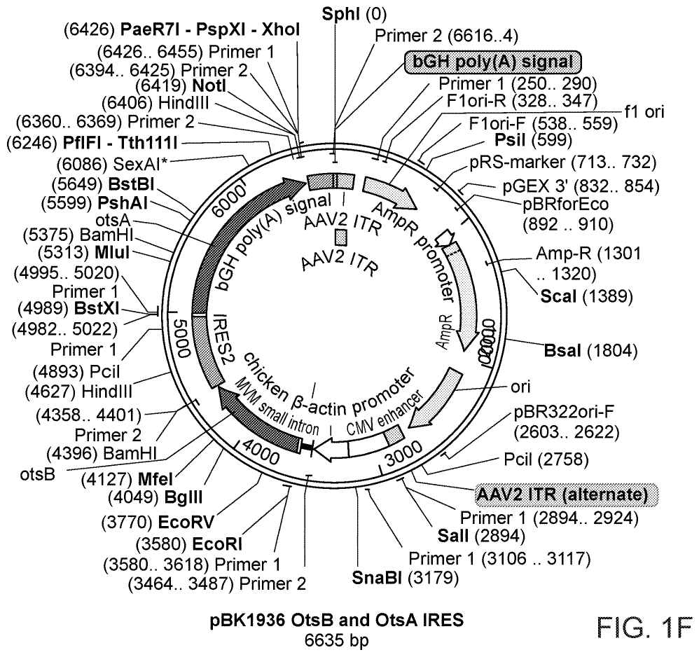

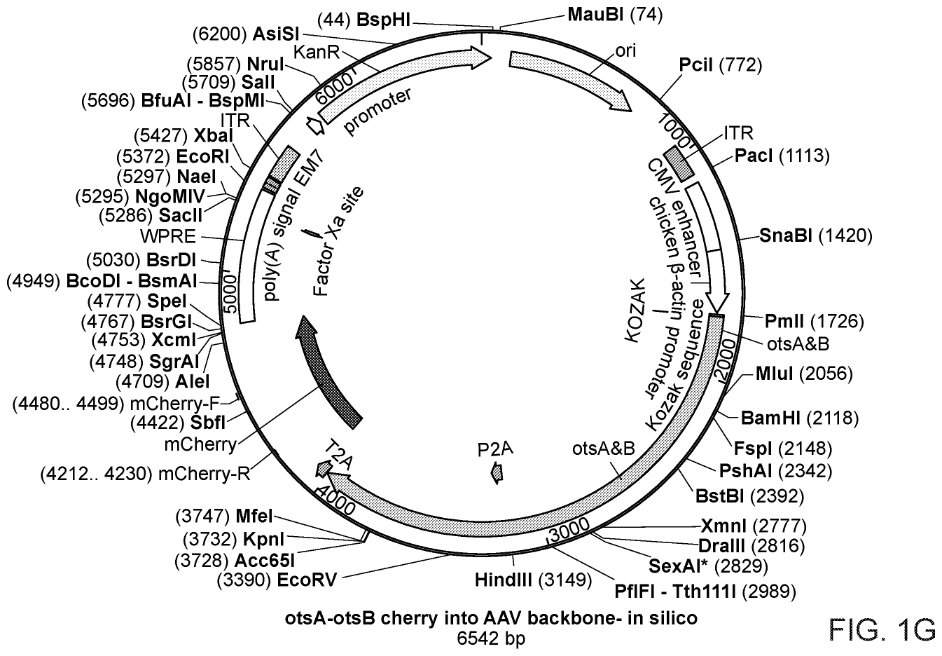

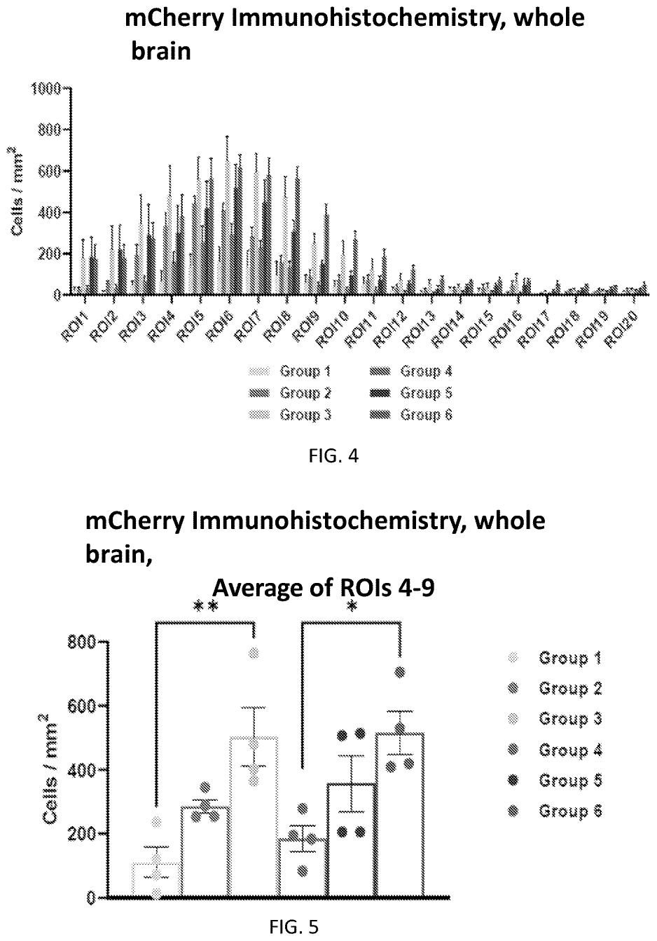

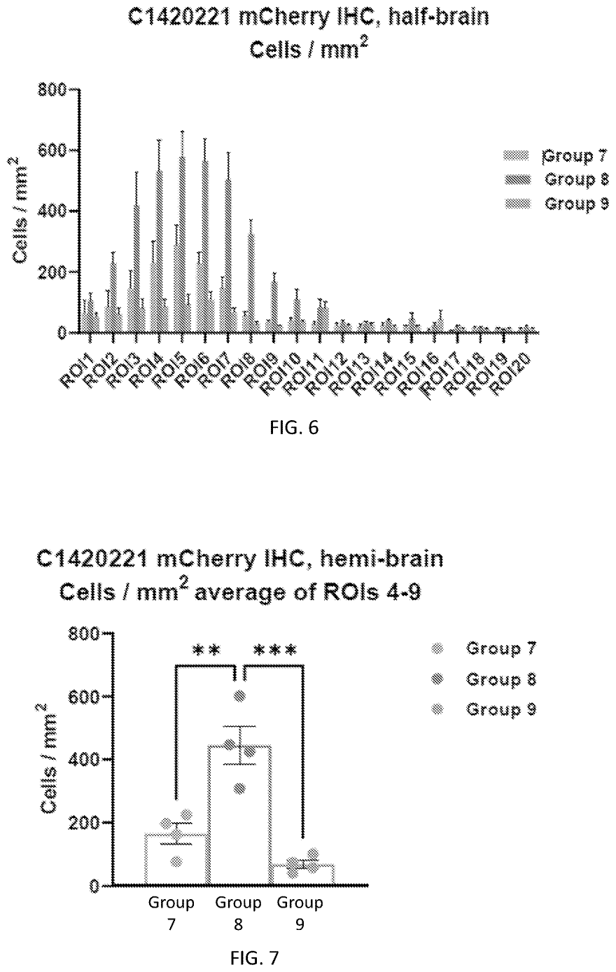

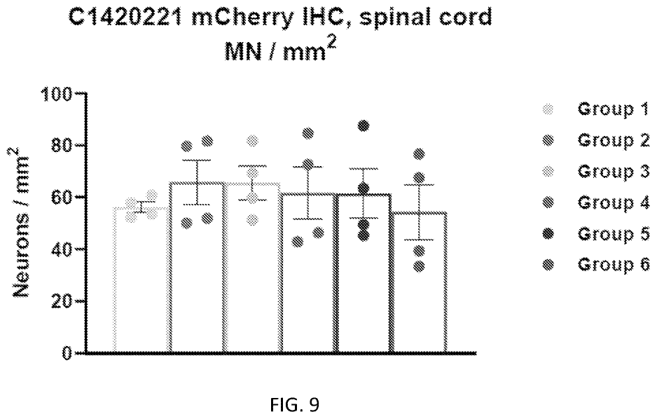

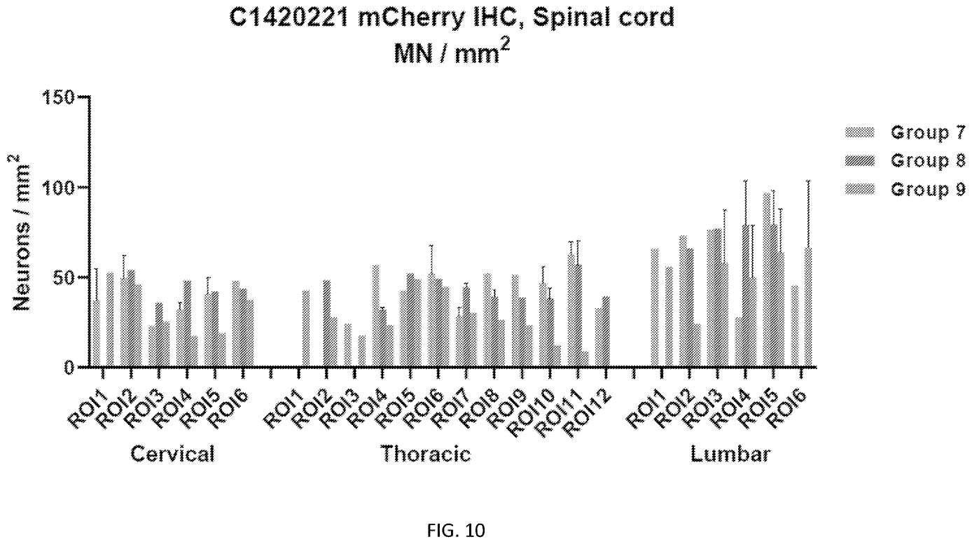

Analysis for the Brain—A strong mCherry signal was observed from the 20 brain ROIs which spanned from the fore brain through mid-brain to the hind brain (FIG. 4). The intensity of mCherry signal showed a trend for an increase towards approaching midbrain sections with highest signals observed around ROIs 4-9 and then a decrease towards hindbrain, as injections were done intra-parenchymal in striatum and signal decreased from there. Overall animal groups 1-6 mCherry showed a dosing titer based signal with animals injected with lowest and highest dose titers demonstrating least and peak signal intensities (FIGS. 4-7).
Analysis of Spinal Cord (SC)—In comparison to the brains the immunointensity of mCherry was fainter in the SC sections. On increasing the concentration of primary antibody to 1:200 (against 1:500 originally) mCherry was visualized within SC cellular structures which were most likely recognized as motor neurons. Unlike the brain, no strong trends for titer dependent signal immunoreactivity was observed within the SCs samples (FIGS. 8-11).
FIG. 12 shows a comparison of total immunointensity distribution between brain and spinal cord tissues.

### CONCLUSION

To summarize mCherry demonstrated a strong signal in the entire 20 brain ROIs with highest signal intensities observed around ROI 4-9. Further there was a strong titer dependent correlation especially in group 1-6.
A faint signal was observed in the SC tissues which was most localized in the motor neurons. Dose dependent titer co-relations were not evident in the SC samples.

### LIST OF ABBREVIATIONS

AP=anterior-posterior
DV=dorsal-ventral
ICV=intracerebroventricular
N=number of animals per group
PFA=paraformaldehyde
STR=striatum
Tx=treatment

### Example 3: Mass Spectrometry Analysis of Cultured Rat Neuronal Samples

This example describes performing mass spectrometry and proteomics on cultured rat neuronal samples that were exposed to AAV trehalose treatment.

### Methods and Materials

### Harvesting Rat Neuronal Samples

Rat primary hippocampal neurons were harvested from three pregnant dams on gestational day E18. Dams were anesthetized with inhaled isoflurane and the uterine horns were externalized under aseptic conditions. Embryos were harvested, decapitated, and embryonic brains were processed for primary neuronal cultures derived from hippocampi. The resulting cells were plated at a density of 30,000 cells per well in poly-D-lysine/laminin treated black 96 well plates with coverglass bottoms suitable for imaging. Half media changes were performed on DIV 3 and DIV 6. On study day 7, cells were treated with AAV Trehalose (pBK1802-AAV9-CAG-otsA-P2A-otsB-T2A-mCherry-WPRE) either at 2.00E+09 viral particles/well (low), or 5.00E+10 viral particles/well (high). On day 14, supernatants were harvested, and cells and cell lysates were fixed as described below.

Briefly, supernatant was collected and 10 μL of 5% formic acid was added. The sample was vortexed, and the sample was frozen at −80° C. Cells were fixed by removing all supernatant, adding 100 μL of 4% PFA to each well and incubated for about 20 minutes. 4% PFA was removed and 200 μL PBS was added to each well for imaging. Cell lysates were lysed by adding 100 μL of lysing buffer (10 ml RIPA+1 tablet of PhosStop+1 tablet of protease inhibitor) and mixing by pipetting. Then, 10 μL of 5% formic acid was added to 90 μL of cell lysate, vortexed, and frozen at −80° C.

### Processing of Neuronal Cells for Proteomic Analysis

The plate containing neurons frozen in lysis buffer was received at Protypia (Inotiv Nashville). Each well contained neurons in lysis buffer. The samples consisted of 6 wells for control with ~30000 cells/well and 5 wells for low dose treatment ~30000 cells/well. Samples were combined lysed, sonicated and centrifuged. The supernatant was removed and analyzed with the Micro BCA assay for protein quantification. The controls yielded 137.36 micrograms total protein and the treated samples yielded 79.87 micrograms total protein. Two samples each containing 21 micrograms protein were taken from the control and treated lysates for analysis.

Proteins were reduced with tris(carboxyethyl)phosphine and alkylated with iodoacetamide. Proteins were precipitated overnight with acetone and the precipitate was dried and resuspended in 100 mM triethylammonium bicarbonate. Samples were digested overnight with trypsin at 37° C.

The digests then were labeled with isobaric TMT reagents (Thermofisher Scientific) using a 10-plex kit (four channels were used and the remaining six were not used. TMT labeling was done for 1 hour at ambient temperature in anhydrous acetonitrile according to the manufacturer's instructions.

The 4 analysis channels were as follows. Numbers refer to the m/z values of the reporter ion specific to each label.
Control 1=127N
Control 2=128C
Treatment 1=129C
Treatment 2=130N

After the labeling reaction, excess TMT reagent was quenched with 5% hydroxylamine. A small sample aliquot (1 microgram protein) from each sample was taken for a single shot LC-MS/MS analysis to check TMT labeling efficiency. The remaining 20 micrograms of each sample were combined in equal amounts and separated into 8 fractions by high pH reverse phase fractionation spin columns (ThermoFisher Scientific). Fractions containing the eluted peptides were evaporated to dryness in a SpeedVac and resuspended HPLC in mobile phase.

Liquid chromatography tandem mass spectrometry (LC-MS/MS) analyses were performed with an Orbitrap Fusion Lumos mass spectrometer coupled to an Ultimate 3000 nano-LC (ThermoFisher Scientific). Peptides were separated over a 3 hour gradient from 4 to 25% acetonitrile in 0.1% formic acid at a flow rate of 300 nL/min on a Thermo Scientific EasySpray 50 cm×75 μm, 2 μm particle size RP C18 column. Full scan MS spectra were acquired in the Orbitrap at a resolution of 120,000. The most intense MS1 ions were selected fragmentation in the ion trap at 35% collision energy and an isolation width of 1.2 Da. Six fragment ions were co-isolated using synchronous precursor selection with an isolation width of 1.3 Da and fragmented by HCD with a normalized collision energy of 65%. The product ions were analyzed using the Orbitrap at a resolution of 50,000.

Peptide identifications are based on search of the MS/MS spectra against a rat protein sequence database with Sequest, a widely used search tool implemented in the Proteome Discoverer 2.5 package (ThermoFisher Scientific). Criteria for protein identification yielded a protein level FDR <1%.

### Results

Global TMT proteomic analysis of duplicates of two samples of neuronal cell cultures yielded an inventory of 2631 identified proteins. For this analysis, conservative criteria for the peptide/protein identification from the dataset were used. The tight stringency analyses employed provided a protein identification list with most proteins represented in all samples.

Quantitative comparisons between control and treated neurons are based on sample-specific “reporter” signals from the TMT tags on the individual peptides. Ratios of these signals are used to compare relative abundances and abundance ratios. These can be expressed as abundance ratios or log 2 normalized ratios (see Table 16).

TABLE 16
Mass Spectrometry Fold Change and Protein Identification.

| Pro- | | | | | | | | | | | | |
|---|---|---|---|---|---|---|---|---|---|---|---|---|
| tein | | | | | | | | | | | | |
| FDR | | | | | | | | | | | | |
| Confi- | | Hall- | | | | | Abun- | Abun- | Abun- | | | |
| dence: | | mark | | | # | Abun- | dance | dance | dance | | | |
| Com- | Symbol | apo- | Acces- | | Pep- | dance | Ratio | (Grouped): | (Grouped): | | | |
| bined | Gene | ptosis | sion | Description | tides | Ratio | (log2) | Control | Sample | Biological Process | Cellular Component | Molecular Function |
| High | Cyld | x | Q66H62 | Ubiquitin | 1 | | | | | cell-cell signaling; cell | plasma membrane; other | other molecular function |
| | | | | carboxyl- | | | | | | organization and | membranes; cytosol; | |
| | | | | terminal | | | | | | biogenesis; protein | cytoskeleton; other cell | |
| | | | | hydrolase | | | | | | metabolism; other metabolic | component | |
| | | | | CYLD | | | | | | processes; stress | | |
| | | | | [OS = Rattus | | | | | | response; signal | | |
| | | | | norvegicus] | | | | | | transduction; other biological | | |
| | | | | | | | | | | processes | | |
| High | Anxa1 | x | P07150 | Annexin A1 | 1 | 5.937 | 2.57 | 28.8 | 171.2 | cell-cell signaling; cell | non-structural | enzyme regulator |
| | | | | [OS = Rattus | | | | | | organization and | extracellular; plasma | activity; nucleic acid binding |
| | | | | norvegicus] | | | | | | biogenesis; protein | membrane; other | activity; other molecular |
| | | | | | | | | | | metabolism; other metabolic | membranes; cytoskeleton; | function |
| | | | | | | | | | | processes; stress | mitochondrion; nucleus; other | |
| | | | | | | | | | | response; transport; | cytoplasmic organelle; other | |
| | | | | | | | | | | developmental processes; signal | cell component | |
| | | | | | | | | | | transduction; other biological | | |
| | | | | | | | | | | processes | | |
| High | Bid | x | Q9JLT6 | BH3- | 2 | 3.601 | 1.85 | 43.5 | 156.5 | cell organization and | other | other molecular function |
| | | | | interacting | | | | | | biogenesis; other metabolic | membranes; cytosol; | |
| | | | | domain death | | | | | | processes; stress | mitochondrion | |
| | | | | agonist | | | | | | response; transport; | | |
| | | | | [OS = Rattus | | | | | | developmental processes; signal | | |
| | | | | norvegicus] | | | | | | transduction; other biological | | |
| | | | | | | | | | | processes | | |
| High | Hmox1 | x | P06762 | Heme | 2 | 2.147 | 1.1 | 63.5 | 136.5 | other metabolic | plasma membrane; other | other molecular function |
| | | | | oxygenase 1 | | | | | | processes; stress | membranes; cytosol; ER/Golgi; | |
| | | | | [OS = Rattus | | | | | | response; developmental | nucleus; other cell component | |
| | | | | norvegicus] | | | | | | processes; signal | | |
| | | | | | | | | | | transduction; other biological | | |
| | | | | | | | | | | processes | | |
| High | Cd44 | x | P26051 | CD44 antigen | 2 | 2.046 | 1.03 | 65.7 | 134.3 | cell adhesion; cell-cell | plasma membrane; other | signal transduction activity |
| | | | | [OS = Rattus | | | | | | signaling; cell organization and | membranes; nucleus; other cell | or receptor binding; other |
| | | | | norvegicus] | | | | | | biogenesis; other metabolic | component | molecular function |
| | | | | | | | | | | processes; stress | | |
| | | | | | | | | | | response; transport; | | |
| | | | | | | | | | | developmental processes; signal | | |
| | | | | | | | | | | transduction; other biological | | |
| | | | | | | | | | | processes | | |
| High | Psen1 | x | P97887 | Presenilin-1 | 1 | 1.615 | 0.69 | 76.5 | 123.5 | cell adhesion; cell-cell | plasma membrane; other | signal transduction activity |
| | | | | [OS = Rattus | | | | | | signaling; cell cycle OR cell | membranes; cytoskeleton; | or receptor |
| | | | | norvegicus] | | | | | | proliferation; cell organization | mitochondrion; ER/Golgi; nucleus; | binding; transporter |
| | | | | | | | | | | and biogenesis; protein | other cytoplasmic | activity; other molecular |
| | | | | | | | | | | metabolism; other metabolic | organelle; other cell component | function |
| | | | | | | | | | | processes; stress | | |
| | | | | | | | | | | response; transport; | | |
| | | | | | | | | | | developmental processes; signal | | |
| | | | | | | | | | | transduction; other biological | | |
| | | | | | | | | | | processes | | |
| High | Timp2 | x | P30121 | Metalloproteinase | 4 | 1.552 | 0.63 | 78.4 | 121.6 | developmental processes; other | non-structural | signal transduction activity |
| | | | | inhibitor 2 | | | | | | biological processes | extracellular; extracellular | or receptor binding; enzyme |
| | | | | [OS = Rattus | | | | | | | matrix; other cell component | regulator activity; other |
| | | | | norvegicus] | | | | | | | | molecular function |
| High | Sod2 | x | P07895 | Superoxide | 4 | 1.395 | 0.48 | 83.5 | 116.5 | cell organization and | mitochondrion; other cell | nucleic acid binding |
| | | | | dismutase [Mn], | | | | | | biogenesis; other metabolic | component | activity; other molecular |
| | | | | mitochondrial | | | | | | processes; stress | | function |
| | | | | [OS = Rattus | | | | | | response; developmental | | |
| | | | | norvegicus] | | | | | | processes; signal | | |
| | | | | | | | | | | transduction; other biological | | |
| | | | | | | | | | | processes | | |
| High | Bax | x | Q63690 | Apoptosis | 3 | 1.377 | 0.46 | 84.2 | 115.8 | cell cycle OR cell | other membranes; | transporter activity; other |
| | | | | regulator BAX | | | | | | proliferation; cell organization | cytosol; mitochondrion; | molecular function |
| | | | | [OS = Rattus | | | | | | and biogenesis; protein | ER/Golgi; nucleus; other | |
| | | | | norvegicus] | | | | | | metabolism; other metabolic | cell component | |
| | | | | | | | | | | processes; stress | | |
| | | | | | | | | | | response; transport; | | |
| | | | | | | | | | | developmental processes; signal | | |
| | | | | | | | | | | transduction; other biological | | |
| | | | | | | | | | | processes | | |
| High | Dnaja1 | x | P63036 | DnaJ homolog | 9 | 1.303 | 0.38 | 86.8 | 113.2 | stress response; transport; signal | other | signal transduction activity |
| | | | | subfamily A | | | | | | transduction; other biological | membranes; cytosol; cytoskeleton; | or receptor binding; other |
| | | | | member 1 | | | | | | processes | mitochondrion; ER/Golgi; | molecular function |
| | | | | [OS = Rattus | | | | | | | nucleus; other cell component | |
| | | | | norvegicus] | | | | | | | | |
| High | App | x | P08592 | Amyloid-beta | 11 | 1.266 | 0.34 | 88.3 | 111.7 | cell adhesion; cell cycle OR | non-structural | signal transduction activity |
| | | | | precursor protein | | | | | | cell proliferation; cell | extracellular; plasma | or receptor binding; enzyme |
| | | | | [OS = Rattus | | | | | | organization and | membrane; other | regulator activity; nucleic |
| | | | | norvegicus] | | | | | | biogenesis; protein | membranes; cytoskeleton; ER/ | acid binding activity; other |
| | | | | | | | | | | metabolism; RNA metabolism | Golgi; nucleus; other cytoplasmic | molecular function |
| | | | | | | | | | | OR transcription; other | organelle; other cell component | |
| | | | | | | | | | | metabolic processes; stress | | |
| | | | | | | | | | | response; transport; | | |
| | | | | | | | | | | developmental processes; signal | | |
| | | | | | | | | | | transduction; other biological | | |
| | | | | | | | | | | processes | | |
| High | Eno2 | x | P07323 | Gamma- | 17 | 1.241 | 0.31 | 89.2 | 110.8 | other metabolic | plasma membrane; other | other molecular function |
| | | | | enolase | | | | | | processes; other biological | membranes; cytosol; other cell | |
| | | | | [OS = Rattus | | | | | | processes | component | |
| | | | | norvegicus] | | | | | | | | |
| High | Dnajc3 | x | Q9R0T3 | DnaJ homolog | 2 | 1.232 | 0.3 | 89.6 | 110.4 | protein metabolism; other | cytosol; ER/Golgi; other cell | enzyme regulator |
| | | | | subfamily C | | | | | | metabolic processes; stress | component | activity; other molecular |
| | | | | member 3 | | | | | | response; other biological | | function |
| | | | | [OS = Rattus | | | | | | processes | | |
| | | | | norvegicus] | | | | | | | | |
| High | Sptan1 | x | P16086 | Spectrin alpha | 119 | 1.205 | 0.27 | 90.7 | 109.3 | cell organization and | plasma membrane; other | cytoskeletal activity; other |
| | | | | chain, non- | | | | | | biogenesis | membranes; cytosol; | molecular function |
| | | | | erythrocytic 1 | | | | | | | cytoskeleton; other cell component | |
| | | | | [OS = Rattus | | | | | | | | |
| | | | | norvegicus] | | | | | | | | |
| High | Dpyd | x | O89000 | Dihydro- | 2 | 1.128 | 0.17 | 94 | 106 | other metabolic | cytosol; other cell component | other molecular function |
| | | | | pyrimidine | | | | | | processes; other biological | | |
| | | | | dehydrogenase | | | | | | processes | | |
| | | | | [NADP(+)] | | | | | | | | |
| | | | | [OS = Rattus | | | | | | | | |
| | | | | norvegicus] | | | | | | | | |
| High | Ppp3r1 | x | P63100 | Calcineurin | 4 | 1.06 | 0.08 | 97.1 | 102.9 | protein metabolism; other | plasma membrane; other | enzyme regulator |
| | | | | subunit B type | | | | | | metabolic | membranes; cytosol; other cell | activity; other molecular |
| | | | | 1 [OS = Rattus | | | | | | processes; transport; | component | function |
| | | | | norvegicus] | | | | | | developmental processes; signal | | |
| | | | | | | | | | | transduction; other biological | | |
| | | | | | | | | | | processes | | |
| High | Clu | x | P05371 | Clusterin | 1 | 1.037 | 0.05 | 98.2 | 101.8 | cell cycle OR cell | non-structural | signal transduction activity |
| | | | | [OS = Rattus | | | | | | proliferation; cell organization | extracellular; other | or receptor |
| | | | | norvegicus] | | | | | | and biogenesis; protein | membranes; cytosol; | binding; cytoskeletal |
| | | | | | | | | | | metabolism; other metabolic | mitochondrion; | activity; other molecular |
| | | | | | | | | | | processes; stress | ER/Golgi; nucleus; other | function |
| | | | | | | | | | | response; transport; | cell component | |
| | | | | | | | | | | developmental processes; signal | | |
| | | | | | | | | | | transduction; other biological | | |
| | | | | | | | | | | processes | | |
| High | Vdac2 | x | P81155 | Voltage- | 14 | 0.978 | −0.03 | 101.1 | 98.9 | cell organization and | other | transporter activity; other |
| | | | | dependent | | | | | | biogenesis; transport; signal | membranes; cytoskeleton; | molecular function |
| | | | | anion-selective | | | | | | transduction; other biological | mitochondrion; ER/Golgi; other | |
| | | | | channel protein | | | | | | processes | cell component | |
| | | | | 2 [OS = Rattus | | | | | | | | |
| | | | | norvegicus] | | | | | | | | |
| High | Hmgb2 | x | P52925 | High mobility | 6 | 0.963 | −0.05 | 101.9 | 98.1 | cell organization and | non-structural | signal transduction activity |
| | | | | group protein | | | | | | biogenesis; DNA | extracellular; nucleus; other | or receptor binding; nucleic |
| | | | | B2 [OS = Rattus | | | | | | metabolism; RNA metabolism | cell component | acid binding activity; other |
| | | | | norvegicus] | | | | | | OR transcription; other | | molecular function |
| | | | | | | | | | | metabolic processes; stress | | |
| | | | | | | | | | | response; developmental | | |
| | | | | | | | | | | processes; signal | | |
| | | | | | | | | | | transduction; other biological | | |
| | | | | | | | | | | processes | | |
| High | Rhob | x | P62747 | Rho-related | 7 | 0.961 | −0.06 | 102 | 98 | cell adhesion; cell cycle OR | plasma membrane; other | other molecular function |
| | | | | GTP-binding | | | | | | cell proliferation; cell | membranes; cytosol; cytoskeleton; | |
| | | | | protein RhoB | | | | | | organization and | nucleus; other cytoplasmic | |
| | | | | [OS = Rattus | | | | | | biogenesis; stress | organelle; other cell component | |
| | | | | norvegicus] | | | | | | response; transport; | | |
| | | | | | | | | | | developmental processes; signal | | |
| | | | | | | | | | | transduction; other biological | | |
| | | | | | | | | | | processes | | |
| High | Ctnnb1 | x | Q9WU82 | Catenin beta-1 | 23 | 0.947 | −0.08 | 102.7 | 97.3 | cell adhesion; cell-cell | plasma membrane; other | signal transduction activity |
| | | | | [OS = Rattus | | | | | | signaling; cell cycle OR cell | membranes; cytosol; cytoskeleton; | or receptor binding; other |
| | | | | norvegicus] | | | | | | proliferation; cell organization | nucleus; other cell | molecular function |
| | | | | | | | | | | and biogenesis; protein | component | |
| | | | | | | | | | | metabolism; RNA metabolism | | |
| | | | | | | | | | | OR transcription; other | | |
| | | | | | | | | | | metabolic processes; stress | | |
| | | | | | | | | | | response; transport; | | |
| | | | | | | | | | | developmental processes; signal | | |
| | | | | | | | | | | transduction; other biological | | |
| | | | | | | | | | | processes | | |
| High | Top2a | x | P41516 | DNA | 3 | 0.936 | −0.1 | 103.3 | 96.7 | cell cycle OR cell | nucleus; other cell component | nucleic acid binding |
| | | | | topoisomerase | | | | | | proliferation; cell organization | | activity; other molecular |
| | | | | 2-alpha | | | | | | and biogenesis; DNA | | function |
| | | | | [OS = Rattus | | | | | | metabolism; other metabolic | | |
| | | | | norvegicus] | | | | | | processes; stress | | |
| | | | | | | | | | | response; developmental | | |
| | | | | | | | | | | processes; other biological | | |
| | | | | | | | | | | processes | | |
| High | Pak1 | x | P35465 | Serine/threonine- | 8 | 0.926 | −0.11 | 103.9 | 96.1 | cell-cell signaling; cell | plasma membrane; other | cytoskeletal activity; kinase |
| | | | | protein kinase | | | | | | organization and | membranes; cytosol; cytoskeleton; | activity; other molecular |
| | | | | PAK 1 | | | | | | biogenesis; protein | nucleus; other cell | function |
| | | | | [OS = Rattus | | | | | | metabolism; other metabolic | component | |
| | | | | norvegicus] | | | | | | processes; stress | | |
| | | | | | | | | | | response; transport; | | |
| | | | | | | | | | | developmental processes; signal | | |
| | | | | | | | | | | transduction; other biological | | |
| | | | | | | | | | | processes | | |
| High | Sod1 | x | P07632 | Superoxide | 7 | 0.923 | −0.12 | 104 | 96 | cell organization and | non-structural | other molecular function |
| | | | | dismutase | | | | | | biogenesis; other metabolic | extracellular; plasma | |
| | | | | [Cu—Zn] | | | | | | processes; stress response; | membrane; other membranes; | |
| | | | | [OS = Rattus | | | | | | transport; developmental | cytosol; mitochondrion; | |
| | | | | norvegicus] | | | | | | processes; other biological | nucleus; other cytoplasmic | |
| | | | | | | | | | | processes | organelle; other cell component | |
| High | Casp3 | x | P55213 | Caspase-3 | 2 | 0.894 | −0.16 | 105.6 | 94.4 | cell organization and | plasma membrane; other | signal transduction activity |
| | | | | [OS = Rattus | | | | | | biogenesis; protein | membranes; cytosol; nucleus; | or receptor binding; enzyme |
| | | | | norvegicus] | | | | | | metabolism; other metabolic | other cell component | regulator activity; other |
| | | | | | | | | | | processes; stress | | molecular function |
| | | | | | | | | | | response; developmental | | |
| | | | | | | | | | | processes; signal | | |
| | | | | | | | | | | transduction; other biological | | |
| | | | | | | | | | | processes | | |
| High | Erbb2 | x | P06494 | Receptor | 1 | 0.888 | −0.17 | 105.9 | 94.1 | cell cycle OR cell | plasma membrane; other | signal transduction activity |
| | | | | tyrosine-protein | | | | | | proliferation; cell organization | membranes; nucleus; other | or receptor binding; enzyme |
| | | | | kinase erbB-2 | | | | | | and biogenesis; protein | cytoplasmic organelle; other | regulator activity; nucleic |
| | | | | [OS = Rattus | | | | | | metabolism; other metabolic | cell component | acid binding activity; kinase |
| | | | | norvegicus] | | | | | | processes; stress | | activity; other molecular |
| | | | | | | | | | | response; developmental | | function |
| | | | | | | | | | | processes; signal | | |
| | | | | | | | | | | transduction; other biological | | |
| | | | | | | | | | | processes | | |
| High | Add1 | x | Q63028 | Alpha-adducin | 11 | 0.871 | −0.2 | 106.9 | 93.1 | cell organization and | plasma membrane; other | signal transduction activity |
| | | | | [OS = Rattus | | | | | | biogenesis; protein | membranes; cytoskeleton; | or receptor |
| | | | | norvegicus] | | | | | | metabolism; other metabolic | nucleus; other cell component | binding; cytoskeletal |
| | | | | | | | | | | processes; transport; | | activity; other molecular |
| | | | | | | | | | | developmental processes; other | | function |
| | | | | | | | | | | biological processes | | |
| High | Etf1 | x | Q5U2Q7 | Eukaryotic | 10 | 0.832 | −0.26 | 109.2 | 90.8 | cell organization and | cytosol; other cell component | nucleic acid binding activity |
| | | | | peptide chain | | | | | | biogenesis; protein | | |
| | | | | release factor | | | | | | metabolism; RNA metabolism | | |
| | | | | subunit 1 | | | | | | OR transcription; other | | |
| | | | | [OS = Rattus | | | | | | metabolic processes; other | | |
| | | | | norvegicus] | | | | | | biological processes | | |
| High | Rhot2 | x | Q7TSA0 | Mitochondrial | 2 | 0.816 | −0.29 | 110.1 | 89.9 | cell organization and | | other molecular function |
| | | | | Rho GTPase 2 | | | | | | biogenesis; transport; signal | | |
| | | | | [OS = Rattus | | | | | | transduction; other biological | | |
| | | | | norvegicus] | | | | | | processes | | |
| High | Ptk2 | x | O35346 | Focal adhesion | 5 | 0.806 | −0.31 | 110.7 | 89.3 | cell organization and | plasma membrane; other | signal transduction activity |
| | | | | kinase 1 | | | | | | biogenesis; protein | membranes; cytosol; cytoskeleton; | or receptor |
| | | | | [OS = Rattus | | | | | | metabolism; other metabolic | nucleus; other cell | binding; cytoskeletal |
| | | | | norvegicus] | | | | | | processes; stress | component | activity; kinase activity; other |
| | | | | | | | | | | response; transport; | | molecular function |
| | | | | | | | | | | developmental processes; signal | | |
| | | | | | | | | | | transduction; other biological | | |
| | | | | | | | | | | processes | | |
| High | Pea15 | x | Q5U318 | Astrocytic | 6 | 0.791 | −0.34 | 111.7 | 88.3 | transport; signal | cytosol; nucleus | |
| | | | | phosphoprotein | | | | | | transduction; other biological | | |
| | | | | PEA-15 | | | | | | processes | | |
| | | | | [OS-Rattus | | | | | | | | |
| | | | | norvegicus] | | | | | | | | |
| High | Fdxr | x | P56522 | NADPH:adre- | 3 | 0.771 | −0.37 | 112.9 | 87.1 | other metabolic processes | other | other molecular function |
| | | | | nodoxin | | | | | | | membranes; mitochondrion | |
| | | | | oxidoreductase, | | | | | | | | |
| | | | | mitochondrial | | | | | | | | |
| | | | | [OS = Rattus | | | | | | | | |
| | | | | norvegicus] | | | | | | | | |
| High | Ppt1 | x | P45479 | Palmitoyl- | 4 | 0.738 | −0.44 | 115.1 | 84.9 | cell-cell signaling; cell | non-structural | other molecular function |
| | | | | protein | | | | | | organization and | extracellular; other | |
| | | | | thioesterase 1 | | | | | | biogenesis; protein | membranes; cytosol; ER/Golgi; | |
| | | | | [OS = Rattus | | | | | | metabolism; other metabolic | nucleus; other cytoplasmic | |
| | | | | norvegicus] | | | | | | processes; transport; | organelle; other cell component | |
| | | | | | | | | | | developmental processes; other | | |
| | | | | | | | | | | biological processes | | |
| High | Dnm1l | x | O35303 | Dynamin-1-like | 28 | 0.728 | −0.46 | 115.7 | 84.3 | cell organization and | plasma membrane; other | cytoskeletal activity; other |
| | | | | protein | | | | | | biogenesis; stress | membranes; cytosol; cytoskeleton; | molecular function |
| | | | | [OS = Rattus | | | | | | response; transport; signal | mitochondrion; ER/Golgi; | |
| | | | | norvegicus] | | | | | | transduction; other biological | other cytoplasmic | |
| | | | | | | | | | | processes | organelle; other cell component | |
| High | Ppp2r5b | x | Q80W83 | Serine/threonine- | 5 | 0.717 | −0.48 | 116.5 | 83.5 | protein metabolism; other | cytosol; nucleus; other cell | enzyme regulator activity |
| | | | | protein | | | | | | metabolic processes; signal | component | |
| | | | | phosphatase 2A | | | | | | transduction; other biological | | |
| | | | | 56 kDa | | | | | | processes | | |
| | | | | regulatory | | | | | | | | |
| | | | | subunit beta | | | | | | | | |
| | | | | isoform | | | | | | | | |
| | | | | [OS = Rattus | | | | | | | | |
| | | | | norvegicus] | | | | | | | | |
| High | Gpx4 | x | P36970 | Phospholipid | 4 | 0.701 | −0.51 | 117.5 | 82.5 | cell organization and | cytosol; mitochondrion; nucleus; | other molecular function |
| | | | | hydroperoxide | | | | | | biogenesis; other metabolic | other cell component | |
| | | | | glutathione | | | | | | processes; stress response; other | | |
| | | | | peroxidase | | | | | | biological processes | | |
| | | | | [OS = Rattus | | | | | | | | |
| | | | | norvegicus] | | | | | | | | |
| High | Lmna | x | P48679 | Prelamin-A/C | 10 | 0.688 | −0.54 | 118.4 | 81.6 | cell cycle OR cell | other | cytoskeletal activity; other |
| | | | | [OS = Rattus | | | | | | proliferation; cell organization | membranes; cytoskeleton; | molecular function |
| | | | | norvegicus] | | | | | | and biogenesis; other metabolic | nucleus; other cell component | |
| | | | | | | | | | | processes; stress | | |
| | | | | | | | | | | response; transport; | | |
| | | | | | | | | | | developmental processes; other | | |
| | | | | | | | | | | biological processes | | |
| High | Gstm1 | x | P04905 | Glutathione S- | 9 | 0.662 | −0.6 | 120.3 | 79.7 | other metabolic | non-structural | other molecular function |
| | | | | transferase Mu | | | | | | processes; stress response; other | extracellular; cytosol; other cell | |
| | | | | 1 [OS = Rattus | | | | | | biological processes | component | |
| | | | | norvegicus] | | | | | | | | |
| High | Madd | x | O08873 | MAP kinase- | 9 | 0.618 | −0.69 | 123.6 | 76.4 | transport; other biological | plasma membrane; other | enzyme regulator activity |
| | | | | activating death | | | | | | processes | membranes; cytosol; ER/Golgi; | |
| | | | | domain protein | | | | | | | nucleus; other cell component | |
| | | | | [OS = Rattus | | | | | | | | |
| | | | | norvegicus] | | | | | | | | |
| High | Fez1 | x | P97577 | Fasciculation | 3 | 0.541 | −0.89 | 129.8 | 70.2 | cell organization and | plasma membrane; other | cytoskeletal activity; other |
| | | | | and elongation | | | | | | biogenesis; developmental | membranes; cytoskeleton; ER/ | molecular function |
| | | | | protein zeta-1 | | | | | | processes; other biological | Golgi; other cell component | |
| | | | | [OS = Rattus | | | | | | processes | | |
| | | | | norvegicus] | | | | | | | | |
| High | Gpx1 | x | P04041 | Glutathione | 1 | 0.541 | −0.89 | 129.7 | 70.3 | cell cycle OR cell | cytosol; mitochondrion; nucleus; | other molecular function |
| | | | | peroxidase 1 | | | | | | proliferation; cell organization | other cell component | |
| | | | | [OS = Rattus | | | | | | and biogenesis; protein | | |
| | | | | norvegicus] | | | | | | metabolism; other metabolic | | |
| | | | | | | | | | | processes; stress | | |
| | | | | | | | | | | response; developmental | | |
| | | | | | | | | | | processes; signal | | |
| | | | | | | | | | | transduction; other biological | | |
| | | | | | | | | | | processes | | |
| High | Krt75 | | Q6IG05 | Keratin, type II | 9 | 45.642 | 5.51 | 4.3 | 195.7 | cell organization and | plasma | bone, tooth or skin structural |
| | | | | cytoskeletal 75 | | | | | | biogenesis; other biological | membrane; cytoskeleton | activity |
| | | | | [OS = Rattus | | | | | | processes | | |
| | | | | norvegicus] | | | | | | | | |
| High | Krt6a | | Q4FZU2 | Keratin, type II | 11 | 31.383 | 4.97 | 6.2 | 193.8 | cell organization and | cytoskeleton | bone, tooth or skin structural |
| | | | | cytoskeletal 6A | | | | | | biogenesis; stress | | activity |
| | | | | [OS = Rattus | | | | | | response; other biological | | |
| | | | | norvegicus] | | | | | | processes | | |
| High | Krt42 | | Q6IFU7 | Keratin, type I | 8 | 20.669 | 4.37 | 9.2 | 190.8 | cell organization and | cytoskeleton; other cell | other molecular function |
| | | | | cytoskeletal 42 | | | | | | biogenesis; other biological | component | |
| | | | | [OS = Rattus | | | | | | processes | | |
| | | | | norvegicus] | | | | | | | | |
| High | Krt14 | | Q6IFV1 | Keratin, type I | 13 | 14.224 | 3.83 | 13.1 | 186.9 | cell organization and | plasma | other molecular function |
| | | | | cytoskeletal 14 | | | | | | biogenesis; other biological | membrane; cytoskeleton; nucleus; | |
| | | | | [OS = Rattus | | | | | | processes | other cell component | |
| | | | | norvegicus] | | | | | | | | |
| High | Ubtf | | P25977 | Nucleolar | 2 | 13.001 | 3.7 | 14.3 | 185.7 | cell organization and | nucleus | nucleic acid binding |
| | | | | transcription | | | | | | biogenesis; RNA metabolism | | activity; other molecular |
| | | | | factor 1 | | | | | | OR transcription; other | | function |
| | | | | [OS = Rattus | | | | | | metabolic processes; other | | |
| | | | | norvegicus] | | | | | | biological processes | | |
| High | Krt17 | | Q6IFU8 | Keratin, type I | 13 | 12.062 | 3.59 | 15.3 | 184.7 | cell organization and | plasma | cytoskeletal activity |
| | | | | cytoskeletal 17 | | | | | | biogenesis; other biological | membrane; cytoskeleton; other | |
| | | | | [OS = Rattus | | | | | | processes | cell component | |
| | | | | norvegicus] | | | | | | | | |
| High | Krt8 | | Q10758 | Keratin, type II | 7 | 11.58 | 3.53 | 15.9 | 184.1 | cell organization and | plasma membrane; other | bone, tooth or skin structural |
| | | | | cytoskeletal 8 | | | | | | biogenesis; stress | membranes; cytoskeleton; nucleus; | activity; other molecular |
| | | | | [OS = Rattus | | | | | | response; developmental | other cell component | function |
| | | | | norvegicus] | | | | | | processes; signal | | |
| | | | | | | | | | | transduction; other biological | | |
| | | | | | | | | | | processes | | |
| High | Atp5mf | | D3ZAF6 | ATP synthase | 2 | 10.828 | 3.44 | 16.9 | 183.1 | other metabolic | other | |
| | | | | subunit f, | | | | | | processes; transport | membranes; mitochondrion | |
| | | | | mitochondrial | | | | | | | | |
| | | | | [OS = Rattus | | | | | | | | |
| | | | | norvegicus] | | | | | | | | |
| High | Krt1 | | Q6IMF3 | Keratin, type II | 6 | 9.681 | 3.28 | 18.7 | 181.3 | cell organization and | plasma membrane; other | bone, tooth or skin structural |
| | | | | cytoskeletal 1 | | | | | | biogenesis; protein | membranes; cytoskeleton; other | activity; other molecular |
| | | | | [OS = Rattus | | | | | | metabolism; other metabolic | cell component | function |
| | | | | norvegicus] | | | | | | processes; stress response; other | | |
| | | | | | | | | | | biological processes | | |
| High | Atp5me | | P29419 | ATP synthase | 3 | 6.707 | 2.75 | 25.9 | 174.1 | other metabolic | other | transporter activity; other |
| | | | | subunit e, | | | | | | processes; transport | membranes; mitochondrion | molecular function |
| | | | | mitochondrial | | | | | | | | |
| | | | | [OS = Rattus | | | | | | | | |
| | | | | norvegicus] | | | | | | | | |
| High | Krt10 | | Q6IFW6 | Keratin, type I | 7 | 6.655 | 2.73 | 26.1 | 173.9 | cell organization and | plasma | bone, tooth or skin structural |
| | | | | cytoskeletal 10 | | | | | | biogenesis; protein | membrane; cytoskeleton; other | iactivity; cytoskeletal |
| | | | | [OS = Rattus | | | | | | metabolism; other metabolic | cell component | activity; other molecular |
| | | | | norvegicus] | | | | | | processes; other biological | | function |
| | | | | | | | | | | processes | | |
| High | Ssrp1 | | Q04931 | FACT complex | 11 | 6.153 | 2.62 | 28 | 172 | cell organization and | nucleus | nucleic acid binding |
| | | | | subunit SSRP1 | | | | | | biogenesis; DNA | | activity; other molecular |
| | | | | [OS = Rattus | | | | | | metabolism; other metabolic | | function |
| | | | | norvegicus] | | | | | | processes; stress response; other | | |
| | | | | | | | | | | biological processes | | |
| High | Krt72 | | Q6IG04 | Keratin, type II | 4 | 5.902 | 2.56 | 29 | 171 | cell organization and | cytoskeleton | bone, tooth or skin structural |
| | | | | cytoskeletal 72 | | | | | | biogenesis; other biological | | activity |
| | | | | [OS = Rattus | | | | | | processes | | |
| | | | | norvegicus] | | | | | | | | |
| High | Hspa1a | | P0DMW0 | Heat shock 70 | 14 | 5.782 | 2.53 | 29.5 | 170.5 | cell organization and | non-structural | signal transduction activity |
| | | | | kDa protein 1A | | | | | | biogenesis; DNA | extracellular; plasma | or receptor binding; other |
| | | | | [OS = Rattus | | | | | | metabolism; RNA metabolism | membrane; other | molecular function |
| | | | | norvegicus] | | | | | | OR transcription; other | membranes; cytosol; cytoskeleton; | |
| | | | | | | | | | | metabolic processes; stress | mitochondrion; nucleus; | |
| | | | | | | | | | | response; transport; | other cell component | |
| | | | | | | | | | | developmental processes; other | | |
| | | | | | | | | | | biological processes | | |
| High | Krt5 | | Q6P6Q2 | Keratin, type II | 15 | 5.587 | 2.48 | 30.4 | 169.6 | cell organization and | plasma membrane; other | bone, tooth or skin structural |
| | | | | cytoskeletal 5 | | | | | | biogenesis; other biological | membranes; cytoskeleton; other | activity; other molecular |
| | | | | [OS = Rattus | | | | | | processes | cell component | function |
| | | | | norvegicus] | | | | | | | | |
| High | Dek | | Q6AXS3 | Protein DEK | 7 | 5.085 | 2.35 | 32.9 | 167.1 | cell organization and | cytoskeleton; nucleus | nucleic acid binding |
| | | | | [OS = Rattus | | | | | | biogenesis; other biological | | activity; other molecular |
| | | | | norvegicus] | | | | | | processes | | function |
| High | Npm1 | | P13084 | Nucleophosmin | 11 | 4.858 | 2.28 | 34.1 | 165.9 | cell cycle OR cell | cytosol; cytoskeleton; | enzyme regulator |
| | | | | [OS = Rattus | | | | | | proliferation; cell organization | translational apparatus; nucleus; | activity; translation |
| | | | | norvegicus] | | | | | | and biogenesis; DNA | other cell component | activity; nucleic acid binding |
| | | | | | | | | | | metabolism; RNA metabolism | | activity; other molecular |
| | | | | | | | | | | OR transcription; other | | function |
| | | | | | | | | | | metabolic processes; stress | | |
| | | | | | | | | | | response; transport; | | |
| | | | | | | | | | | developmental processes; other | | |
| | | | | | | | | | | biological processes | | |
| High | Hnrnpc | | G3V9R8 | Heterogeneous | 14 | 4.653 | 2.22 | 35.4 | 164.6 | RNA metabolism OR | cytosol; cytoskeleton; nucleus; | enzyme regulator |
| | | | | nuclear | | | | | | transcription; other metabolic | other cell component | activity; nucleic acid binding |
| | | | | ribonucleo- | | | | | | processes; other biological | | activity; other molecular |
| | | | | protein C | | | | | | processes | | function |
| | | | | [OS = Rattus | | | | | | | | |
| | | | | norvegicus] | | | | | | | | |
| High | Tpt1 | | P63029 | Translationally- | 3 | 4.635 | 2.21 | 35.5 | 164.5 | cell cycle OR cell | non-structural | other molecular function |
| | | | | controlled | | | | | | proliferation; other biological | extracellular; cytoskeleton; | |
| | | | | tumor protein | | | | | | processes | nucleus; other cytoplasmic | |
| | | | | [OS = Rattus | | | | | | | organelle; other cell component | |
| | | | | norvegicus] | | | | | | | | |
| High | Krt2 | | Q6IG02 | Keratin, type II | 8 | 4.274 | 2.1 | 37.9 | 162.1 | cell cycle OR cell | plasma | bone, tooth or skin structural |
| | | | | cytoskeletal 2 | | | | | | proliferation; cell organization | membrane; cytoskeleton; other | activity; cytoskeletal activity |
| | | | | epidermal | | | | | | and biogenesis; protein | cell component | |
| | | | | [OS = Rattus | | | | | | metabolism; other metabolic | | |
| | | | | norvegicus] | | | | | | processes; other biological | | |
| | | | | | | | | | | processes | | |
| High | Atp5if1 | | Q03344 | ATPase | 2 | 4.219 | 2.08 | 38.3 | 161.7 | cell organization and | mitochondrion; other cell | other molecular function |
| | | | | inhibitor, | | | | | | biogenesis; other metabolic | component | |
| | | | | mitochondrial | | | | | | processes; stress | | |
| | | | | [OS = Rattus | | | | | | response; transport; other | | |
| | | | | norvegicus] | | | | | | biological processes | | |
| High | Cyb5r1 | | Q5EB81 | NADH- | 3 | 4.084 | 2.03 | 39.3 | 160.7 | other metabolic processes | other membranes | other molecular function |
| | | | | cytochrome b5 | | | | | | | | |
| | | | | reductase 1 | | | | | | | | |
| | | | | [OS = Rattus | | | | | | | | |
| | | | | norvegicus] | | | | | | | | |
| High | Set | | Q63945 | Protein SET | 8 | 3.653 | 1.87 | 43 | 157 | cell organization and | cytosol; ER/Golgi; nucleus; | nucleic acid binding |
| | | | | [OS = Rattus | | | | | | biogenesis; other biological | other cell component | activity; other molecular |
| | | | | norvegicus | | | | | | processes | | function |
| High | Thrap3 | | Q5M7V8 | Thyroid | 4 | 3.645 | 1.87 | 43.1 | 156.9 | RNA metabolism OR | nucleus | nucleic acid binding |
| | | | | hormone | | | | | | transcription; other metabolic | | activity; other molecular |
| | | | | receptor- | | | | | | processes; other biological | | function |
| | | | | associated | | | | | | processes | | |
| | | | | protein 3 | | | | | | | | |
| | | | | [OS = Rattus | | | | | | | | |
| | | | | norvegicus] | | | | | | | | |
| High | Tf | | P12346 | Serotransferrin | 2 | 3.606 | 1.85 | 43.4 | 156.6 | cell organization and | non-structural | molecular function |
| | | | | [OS = Rattus | | | | | | biogenesis; stress | extracellular; extracellular | signal transduction activity |
| | | | | norvegicus] | | | | | | response; transport; signal | matrix; plasma membrane; other | or receptor binding; other |
| | | | | | | | | | | transduction; other biological | membranes; other cytoplasmic | |
| | | | | | | | | | | processes | organelle; other cell component | |
| High | Ndufb1 | | P0DN35 | NADH | 2 | 3.561 | 1.83 | 43.9 | 156.1 | cell organization and | other | |
| | | | | dehydrogenase | | | | | | biogenesis | membranes; mitochondrion | |
| | | | | [ubiquinone] 1 | | | | | | | | |
| | | | | beta | | | | | | | | |
| | | | | subcomplex | | | | | | | | |
| | | | | subunit 1 | | | | | | | | |
| | | | | [OS = Rattus | | | | | | | | |
| | | | | norvegicus] | | | | | | | | |
| High | Tra2b | | P62997 | Transformer-2 | 4 | 3.479 | 1.8 | 44.7 | 155.3 | RNA metabolism OR | other | nucleic acid binding |
| | | | | protein | | | | | | transcription; other metabolic | membranes; nucleus; other cell | activity; other molecular |
| | | | | homolog beta | | | | | | processes; stress | component | function |
| | | | | [OS = Rattus | | | | | | response; developmental | | |
| | | | | norvegicus] | | | | | | processes; other biological | | |
| | | | | | | | | | | processes | | |
| High | Cd200 | | P04218 | OX-2 | 3 | 3.225 | 1.69 | 47.3 | 152.7 | cell adhesion; other biological | plasma membrane; other | other molecular function |
| | | | | membrane | | | | | | processes | membranes; other cell | |
| | | | | glycoprotein | | | | | | | component | |
| | | | | [OS = Rattus | | | | | | | | |
| | | | | norvegicus] | | | | | | | | |
| High | Anxa5 | | P14668 | Annexin A5 | 11 | 3.164 | 1.66 | 48 | 152 | stress response; transport; other | non-structural | signal transduction activity |
| | | | | [OS = Rattus | | | | | | biological processes | extracellular; plasma | or receptor |
| | | | | norvegicus] | | | | | | | membrane; other membranes; | binding; transporter |
| | | | | | | | | | | | cytosol; cytoskeleton; | activity; other molecular |
| | | | | | | | | | | | ER/Golgi; nucleus; other cell | function |
| | | | | | | | | | | | component | |
| High | Anxa6 | | P48037 | Annexin A6 | 22 | 3.149 | 1.65 | 48.2 | 151.8 | cell organization and | plasma membrane; other | transporter |
| | | | | [OS = Rattus | | | | | | biogenesis; stress | membranes; mitochondrion; | activity; cytoskeletal |
| | | | | norvegicus] | | | | | | response; transport; | nucleus; other cytoplasmic | activity; other molecular |
| | | | | | | | | | | developmental processes; signal | organelle; other cell component | function |
| | | | | | | | | | | transduction; other biological | | |
| | | | | | | | | | | processes | | |
| High | Gpm6b | | Q9JJK1 | Neuronal | 4 | 3.085 | 1.63 | 49 | 151 | cell organization and | plasma membrane; other | |
| | | | | membrane | | | | | | biogenesis; transport; | membranes | |
| | | | | glycoprotein | | | | | | developmental processes; other | | |
| | | | | M6-b | | | | | | biological processes | | |
| | | | | [OS = Rattus | | | | | | | | |
| | | | | norvegicus] | | | | | | | | |
| High | Anxa2 | | Q07936 | Annexin A2 | 2 | 3.096 | 1.63 | 48.8 | 151.2 | cell organization and | non-structural | transporter activity; enzyme |
| | | | | [OS = Rattus | | | | | | biogenesis; RNA metabolism | extracellular; extracellular | regulator |
| | | | | norvegicus] | | | | | | OR transcription; other | matrix; plasma membrane; other | activity; cytoskeletal |
| | | | | | | | | | | metabolic | membranes; cytosol; nucleus; | activity; other molecular |
| | | | | | | | | | | processes; transport; | otther cytoplasmic | function |
| | | | | | | | | | | developmeintal processes; other | organelle; other cell component | |
| | | | | | | | | | | biological processes | | |
| High | H3-3b | | P84245 | Histone H3.3 | 6 | 3.056 | 1.61 | 49.3 | 150.7 | cell cycle OR cell | nucleus; other cell component | nucleic acid binding |
| | | | | [OS = Rattus | | | | | | proliferation; cell organization | | activity; other molecular |
| | | | | norvegicus] | | | | | | and biogenesis; developmental | | function |
| | | | | | | | | | | processes; other biological | | |
| | | | | | | | | | | processes | | |
| High | Myl12b | | P18666 | Myosin | 2 | 3.033 | 1.6 | 49.6 | 150.4 | other biological processes | cytoskeleton; other cell | cytoskeletal activity; other |
| | | | | regulatory light | | | | | | | component | molecular function |
| | | | | chain 12B | | | | | | | | |
| | | | | [OS = Rattus | | | | | | | | |
| | | | | norvegicus] | | | | | | | | |
| High | Nap1l1 | | Q9Z2G8 | Nucleosome | 4 | 2.925 | 1.55 | 51 | 149 | cell cycle OR cell | nucleus; other cell component | other molecular function |
| | | | | assembly | | | | | | proliferation; cell organization | | |
| | | | | protein 1-like 1 | | | | | | and biogenesis; developmental | | |
| | | | | [OS = Rattus | | | | | | processes; other biological | | |
| | | | | norvegicus] | | | | | | processes | | |
| High | Vps33a | | Q63615 | Vacuolar | 2 | 2.901 | 1.54 | 51.3 | 148.7 | cell organization and | other membranes; other | other molecular function |
| | | | | protein sorting- | | | | | | biogenesis; other metabolic | cytoplasmic organelle; other | |
| | | | | associated | | | | | | processes; transport; other | cell component | |
| | | | | protein 33A | | | | | | biological processes | | |
| | | | | [OS = Rattus | | | | | | | | |
| | | | | norvegicus] | | | | | | | | |
| High | Hpx | | P20059 | Hemopexin | 2 | 2.888 | 1.53 | 51.4 | 148.6 | protein metabolism; other | non-structural extracellular | transporter activity; other |
| | | | | [OS = Rattus | | | | | | metabolic processes; stress | | molecular function |
| | | | | norvegicus] | | | | | | response; transport; signal | | |
| | | | | | | | | | | transduction; other biological | | |
| | | | | | | | | | | processes | | |
| High | Alb | | P02770 | Albumin | 7 | 2.785 | 1.48 | 52.8 | 147.2 | stress response; other biological | non-structural | nucleic acid binding |
| | | | | [OS = Rattus | | | | | | processes | extracellular; extracellular | activity; other molecular |
| | | | | norvegicus] | | | | | | | matrix; other cell component | function |
| High | Pfn2 | | Q9EPC6 | Profilin-2 | 3 | 2.728 | 1.45 | 53.7 | 146.3 | cell organization and | cytoskeleton; other cell | cytoskeletal activity; other |
| | | | | [OS = Rattus | | | | | | biogenesis; other biological | component | molecular function |
| | | | | norvegicus] | | | | | | processes | | |
| High | Vac14 | | Q80W92 | Protein VAC14 | 9 | 2.694 | 1.43 | 54.1 | 145.9 | other metabolic | other | other molecular function |
| | | | | homolog | | | | | | processes; other biological | membranes; cytosol; ER/Golgi; | |
| | | | | [OS = Rattus | | | | | | processes | other cytoplasmic organelle | |
| | | | | norvegicus] | | | | | | | | |
| High | Snrpb | | P17136 | Small nuclear | 5 | 2.522 | 1.33 | 56.8 | 143.2 | cell organization and | cytosol; nucleus; other cell | nucleic acid binding |
| | | | | ribonucleo- | | | | | | biogenesis; protein | component | activity; other molecular |
| | | | | protein- | | | | | | metabolism; RNA metabolism | | function |
| | | | | associated | | | | | | OR transcription; other | | |
| | | | | protein B | | | | | | metabolic | | |
| | | | | [OS = Rattus | | | | | | processes; developmental | | |
| | | | | norvegicus] | | | | | | processes | | |
| High | Jup | | Q6P0K8 | Junction | 7 | 2.49 | 1.32 | 57.3 | 142.7 | cell adhesion; cell-cell | plasma membrane; other | other molecular function |
| | | | | plakoglobin | | | | | | signaling; cell organization and | membranes; cytosol; cytoskeleton; | |
| | | | | [OS = Rattus | | | | | | biogenesis; RNA metabolism | nucleus; other cell | |
| | | | | norvegicus] | | | | | | OR transcription; signal | component | |
| | | | | | | | | | | transduction; other biological | | |
| | | | | | | | | | | processes | | |
| High | Slit1 | | O88279 | Slit homolog 1 | 5 | 2.487 | 1.31 | 57.4 | 142.6 | cell organization and | non-structural extracellular | signal transduction activity |
| | | | | protein | | | | | | biogenesis; transport; | | or receptor binding; other |
| | | | | [OS = Rattus | | | | | | developmental processes; other | | molecular function |
| | | | | norvegicus] | | | | | | biological processes | | |
| High | Csnk2a1 | | P19139 | Casein kinase II | 12 | 2.466 | 1.3 | 57.7 | 142.3 | cell-cell signaling; cell cycle | plasma membrane; other | enzyme regulator |
| | | | | subunit alpha | | | | | | OR cell proliferation; protein | membranes; cytosol; nucleus; | activity; kinase activity; other |
| | | | | [OS = Rattus | | | | | | metabolism; other metabolic | other cell component | molecular function |
| | | | | norvegicus] | | | | | | processes; developmental | | |
| | | | | | | | | | | processes; signal | | |
| | | | | | | | | | | transduction; other biological | | |
| | | | | | | | | | | processes | | |
| High | Col1a2 | | P02466 | Collagen alpha- | 2 | 2.463 | 1.3 | 57.8 | 142.2 | cell organization and | non-structural | extracellular structural |
| | | | | 2(I) chain | | | | | | biogenesis; other metabolic | extracellular; extracellular | activity; other molecular |
| | | | | [OS = Rattus | | | | | | processes; developmental | matrix; other cell component | function |
| | | | | norvegicus] | | | | | | processes; signal | | |
| | | | | | | | | | | transduction; other biological | | |
| | | | | | | | | | | processes | | |
| High | Ina | | P23565 | Alpha- | 22 | 2.39 | 1.26 | 59 | 141 | cell organization and | cytoskeleton; other cell | cytoskeletal activity; other |
| | | | | internexin | | | | | | biogenesis; developmental | component | molecular function |
| | | | | [OS = Rattus | | | | | | processes; other biological | | |
| | | | | norvegicus] | | | | | | processes | | |
| High | Pfdn2 | | B0BN18 | Prefoldin | 4 | 2.38 | 1.25 | 59.2 | 140.8 | other biological processes | mitochondrion; nucleus; other | other molecular function |
| | | | | subunit 2 | | | | | | | cell component | |
| | | | | [OS = Rattus | | | | | | | | |
| | | | | norvegicus] | | | | | | | | |
| High | Gpm6a | | Q812E9 | Neuronal | 6 | 2.356 | 1.24 | 59.6 | 140.4 | cell organization and | plasma membrane; other | transporter activity |
| | | | | membrane | | | | | | biogenesis; transport; | membranes; other cell | |
| | | | | glycoprotein | | | | | | developmental processes; other | component | |
| | | | | M6-a | | | | | | biological processes | | |
| | | | | [OS = Rattus | | | | | | | | |
| | | | | norvegicus] | | | | | | | | |
| High | Hnrnpu | | Q6IMY8 | Heterogeneous | 20 | 2.344 | 1.23 | 59.8 | 140.2 | cell cycle OR cell | cytosol; cytoskeleton; nucleus; o | cytoskeletal activity; nucleic |
| | | | | nuclear | | | | | | proliferation; cell organization | ither cell component | acid binding activity; other |
| | | | | ribonucleo- | | | | | | and biogenesis; RNA | | molecular function |
| | | | | protein U | | | | | | metabolism OR | | |
| | | | | [OS = Rattus | | | | | | transcription; other metabolic | | |
| | | | | norvegicus] | | | | | | processes; transport; | | |
| | | | | | | | | | | developmental processes; other | | |
| | | | | | | | | | | biological processes | | |
| High | Aldh1a1 | | P51647 | Aldehyde | 3 | 2.329 | 1.22 | 60.1 | 139.9 | other metabolic | cytosol; nucleus; other cell | other molecular function |
| | | | | dehydrogenase | | | | | | processes; stress | component | |
| | | | | 1A1 | | | | | | response; developmental | | |
| | | | | [OS = Rattus | | | | | | processes; other biological | | |
| | | | | norvegicus] | | | | | | processes | | |
| High | Pacc1 | | Q66H28 | Proton- | 2 | 2.317 | 1.21 | 60.3 | 139.7 | transport | plasma membrane; other | transporter activity |
| | | | | activated chloride | | | | | | | membranes | |
| | | | | channel | | | | | | | | |
| | | | | [OS-Rattus | | | | | | | | |
| | | | | norvegicus] | | | | | | | | |
| High | Fxyd6 | | Q91XV6 | FXYD domain- | 4 | 2.287 | 1.19 | 60.8 | 139.2 | transport; other biological | other cell component | other molecular function |
| | | | | containing ion | | | | | | processes | | |
| | | | | transport | | | | | | | | |
| | | | | regulator 6 | | | | | | | | |
| | | | | [OS = Rattus | | | | | | | | |
| | | | | norvegicus] | | | | | | | | |
| High | Nme1 | | Q05982 | Nucleoside | 9 | 2.252 | 1.17 | 61.5 | 138.5 | DNA metabolism; other | plasma membrane; other | cytoskeletal |
| | | | | diphosphate | | | | | | metabolic | membranes; cytosol; cytoskeleton; | activity; translation |
| | | | | kinase A | | | | | | processes; transport; | mitochondrion; nucleus; other | activity; nucleic acid binding |
| | | | | [OS = Rattus | | | | | | developmental processes; other | cell component | activity; kinase activity; other |
| | | | | norvegicus] | | | | | | biological processes | | molecular function |
| High | Dnajc5 | | P60905 | DnaJ homolog | 6 | 2.224 | 1.15 | 62 | 138 | cell-cell signaling; other | plasma membrane; other | other molecular function |
| | | | | subfamily C | | | | | | biological processes | membranes; cytosol; ER/Golgi; | |
| | | | | member 5 | | | | | | | other cell component | |
| | | | | [OS = Rattus | | | | | | | | |
| | | | | norvegicus] | | | | | | | | |
| High | Cacna2d1 | | P54290 | Voltage- | 25 | 2.182 | 1.13 | 62.8 | 137.2 | cell-cell | plasma membrane; other | transporter activity; other |
| | | | | dependent | | | | | | signaling; transport; other | membranes; cytosol; ER/Golgi; | molecular function |
| | | | | calcium | | | | | | biological processes | other cell component | |
| | | | | channel subunit | | | | | | | | |
| | | | | alpha-2/delta-1 | | | | | | | | |
| | | | | [OS = Rattus | | | | | | | | |
| | | | | norvegicus] | | | | | | | | |
| High | Macroh2a1 | | Q02874 | Core histone | 6 | 2.193 | 1.13 | 62.6 | 137.4 | cell organization and | nucleus; other cell component | enzyme regulator |
| | | | | macro-H2A.1 | | | | | | biogenesis; other biological | | activity; nucleic acid binding |
| | | | | [OS = Rattus | | | | | | processes | | activity; other molecular |
| | | | | norvegicus] | | | | | | | | function |
| High | Srsf6 | | G3V6S8 | Serine/arginine- | 6 | 2.181 | 1.13 | 62.9 | 137.1 | cell organization and | nucleus | nucleic acid binding activity |
| | | | | rich splicing | | | | | | biogenesis; RNA metabolism | | |
| | | | | factor 6 | | | | | | OR transcription; other | | |
| | | | | [OS = Rattus | | | | | | metabolic processes; other | | |
| | | | | norvegicus] | | | | | | biological processes | | |
| High | Hsp90b1 | | Q66HD0 | Endoplasmin | 32 | 2.171 | 1.12 | 63.1 | 136.9 | cell organization and | plasma membrane; other | signal transduction activity |
| | | | | [OS = Rattus | | | | | | biogenesis; protein | membranes; cytosol; ER/Golgi; | or receptor binding; nucleic |
| | | | | norvegicus] | | | | | | metabolism; other metabolic | other cell component | acid binding activity; other |
| | | | | | | | | | | processes; stress | | molecular function |
| | | | | | | | | | | response; transport; other | | |
| | | | | | | | | | | biological processes | | |
| High | Vim | | P31000 | Vimentin | 21 | 2.18 | 1.12 | 62.9 | 137.1 | cell organization and | plasma membrane; other | cytoskeletal activity; nucleic |
| | | | | [OS = Rattus | | | | | | biogenesis; stress | membranes; cytosol; cytoskeleton; | acid binding activity; other |
| | | | | norvegicus] | | | | | | response; developmental | nucleus; other cytoplasmic | molecular function |
| | | | | | | | | | | processes; signal | organelle; other cell component | |
| | | | | | | | | | | transduction; other biological | | |
| | | | | | | | | | | processes | | |
| High | Opcml | | P32736 | Opioid-binding | 9 | 2.181 | 1.12 | 62.9 | 137.1 | cell adhesion | plasma membrane; other | |
| | | | | protein/cell | | | | | | | membranes | |
| | | | | adhesion | | | | | | | | |
| | | | | molecule | | | | | | | | |
| | | | | [OS = Rattus | | | | | | | | |
| | | | | norvegicus] | | | | | | | | |
| High | Serinc1 | | Q7TNK0 | Serine | 2 | 2.157 | 1.11 | 63.4 | 136.6 | other metabolic | plasma membrane; other | other molecular function |
| | | | | incorporator 1 | | | | | | processes; other biological | membranes; ER/Golgi | |
| | | | | [OS = Rattus | | | | | | processes | | |
| | | | | norvegicus] | | | | | | | | |
| High | Hba1 | | P01946 | Hemoglobin | 6 | 2.128 | 1.09 | 63.9 | 136.1 | other metabolic | cytosol; other cell component | signal transduction activity |
| | | | | subunit alpha- | | | | | | processes; transport; | | or receptor binding; other |
| | | | | 1/2 [OS = Rattus | | | | | | developmental processes; other | | molecular function |
| | | | | norvegicus] | | | | | | biological processes | | |
| High | Arfgap2 | | Q3MID3 | ADP- | 2 | 2.084 | 1.06 | 64.8 | 135.2 | cell organization and | other membranes; ER/Golgi | enzyme regulator |
| | | | | ribosylation | | | | | | biogenesis; transport | | activity; other molecular |
| | | | | factor GTPase- | | | | | | | | function |
| | | | | activating | | | | | | | | |
| | | | | protein 2 | | | | | | | | |
| | | | | [OS = Rattus | | | | | | | | |
| | | | | norvegicus] | | | | | | | | |
| High | Nasp | | Q66HD3 | Nuclear | 13 | 2.07 | 1.05 | 65.2 | 134.8 | cell cycle OR cell | nucleus; other cell component | fother molecular function |
| | | | | autoantigenic | | | | | | proliferation; cell organization | | |
| | | | | sperm protein | | | | | | and biogenesis; DNA | | |
| | | | | [OS = Rattus | | | | | | metabolism; other metabolic | | |
| | | | | norvegicus] | | | | | | processes; transport; | | |
| | | | | | | | | | | developmental processes; other | | |
| | | | | | | | | | | biological processes | | |
| High | Tfrc | | Q99376 | Transferrin | 18 | 2.051 | 1.04 | 65.5 | 134.5 | stress response; transport; signal | non-structural | nucleic acid binding |
| | | | | receptor protein | | | | | | transduction; other biological | extracellular; plasma | activity; other molecular |
| | | | | 1 [OS = Rattus | | | | | | processes | membrane; other | function |
| | | | | norvegicus] | | | | | | | membranes; mitochondrion; | |
| | | | | | | | | | | | nucleus; other cytoplasmic | |
| | | | | | | | | | | | organelle; other cell component | |
| High | Tmem30a | | Q6AY41 | Cell cycle | 5 | 2.049 | 1.03 | 65.6 | 134.4 | cell organization and | plasma membrane; other | transporter activity |
| | | | | control protein | | | | | | biogenesis; transport; other | membranes; ER/Golgi; other | |
| | | | | 50A | | | | | | biological processes | cytoplasmic organelle; other | |
| | | | | [OS = Rattus | | | | | | | cell component | |
| | | | | norvegicus] | | | | | | | | |
| High | Ppm1g | | F1LNI5 | Protein | 4 | 2.038 | 1.03 | 65.8 | 134.2 | cell cycle OR cell | other | other molecular function |
| | | | | phosphatase 1G | | | | | | proliferation; protein | membranes; nucleus; other cell | |
| | | | | [OS = Rattus | | | | | | metabolism; other metabolic | component | |
| | | | | norvegicus] | | | | | | processes; other biological | | |
| | | | | | | | | | | processes | | |
| High | Banf1 | | Q9R1T1 | Barrier-to- | 3 | 2.041 | 1.03 | 65.8 | 134.2 | cell cycle OR cell | nucleus; other cell component | nucleic acid binding |
| | | | | autointegration | | | | | | proliferation; cell organization | | activity; other molecular |
| | | | | factor | | | | | | and biogenesis; DNA | | function |
| | | | | [OS = Rattus | | | | | | metabolism; other metabolic | | |
| | | | | norvegicus] | | | | | | processes; stress response; other | | |
| | | | | | | | | | | biological processes | | |
| High | Ero1a | | Q8R4A1 | ERO1-like | 2 | 2.038 | 1.03 | 65.8 | 134.2 | cell organization and | non-structural | other molecular function |
| | | | | protein alpha | | | | | | biogenesis; protein | extracellular; other | |
| | | | | [OS = Rattus | | | | | | metabolism; other metabolic | membranes; ER/Golgi; other | |
| | | | | norvegicus] | | | | | | processes; stress | cell component | |
| | | | | | | | | | | response; transport; signal | | |
| | | | | | | | | | | transduction; other biological | | |
| | | | | | | | | | | processes | | |
| High | Map1lc3b | | Q62625 | Microtubule- | 3 | 2.026 | 1.02 | 66.1 | 133.9 | cell organization and | other | cytoskeletal activity; other |
| | | | | associated | | | | | | biogenesis; other metabolic | membranes; cytosol; cytoskeleton; | molecular function |
| | | | | proteins 1A/1B | | | | | | processes; stress | mitochondrion; other | |
| | | | | light chain 3B | | | | | | response; transport; other | cytoplasmic organelle; other | |
| | | | | [OS = Rattus | | | | | | biological processes | cell component | |
| | | | | norvegicus] | | | | | | | | |
| High | Csnk2b | | P67874 | Casein kinase II | 5 | 2.016 | 1.01 | 66.3 | 133.7 | cell-cell signaling; protein | plasma membrane; other | signal transduction activity |
| | | | | subunit beta | | | | | | metabolism; other metabolic | membranes; nucleus; other cell | or receptor binding; enzyme |
| | | | | [OS-Rattus | | | | | | processes; developmental | component | regulator activity; kinase |
| | | | | norvegicus] | | | | | | processes; signal | | activity; other molecular |
| | | | | | | | | | | transduction; other biological | | function |
| | | | | | | | | | | processes | | |
| High | P4ha1 | | P54001 | Prolyl 4- | 3 | 2.015 | 1.01 | 66.3 | 133.7 | cell organization and | ER/Golgi | other molecular function |
| | | | | hydroxylase | | | | | | biogenesis; protein | | |
| | | | | subunit alpha-1 | | | | | | metabolism; other metabolic | | |
| | | | | [OS-Rattus | | | | | | processes | | |
| | | | | norvegicus] | | | | | | | | |
| High | Stx8 | | Q9Z2Q7 | Syntaxin-8 | 2 | 2.008 | 1.01 | 66.5 | 133.5 | cell organization and | other | other molecular function |
| | | | | [OS = Rattus | | | | | | biogenesis; stress | membranes; cytosol; ER/Golgi; | |
| | | | | norvegicus] | | | | | | response; transport; other | other cytoplasmic | |
| | | | | | | | | | | biological processes | organelle; other cell component | |
| High | Vgf | | P20156 | Neurosecretory | 7 | 2.004 | 1 | 66.6 | 133.4 | cell-cell signaling; other | non-structural | signal transduction activity |
| | | | | protein VGF | | | | | | metabolic processes; stress | extracellular; other | or receptor binding |
| | | | | [OS = Rattus | | | | | | response; transport; | membranes; ER/Golgi; other | |
| | | | | norvegicus] | | | | | | developmental processes; signal | cell component | |
| | | | | | | | | | | transduction; other biological | | |
| | | | | | | | | | | processes | | |
| High | Eif4h | | Q5X172 | Eukaryotic | 3 | 0.5 | −1 | 133.3 | 66.7 | cell organization and | other cell component | translation activity; nucleic |
| | | | | translation | | | | | | biogenesis; protein | | acid binding activity |
| | | | | initiation factor | | | | | | metabolism; other metabolic | | |
| | | | | 4H [OS = Rattus | | | | | | processes; other biological | | |
| | | | | norvegicus] | | | | | | processes | | |
| High | GLTP | | B0BNM9 | Glycolipid | 2 | 0.5 | −1 | 133.4 | 66.6 | cell organization and | cytosol | transporter activity; other |
| | | | | transfer protein | | | | | | biogenesis; transport | | molecular function |
| | | | | [OS = Rattus | | | | | | | | |
| | | | | norvegicus] | | | | | | | | |
| High | H1-1 | | D4A3K5 | Histone H1.1 | 7 | 0.495 | −1.01 | 133.8 | 66.2 | cell organization and | nucleus; other cell component | nucleic acid binding |
| | | | | [OS = Rattus | | | | | | biogenesis; other biological | | activity; other molecular |
| | | | | norvegicus] | | | | | | processes | | function |
| High | Qki | | Q91XU1 | Protein quaking | 4 | 0.496 | −1.01 | 133.7 | 66.3 | RNA metabolism OR | nucleus; other cell component | nucleic acid binding |
| | | | | [OS = Rattus | | | | | | transcription; other metabolic | | activity; other molecular |
| | | | | norvegicus] | | | | | | processes; transport; | | function |
| | | | | | | | | | | developmental processes; other | | |
| | | | | | | | | | | biological processes | | |
| High | Mcrip1 | | B0BN72 | Mapk-regulated | 2 | 0.497 | −1.01 | 133.6 | 66.4 | other biological processes | nucleus; other cell component | |
| | | | | corepressor- | | | | | | | | |
| | | | | interacting | | | | | | | | |
| | | | | protein 1 | | | | | | | | |
| | | | | [OS = Rattus | | | | | | | | |
| | | | | norvegicus] | | | | | | | | |
| High | Neo1 | | P97603 | Neogenin | 10 | 0.493 | −1.02 | 133.9 | 66.1 | cell adhesion; cell organization | plasma membrane; other | signal transduction activity |
| | | | | [OS = Rattus | | | | | | and biogenesis; transport; | membranes; other cell | or receptor binding; other |
| | | | | norvegicus | | | | | | developmental processes; other | component | molecular function |
| | | | | | | | | | | biological processes | | |
| High | Pnp | | P85973 | Purine | 7 | 0.493 | −1.02 | 134 | 66 | other metabolic | non-structural | other molecular function |
| | | | | nucleoside | | | | | | processes; other biological | extracellular; cytosol; nucleus; | |
| | | | | phosphorylase | | | | | | processes | other cell component | |
| | | | | [OS = Rattus | | | | | | | | |
| | | | | norvegicus] | | | | | | | | |
| High | Fkbp8 | | Q3B7U9 | Peptidyl-prolyl | 4 | 0.492 | −1.02 | 134 | 66 | developmental | other membranes; | other molecular function |
| | | | | cis-trans | | | | | | processes; signal | cytosol; mitochondrion; | |
| | | | | isomerase | | | | | | transduction; other biological | other cell component | |
| | | | | FKBP8 | | | | | | processes | | |
| | | | | [OS = Rattus | | | | | | | | |
| | | | | norvegicus] | | | | | | | | |
| High | Zfr | | Q562A2 | Zinc finger | 4 | 0.493 | −1.02 | 134 | 66 | | nucleus; other cell component | nucleic acid binding |
| | | | | RNA-binding | | | | | | | | activity; other molecular |
| | | | | protein | | | | | | | | function |
| | | | | [OS = Rattus | | | | | | | | |
| | | | | norvegicus | | | | | | | | |
| High | Map2 | | P15146 | Microtubule- | 74 | 0.49 | −1.03 | 134.2 | 65.8 | cell organization and | plasma membrane; other | cytoskeletal activity; other |
| | | | | associated | | | | | | biogenesis; developmental | membranes; cytoskeleton; ER/ | molecular function |
| | | | | protein 2 | | | | | | processes; other biological | Golgi; nucleus; other cell | |
| | | | | [OS = Rattus | | | | | | processes | component | |
| | | | | norvegicus] | | | | | | | | |
| High | Nmt1 | | Q8K1Q0 | Glycylpeptide | 5 | 0.489 | −1.03 | 134.3 | 65.7 | protein metabolism; other | other membranes; cytosol; other | other molecular function |
| | | | | N-tetradecanoyl- | | | | | | metabolic | cell component | |
| | | | | transferase 1 | | | | | | processes; developmental | | |
| | | | | [OS = Rattus | | | | | | processes | | |
| | | | | norvegicus] | | | | | | | | |
| High | Rps12 | | P63324 | 40S ribosomal | 5 | 0.491 | −1.03 | 134.2 | 65.8 | protein metabolism; other | cytosol; translational apparatus | other molecular function |
| | | | | protein S12 | | | | | | metabolic processes; other | | |
| | | | | [OS = Rattus | | | | | | biological processes | | |
| | | | | norvegicus] | | | | | | | | |
| High | Ss18l1 | | Q91XJ0 | Calcium- | 2 | 0.491 | −1.03 | 134.1 | 65.9 | cell organization and | nucleus; other cell component | other molecular function |
| | | | | responsive | | | | | | biogenesis; RNA metabolism | | |
| | | | | transcription | | | | | | OR | | |
| | | | | coactivator | | | | | | transcription; developmental | | |
| | | | | [OS = Rattus | | | | | | processes; other biological | | |
| | | | | norvegicus] | | | | | | processes | | |
| High | Serbp1 | | Q6AXS5 | Plasminogen | 11 | 0.485 | −1.04 | 134.7 | 65.3 | cell organization and | plasma membrane; other | translation activity; nucleic |
| | | | | activator | | | | | | biogenesis; other biological | membranes; nucleus; other cell | acid binding activity; other |
| | | | | inhibitor 1 | | | | | | processes | component | molecular function |
| | | | | RNA-binding | | | | | | | | |
| | | | | protein | | | | | | | | |
| | | | | [OS = Rattus | | | | | | | | |
| | | | | norvegicus] | | | | | | | | |
| High | Cspg4 | | Q00657 | Chondroitin | 2 | 0.486 | −1.04 | 134.6 | 65.4 | cell cycle OR cell | plasma membrane; other | other molecular function |
| | | | | sulfate | | | | | | proliferation; cell organization | membranes; other cell | |
| | | | | proteoglycan 4 | | | | | | and biogenesis; stress | component | |
| | | | | [OS = Rattus | | | | | | response; developmental | | |
| | | | | norvegicus] | | | | | | processes; signal | | |
| | | | | | | | | | | transduction; other biological | | |
| | | | | | | | | | | processes | | |
| High | Pi4kb | | O08561 | Phosphatidyl- | 2 | 0.487 | −1.04 | 134.5 | 65.5 | cell organization and | plasma membrane; other | kinase activity; other |
| | | | | inositol 4-kinase | | | | | | biogenesis; other metabolic | membranes; cytosol; | molecular function |
| | | | | beta | | | | | | processes; signal transduction | mitochondrion; ER/Golgi; other | |
| | | | | [OS = Rattus | | | | | | | cell component | |
| | | | | norvegicus] | | | | | | | | |
| High | Parp1 | | P27008 | Poly [ADP- | 5 | 0.481 | −1.05 | 135 | 65 | cell organization and | cytosol; mitochondrion; nucleus; | nucleic acid binding |
| | | | | ribose] | | | | | | biogenesis; protein | other cell component | activity; other molecular |
| | | | | polymerase 1 | | | | | | metabolism; DNA | | function |
| | | | | [OS = Rattus | | | | | | metabolism; other metabolic | | |
| | | | | norvegicus] | | | | | | processes; stress | | |
| | | | | | | | | | | response; signal | | |
| | | | | | | | | | | transduction; other biological | | |
| | | | | | | | | | | processes | | |
| High | Kars1 | | Q5XIM7 | Lysine--tRNA | 10 | 0.479 | −1.06 | 135.2 | 64.8 | protein metabolism; RNA | non-structural | nucleic acid binding |
| | | | | ligase | | | | | | metabolism OR | extracellular; plasma | activity; other molecular |
| | | | | [OS = Rattus | | | | | | transcription; other metabolic | membrane; other | function |
| | | | | norvegicus] | | | | | | processes; signal | membranes; cytosol; | |
| | | | | | | | | | | transduction; other biological | mitochondrion; nucleus; other | |
| | | | | | | | | | | processes | cell component | |
| High | Rnmt | | Q5U2U7 | mRNA cap | 8 | 0.479 | −1.06 | 135.2 | 64.8 | RNA metabolism OR | nucleus; other cell component | nucleic acid binding |
| | | | | guanine-N7 | | | | | | transcription; other metabolic | | activity; other molecular |
| | | | | methyltransferase | | | | | | processes; other biological | | function |
| | | | | [OS = Rattus | | | | | | processes | | |
| | | | | norvegicus] | | | | | | | | |
| High | Nlgn3 | | Q62889 | Neuroligin-3 | 6 | 0.476 | −1.07 | 135.5 | 64.5 | cell adhesion; cell-cell | plasma membrane; other | signal transduction activity |
| | | | | [OS = Rattus | | | | | | signaling; cell organization and | membranes; other cell | or receptor binding; other |
| | | | | norvegicus] | | | | | | biogenesis; transport; | component | molecular function |
| | | | | | | | | | | developmental processes; signal | | |
| | | | | | | | | | | transduction; other biological | | |
| | | | | | | | | | | processes | | |
| High | Aldoc | | P09117 | Fructose- | 16 | 0.474 | −1.08 | 135.7 | 64.3 | other metabolic | cytosol; other cell component | cytoskeletal activity; other |
| | | | | bisphosphate | | | | | | processes; stress response; other | | molecular function |
| | | | | aldolase C | | | | | | biological processes | | |
| | | | | [OS = Rattus | | | | | | | | |
| | | | | norvegicus] | | | | | | | | |
| High | Etfa | | P13803 | Electron | 8 | 0.473 | −1.08 | 135.8 | 64.2 | other metabolic processes | mitochondrion; other cell | other molecular function |
| | | | | transfer | | | | | | | component | |
| | | | | flavoprotein | | | | | | | | |
| | | | | subunit alpha, | | | | | | | | |
| | | | | mitochondrial | | | | | | | | |
| | | | | [OS = Rattus | | | | | | | | |
| | | | | norvegicus] | | | | | | | | |
| High | Ak4 | | Q9WUS0 | Adenylate | 4 | 0.472 | −1.08 | 135.9 | 64.1 | other metabolic | mitochondrion; other cell | kinase activity; other |
| | | | | kinase 4, | | | | | | processes; stress | component | molecular function |
| | | | | mitochondrial | | | | | | response; developmental | | |
| | | | | [OS = Rattus | | | | | | processes; other biological | | |
| | | | | norvegicus] | | | | | | processes | | |
| High | Qars1 | | Q66H61 | Glutamine-- | 6 | 0.467 | −1.1 | 136.3 | 63.7 | protein metabolism; RNA | cytosol; other cell component | enzyme regulator |
| | | | | tRNA ligase | | | | | | metabolism OR | | activity; other molecular |
| | | | | [OS = Rattus | | | | | | transcription; other metabolic | | function |
| | | | | norvegicus] | | | | | | processes; developmental | | |
| | | | | | | | | | | processes; other biological | | |
| | | | | | | | | | | processes | | |
| High | Pdlim5 | | Q62920 | PDZ and LIM | 6 | 0.464 | −1.11 | 136.6 | 63.4 | cell organization and | other | cytoskeletal activity; other |
| | | | | domain protein | | | | | | biogenesis; developmental | membranes; cytosol; cytoskeleton; | molecular function |
| | | | | 5 [OS = Rattus | | | | | | processes; other biological | other cell component | |
| | | | | norvegicus] | | | | | | processes | | |
| High | Hibadh | | P29266 | 3-hydroxy- | 4 | 0.464 | −1.11 | 136.6 | 63.4 | other metabolic processes | mitochondrion | other molecular function |
| | | | | isobutyrate | | | | | | | | |
| | | | | dehydrogenase, | | | | | | | | |
| | | | | mitochondrial | | | | | | | | |
| | | | | [OS = Rattus | | | | | | | | |
| | | | | norvegicus] | | | | | | | | |
| High | Gamt | | P10868 | Guanidinoacetate | 2 | 0.463 | −1.11 | 136.7 | 63.3 | other metabolic | nucleus; other cell component | other molecular function |
| | | | | N-methyl- | | | | | | processes; developmental | | |
| | | | | transferase | | | | | | processes; other biological | | |
| | | | | [OS = Rattus | | | | | | processes | | |
| | | | | norvegicus] | | | | | | | | |
| High | Snd1 | | Q66X93 | Staphylococcal | 16 | 0.458 | −1.13 | 137.2 | 62.8 | RNA metabolism OR | cytosol; nucleus; other cell | nucleic acid binding |
| | | | | nuclease domain- | | | | | | transcription; other metabolic | component | activity; other molecular |
| | | | | containing | | | | | | processes; other biological | | function |
| | | | | protein 1 | | | | | | processes | | |
| | | | | [OS = Rattus | | | | | | | | |
| | | | | norvegicus] | | | | | | | | |
| High | Pc | | P52873 | Pyruvate | 11 | 0.456 | −1.13 | 137.4 | 62.6 | other metabolic | mitochondrion; other cell | other molecular function |
| | | | | carboxylase, | | | | | | processes; other biological | component | |
| | | | | mitochondrial | | | | | | processes | | |
| | | | | [OS = Rattus | | | | | | | | |
| | | | | norvegicus] | | | | | | | | |
| High | Eef1d | | Q68FR9 | Elongation | 10 | 0.457 | −1.13 | 137.3 | 62.7 | protein metabolism; other | cytosol; nucleus; other cell | enzyme regulator |
| | | | | factor 1-delta | | | | | | metabolic processes; other | component | activity; nucleic acid binding |
| | | | | [OS = Rattus | | | | | | biological processes | | activity |
| | | | | norvegicus] | | | | | | | | |
| High | Ptbp2 | | Q66H20 | Polypyrimidine | 6 | 0.458 | −1.13 | 137.2 | 62.8 | cell organization and | nucleus; other cell component | nucleic acid binding activity |
| | | | | tract-binding | | | | | | biogenesis; RNA metabolism | | |
| | | | | protein 2 | | | | | | OR transcription; other | | |
| | | | | [OS = Rattus | | | | | | metabolic | | |
| | | | | norvegicus] | | | | | | processes; developmental | | |
| | | | | | | | | | | processes; other biological | | |
| | | | | | | | | | | processes | | |
| High | Ivd | | P12007 | Isovaleryl-CoA | 5 | 0.457 | −1.13 | 137.3 | 62.7 | other metabolic processes | other | other molecular function |
| | | | | dehydrogenase, | | | | | | | membranes; mitochondrion; | |
| | | | | mitochondrial | | | | | | | nucleus | |
| | | | | [OS = Rattus | | | | | | | | |
| | | | | norvegicus] | | | | | | | | |
| High | Pygb | | P53534 | Glycogen | 25 | 0.444 | −1.17 | 138.5 | 61.5 | other metabolic processes | other cell component | other molecular function |
| | | | | phosphorylase, | | | | | | | | |
| | | | | brain form | | | | | | | | |
| | | | | [OS = Rattus | | | | | | | | |
| | | | | norvegicus] | | | | | | | | |
| High | Camk2a | | P11275 | Calcium/ | 21 | 0.444 | −1.17 | 138.5 | 61.5 | cell cycle OR cell | plasma membrane; other | signal transduction activity |
| | | | | calmodulin- | | | | | | proliferation; cell organization | membranes; cytosol; | or receptor binding; kinase |
| | | | | dependent | | | | | | and biogenesis; protein | mitochondrion; nucleus; other | activity; other molecular |
| | | | | protein kinase | | | | | | metabolism; other metabolic | cell component | function |
| | | | | type II subunit | | | | | | processes; stress | | |
| | | | | alpha | | | | | | response; transport; | | |
| | | | | [OS = Rattus | | | | | | developmental processes; signal | | |
| | | | | norvegicus] | | | | | | transduction; other biological | | |
| | | | | | | | | | | processes | | |
| High | Ubxn4 | | Q5HZY0 | UBX domain- | 5 | 0.442 | −1.18 | 138.7 | 61.3 | protein metabolism; other | other | |
| | | | | containing | | | | | | metabolic processes; stress | membranes; ER/Golgi; nucleus | |
| | | | | protein 4 | | | | | | response | | |
| | | | | [OS = Rattus | | | | | | | | |
| | | | | norvegicus] | | | | | | | | |
| High | Slc32a1 | | O35458 | Vesicular | 7 | 0.439 | −1.19 | 139 | 61 | transport; developmental | other | transporter activity |
| | | | | inhibitory | | | | | | processes; other biological | membranes; ER/Golgi; other | |
| | | | | amino acid | | | | | | processes | cell component | |
| | | | | transporter | | | | | | | | |
| | | | | [OS = Rattus | | | | | | | | |
| | | | | norvegicus] | | | | | | | | |
| High | Irf2bpl | | Q5EIC4 | Probable E3 | 2 | 0.439 | −1.19 | 139 | 61 | protein metabolism; RNA | nucleus | other molecular function |
| | | | | ubiquitin- | | | | | | metabolism OR | | |
| | | | | protein ligase | | | | | | transcription; other metabolic | | |
| | | | | IRF2BPL | | | | | | processes; developmental | | |
| | | | | [OS = Rattus | | | | | | processes; other biological | | |
| | | | | norvegicus] | | | | | | processes | | |
| High | Gmpr | | Q9Z244 | GMP reductase | 3 | 0.435 | −1.2 | 139.4 | 60.6 | other metabolic | other cell component | other molecular function |
| | | | | 1 [OS = Rattus | | | | | | processes; stress response | | |
| | | | | norvegicus] | | | | | | | | |
| High | Mt3 | | P37361 | Metallothionein- | 3 | 0.435 | −1.2 | 139.4 | 60.6 | cell organization and | non-structural | enzyme regulator |
| | | | | 3 [OS-Rattus | | | | | | biogenesis; protein | extracellular; plasma | activity; other molecular |
| | | | | norvegicus] | | | | | | metabolism; other metabolic | membrane; other | function |
| | | | | | | | | | | processes; stress | membranes; cytoskeleton; | |
| | | | | | | | | | | response; transport; | mitochondrion; ER/Golgi; | |
| | | | | | | | | | | developmental processes; signal | translational apparatus; nucleus; | |
| | | | | | | | | | | transduction; other biological | other cell component | |
| | | | | | | | | | | processes | | |
| High | Rpl36a | | P83883 | 60S ribosomal | 2 | 0.435 | −1.2 | 139.4 | 60.6 | protein metabolism; other | cytosol; translational | other molecular function |
| | | | | protein L36a | | | | | | metabolic processes; other | apparatus; nucleus | |
| | | | | [OS = Rattus | | | | | | biological processes | | |
| | | | | norvegicus] | | | | | | | | |
| High | Rpl5 | | P09895 | 60S ribosomal | 8 | 0.433 | −1.21 | 139.6 | 60.4 | cell organization and | cytosol; translational | enzyme regulator |
| | | | | protein L5 | | | | | | biogenesis; protein | apparatus; nucleus; other cell | activity; nucleic acid binding |
| | | | | [OS = Rattus | | | | | | metabolism; RNA metabolism | component | activity; other molecular |
| | | | | norvegicus] | | | | | | OR transcription; other | | function |
| | | | | | | | | | | metabolic processes; other | | |
| | | | | | | | | | | biological processes | | |
| High | Acsl6 | | P33124 | Long-chain- | 8 | 0.43 | −1.22 | 139.9 | 60.1 | cell cycle OR cell | other | other molecular function |
| | | | | fatty-acid--CoA | | | | | | proliferation; other metabolic | membranes; mitochondrion; | |
| | | | | ligase 6 | | | | | | processes; stress | ER/Golgi; nucleus; other | |
| | | | | [OS = Rattus | | | | | | response; transport; | cytoplasmic organelle | |
| | | | | norvegicus] | | | | | | developmental processes; other | | |
| | | | | | | | | | | biological processes | | |
| High | Apex1 | | P43138 | DNA-(apurinic | 5 | 0.429 | −1.22 | 140 | 60 | cell organization and | cytoskeleton; mitochondrion; | nucleic acid binding |
| | | | | or apyrimidinic | | | | | | biogenesis; DNA | ER/Golgi; nucleus; other cell | activity; other molecular |
| | | | | site) | | | | | | metabolism; RNA metabolism | component | function |
| | | | | endonuclease | | | | | | OR transcription; other | | |
| | | | | [OS-Rattus | | | | | | metabolic processes; stress | | |
| | | | | norvegicus] | | | | | | response; other biological | | |
| | | | | | | | | | | processes | | |
| High | Eef1a2 | | P62632 | Elongation | 15 | 0.422 | −1.24 | 140.6 | 59.4 | protein metabolism; other | other cell component | nucleic acid binding |
| | | | | factor 1-alpha 2 | | | | | | metabolic processes; other | | activity; other molecular |
| | | | | [OS = Rattus | | | | | | biological processes | | function |
| | | | | norvegicus] | | | | | | | | |
| High | Rpl31 | | P62902 | 60S ribosomal | 6 | 0.425 | −1.24 | 140.4 | 59.6 | protein metabolism; other | cytosol; translational | other molecular function |
| | | | | protein L31 | | | | | | metabolic processes | apparatus; nucleus; other cell | |
| | | | | [OS = Rattus | | | | | | | component | |
| | | | | norvegicus] | | | | | | | | |
| High | Numbl | | A1L1I3 | Numb-like | 3 | 0.425 | −1.24 | 140.4 | 59.6 | cell cycle OR cell | other cell component | |
| | | | | protein | | | | | | proliferation; cell organization | | |
| | | | | [OS = Rattus | | | | | | and biogenesis; protein | | |
| | | | | norvegicus] | | | | | | metabolism; other metabolic | | |
| | | | | | | | | | | processes; developmental | | |
| | | | | | | | | | | processes; signal | | |
| | | | | | | | | | | transduction; other biological | | |
| | | | | | | | | | | processes | | |
| High | Khsrp | | Q99PF5 | Far upstream | 15 | 0.417 | −1.26 | 141.1 | 58.9 | RNA metabolism OR | nucleus; other cell component | nucleic acid binding activity |
| | | | | element- | | | | | | transcription; other metabolic | | |
| | | | | binding protein | | | | | | processes; transport; other | | |
| | | | | 2 [OS = Rattus | | | | | | biological processes | | |
| | | | | norvegicus] | | | | | | | | |
| High | Rpl23a | | P62752 | 60S ribosomal | 8 | 0.419 | −1.26 | 141 | 59 | cell organization and | cytosol; translational | nucleic acid binding |
| | | | | protein L23a | | | | | | biogenesis; protein | apparatus; nucleus | activity; other molecular |
| | | | | [OS = Rattus | | | | | | metabolism; other metabolic | | function |
| | | | | norvegicus] | | | | | | processes | | |
| High | Rps19 | | P17074 | 40S ribosomal | 6 | 0.418 | −1.26 | 141.1 | 58.9 | cell organization and | cytosol; translational | nucleic acid binding |
| | | | | protein S19 | | | | | | biogenesis; protein | apparatus; nucleus; other cell | activity; other molecular |
| | | | | [OS = Rattus | | | | | | metabolism; RNA metabolism | component | function |
| | | | | norvegicus] | | | | | | OR transcription; other | | |
| | | | | | | | | | | metabolic processes; stress | | |
| | | | | | | | | | | response; signal | | |
| | | | | | | | | | | transduction; other biological | | |
| | | | | | | | | | | processes | | |
| High | Nudt21 | | Q4KM65 | Cleavage and | 5 | 0.418 | −1.26 | 141 | 59 | cell organization and | cytoskeleton; nucleus; other cell | nucleic acid binding |
| | | | | polyadenylation | | | | | | biogenesis; RNA metabolism | component | activity; other molecular |
| | | | | specificity | | | | | | OR transcription; other | | function |
| | | | | factor subunit 5 | | | | | | metabolic processes; other | | |
| | | | | [OS = Rattus | | | | | | biological processes | | |
| | | | | norvegicus] | | | | | | | | |
| High | Strbp | | Q9JKU6 | Spermatid | 4 | 0.417 | −1.26 | 141.1 | 58.9 | other biological processes | cytoskeleton; other cell | cytoskeletal activity; nucleic |
| | | | | perinuclear | | | | | | | component | acid binding activity |
| | | | | RNA-binding | | | | | | | | |
| | | | | protein | | | | | | | | |
| | | | | [OS = Rattus | | | | | | | | |
| | | | | norvegicus] | | | | | | | | |
| High | Acads | | P15651 | Short-chain | 2 | 0.415 | −1.27 | 141.4 | 58.6 | other metabolic | other | other molecular function |
| | | | | specific acyl- | | | | | | processes; stress response; other | membranes; mitochondrion | |
| | | | | CoA | | | | | | biological processes | | |
| | | | | dehydrogenase, | | | | | | | | |
| | | | | mitochondrial | | | | | | | | |
| | | | | [OS = Rattus | | | | | | | | |
| | | | | norvegicus] | | | | | | | | |
| High | Edc4 | | Q3ZAV8 | Enhancer of | 6 | 0.413 | −1.28 | 141.6 | 58.4 | RNA metabolism OR | nucleus; other cell component | |
| | | | | mRNA- | | | | | | transcription; other metabolic | | |
| | | | | decapping | | | | | | processes | | |
| | | | | protein 4 | | | | | | | | |
| | | | | [OS = Rattus | | | | | | | | |
| | | | | norvegicus] | | | | | | | | |
| High | Gtf2i | | Q5U2Y1 | General | 5 | 0.409 | −1.29 | 141.9 | 58.1 | RNA metabolism OR | nucleus; other cell component | nucleic acid binding |
| | | | | transcription | | | | | | transcription; other metabolic | | activity; other molecular |
| | | | | factor II-I | | | | | | processes; other biological | | function |
| | | | | [OS = Rattus | | | | | | processes | | |
| | | | | norvegicus] | | | | | | | | |
| High | Cmas | | P69060 | N- | 7 | 0.405 | −1.3 | 142.3 | 57.7 | other metabolic processes | nucleus | other molecular function |
| | | | | acylneuraminate | | | | | | | | |
| | | | | cytidylyl- | | | | | | | | |
| | | | | transferase | | | | | | | | |
| | | | | [OS = Rattus | | | | | | | | |
| | | | | norvegicus] | | | | | | | | |
| High | Acadl | | P15650 | Long-chain | 6 | 0.406 | −1.3 | 142.3 | 57.7 | other metabolic | other | other molecular function |
| | | | | specific acyl- | | | | | | processes; stress response; other | membranes; mitochondrion; | |
| | | | | CoA | | | | | | biological processes | other cell component | |
| | | | | dehydrogenase, | | | | | | | | |
| | | | | mitochondrial | | | | | | | | |
| | | | | [OS = Rattus | | | | | | | | |
| | | | | norvegicus] | | | | | | | | |
| High | Gad2 | | Q05683 | Glutamate | 4 | 0.407 | −1.3 | 142.1 | 57.9 | cell-cell signaling; other | plasma membrane; other | other molecular function |
| | | | | decarboxylase 2 | | | | | | metabolic processes; other | membranes; cytosol; ER/Golgi; | |
| | | | | [OS = Rattus | | | | | | biological processes | other cell component | |
| | | | | norvegicus] | | | | | | | | |
| High | Pcp4 | | P63055 | Calmodulin | 4 | 0.403 | −1.31 | 142.6 | 57.4 | stress response; signal | cytoskeleton; other cell | nucleic acid binding |
| | | | | regulator | | | | | | transduction; other biological | component | activity; other molecular |
| | | | | protein PCP4 | | | | | | processes | | function |
| | | | | [OS = Rattus | | | | | | | | |
| | | | | norvegicus] | | | | | | | | |
| High | Cplx2 | | P84087 | Complexin-2 | 4 | 0.398 | −1.33 | 143.1 | 56.9 | cell-cell | other | other molecular function |
| | | | | [OS = Rattus | | | | | | signaling; transport; other | membranes; cytosol; nucleus; | |
| | | | | norvegicus] | | | | | | biological processes | other cell component | |
| High | Scd2 | | Q6P7B9 | Stearoyl-CoA | 4 | 0.392 | −1.35 | 143.6 | 56.4 | other metabolic | other membranes; ER/Golgi | other molecular function |
| | | | | desaturase 2 | | | | | | processes; developmental | | |
| | | | | [OS = Rattus | | | | | | processes; other biological | | |
| | | | | norvegicus] | | | | | | processes | | |
| High | Farsa | | Q505J8 | Phenylalanine-- | 11 | 0.388 | −1.37 | 144.1 | 55.9 | cell organization and | other cell component | nucleic acid binding |
| | | | | tRNA ligase | | | | | | biogenesis; protein | | activity; other molecular |
| | | | | alpha subunit | | | | | | metabolism; RNA metabolism | | function |
| | | | | [OS = Rattus | | | | | | OR transcription; other | | |
| | | | | norvegicus] | | | | | | metabolic processes | | |
| High | Gad1 | | P18088 | Glutamate | 8 | 0.387 | −1.37 | 144.2 | 55.8 | protein metabolism; other | other cell component | other molecular function |
| | | | | decarboxylase 1 | | | | | | metabolic processes; other | | |
| | | | | [OS = Rattus | | | | | | biological processes | | |
| | | | | norvegicus] | | | | | | | | |
| High | Cd59 | | P27274 | CD59 | 2 | 0.386 | −1.37 | 144.3 | 55.7 | other biological processes | non-structural | other molecular function |
| | | | | glycoprotein | | | | | | | extracellular; plasma | |
| | | | | [OS = Rattus | | | | | | | membrane; other | |
| | | | | norvegicus] | | | | | | | membranes; other cell | |
| | | | | | | | | | | | component | |
| High | Ndrg2 | | Q8VBU2 | Protein NDRG2 | 4 | 0.383 | −1.38 | 144.6 | 55.4 | cell-cell signaling; cell cycle | nucleus; other cell component | |
| | | | | [OS = Rattus | | | | | | OR cell proliferation; cell | | |
| | | | | norvegicus] | | | | | | organization and | | |
| | | | | | | | | | | biogenesis; developmental | | |
| | | | | | | | | | | processes; signal | | |
| | | | | | | | | | | transduction; other biological | | |
| | | | | | | | | | | processes | | |
| High | Purb | | Q68A21 | Transcriptional | 8 | 0.38 | −1.39 | 144.9 | 55.1 | cell cycle OR cell | nucleus | nucleic acid binding |
| | | | | activator | | | | | | proliferation; RNA metabolism | | activity; other molecular |
| | | | | protein Pur-beta | | | | | | OR transcription; other | | function |
| | | | | [OS = Rattus | | | | | | biological processes | | |
| | | | | norvegicus] | | | | | | | | |
| High | Mat2a | | P18298 | S-adenosyl- | 7 | 0.382 | −1.39 | 144.7 | 55.3 | cell organization and | cytosol; other cell component | other molecular function |
| | | | | methionine | | | | | | biogenesis; other metabolic | | |
| | | | | synthase | | | | | | processes; other biological | | |
| | | | | isoform type-2 | | | | | | processes | | |
| | | | | [OS = Rattus | | | | | | | | |
| | | | | norvegicus] | | | | | | | | |
| High | Cpt1a | | P32198 | Carnitine O- | 4 | 0.381 | −1.39 | 144.9 | 55.1 | other metabolic | other | other molecular function |
| | | | | palmitoyl- | | | | | | processes; developmental | membranes; mitochondrion; | |
| | | | | transferase | | | | | | processes; other biological | other cell component | |
| | | | | 1, liver | | | | | | processes | | |
| | | | | isoform | | | | | | | | |
| | | | | [OS = Rattus | | | | | | | | |
| | | | | norvegicus] | | | | | | | | |
| High | Coro7 | | O35828 | Coronin-7 | 3 | 0.383 | −1.39 | 144.6 | 55.4 | cell organization and | other | cytoskeletal activity |
| | | | | [OS = Rattus | | | | | | biogenesis; transport; other | membranes; cytosol; ER/Golgi; | |
| | | | | norvegicus] | | | | | | biological processes | other cell component | |
| High | Acsf2 | | Q499N5 | Medium-chain | 5 | 0.377 | −1.41 | 145.2 | 54.8 | other metabolic processes | mitochondrion | other molecular function |
| | | | | acyl-CoA ligase | | | | | | | | |
| | | | | ACSF2, | | | | | | | | |
| | | | | mitochondrial | | | | | | | | |
| | | | | [OS = Rattus | | | | | | | | |
| | | | | norvegicus] | | | | | | | | |
| High | Asrgl1 | | Q8VI04 | Isoaspartyl | 10 | 0.373 | −1.42 | 145.6 | 54.4 | protein metabolism; other | other cell component | other molecular function |
| | | | | peptidase/L- | | | | | | metabolic processes | | |
| | | | | asparaginase | | | | | | | | |
| | | | | [OS = Rattus | | | | | | | | |
| | | | | norvegicus] | | | | | | | | |
| High | Rps24 | | P62850 | 40S ribosomal | 6 | 0.374 | −1.42 | 145.6 | 54.4 | protein metabolism; RNA | cytosol; translational apparatus | other molecular function |
| | | | | protein S24 | | | | | | metabolism OR | | |
| | | | | [OS = Rattus | | | | | | transcription; other metabolic | | |
| | | | | norvegicus] | | | | | | processes; developmental | | |
| | | | | | | | | | | processes; other biological | | |
| | | | | | | | | | | processes | | |
| High | Rpl14 | | Q63507 | 60S ribosomal | 6 | 0.368 | −1.44 | 146.2 | 53.8 | protein metabolism; RNA | cytosol; translational | nucleic acid binding |
| | | | | protein L14 | | | | | | metabolism OR | apparatus; other cell component | activity; other molecular |
| | | | | [OS = Rattus | | | | | | transcription; other metabolic | | function |
| | | | | norvegicus] | | | | | | processes; other biological | | |
| | | | | | | | | | | processes | | |
| High | Mybbp1a | | O35821 | Myb-binding | 10 | 0.367 | −1.45 | 146.4 | 53.6 | RNA metabolism OR | nucleus; other cell component | nucleic acid binding |
| | | | | protein 1A | | | | | | transcription; other metabolic | | activity; other molecular |
| | | | | [OS = Rattus | | | | | | processes; stress | | function |
| | | | | norvegicus] | | | | | | response; signal | | |
| | | | | | | | | | | transduction; other biological | | |
| | | | | | | | | | | processes | | |
| High | Ak3 | | P29411 | GTP:AMP | 2 | 0.365 | −1.46 | 146.6 | 53.4 | other metabolic | mitochondrion; other cell | kinase activity; other |
| | | | | phospho- | | | | | | processes; stress | component | molecular function |
| | | | | transferase AK3, | | | | | | response; developmental | | |
| | | | | mitochondrial | | | | | | processes; other biological | | |
| | | | | [OS = Rattus | | | | | | processes | | |
| | | | | norvegicus] | | | | | | | | |
| High | Synrg | | Q9JKC9 | Synergin | 2 | 0.363 | −1.46 | 146.8 | 53.2 | transport | other | |
| | | | | gamma | | | | | | | membranes; ER/Golgi; other | |
| | | | | [OS = Rattus | | | | | | | cell component | |
| | | | | norvegicus] | | | | | | | | |
| High | Hcfc1 | | D3ZN95 | Host cell factor | 13 | 0.351 | −1.51 | 148 | 52 | cell cycle OR cell | nucleus; other cell component | nucleic acid binding |
| | | | | 1 [OS = Rattus | | | | | | proliferation; cell organization | | activity; other molecular |
| | | | | norvegicus] | | | | | | and biogenesis; protein | | function |
| | | | | | | | | | | metabolism; RNA metabolism | | |
| | | | | | | | | | | OR transcription; other | | |
| | | | | | | | | | | metabolic | | |
| | | | | | | | | | | processes; developmental | | |
| | | | | | | | | | | processes; other biological | | |
| | | | | | | | | | | processes | | |
| High | Pip5k1C | | Q5I6B8 | Phosphatidyl- | 5 | 0.351 | −1.51 | 148.1 | 51.9 | cell adhesion; cell organization | plasma membrane; other | kinase activity; other |
| | | | | inositol 4- | | | | | | and biogenesis; other metabolic | membranes; cytosol; other | molecular function |
| | | | | phosphate 5- | | | | | | processes; stress | cytoplasmic organelle; other | |
| | | | | kinase type-1 | | | | | | response; transport; | cell component | |
| | | | | gamma | | | | | | developmental processes; other | | |
| | | | | [OS = Rattus | | | | | | biological processes | | |
| | | | | norvegicus] | | | | | | | | |
| High | Tmpo | | Q62733 | Lamina- | 7 | 0.349 | −1.52 | 148.2 | 51.8 | cell organization and | other | nucleic acid binding |
| | | | | associated | | | | | | biogenesis; other biological | membranes; nucleus; other cell | activity; other molecular |
| | | | | polypeptide 2, | | | | | | processes | component | function |
| | | | | isoform beta | | | | | | | | |
| | | | | [OS-Rattus | | | | | | | | |
| | | | | norvegicus] | | | | | | | | |
| High | Cnp | | P13233 | 2′,3′-cyclic- | 8 | 0.339 | −1.56 | 149.3 | 50.7 | cell organization and | non-structural | nucleic acid binding |
| | | | | nucleotide 3′- | | | | | | biogenesis; other metabolic | extracellular; plasma | activity; other molecular |
| | | | | phosphodiesterase | | | | | | processes; transport; | membrane; other | function |
| | | | | [OS = Rattus | | | | | | developmental processes; other | membranes; mitochondrion; | |
| | | | | norvegicus] | | | | | | biological processes | other cell component | |
| High | Cbr3 | | B2GV72 | Carbonyl | 4 | 0.339 | −1.56 | 149.4 | 50.6 | other metabolic | cytosol | other molecular function |
| | | | | reductase | | | | | | processes; other biological | | |
| | | | | [NADPH] 3 | | | | | | processes | | |
| | | | | [OS = Rattus | | | | | | | | |
| | | | | norvegicus] | | | | | | | | |
| High | Akap2 | | Q5U301 | A-kinase | 3 | 0.338 | −1.57 | 149.5 | 50.5 | cell organization and | other cell component | other molecular function |
| | | | | anchor protein | | | | | | biogenesis; signal | | |
| | | | | 2 [OS = Rattus | | | | | | transduction; other biological | | |
| | | | | norvegicus] | | | | | | processes | | |
| High | Hdgfl3 | | Q923W4 | Hepatoma- | 6 | 0.334 | −1.58 | 149.9 | 50.1 | cell organization and | cytosol; nucleus; other cell | signal transduction activity |
| | | | | derived growth | | | | | | biogenesis; RNA metabolism | component | or receptor |
| | | | | factor-related | | | | | | OR | | binding; cytoskeletal |
| | | | | protein 3 | | | | | | transcription; developmental | | activity; nucleic acid binding |
| | | | | [OS = Rattus | | | | | | processes; signal | | activity; other molecular |
| | | | | norvegicus] | | | | | | transduction; other biological | | function |
| | | | | | | | | | | processes | | |
| High | Rps21 | | P05765 | 40S ribosomal | 3 | 0.33 | −1.6 | 150.3 | 49.7 | protein metabolism; RNA | cytosol; ER/Golgi; translational | translation activity; other |
| | | | | protein S21 | | | | | | metabolism OR | apparatus | molecular function |
| | | | | [OS-Rattus | | | | | | transcription; other metabolic | | |
| | | | | norvegicus] | | | | | | processes | | |
| High | Nup98 | | P49793 | Nuclear pore | 2 | 0.327 | −1.61 | 150.7 | 49.3 | cell organization and | other | nucleic acid binding |
| | | | | complex | | | | | | biogenesis; protein | membranes; nucleus; other cell | activity; other molecular |
| | | | | protein Nup98- | | | | | | metabolism; RNA metabolism | component | function |
| | | | | Nup96 | | | | | | OR | | |
| | | | | [OS = Rattus | | | | | | transcription; transport; other | | |
| | | | | norvegicus] | | | | | | biological processes | | |
| High | Tppp | | D3ZQL7 | Tubulin | 6 | 0.325 | −1.62 | 151 | 49 | cell cycle OR cell | cytoskeleton; ER/Golgi; nucleus; | cytoskeletal activity; other |
| | | | | polymerization- | | | | | | proliferation; cell organization | other cell component | molecular function |
| | | | | promoting | | | | | | and biogenesis; developmental | | |
| | | | | protein | | | | | | processes; other biological | | |
| | | | | [OS = Rattus | | | | | | processes | | |
| | | | | norvegicus] | | | | | | | | |
| High | Spon1 | | P35446 | Spondin-1 | 3 | 0.322 | −1.63 | 151.2 | 48.8 | cell adhesion; protein | non-structural | other molecular function |
| | | | | [OS = Rattus | | | | | | metabolism; other metabolic | extracellular; extracellular | |
| | | | | norvegicus] | | | | | | processes; other biological | matrix | |
| | | | | | | | | | | processes | | |
| High | Aak1 | | P0C1X8 | AP2-associated | 9 | 0.32 | −1.64 | 151.5 | 48.5 | protein metabolism; other | plasma membrane; other | signal transduction activity |
| | | | | protein kinase 1 | | | | | | metabolic | membranes; other cell | or receptor binding; kinase |
| | | | | [OS = Rattus | | | | | | processes; transport; other | component | activity; other molecular |
| | | | | norvegicus] | | | | | | biological processes | | function |
| High | Anp32b | | Q9EST6 | Acidic leucine- | 5 | 0.318 | −1.65 | 151.7 | 48.3 | cell organization and | nucleus; other cell component | other molecular function |
| | | | | rich nuclear | | | | | | biogenesis; developmental | | |
| | | | | phosphoprotein | | | | | | processes; other biological | | |
| | | | | 32 family | | | | | | processes | | |
| | | | | member B | | | | | | | | |
| | | | | [OS = Rattus | | | | | | | | |
| | | | | norvegicus] | | | | | | | | |
| High | Arfgap1 | | Q62848 | ADP- | 2 | 0.319 | −1.65 | 151.7 | 48.3 | transport; other biological | other | enzyme regulator |
| | | | | ribosylation | | | | | | processes | membranes; ER/Golgi; other | activity; other molecular |
| | | | | factor GTPase- | | | | | | | cell component | function |
| | | | | activating | | | | | | | | |
| | | | | protein 1 | | | | | | | | |
| | | | | [OS = Rattus | | | | | | | | |
| | | | | norvegicus] | | | | | | | | |
| High | Serpine2 | | P07092 | Glia-derived | 5 | 0.315 | −1.67 | 152.1 | 47.9 | cell organization and | non-structural | signal transduction activity |
| | | | | nexin | | | | | | biogenesis; protein | extracellular; extracellular | or receptor binding; enzyme |
| | | | | [OS = Rattus | | | | | | metabolism; other metabolic | matrix; plasma membrane; other | regulator activity; other |
| | | | | norvegicus] | | | | | | processes; stress | membranes; cytosol; other cell | molecular function |
| | | | | | | | | | | response; transport; | component | |
| | | | | | | | | | | developmental processes; other | | |
| | | | | | | | | | | biological processes | | |
| High | Scp2 | | P11915 | Sterol carrier | 9 | 0.305 | −1.71 | 153.3 | 46.7 | cell organization and | other membranes; | signal transduction activity |
| | | | | protein 2 | | | | | | biogenesis; other metabolic | cytosol; mitochondrion; | or receptor |
| | | | | [OS = Rattus | | | | | | processes; transport; other | ER/Golgi; nucleus; other | binding; transporter |
| | | | | norvegicus] | | | | | | biological processes | cytoplasmic organelle; other | activity; other molecular |
| | | | | | | | | | | | cell component | function |
| High | Crtc1 | | Q157S1 | CREB- | 2 | 0.296 | −1.75 | 154.3 | 45.7 | cell-cell signaling; cell | plasma membrane; other | other molecular function |
| | | | | regulated | | | | | | organization and | membranes; cytosol; nucleus; | |
| | | | | transcription | | | | | | biogenesis; signal | other cell component | |
| | | | | coactivator 1 | | | | | | transduction; other biological | | |
| | | | | [OS = Rattus | | | | | | processes | | |
| | | | | norvegicus] | | | | | | | | |
| High | Phgdh | | O08651 | D-3- | 14 | 0.288 | −1.79 | 155.2 | 44.8 | cell cycle OR cell | | other molecular function |
| | | | | phosphoglycerate | | | | | | proliferation; cell organization | | |
| | | | | dehydrogenase | | | | | | and biogenesis; other metabolic | | |
| | | | | [OS = Rattus | | | | | | processes; developmental | | |
| | | | | norvegicus] | | | | | | processes; other biological | | |
| | | | | | | | | | | processes | | |
| High | Nup35 | | Q68FY1 | Nucleoporin | 3 | 0.283 | −1.82 | 155.9 | 44.1 | cell organization and | other membranes; nucleus | nucleic acid binding |
| | | | | NUP35 | | | | | | biogenesis; transport; other | | activity; other molecular |
| | | | | [OS = Rattus | | | | | | biological processes | | function |
| | | | | norvegicus] | | | | | | | | |
| High | Decr1 | | Q64591 | 2,4-dienoyl- | 3 | 0.268 | −1.9 | 157.7 | 42.3 | other metabolic | mitochondrion; other cell | other molecular function |
| | | | | CoA reductase | | | | | | processes; other biological | component | |
| | | | | [(3E)-enoyl- | | | | | | processes | | |
| | | | | CoA- | | | | | | | | |
| | | | | producing], | | | | | | | | |
| | | | | mitochondrial | | | | | | | | |
| | | | | [OS = Rattus | | | | | | | | |
| | | | | norvegicus] | | | | | | | | |
| High | Rbmxrtl | | P84586 | RNA-binding | 9 | 0.266 | −1.91 | 158 | 42 | cell organization and | non-structural | nucleic acid binding |
| | | | | motif protein, X | | | | | | biogenesis; protein | extracellular; nucleus; other cell | activity; other molecular |
| | | | | chromosome | | | | | | metabolism; RNA metabolism | component | function |
| | | | | retrogene-like | | | | | | OR transcription; other | | |
| | | | | [OS = Rattus | | | | | | metabolic processes; other | | |
| | | | | norvegicus] | | | | | | biological processes | | |
| High | Tst | | P24329 | Thiosulfate | 4 | 0.261 | −1.94 | 158.6 | 41.4 | cell organization and | mitochondrion | nucleic acid binding |
| | | | | sulfurtransferase | | | | | | biogenesis; other metabolic | | activity; other molecular |
| | | | | [OS = Rattus | | | | | | processes; transport; other | | function |
| | | | | norvegicus] | | | | | | biological processes | | |
| High | Pspc1 | | Q4KLH4 | Paraspeckle | 8 | 0.257 | −1.96 | 159.1 | 40.9 | stress response; other biological | nucleus; other cell component | nucleic acid binding activity |
| | | | | component 1 | | | | | | processes | | |
| | | | | [OS = Rattus | | | | | | | | |
| | | | | norvegicus] | | | | | | | | |
| High | Fabp7 | | P55051 | Fatty acid- | 8 | 0.247 | −2.02 | 160.4 | 39.6 | cell cycle OR cell | cytosol; nucleus; other cell | other molecular function |
| | | | | binding protein, | | | | | | proliferation; transport; | component | |
| | | | | brain | | | | | | developmental processes; other | | |
| | | | | [OS = Rattus | | | | | | biological processes | | |
| | | | | norvegicus] | | | | | | | | |
| High | Ensa | | P60841 | Alpha- | 5 | 0.245 | −2.03 | 160.7 | 39.3 | cell cycle OR cell | other cell component | signal transduction activity |
| | | | | endosulfine | | | | | | proliferation; other biological | | or receptor binding; enzyme |
| | | | | [OS-Rattus | | | | | | processes | | regulator activity; other |
| | | | | norvegicus] | | | | | | | | molecular function |
| High | Glul | | P09606 | Glutamine | 10 | 0.243 | −2.04 | 160.8 | 39.2 | cell cycle OR cell | plasma membrane; other | other molecular function |
| | | | | synthetase | | | | | | proliferation; protein | membranes; cytosol; | |
| | | | | [OS = Rattus | | | | | | metabolism; other metabolic | mitochondrion; ER/Golgi; | |
| | | | | norvegicus] | | | | | | processes; stress | other cell component | |
| | | | | | | | | | | response; developmental | | |
| | | | | | | | | | | processes; other biological | | |
| | | | | | | | | | | processes | | |
| High | Dbi | | P11030 | Acyl-CoA- | 4 | 0.237 | −2.08 | 161.7 | 38.3 | cell cycle OR cell | non-structural | signal transduction activity |
| | | | | binding protein | | | | | | proliferation; other metabolic | extracellular; plasma | or receptor binding; other |
| | | | | [OS = Rattus | | | | | | processes; stress | membrane; other | molecular function |
| | | | | norvegicus] | | | | | | response; transport; | membranes; cytoskeleton; | |
| | | | | | | | | | | developmental processes; other | ER/Golgi; nucleus; | |
| | | | | | | | | | | biological processes | other cell component | |
| High | Sarnp | | Q498U4 | SAP domain- | 4 | 0.225 | −2.15 | 163.3 | 36.7 | other metabolic | nucleus | nucleic acid binding activity |
| | | | | containing | | | | | | processes; transport; other | | |
| | | | | ribonucleo- | | | | | | biological processes | | |
| | | | | protein | | | | | | | | |
| | | | | [OS = Rattus | | | | | | | | |
| | | | | norvegicus] | | | | | | | | |
| High | Nrgn | | Q04940 | Neurogranin | 2 | 0.212 | −2.24 | 165 | 35 | developmental processes; other | plasma membrane; other | other molecular function |
| | | | | [OS = Rattus | | | | | | biological processes | membranes; cytosol; | |
| | | | | norvegicus] | | | | | | | mitochondrion; ER/Golgi; | |
| | | | | | | | | | | | nucleus; other cell component | |
| High | Pdgfra | | P20786 | Platelet-derived | 3 | 0.185 | −2.44 | 168.8 | 31.2 | cell adhesion; cell organization | plasma membrane; other | signal transduction activity |
| | | | | growth factor | | | | | | and biogenesis; protein | membranes; ER/Golgi; nucleus; | or receptor binding; kinase |
| | | | | receptor alpha | | | | | | metabolism; other metabolic | other cell component | activity; other molecular |
| | | | | [OS-Rattus | | | | | | processes; stress | | function |
| | | | | norvegicus] | | | | | | response; developmental | | |
| | | | | | | | | | | processes; signal | | |
| | | | | | | | | | | transduction; other biological | | |
| | | | | | | | | | | processes | | |
| High | Hdac4 | | Q99P99 | Histone | 2 | 0.159 | −2.65 | 172.6 | 27.4 | cell cycle OR cell | cytosol; cytoskeleton; nucleus; | nucleic acid binding |
| | | | | deacetylase 4 | | | | | | proliferation; cell organization | other cell component | activity; other molecular |
| | | | | [OS = Rattus | | | | | | and biogenesis; protein | | function |
| | | | | norvegicus] | | | | | | metabolism; RNA metabolism | | |
| | | | | | | | | | | OR transcription; other | | |
| | | | | | | | | | | metabolic | | |
| | | | | | | | | | | processes; developmental | | |
| | | | | | | | | | | processes; signal | | |
| | | | | | | | | | | transduction; other biological | | |
| | | | | | | | | | | processes | | |

### Example 4a: Using an Induced Pluripotent Stem Cell (iPSC) Model to Recapitulate ALS Pathology

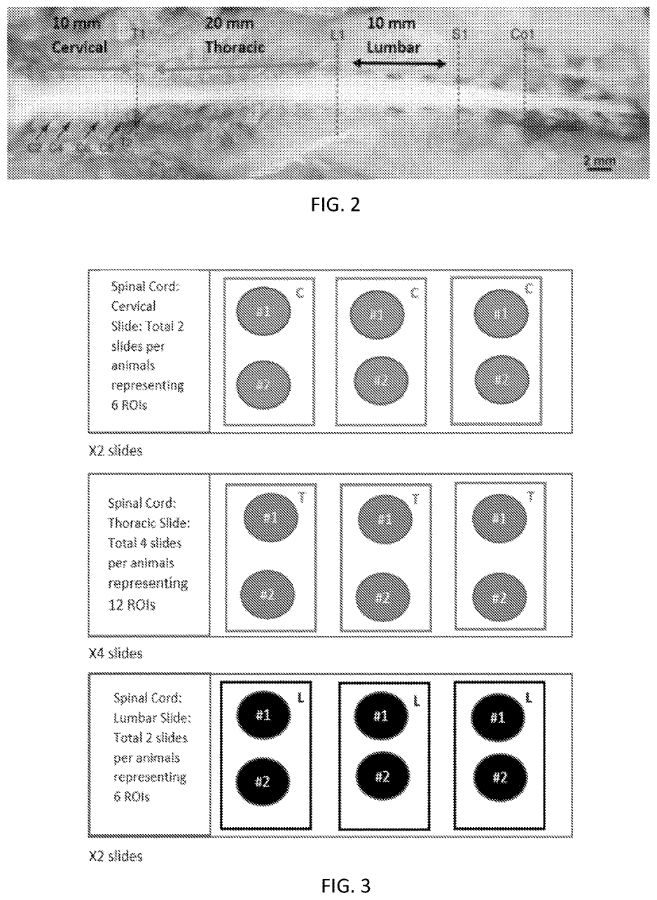

ALS and control iPSC-derived motor neurons were generated as described elsewhere, with some modifications. See, for example, Du et al., Nat Commun. 2015; 6:6626. Neurons were be treated as describe in FIG. 7 with trehalose in solution. At day 35 of culture, ALS iPSC motor neurons exhibited typical ALS pathology, including increased motor neuron death, TDP-43 mislocalisation, and altered mitochondrial networks when compared to control iPSC motor neurons (FIG. 12B-12C). Treatment of ALS iPSC-derived motor neurons with unmodified trehalose neuronal connectivity, as shown by the β3-tublin staining. (FIG. 12D).

### Example 4b: Identifying Mechanisms of Neuroprotection by In Vitro Trehalose in an iPSC System

To identify mechanisms that underpin neuroprotection by in vitro trehalose in an ALS mouse model, induced pluripotent stem cell (iPSC) motor neuron survival and morphology, oxidative stress, autophagy, mitochondrial function, and TDP-43 accumulation upon treatment with pBK1928-AAV9-otsA-P2A-otsB (with or without riluzole) and vehicle control will be assessed. ALS and control iPSC-derived motor neurons will be generated as described elsewhere, with some modifications. See, for example, Du et al., Nat Commun. 2015; 6:6626. Neurons will be treated as describe in FIG. 12E.

Methods: Motor neuron survival, morphology, and TDP-43: ALS and control iPSC-derived motor neurons will be stained for ChAT (choline acetyltransferase), TDP-43, and b-tubulin at day 35 of culture. Neurons will be imaged using an Operetta® high-content imaging system (PerkinElmer). Harmony® high-content analysis software will be used to determine the number of ChAT positive motor neurons present in cultures treated with the lead formulation (with or without riluzole), and vehicle control. TDP-43 localization will be quantified in the nucleus and cytoplasm. Assessment of neuronal morphology will be quantified by evaluating b-tubulin. ALS pathology: Parallel cultures of control and ALS iPSC-derived motor neurons will be grown to day 30. Autophagy will be assessed (LC3II relative to LC3I) using Immunoblot or Immunofluorescence, oxidative stress using dihydroethidium/dichlorodihydrofluorescein diacetate or MitoSox, mitochondrial function using Pheno Vue to assess fission-fusion dynamics, size and shape, and tetramethylrhodamine, methyl ester (TMRM)/tetraethylbenzimidazolylcarbocyanine iodide (JC-1) to assess mitochondrial membrane potential. Parameters will be used to determine efficacy of the lead formulation of trehalose on attenuating these cellular features in ALS iPSC-derived motor neurons relative to control iPSC-derived motor neurons.

### Example 4c: Pre-Clinical Validation of AAV Trehalose for Treating ALS Using Mouse Models

Multiple mouse models of ALS (SOD1G93A (Jackson Lab; stock number: 004435; (20)) and TDP-43rNLS (Jackson Lab; strain: 028412; (21), backcrossed at UQ onto C56/B16J) will be used to assess the effect of pBK1928-AAV9-otsA-P2A-otsB. These mice share the same background, exhibit ALS phenotypes and pathologies (i.e. muscle weakness, neuromuscular denervation, motor neuron loss, protein aggregates), and have different disease course yet an overall lifespan that is suited for the achievement of project goals within the 3-year timeline.

Data obtained from these models will provide insights for fALS (SOD1), but also sALS; TDP-43rNLS mice have TDP-43 protein aggregates, which a histopathological feature is observed in ~97% of all human ALS cases. See, for example, Scotter et al. TDP-43 Proteinopathy and ALS: Insights into Disease Mechanisms and Therapeutic Targets. Neurotherapeutics. 2015; 12(2):352-63. Experiments are designed to use the minimum number of animals required for reliable statistical analyses. Studies in mice will follow principles for rigorous study design and reporting including; size estimation, randomization, sex balance, and blinded conduct.

SOD1G93A and TDP-43rNLS mice will be treated with a dosing approach of pBK1928-AAV9-otsA-P2A-otsB to evaluate disease progression and survival. Litter- and sex-matched ALS mice will be treated with the pBK1928-AAV9-otsA-P2A-otsB (with or without riluzole) when each transgenic model shows early signs of hind limb tremors and weakness (SOD1G93A mice: 11 wks; TDP-43rNLS mice: 2 wks off doxycycline), and compared to vehicle treated mice (N=12 per group/sex). Body weight and disease progression will be monitored until death assessing the effect of trehalose on disease progression and survival in SOD1 mice. A parallel group of mice treated with the AAV-SLS-005 or vehicle control (N=12 per group/sex), and subsequently evaluated at the mid-symptomatic stage (SOD1G93A=17 wks; TDP-43rNLS mice: 4 wks off doxycycline) to assess the effect of the lead formulation on pathological features of disease including, autophagy, oxidative stress, protein accumulation, motor neuron survival, and neuromuscular innervation.

Methods: Animals: SOD1G93A and TDP-43rNLS mice. In vivo treatment: AAV-SLS-005 will be administered to animals at a dose strategy established through finalized PK/PD data. Behavior analysis: Transgenic mice and controls will be monitored weekly by a single experimenter in a blinded fashion until survival endpoint. Body weight, motor performance (i.e. grip strength, hanging wire test), and survival will be evaluated as described elsewhere. See, for example, Castillo et al., Autophagy. 2013; 9(9):1308-1320; Zhang et al., Autophagy. 2014; 10(4):588-602; or Scaricamazza et al., Br. J. Pharmacol. 2022; 179(8):1732-1752. Neurological scores will be assess as described elsewhere. See, for example, Hatzipetros et al., J. Vis. Exp. 2015(104). The onset of disease will be determined as the time at which the onset of hind limb weakness first manifests in each transgenic mouse strain. The onset of hind limb force motor deficit will be defined as the time at which mice show the first signs of impairment on the grip strength test and/or hanging wire test. For survival, disease end stage will be defined as the age at which animals are unable to right themselves within ten seconds after being placed on each side. Age in days at the end stage time point will be recorded as the survival duration for each animal. Disease duration will be calculated as the difference in days between the onset of motor deficit and the end stage/survival endpoint for each animal. Tissue/sample analysis: SOD1G93A and TDP-43rNLS nice will be treated until midsymptomatic stage of disease (SOD1G93A; 17 weeks, TDP-43rNLS; 4 weeks off dox). Pathological features, including autophagy, oxidative stress, protein accumulation, motor neuron survival, and neuromuscular innervation will be evaluated through Immunoblot, Immunohistochemistry, or ELISA as described elsewhere. See, for example, Castillo et al., Autophagy. 2013; 9(9):1308-1320; Zhang et al., Autophagy. 2014; 10(4):588-602; Scaricamazza et al., Br. J. Pharmacol. 2022; 179(8):1732-1752; or Ngo et al., Clin. Neurophysiol. 2012; 123(8):2080-91. Levels of oxidative stress will be determined by assessing urinary 8-Hydroxydeoxyguanosine (8-OHdG) using a commercial ELISA. Levels of neurofilament will be measured as in WP1 by the Quanterix Simoa™ NF-Light Advantage Kit according to manufacture's instructions.

### Example 5: Testing Efficacy of AAV Trehalose in R6/2 Mouse Model of Huntington's Disease

The objective of this study was to assess the effects of intrastriatal infusion of AAV1-OtsAB-mCherry and AAV9-CAG-otsA-P2A-otsB-T2A-mCherry-WPRE (7.4×10¹² vector genomes (vg) per ml), (pBK1802), in the R6/2 mouse model of Huntington's disease.

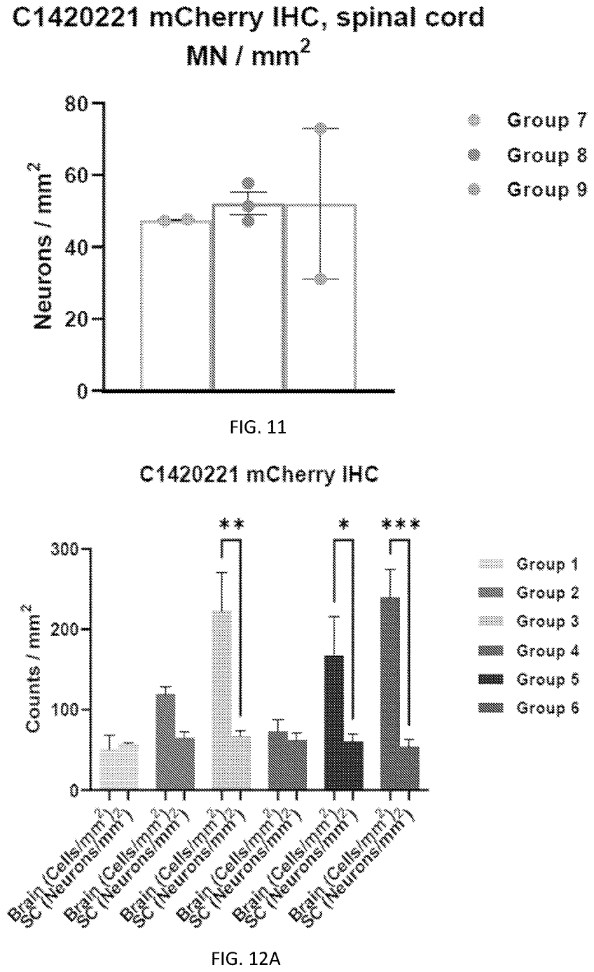

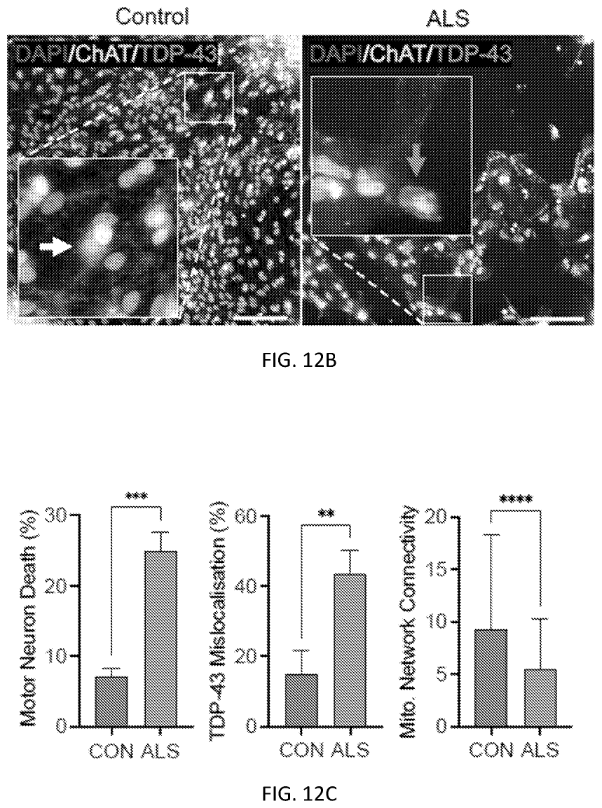

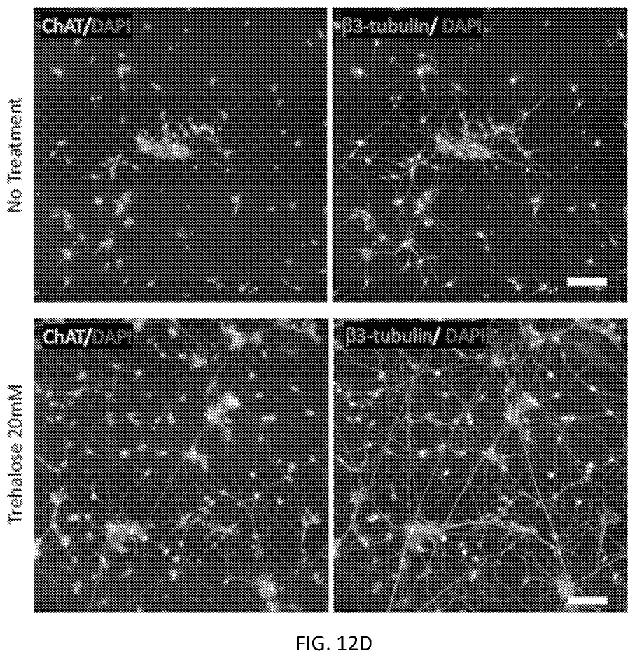

Phase I: In the main study arm, 36 R6/2 transgenic (TG) and 12 wild-type (WT) male mice were assigned to empty adenoviral vector (AAV1-mCherry) groups (WT, n=12, TG, n=12) and AAV1-OtsAB-mCherry treatment groups with low dose (n=12, 0.5×10¹² GP/ml) and high dose (n=12, 2.0×10¹² GP/ml). At 6 weeks of age, mice were administered with vehicle or therapeutic AAV1 by a single bilateral intrastriatal infusion (3 μl/hemisphere). Open field test and kinematic gait analysis were performed at pre-treatment baseline, age week 8 and age week 12. Two-choice swim test were performed at age week 9. Striatal, cortical and whole brain volumes and striatal metabolites were measured at age week 12 by magnetic resonance imaging (MRI) and magnetic resonance spectroscopy (MRS), respectively. Body weights were recorded twice per week. At 12 weeks of age, the mice were euthanized and the following samples were collected: plasma, left hemisphere brain and right hemisphere striatum. mRNA expression of HTT KI, Darpp-32, IL-ß and TNF-α were assessed from striatum samples. The other hemisphere striatal region was used for immunohistochemical analysis of Em48 and Darpp-32. Neurofilament light chain (NfL) levels in the plasma were measured. The survival study arm included the same experimental groups, group sizes and dosing regimen as in the main study arm. However, the animals were followed up until 25 weeks of age and only body weights were measured twice per week. No other tests or sample collections were performed. See FIGS. 13 and 14.

Phase II: At ~4 weeks of age (P28-P33), 22 R6/2 mice were administered with AAV9-CAG-otsA-P2A-otsB-T2A-mCherry-WPRE (7.4×10¹² vg/ml), (pBK1802), 6 μl to both striata (in total 12 μl/animal) and assigned to groups 5 and 6 (n=11/group) and group 9 (1 animal). In addition to the intrastriatal AAV infusion, group 6 mice were dosed with trehalose 5 times per week (i.p., 5 g/kg, 25 ml/kg). Group 7 mice (n=11) were dosed only with trehalose similarly as group 6 mice. Group 5-7 animals were weighed twice per week throughout the study until 8 post infusion, i.e., 12 weeks of age (n=2-9/group) or followed until survival (n=2-9/group). One group 4 animal was sacrificed at age week 21. Group 9 mouse were euthanized 4 weeks after infusion at 8 weeks of age. The following tissues were collected from mice at the scheduled end point: blood, brain (hemisphere 1 for IHC), and tail. Groups 6 and 7 animals were taken down 30 min post last trehalose i.p. dose. The group 9 mouse was similarly subjected to sampling at 4 weeks post infusion (at 8 weeks of age). Only tail samples were collected from survival study arm mice. EM-47 and Darpp-32 immunohistochemistry (IHC) analysis was performed on one brain hemisphere.

### Materials

### Reagents and Solutions

Reagents used for phase I and phase II immunohistochemical stainings are shown in Table 17.

TABLE 17
Reagents for immunohistochemical stainings.

| Reagent | Company | Cat. no. | Lot no. |
|---|---|---|---|
| 10X Citrate Buffer | Sigma | C9999 | MKCR1504 |
| 10X Tris-Buffered Saline | Fisher | BP2471-1 | 213149 |
| | Scientific | | 207039 |
| | | | 186187 |
| 30% H₂O₂ | Sigma | H1009 | STBJ8210 |
| ABC kit | Vector | PK-6100 | ZH0426 |
| Anti-DARPP32, rabbit | Millipore | AB10518 | 3729338 |
| Anti-GFAP, chicken | Abcam | ab4674 | GR3424848-1 |
| Anti-Iba1, rabbit | Wako | 019-19741 | WTQ1615 |
| Biotinylated anti-rabbit | Vector | BA-1000 | ZH0818 |
| BSA | Sigma | A9647- | SLCH3830 |
| | | 100G | |
| CryoSpray | HistoLab | 00211 | 3 |
| Etax Aa | Altia Oyj | 12110143 | 21192 |
| | | | 22205 |
| | | | 21473 |
| Goat anti-chicken AF568 | Abcam | ab175477 | GR341054-1 |
| Goat anti-rabbit AF488 | Invitrogen | A11008 | 2420730 |
| ImmEdge Pen | Vector | H-4000 | ZH1011 |
| | | | ZJ010 |
| Immpact DAB kit | Vector | SK-4105 | ZJ0113 |
| Methanol | J. T. Baker | 8045 | 1827001865 |
| Anti-EM48, mouse | Millipore | MAB5374 | 3760520 |
| Normal Goat Serum | Millipore | S26 | 3754386 |
| O.C.T Compound | Leica | 3801480 | 091420 |
| ProLong Gold with | Life | P36931 | 2433886 |
| DAPI | Technologies | | 2415906 |
| Roticlear | Carl Roth | A538.2 | 021304706 |
| | | | 021304708 |
| Roti-Mount | Carl Roth | HP68.2 | 281312050 |
| Superfrost Plus Adhesion | Epredia | J1800AMNZ | 021022-9 |
| Microscope Slides | | | |
| Triton X-100 | Sigma | 93418 | SLCJ7494 |
| | | | WXBD6187 |
| | | | STBH4686 |

### Animals and Animal Husbandry

Male R6/2 (B6CBA-Tg(HDexon1)62Gpb/3J) (The Jackson Laboratory, USA, JAX stock: 006494) mice (Mus musculus) between the ages of 28-33 days were used in in this study. According to the animal vendor, CAG repeat numbers were within the range of 120±5. Each animal was tested for the repeat numbers prior to shipping of animals to the Test Laboratory.

Mice were social house with 2-5 mice per cage with individual ventilation caging systems in polycarbonate Type II long cages (Allentown Inc.) Environmental enrichment was provided as was a plastic igloo for shelter, gnawing material (aspen sticks), and nesting material (corn cob bedding). Mice were fed a standard chow of Teklad Global 2016 pellet Ad libitum. Standard chow was provided on cage floor to all mice and replaced daily. Tap water was provided Ad libitum. Room temperature was 21.5±1.5° C. Room lights were on from 7 AM to 8 PM. Mice were only handled together with husbandry routines prior to beginning of the study in vivo activities.

### Humane End Point Criteria

The following were the general humane end point criteria applied in the study:

If the animal had mild or moderate impairment of general condition (welfare scoring: activity, posture, piloerection, orbital tightening, breathing problems, diarrhea; dehydration, decreased body weight or body condition; other signs of pain, other abnormal behavior, inability to feed, drink, urinate, defecate or groom itself) it was followed and treated according to Veterinary Care SOP for maximum of 2 days, within what time the animal's condition should have return to normal, otherwise the animal was euthanized. If animal's general condition was impaired at severe extend, reaching total score of 10 in Welfare Scoring, or receiving score 3 in any if the individual aspects of welfare scoring, or receiving score 2 in breathing or diarrhea category (marked dyspnea or profuse diarrhea), it was euthanized immediately.

Other criteria included for euthanasia included: >5 s delay in righting reflex, body temperature cannot be maintained within 24 hours, hydrocephalus, unsolvable malocclusion, severe injury or chronic wound, automutilation, >1 cm tumor or swelling.

### Model-Related Humane Endpoint Criteria:

25% body weight loss, compared to the maximum bodyweight in the study, treatment unresponsive penis prolapse involving inflammation.

### Procedure-Specific Criteria:

Surgical procedures: Poor or partial recovery after surgical operation (direct complications from the surgery), or ulcerations and infections of surgical site during the experiment, when they did not improve for supportive treatment within 1-2 days. Opened stitches after the one reoperation: stitches could be re-sutured once after the operation and if surgical wound re-opened second time, the wound was be treated and animal followed according to “general criteria”.

### Molecular Trehalose (MT) Preparation

Trehalose dihydrate was dissolved in water for injection once a week. Correction factor 1.105 was applied when preparing trehalose dihydrate solution to achieve the dose 5 g/kg of pure molecular trehalose. Heating was used if needed. After clear solution was achieved, MT formulation was divided into daily aliquots in plastic Eppendorf tubes and stored at 4° C. until dosing. Thirty minutes before dosing MT formulation was warmed to room temperature.

### Experimental Groups

Before the initiation of in-life phase, any animals considered unsuitable for use in the study were replaced by alternate animals obtained from the same shipment and maintained under the same environmental conditions.

Animals were assigned to groups by a stratified randomization scheme designed to achieve balanced groups based on body weight.

Animals were grouped as follows:

### Phase I:

Group 1: 12 WT mice treated with vehicle (Control)
Group 2: 12 TG mice treated with vehicle (Control)
Group 3: 12 TG mice treated with AAV1-OtsAB-mCherry Low Dose
Group 4: 12 TG mice treated with AAV1-OtsAB-mCherry High Dose

### Phase II:

Group 5:11 TG male mice infused at 4 weeks of age with AAV9-CAG-otsA-P2A-otsB-T2A-mCherry-WPRE (pBK1802, 7.4×10¹² vg/ml) to both striata (6 μl/hemisphere). Infusion rate: 0.4 μl/min. Euthanized at 8 weeks after infusion (12 weeks of age, n=4). n=7 monitored until survival.
Group 6: 11 TG male mice infused at 4 weeks of age with AAV9-CAG-otsA-P2A-otsB-T2A-mCherry-WPRE (pBK1802, 7.4×10¹² vg/ml) to both striata (6 μl/hemisphere). Infusion rate: 0.4 μl/min. Treated also with trehalose dehydrate (molecular trehalose or MT) 5 g/kg (i.p., 25 ml/kg, trehalose dose formulation concentration: 0.2 g/ml), 5 times per week. Euthanized at 8 weeks after infusion (12 weeks of age, n=2). n=9 monitored until survival.
Group 7: 18 TG male mice treated with trehalose 5 g/kg (i.p., 25 ml/kg, trehalose dose formulation concentration: 0.2 g/ml), 5 times per week starting at 4 weeks of age. Euthanized at 8 weeks after first trehalose dose (12 weeks of age, n=9). n=9 monitored until survival.
Group 9: 1 TG naïve male mouse infused at 4 weeks of age with AAV9-CAG-otsA-P2A-otsB-T2A-mCherry-WPRE (pBK1802, 7.4×10¹² vg/ml) to both striata (6 μl/hemisphere). Infusion rate: 0.4 μl/min. Euthanized 4 weeks after infusion (8 weeks of age).
   The doses, dosing paradigm, and route of administration for each teach article was summarized in Table 18 and Table 19 below.

TABLE 18
AAV administration:

| | | Dose | | | | Total doses |
|---|---|---|---|---|---|---|
| | | | Volume | Dosing | Group | per animal |
| Group | Test Article | Concentration | (# doses) | route | size | (total volume) |
| 1 | Vehicle | — | 3 μl | IS | 8 | 2 (6 μl) |
| 2 | Vehicle | — | 3 μl | IS | 8 | 2 (6 μl) |
| 3 | AAV1-OtsAB-mCherry | 0.5 × 10¹² | 3 μl (2) | IS | 8 | 2 (6 μl) |
| | | (Low) | | | | |
| 4 | AAV1-OtsAB-mCherry | 2.0 × 10¹² | 3 μl (2) | IS | 8 | 2 (6 μl) |
| | | (High) | | | | |
| 5 | AAV9-CAG-otsA-P2A-otsB- | 7.4 × 10¹² vg/ml | 6 μl (2) | IS | 11 | 2 (12 μl) |
| | T2A-mCherry-WPRE | | | | | |
| | (pBK1802) | | | | | |
| 6 | AAV9-CAG-otsA-P2A-otsB- | 7.4 × 10¹² vg/ml | 6 μl (2) | IS | 11 | 2 (12 μl) |
| | T2A-mCherry-WPRE | | | | | |
| | (pBK1802) | | | | | |
| 7 | — | — | — | — | — | — |
| 9 | AAV9-CAG-otsA-P2A-otsB- | 7.4 × 10¹² vg/ml | 6 μl × 2 | IS | 1 | 1 (12 μl) |
| | T2A-mCherry-WPRE | | | | | |
| | (pBK1802) | | | | | |

TABLE 19
Trehalose administration:

| | | Dose | | | | |
|---|---|---|---|---|---|---|
| | | | Volume | Dosing | Group | Total doses |
| Group | Test Article | Concentration | (# doses) | route | size | per animal |
| 1 | — | — | — | — | — | — |
| 2 | — | — | — | — | — | — |
| 3 | — | — | — | — | — | — |
| 4 | — | — | — | — | — | — |
| 5 | — | — | — | — | — | — |
| 6 | Trehalose dehydrate (MT) | 5 g/kg* | 25 ml/kg | i.p. | 11 | 41/until survival |
| 7 | Trehalose dehydrate (MT) | 5 g/kg* | 25 ml/kg | i.p. | 11 | 41/until survival |
| 9 | — | — | — | — | — | — |

*Active trehalose corresponding to 5.252 g/kg of trehalose dihydrate.

### Methods

### In Vivo Measurements and Sampling

Assessment of the presence of portosystemic shunting in mice by plasma bile acid level measurement (Main Study and Survival Study Arm)-Portosystemic shunts have been identified in significant percentage of C57BL/6J mice, resulting in major alteration in brain morphometry, brain metabolites, physiological readouts (e.g. body weight, liver enzymes), and cognitive deficits. Prior to study start, plasma bile acid analysis were performed to exclude animals with abnormally high bile acid concentration (>20 μmol/L, in addition cohort-by-cohort evaluation of the data for possible analytical shifts) which is a surrogate marker of portosystemic liver shunt. See, for example, Cudalbu et al. 2013. Blood samples for bile acid analysis were collected from saphenous vein into Li-Hep tubes. Plasma was separated by centrifugation at 2000 g (4° C.) within 30 minutes from collection. About 30 μl of Plasma was aliquoted into pre-cooled polypropylene tubes and stored at −80° C. until analysis. Plasma bile acid concentrations were analyzed using a Thermofisher Konelab Xti 20 analyzer according to manufactures instructions.

Body Weights-Body weights were monitored twice per week throughout the study for all animals. Pre-trehalose treatment onset baseline body weight was used to assign the animals to experimental groups. Terminal body weights were not be registered from animals found dead or euthanized moribund.

Drug Delivery—Administration of MT was performed by investigators blinded to the treatment.

### Molecular Trehalose Administration

MT was administered to mice i.p. 5 times per week (from Monday to Friday). Dose volume was 25 ml/kg. Dose was 5 g/kg. Pure trehalose dose formulation concentration was 0.2 g/ml. The last dose of animals euthanized at 8 weeks post infusion (age week 12) was 30 min prior to terminal sampling.

### Intrastriatal Infusion Procedures

Animals in groups 1-4 (Phase I) and groups 5-6 and 9 (Phase II) were anesthetized in the induction chamber (2.0-5.0% Isoflurane, in 70% N₂O and 30% O2; flow 600 mL/min) and set on its back on the surgery platform on the heating pad. The heating pad was set to 37° C. and the rectal probe inserted into the animal with the help of a lubricant. Anesthesia was maintained at 1.5-2.5%. Ointment was applied on animal's eyes to prevent drying, infections and fiber optic light damage during the surgery. Intracranial infusion of the test article was performed using stereotactically guided Hamilton syringe and infusion system (Elevated U-Frame Stereotaxic Instrument, Mouse, Single Manipulator, cat no: 75-1808, Harvard Apparatus). The coordinates for bilateral infusion to the striatum in respect to bregma are listed in Table 20.

TABLE 20
Coordinates for bilateral infusion.

| Coordinate | Phase I (groups 1-4) | Phase II (groups 5, 6, and 9) |
|---|---|---|
| AP | +0.5 (anterior from | +0.8 mm (anterior from |
| | bregma) | bregma) |
| ML | +/−2.0 mm | +/−1.8 mm |
| DV | −3.0 mm (from the dura | −3.0 mm (from the dura |
| | surface) | surface) |

Scalp hair was shaved and skin was disinfected by scrubbing with 10% povidone-iodine (Betadine®). An approx. 10 mm midline skin incision on the scalp was cut and two membrane layers were removed on the sides by detaching them from the skull. Skull was exposed by using skin retractors. The animal's head was fixed to the stereotactic frame by the upper front teeth and by placing ear bars. A burr hole (approx. 0.9 mm in diameter) was drilled, to penetrate the bone (~1 mm) at selected coordinates by using stereotaxic coordinates in respect to bregma and a dental drill. Saline was placed on the top of the skull.

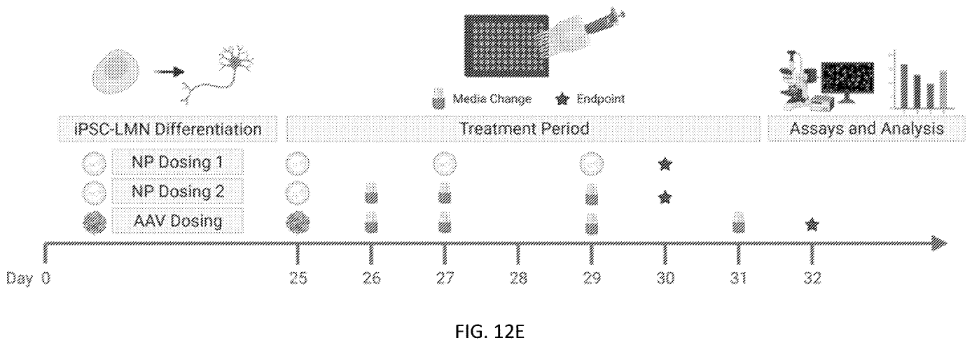

Phase I: The needle was lowered to the specified depth to reach the desired location. Prior to injections, Hamilton syringes were pre-conditioned with the associated infusion needles for 3 min with solution before infusing compounds. Prior to penetrating brain with injection needle, a minimal amount of solution was ejected from the syringe to visually confirm that injection needle was not obstructed. The Test Article was infused at the speed of 0.5 μl/min using a 25 μl Hamilton syringe with an angled 33 G needle (FIG. 16, point style 4, 45°) using Pump 11 Elite Nanomite (Harvard Apparatus). After the infusion, the needle was left in place for a stabilization period of 5 min prior to withdrawal of the needle. The infusion was performed similarly to both hemispheres only changing the medial-lateral coordinate.

Phase II: The needle was slowly lowered (over approximately 1 minute) to DV −0.1 mm below the infusion coordinates (−3.1 mm and then raised it to DV −3.0 mm). The needle was allowed to equilibrate for 1 minute. Prior to injections, Hamilton syringes were pre-conditioned with the associated infusion needles for 3 min with solution before infusing compounds. Prior to penetrating brain with injection needle, a minimal amount of solution was ejected from the syringe to visually confirm that injection needle was not obstructed. Thereafter Test Article (pBK1802) was delivered using a 10 μl Hamilton (model #80308) syringe with a 30 G blunt needle (FIG. 16). Dura membrane was not removed. The needle was lowered through the arachnoid membrane to the specified depth to reach the desired location (specified stereotactic coordinates above) and Test Article was infused at the speed of 0.4 μl/min, 6 μl total infusion volume (Pump 11 Elite Nanomite, Harvard Apparatus). The infusion was performed similarly to both hemispheres only changing the medial-lateral coordinate.

The volume in the syringe was noted and injection started by pressing RUN button. Volume in the syringe was checked after completion of the run. When delivery was completed, there was a 10 min wait before withdrawal of the needle such that it took approximately 1 min to be fully withdrawn. It was verified that the liquid was still able to flow from the syringe needle. The skin was thereafter closed using sutures (4-0, absorbable) and disinfected by scrubbing the wound with 10% povidone-iodine solution (Betadine®).

### Pre- and Post-Operative Care

Buprenorphine (0.05 mg/kg, s.c.) was administered 30 min before the surgery and twice a day for additional 2 days.

Carprofen (5 mg/kg, s.c.) was administered before the surgery and daily for additional 2 days.

Animals were carefully monitored by laboratory personnel at surgery day and twice a day for additional 2 days after surgery. Animals prematurely fulfilling the criteria for humane end point were euthanized.

### Motor Tests

### Phase I: Two-Choice Swim Test

Test performed on age week 9
Main study animals of phase I were tested only.

The swim tank (dimensions: 70 cm×30 cm×30 cm) was filled with water (25±1° C.) no higher than 1 cm from the top of a hidden escape platform. The hidden cylindrical platform (diameter, 6.0 cm, height, ~9 cm) was placed either at right or left end of the tank. During testing only red lights were used in the room. During acquisition of the task, each mouse was given 6 training trials per day to swim towards or away from the right end (counterbalanced across subjects) to escape onto the platform. On each trial, mice were released in the center of the tank facing the experimenter and allowed to swim for up to 60 seconds or until they found the platform. A choice was recorded when a mouse moved towards one end and swam beyond 20 cm from the center. If the mouse chose first the end of the tank with the platform “correct choice” was recorded, if the end without the platform “incorrect choice” was recorded. If the mouse failed to make a choice within 60 sec a “no choice” was recorded. Similarly, if the mouse chose the goal arm but did not find the platform before leaving the goal arm a “no choice” was recorded. At the end of the trial, once the platform was located the mouse was left on the platform for 5 s or after 60 sec had elapsed, the mouse was put on the platform for 10 s before being returned to its holding cage for an inter-trial interval of about 30-45 min. All animals were trained for 5 consecutive days. In all trials, choice, latency and “no choice” were recorded.

### Open Field Test (Main Study Arm)

Test performed at: age weeks 5-6 (baseline), 8 and 12
Phase I main study animals were tested only.

The mice were brought to the experimental room for at least 1 h acclimation period prior to testing. Activity chambers (Med Associates Inc, St Albans, VT; 27×27×20.3 cm) were equipped with IR beams. Mice were placed in the center of the chamber and their behavior was recorded for 30 min in 5-min bins. Quantitative analysis was performed on the following five dependent measures: total locomotion, locomotion in the center of the open field, rearing rate in the center, total rearing frequency and velocity. Animals were tested and acclimated at low-stress conditions with light intensity lowered to approximately 10-30 lux of red light.

### Fine Motor Kinematic Gait Analysis

Test performed at: age weeks 5-6 (baseline), 8 and 12
Main study animals of Phase I were tested only.

On the day of testing, appropriate point on animals' body were marked. Those included joints of limbs and parts of tail to ease the data analysis process. The movement data was captured using a high speed camera (300 fps) from three different dimensions, from below and both sides. The captured videos of each animal were first converted to SimiMotion software to track the marked points of the body to have the raw data i.e. the movement of the different body points in coordinates in relation to the ground, and correlation of the three dimensions. Different gait patterns and movements were analyzed using a custom-made automated analysis system. The analyzed parameters include:
General gait pattern parameters (stride time and speed, step width, stance and swing time during a stride, interlimb coordination)
Body posture and balance (toe clearance, iliac crest and hip height, hind limb protraction and retraction, tail position and movement).
Fine motor skills (swing speed during a stride, jerk metric during swing phase, angle ranges and deviations of different joints, vertical and horizontal head movement).
   Prior to any procedure, all animals were allowed a 30-min habituation to the test room.

### Phase I: MRI for Brain Volumetric Analysis

Performed at: 12 weeks of age, after open field and kinematic gait analysis 12 week's testing
Main study animals of Phase I were tested only

MRI was performed in a horizontal Bruker BioSpec 11.7T high-field preclinical MRI scanner with bore size 160 mm equipped with a gradient set capable of max. gradient strength 750 mT/m and interfaced to a Bruker Avance III console (Bruker Biospin GmbH, Ettlingen, Germany). A volume coil (Bruker Biospin GmbH, Ettlingen, Germany) was used for transmission and a two-element surface array coil for receiving (Rapid Biomedical GmbH, Rimpar, Germany). Animals were anesthetized with 5% isoflurane (in 70% N2 and 30% 02; flow 600 mL/min). During the MRI acquisition the concentration of anesthetic was reduced to 1.0-1.5%. Animals' temperature was monitored and maintained at 37.0±1.0° C. with homeothermic blanket system. Isoflurane-anesthetized animals were fixed to a custom-made holder and positioned in the magnet bore in a standard orientation relative to gradient coils.

Anatomical images were acquired using a TurboRARE sequence with TR/TE=3150/34 ms, matrix size 256×256, FOV 20.0×20.0 mm², 31 contiguous 0.45 mm thick slices (0.6 mm with negative 0.15 mm gap) and 8 averages. Volumetric analysis was performed by manual ROI-analysis for whole brain, cortex and striatum using Paxinos atlas as a reference. Analysis was conducted using in-house built MATLAB based analysis software and observer blinded to the treatment groups.

### Phase I: 1H-MR Spectroscopy (Main Study Arm)

Performed at: 12 weeks of age upon MRI volumetry analysis, after open field and kinematic gait analysis 12 week's testing
Main study animals from Phase I were included only.
   For the 1H-MR spectroscopic measurement, a volume of interest (voxel) was placed in the striatum based on T2-weighted localizer images. Automatic FASTMAP shimming algorithm was used to adjust B₀ homogeneity in the voxel. The water signal was suppressed using variable power RF pulses with optimized relaxation delays (VAPOR) to obtain B₁ and T₁ insensitivity. A PRESS sequence (TE=10 ms) combined with outer volume suppression (OVS) was used for the pre-localization. Three OVS blocks were used interleaved with water suppression pulses. Data were collected by averaging 512 excitations (frequency corrected for each FID) with TR of 4 s, number of points 2048 and spectral width 5 kHz. Excitation frequency was shifted −2 ppm, to minimize the chemical shift phenomenon within the selected voxel. In addition, a reference spectrum without water suppression (NT-8) was collected from the identical voxel using the same acquisition parameters.

Peak areas for metabolites were analyzed using LCModel (Stephen Provencher Inc., Oakville, Canada) and results were given relative to water content in tissue. Following metabolites were included into the analysis: Ala—Alanine, Cr—Creatine, Pcr—Phosphocreatine, GABA-Gamma aminobutyric acid, Glc—Glucose, GLN—Glutamine, GLU—Glutamate, GPC—Glycerophosphocholine, Pch—Phosphocholine, GSH—Glutathione, INS—myo-Inositol, Lac—Lactate, NAA—N-acetyl aspartate, TAU—Taurine, CHO—Choline, NAA+NAAG—N-acetyl aspartate+N-acetyl aspartate glutamate, CR+PCR—Creatine+Phosphocreatine, GLU+GLN—Glutamate+glutamine. Criteria of <20% SD in the Cramer-Rao lower bounds were used in the reporting of metabolite values. In addition, trehalose peak area was analyzed at best effort. Animals were anesthetized with 5% isoflurane (in 70% N2 and 30% 02; flow 600 mL/min). During the MRI acquisition the concentration of anesthetic was reduced to 1.0-1.5%. Animals' temperature was monitored and maintained at 37.0±1.0° C. with a homeothermic blanket system. Isoflurane anesthetized animals were fixed to a custom-made holder and positioned in the magnet bore in a standard orientation relative to gradient coils.

### Survival

A subset of animals from Phase I and Phase II were monitored until survival. Survival age of each animal was defined as the age in days until spontaneous death or until euthanized due to meeting humane end point criteria. Exclusions from survival analysis were done when there was significant evidence that the death of the animal was not directly or indirectly caused by the disease model. Example causes of death leading to exclusion: surgery complications (e.g. intracranial bleeding, confirmed by necropsy) and handling errors leading to injuries. Spontaneous death under investigator's observation and animals found dead in their housing cage were considered as disease model-related unless another cause of death was considered likely upon necropsy.

### Phase II: In-Life Blood Samples Collection

At 8-9 weeks of age, in-life blood samples were collected from Phase II animals from saphenous vein. Briefly, wake animals were restrained for blood draw and approximately 150 μl of blood was collected to K₂EDTA collection tubes and the samples were processed according to the protocol below. The samples were used for trehalose analysis assay.

### Unscheduled Euthanasia and End of In Vivo Procedures

If an animal underwent unscheduled euthanasia for humane reasons as per Testing Facility SOPs or if an animal was found dead, a necropsy was conducted and tail samples were collected. In Phase II, whole brain samples were additionally collected from animals whenever possible. Brain samples were post-fixed in 10% neutral-buffered formalin (NBF) for 48 h after which the samples were immersed in 0.1 M PB and stored at 4° C. until shipped to Sponsor-designated destination. Upon necropsy, all abnormal findings were registered.

Sample Collection (n=8/Group)

At 8 weeks post infusion or trehalose dosing start (12 weeks of age), n=8/group (Groups 1-4) or at 4 weeks post infusion (8 weeks of age) 1 mouse (group 5), mice had their terminal body weight recorded and were euthanized by deeply anesthesia with pentobarbital (180 mg/kg, i.p.) 30 min post last vehicle/trehalose dose.

Next, blood was collected via cardiac puncture and plasma was separated. The animals were transcardially perfused with ice cold heparinized (Heparin 2.5 IU/ml) saline. Next, brain samples were collected and processed for QuantiGene and IHC analysis. See Table 21 for details.

TABLE 21
Biofluids and tissue collection, samples purpose, and processing.

| | | | |
|---|---|---|---|
| Tissue | Assay | Processing and storage | Vial |

Phase I

| | | | |
|---|---|---|---|
| Plasma (200 μl) | Neurofilament light | Frozen on dry ice. Stored at −80° C. | 2 ml |
| | chain (NfL) analysis | | |
| Brain, left | IHC | Post-fixed in 4% PFA in 0.1M PB for 24 h | Fixed and |
| hemisphere | | at +4° C. Cryoprotected in 30% sucrose in | cryoprotected in |
| | | 0.1M PB for 72 h at +4° C. Frozen on top of a | 10 ml tubes → |
| | | plastic cap in liquid nitrogen. Stored at −80° | wrapped in foil |
| | | C. until sectioned. | after frozen. |
| Striatum, right | QuantiGene | No post fix, 5-10 X volume of RNA later, | 2 ml, round |
| hemisphere | | 24 h +4° C., Remove RNA later after 24 h, | bottom |
| | | only freeze tube at −80° C. (no dry ice needed) | |

Phase II

| | | | |
|---|---|---|---|
| Plasma (200 | Trehalose at CRL/ | See processing instructions below Frozen | 2 ml |
| μl) | Shipped to | on dry ice. Stored at −80° C. | |
| | Sponsor | | |
| | designated | | |
| | destination | | |
| Brain, left | IHC | Post-fixed in 4% PFA in 0.1M PB for | Fixed and |
| hemisphere | | 24 h at +4° C. Cryoprotected in 30% | cryoprotected |
| | | sucrose in 0.1M PB for 72 h at +4° C. | in 10 ml tubes → |
| | | Frozen on top of a plastic cap in liquid | wrapped in |
| | | nitrogen. Stored at −80° C. until sectioned. | foil after |
| | | | frozen. |
| Striatum, right | QuantiGene | No post fix, 5-10 X volume of RNA | 2 ml, round |
| hemisphere | | later, 24 h +4° C., Remove RNA later after | bottom |
| | | 24 h, only freeze tube at −80° C. (no dry | |
| | | ice needed) | |
| Sectioned to 4 | Trehalose | See below | 2 ml |
| regions, right | concentration | | |
| hemisphere | | | |

Blood sample processing-Preparation of Solution. An acidification solution of formic acid 5% in Milli-Q Type Water (5:100) was made. In a 25 mL glass bottle, 20 mL of Milli-Q type water or HPLC grade water using a graduated cylinder was added to 1 mL of formic acid using borosilicate pipettor by adding the contents of a commercially available 1 mL ampule of formic acid. The solution was mixed well and stored at room temperature for a maximum of one month.

Study sample handling-0.5 mL of blood was collected into labelled microcontainer tubes containing EDTA K2 and immediately put into an ice-water bath. The microcontainer tubes were centrifuged at 2000×g for 10 minutes at 4° C. as soon as possible following collection, and within one hour recommended. The microcontainer tubes were put into an ice-water bath.

Preparation of study samples for analysis—10 μL of acidification solution was added to a labelled polypropylene cryovial. 50 μL of plasma was transferred into the labelled polypropylene cryovial containing 10 μL of acidification solution and cap the tubes. Volumes were modified to ensure that the plasma/acidification solution ratio remained the same. Each polypropylene tube adequately for 10 seconds using a vortex mixer. The polypropylene cryovials were stored at −80° C. until sample analysis was completed.

### Ex Vivo Samples Analysis

Neurofilament light Chain (NfL) Analysis in Blood-Neurofilament Light Chain (NfL) analysis from total n=39 mouse plasma samples was performed in duplicates determinations according to Quanterix Simoa™ NF-Light Advantage Kit (REF 103186) Manufacturer's protocol.

Cryosectioning-Cryosectioning was performed to striatal (left hemisphere) (Bregma+1.30 mm to −0.8 mm from Bregma, containing the infusion site at +0.5 mm) region and sections were collected as 20 μm thick sections on slides. A series of sections consisted of 8 sections with an interval of 300 μm in-between each section. A total of 5 series were collected from the target region using subsequent slides. For phase I, Series 1 & 3 were used for IHC analysis of EM48, series 2 was used for Darpp-32, series 4 was used for Iba1+GFAP and Series 5 was used for piloting and negative controls. For phase II, A total of 6 series were collected from the target region using subsequent slides. Series 1 was used for IHC analysis of EM48, series 2 was used for Darpp-32, and series 4 was used for Iba1+GFAP. Series 5 was collected as spare samples and series 6 was used for piloting and negative controls.

### Chromogenic Detection/Immunostaining—

The immunohistochemical staining listed below were performed for the cryosectioned slides.

The used antibodies/reagents were:
mouse monoclonal IgG against huntingtin protein, clone mEM48 (Millipore, cat. no. MAB5374) (dilution 1:400)
rabbit polyclonal IgG against dopamine- and cAMP-regulated neuronal phosphoprotein (DARPP-32; Millipore/Sigma-Aldrich, cat. no. AB10518) (dilution 1:5000)
chicken polyclonal antibody against glial fibrillary acidic protein (GFAP; Abcam, cat. no. ab4674) (dilution 1:250)·
rabbit polyclonal IgG against ionized calcium-binding adaptor protein-1 (Iba-1; Wako #019-19741) (dilution 1:500)

For fluorescence immunohistochemistry (GFAP and Iba-1) the following general protocol was applied:

Cryosections were taken to room temperature from storage, dried and rehydrated. Antigen retrieval was performed, where appropriate (for both GFAP and Iba-1). The sections were permeabilized followed by blocking against non-specific binding. The sections were then incubated for overnight in the primary antibody solution. The following day, the sections were washed and incubated in the appropriate secondary antibody for 2 h at RT. (Autofluorescence was blocked using 0.3% Sudan Black B in 70% ethanol.) After washing, the sections were mounted with a water-soluble mounting medium containing 4′-6-Diamidino-2-phenylindole (DAPI) to counterstain the nuclei, coverslipped, cured, sealed and stored at +4° C. until imaged.

For brightfield immunohistochemistry the following general protocol was applied (EM48 and DARPP-32):

Cryosections were taken to room temperature from storage, dried and rehydrated. Endogenous peroxidase activity was blocked, and the sections were carefully washed and permeabilized, and blocked against non-specific binding, followed by an overnight incubation in the primary antibody solution. The following day the sections were washed and incubated in the appropriate secondary antibody (biotinylated) for 2 h at RT. After washing, the sections were incubated with the tertiary reagent (avidin-biotin complex; ABC), washed and incubated with the 3,3′-diaminobenzidine (DAB) chromogen to reach a desired staining intensity (4 min; exactly same conditions for each slide). Finally, the sections were dehydrated, mounted with an organic solvent based mounting medium and coverslipped. The slides were dried and stored at RT until imaged.

Antigen expression analyses—The stained sections were scanned with the Olympus VS120 slide scanner using the proper fluorescent or brightfield channels. The regions of interest (ROIs) (striatum/cortex) were analyzed for positive immunostaining of the respective targets (EM48, DARPP-32, GFAP and Iba1) applying the VIS-Visiopharm Integrator System (Visiopharm, Denmark).

Magnification used for the scanning and exposure times was decided after initial screenings of the stainings.

Positive staining of the respective antigens was detected by applying a suitable threshold which recognizes only truly positive staining. Depending on the antigen, the results are presented as either intensity or percent positive area or the number of positive cells within the ROIs (striatum/cortex), whose total area is also measured (percent positive area=stained area within the ROI/total ROI area*100).

For the individual stainings, the assays were performed as follows:
GFAP: % staining area
Iba-1: % staining area
EM-48: number of positive cells/mm² (striatum and cortex)
DARPP-32: number of positive cells/mm² (striatum)

QuantiGene gene expression assays—The expression of 4 target mRNAs were examined in the striatum (right hemisphere, total n=33) using the branched DNA assay (QuantiGene Plex, Invitrogen). To prepare the tissues for QuantiGene analysis, the tissues (5-10 mg pieces) were homogenized using the QuantiGene Sample Processing Kit for Fresh or Frozen Tissues according to the manufacturer's protocol using TissueLyser II and 5 mm steel beads (Qiagen). The cleared lysates were used in the QuantiGene gene expression assay.

Briefly, gene expression levels were determined using custom-prepared QuantiGene Plex sets and a QuantiGene Plex Assay Kit. The assay was performed according to the instructions provided by the manufacturer (Invitrogen). A small pilot was run separately before the actual assays to determine the optimal sample input to be used in the assays. Target expression levels were normalized using the geometrical mean of at least three different housekeeping genes identified in Table 22.

Mouse gene products to be measured by the QuantiGene (bDNA) method (8plex). Housekeeping genes are marked (*).

TABLE 22
QuantiGene Gene Expression Assay Gene Targets.

| | | Gene ID | |
|---|---|---|---|
| Target | | (Mus | Accession |
| symbol | Name | musculus) | No. |
| HTT_knock_in | HTT (KI) or | | GS00936 |
| | Htt_knock_in or KI | | |
| | mHTT | | |
| Darpp-32 | Protein phosphatase 1 | 19049 | NM_144828 |
| IL-1b | Interleukin 1 beta | 16176 | NM_008361 |
| TNF-a | Tumor necrosis factor | 21926 | NM_013693 |
| | (alpha) | | |
| Atp5b* | ATP synthase, H+ | 11947 | NM_016774 |
| | transporting mitochondrial | | |
| | F1 complex, beta subunit | | |
| Eif4a2* | Eukaryotic translation | | NM_013506 |
| | initiation factor 4A2 | | |
| Rpl13a* | Ribosomal protein L13a | | NM_009438 |
| Canx* | Calnexin | | NM_007597 |

*indicates housekeeping genes.

Trehalose Assay—Trehalose measurements were taken across the entire brain hemisphere 2 for all mice. The hemisphere was divided into anterior, middle, and posterior thirds and the trehalose concentration was measure in each third.

### Statistical Analysis and Data Graphical Representation

Prior to further statistical analysis, data quality check and validations was performed. During that process, potential outliers were identified and assessed. No outliers were removed from data without a clear justification for removal (e.g., identified measurement error in the laboratory notes).

Assumption of normality of each data set was primarily based on experience (e.g., data within a population is known to be approximately Gaussian) and observations during validation phase. Some biological variables are known to follow lognormal distributions—in these cases the data were first transformed to logarithms and then parametric statistical test were used. The same normality assumption were used for series of experiments, or group of similar readouts from an assay. In addition, the D'Agostino-Pearson omnibus normality test was used to support the decision whether parametric or nonparametric test should have been used. A summary of readouts for statistical analysis and visualization is shown in Table 23.

TABLE 23
Statistical Analysis and Visualization Summary.

| Test/Assay | Normalization | Visualization |
|---|---|---|
| BW | no | line (by group + by time) |
| Survival | no | Survival curve |
| QuantiGene | Targets normalized against the | scatter |
| | housekeeper gene(s) | |
| IHC analysis | no | scatter |

Body Weight—Comparisons between two groups used a two-way Mixed-effects model (Mixed Anova) with Geisser-Greenhouse correction (not assuming sphericity), followed by Fisher's LSD test between the two groups performed for each time point. Time, group and the interaction of Time×Group were fixed effects, and the individual subject was the random effect. In cases of longitudinal comparison of three or more groups, similarly, the two-way Mixed-effects model with Geisser-Greenhouse correction, followed by Dunnett's multiple comparisons test was performed. In this case, one family per time point will be set. Time, group and the interaction of Time×Group were fixed effects, and the individual subject was the random effect.

QuantiGene, trehalose assay and IHC—Simple comparisons between two groups (disease model assessment) used unpaired Welch's t-test, or, when the assumption of normality or log-normality was not met, by the Mann-Whitney U-test. Comparison involving three or more groups used the one-way ANOVA followed by Dunnett's multiple comparisons test (treatment vs. control). In case of significantly different standard deviations detected by Brown-Forsythe test, the assumption of equal standard deviations is rejected and Welch's ANOVA followed by Dunnett's T3 multiple comparison test was used. The non-parametric counterpart is the Kruskal-Wallis test followed by Dunn's multiple comparison test.

All values were presented as the mean±standard error (SE) of the mean. In case of two or more comparisons per family, the multiplicity adjusted P values are presented. All statistical analyses were conducted with a significance level of α=0.05, using GraphPad Prism (Version 8.3, GraphPad Software, Inc., San Diego, CA) or the R statistical software environment (R: A Language and Environment for Statistical Computing; R Core Team/R Foundation for Statistical Computing; Vienna, Austria; 2019; R-project.org).

### Results

Measurement of Mouse Body Weight after Treatment with AAV Trehalose

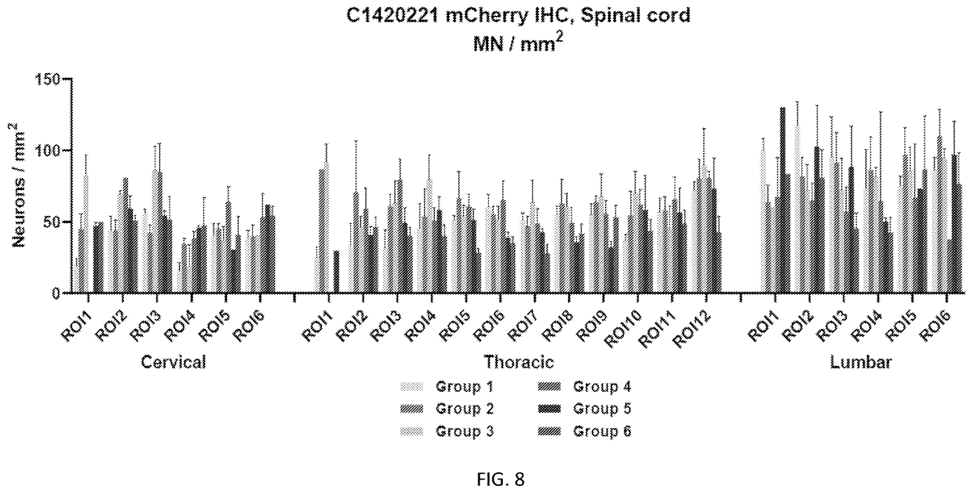

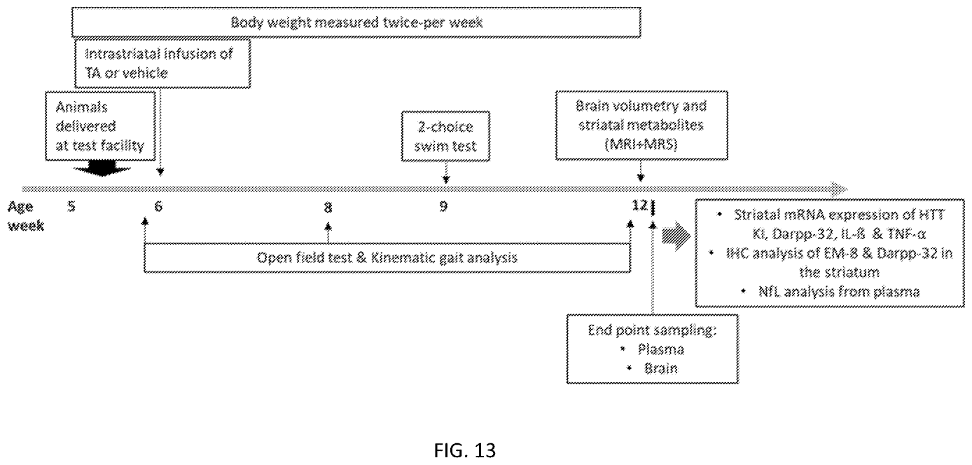

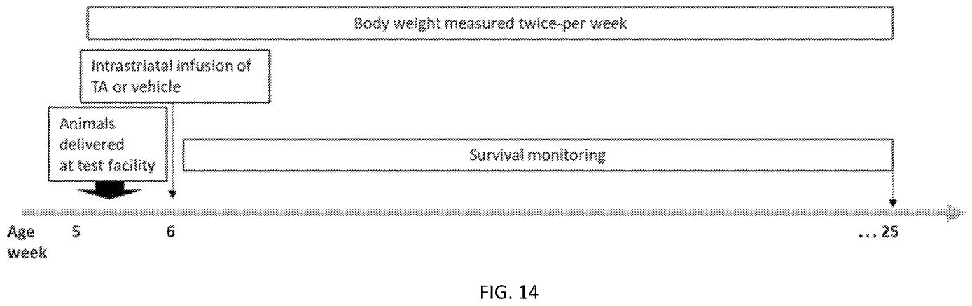

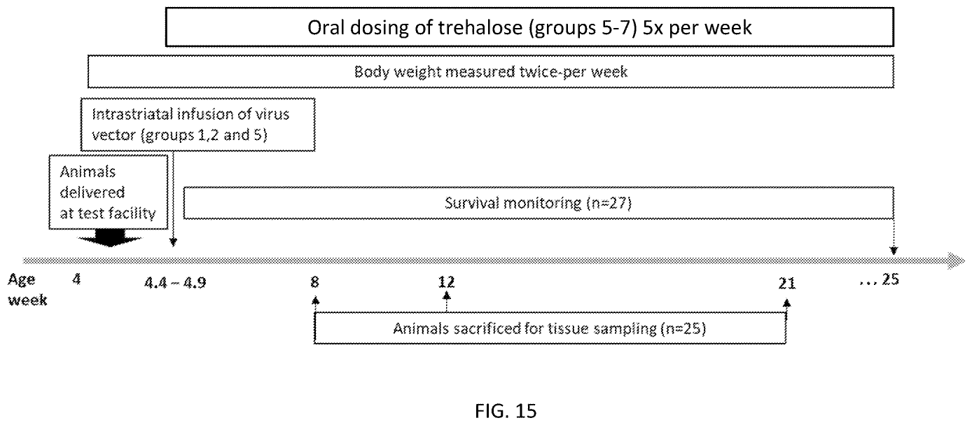

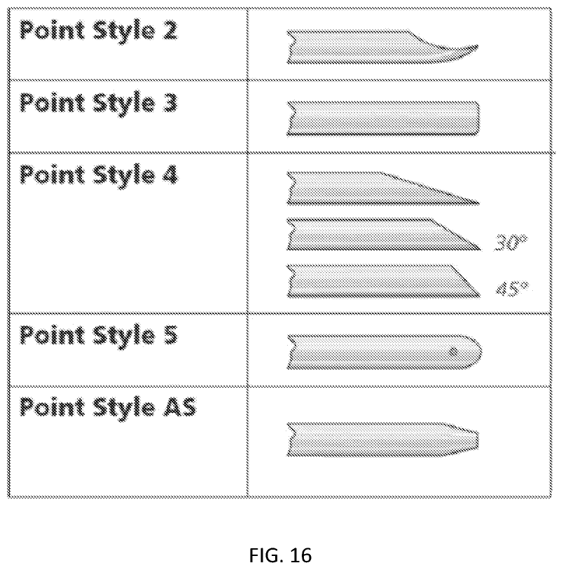

The effects of intrastriatal AAV1-OtsAB-mCherry or AAV9-CAG-otsA-P2A-otsB-T2A-mCherry-WPRE (pBK1802) administration and intraperitoneal trehalose and nano carbon trehalose on the body weights of transgenic R6/2 mice are presented in FIGS. 17-20. In Phase I, body weights of mice administered with AAV1-OtsAB-mCherry developed similarly as those of transgenic controls that were infused with vehicle in both main study arm (FIGS. 17A-17B) and survival arm (FIGS. 18A-10B). Overall, weights of transgenic R6/2 mice developed as expected with body weight decline starting at age week 10 whereas wild-type mice continued to gain weight until the end of the study follow-up period. In Phase II, there were no significant differences between groups administered with pBK1802, pBK1802+trehalose, trehalose alone or CNT alone (FIGS. 19A-19B). Survival group weights between Phase I and Phase II did not seemingly differ between all transgenic groups (FIGS. 20A-20B). However, no statistical comparisons were performed due to significant temporal difference between the execution of the two phases. See Table 24 and Table 25 for model and treatment effect statistics.

TABLE 24
Results for the model effect (Group 2 vs 1):

| | | | Statistically | |
|---|---|---|---|---|
| Fixed effects | | P value | significant | F |
| (type III) | P value | summary | (P < 0.05)? | (DFn, DFd) |
| Measurement | <0.0001 | **** | Yes | F (2.776, 61.07) = |
| | | | | 78.88 |
| Group | 0.0011 | ** | Yes | F (1, 22) = 13.97 |
| Measurement × | <0.0001 | **** | Yes | F (13, 286) = 34.84 |
| Group | | | | |

TABLE 25
Results for the treatment effect (Group 3-4 vs 1)

| | | | Statistically | |
|---|---|---|---|---|
| Fixed effects | | P value | significant | F |
| (type III) | P value | summary | (P < 0.05)? | (DFn, DFd) |
| Measurement | <0.0001 | **** | Yes | F (2.601, 80.84) = |
| | | | | 35.48 |
| Group | 0.7168 | ns | No | F (2, 34) = 0.3363 |
| Measurement × | 0.0016 | ** | Yes | F (26, 404) = 2.087 |
| Group | | | | |

Measurement of Mouse Lifespan after Treatment

To determine the effect of MT treatment on lifespan, transgenic R6/2 mice (a disease model for Huntington's Disease) were treated as described above. Controls were transgenic R6/2 mice that did not receive treatment and wild-type mice. Wild-type mice (n=12) had a median lifespan of 181 days. R6/2 mice treated with vehicle (n=12) had a decreased median lifespan of 101, showing that a severe Huntington's disease mouse model was established. R6/2 mice treated with a low dose of AAV1-OtsAB-mCherry had a median life span of 110 days, and were partially rescued. R6/2 mice treated with a high dose AAV1-OtsAB-mCherry had a median life span of 106 days, and were partially rescued. R6/2 mice treated with MT had a median lifespan of 119 days. Survival was not measured for other groups.

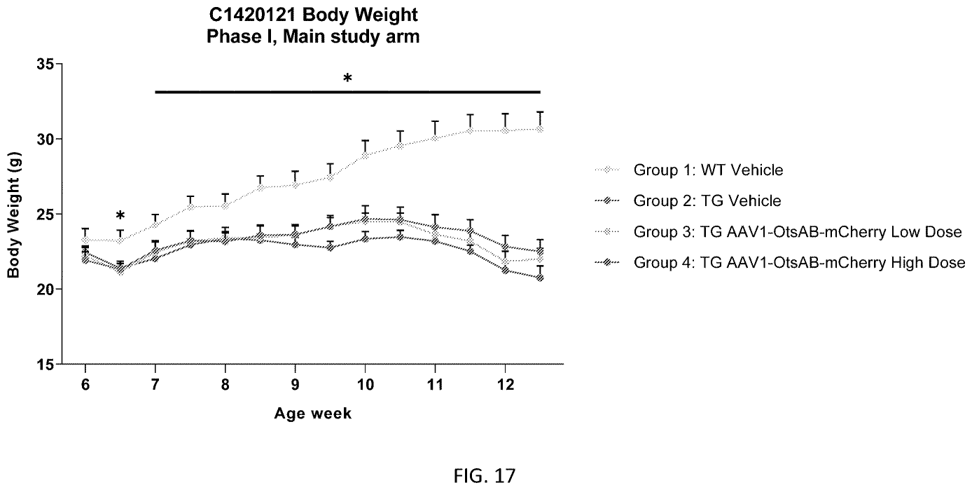

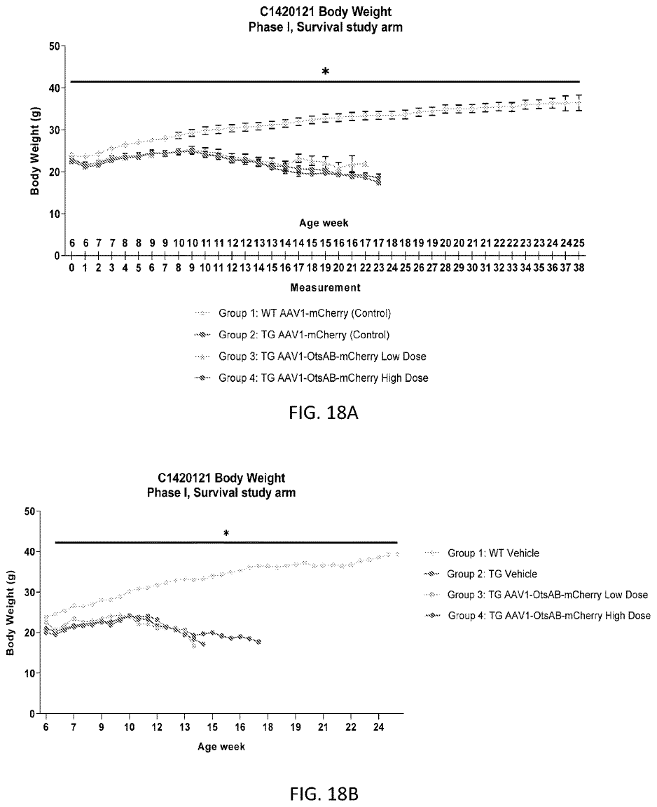

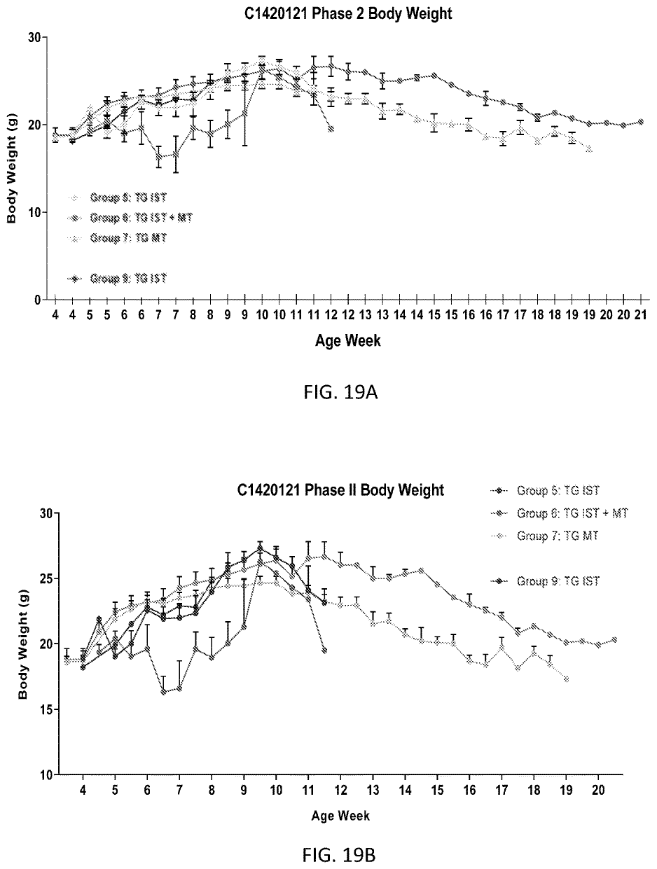

The effects of intrastriatal AAV1-OtsAB-mCherry or AAV9-CAG-otsA-P2A-otsB-T2A-mCherry-WPRE (pBK1802) administration and intraperitoneal trehalose and nano carbon trehalose on the survival of transgenic R6/2 mice are presented in FIGS. 21-23. Survival of transgenic mice administered with AAV1-OtsAB-mCherry (Phase I) did not significantly differ from the transgenic vehicle controls (FIG. 21). In Phase II, groups that were administered with pBK1802 and pBK1802+trehalose lost the majority of the mice at an early age due to spontaneous deaths due to animals meeting humane end point criteria. Thus, the survival of trehalose dosing groups survived considerably longer (FIG. 22). Trehalose and CNT groups' survival did not significantly differ from each other. The survival of Phase I AAV treated groups and Phase II trehalose dosing groups did not seemingly differ from each other (FIG. 23).

Measurement of Behavior after Treatment with AAV Trehalose.

Behavior of mice was tested with an open field trial to measure total locomotion, locomotion in the center of the field, total rearing frequency, rearing rate in the center of the field, and average mouse velocity as described above (data not shown).

In addition, a two-choice swim test was also used to measure latency, and the percentage of correct, incorrect, or no choice as described above (data not shown).

Measurement of Mouse Brain and Blood Features after Treatment with AAV Trehalose

The effects of intrastriatal AAV1-OtsAB-mCherry administration on the whole brain, cortex and striatum volumes of transgenic R6/2 mice at 12 weeks of age were measured as described above (data not shown).

The effects of intrastriatal AAV1-OtsAB-mCherry administration on striatal metabolite concentrations of transgenic R6/2 mice at 12 weeks of age were measured as described above (data not shown).

The effects of intrastriatal AAV1-OtsAB-mCherry administration on the gait characteristics, including overall gait score, mean speed, diagonal interlimb coordination, mean high at tail base, minimum knee angle, and hind leg retraction, of transgenic R6/2 mice at the pre-treatment baseline at 5 weeks and at the post-infusion testing time points at 8 and 12 weeks of age were measured as described above (data not shown).

The effects of intrastriatal AAV1-OtsAB-mCherry administration on neurofilament light chain (NfL) concentration at 12 weeks of age in the plasma of transgenic R6/2 mice were measured as described above (data not shown).

Measurement of DARPP-32, EM48, GFAP, and Iba-1 in the Brain Using IHC after Treatment with AAV Trehalose

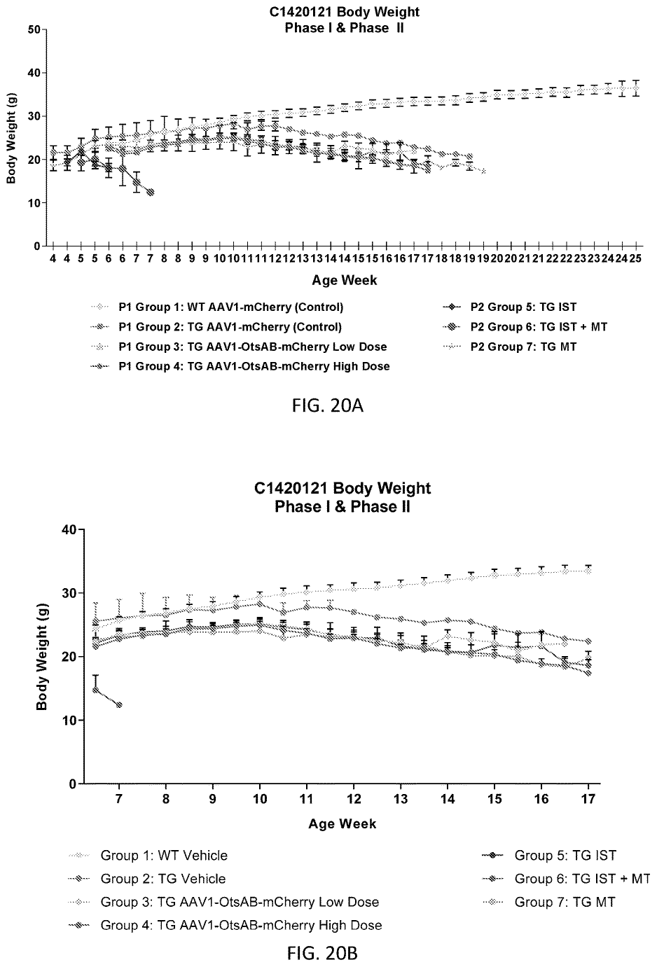

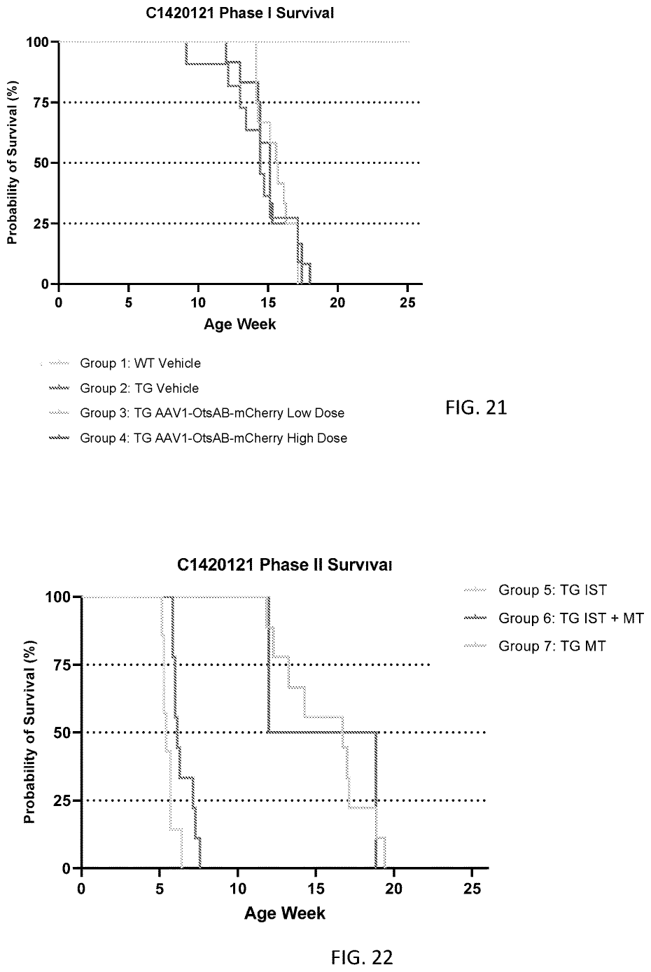

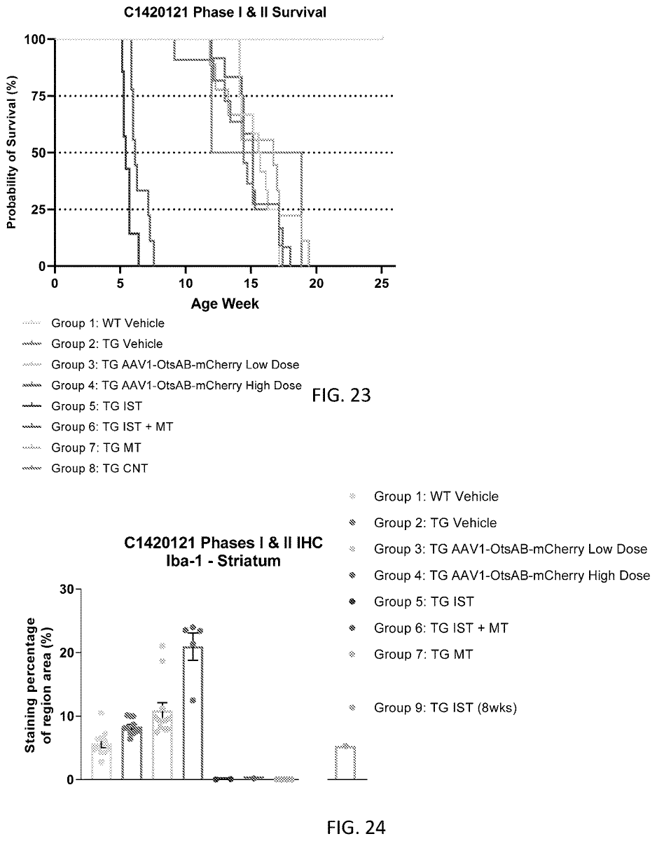

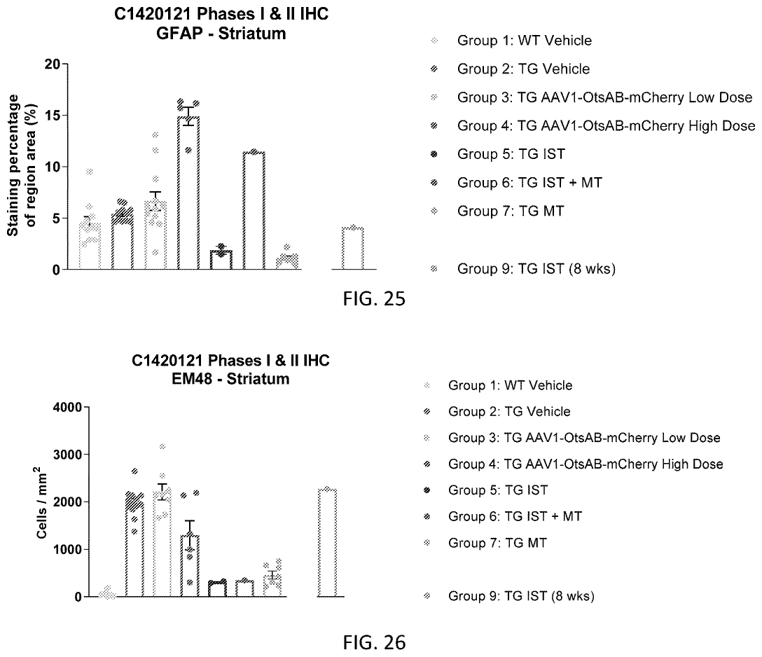

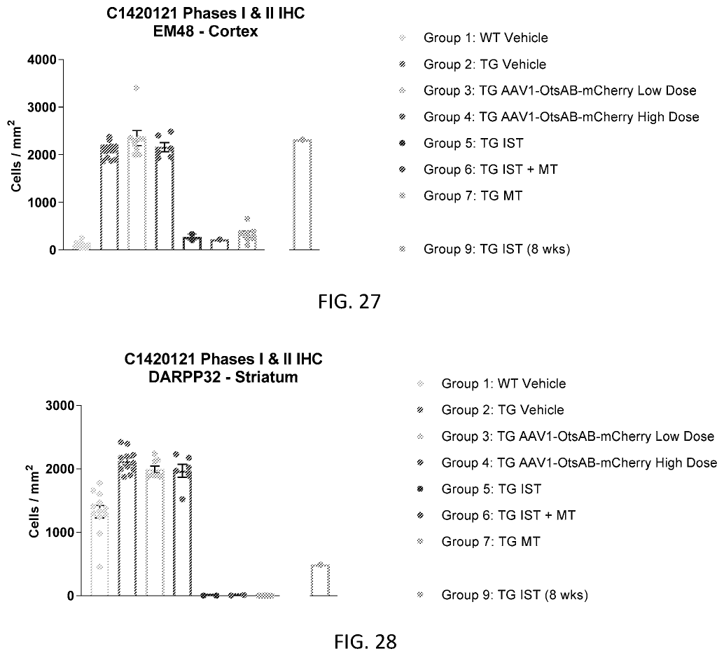

To determine the effect of AAV Trehalose treatment on DARPP-32, EM48, GAFP, and Iba-1, transgenic R6/2 mice (a disease model for Huntington's Disease) were treated with AAV Trehalose as described above. Brain samples were collected, and DARPP-32, EM48, GFAP, and Iba-1 were measured in the striatum or cortex using IHC analysis. Treatment of R6/2 mice with AAV trehalose as compared to untreated transgenic R6/2 mice resulted in lower levels of Iba-1 in the striatum when treated with AAV9-CAG-otsA-P2A-otsB-T2A-mCherry-WPRE (Groups 5 & 6) (FIG. 24), lower levels of GFAP in the striatum when treated with AAV9-CAG-otsA-P2A-otsB-T2A-mCherry-WPRE without Trehalose supplementation (Group 5) (FIG. 25), lower levels of EM48 in the striatum and cortex when treated with AAV9-CAG-otsA-P2A-otsB-T2A-mCherry-WPRE (Groups 5 & 6) or with AAV1-OtsAB-mCherry High Dose (Group 4) (FIGS. 26-27), and lower levels of DARPP-32 in the striatum when treated with AAV9-CAG-otsA-P2A-otsB-T2A-mCherry-WPRE (Groups 5 & 6) (FIG. 28). Therefore, mHTT aggregates in the striatum and in the cortex of the brain of mice treated with AAV Trehalose were reduced compared to mHTT aggregates in the striatum and in the cortex of the brain of mice that were not treated with AAV Trehalose.

### Example 6: Locomotor Activity in a Genetically Modified Rat Model

The purpose of this study is to validate a superoxide dismutase type 1 (SOD1) model of amyotrophic lateral sclerosis (ALS) in a transgenic rat through behavioral assessment and to evaluate a client test article in the same experiment. This study is conducted in accordance with The Guide for the Care & Use of Laboratory Animals (8th Edition) and therefore in accordance with all Inotiv Boulder IACUC approved policies and procedures.

A total of 72 SOD1 Rat model 2148 animals are used in this experiment: 12 non-carrier wild-type females, 12 non-carrier wild-type males, 12 mutant females, and 36 mutant males. At the time of the experiment, animals are 10-13 weeks old. Animals are housed two animals per cage for the duration of the experiment.

On study day 0, animals begins behavioral assessments and cage side observations to track disease progression. Body weights are recorded every other day.

Animals in Group 7 (shown in Table 26) are anesthetized by continuous inhalation of 2-3% isoflurane in oxygen delivered at 3 L/min. After anesthesia induction animals are mounted in a stereotaxic apparatus on a heating pad and the surgical site sterilized with Betadine and 70% ethanol. Using sterile technique, an incision is made over the sagittal suture exposing the skull. Bregma is located at the junction of the sagittal and coronal sutures, and the skull is trephinated using an electric drill equipped with a 1.6 mm burr drill bit. A 28-gauge infusion cannula connected to a 10 μl Hamilton syringe mounted on a syringe pump is lowered into the burr hole to the desired stereotaxic coordinates for AAV administration. Twenty μl of AAV is infused at a rate of 4 μl/min, and infusion cannula remains in place for 10 min before the cannula is retracted. The scalp is closed with surgical staples or suture and buprenorphine XR is administered for post-surgery analgesia (0.65 mg/kg subcutaneous). Animals are removed from anesthesia and recover in a cage placed on a heating pad until they have regained righting reflex. Animals are singly housed for up to 7 days post-surgery and are monitored daily during that time. If animals display any signs of impaired motor function at the 1-hour post-surgery monitoring session (e.g. limb dragging or inability to stay in an upright, quadruped stature) the animal is euthanized according to Inotiv Boulder IACUC-approved methods. Staples are removed 10-14 after surgery.

TABLE 26
Study Group Designations.

| | | | | | | Dose | Dose | |
|---|---|---|---|---|---|---|---|---|
| | | | | Dose Level | Dose Route/ | Vol. | Conc. | Dose Route\ |
| Group | N | Treatment | Sex | (mg/kg) | Frequency | (ml/kg) | (mg/ml) | Frequency |
| 1 | 12 | Wildtype | F | N/A | IP/5X week | 10 | 0 | IP/5X week |
| | | Vehicle | | | | | | |
| 2 | 12 | Wildtype | M | N/A | IP/5X week | 10 | 0 | IP/5X week |
| | | Vehicle | | | | | | |
| 3 | 12 | Mutant | F | N/A | IP/5X week | 10 | 0 | IP/5X week |
| | | Vehicle | | | | | | |
| 4 | 12 | Mutant | M | N/A | IP/5X week | 10 | 0 | IP/5X week |
| | | Vehicle | | | | | | |
| 5 | 8 | Mutant | M | 5000 | IP/5X week | 10 | 500 | IP/5X week |
| | | Trehalose | | | | | | |
| 6 | 8 | Mutant | M | 200 | PO/5X week | 10 | 20 | PO/5X week |
| | | CNT | | | | | | |
| | | (nano | | | | | | |
| | | trehalose) | | | | | | |
| 7 | 8 | Mutant | M | 2e11 viral | ICV/1X | N/A | N/A | ICV/1X |
| | | AAV - ICV | | genomes/animal | | | | |

*5X/week is for duration of the study = 14 weeks

During the trials, animals are observed on a weekly basis and weighed. Animals also undergo a beam walk and rotarod test. Whole blood is taken via intravenous tail bleed at various times (e.g., on a weekly basis).

All animals are sacrificed on Study Day 95 or when any animal is moribund (e.g., Exhibits paralysis. When an animal begins to show paralysis in any portion of the body, a HydroGel pack is placed in the bottom of the home cage to limit dehydration. Monitoring continues until a rat reaches end-stage, defined as loss of the ability to right itself after being placed on its side, or inability to ambulate). Animals are treated with anesthesia of 3-4% isoflurane in oxygen. Animals are then bled to exsanguinate followed by bilateral pneumothoracotomy.

The following samples are taken from sacrifices animals: whole blood, cervical spinal column, kidneys, liver, gastrocnemius, CSF, RT brain hemisphere, LT brain hemisphere, L5 spinal column, L1-L4 spinal column, and thoracic spinal column. SOD1 protein expression is determined in each sample.

In addition, histological sections are prepared for L5 spinal column, kidney, liver, and gastrocnemius samples. Spinal column samples require decalcification. Samples are then trimmed into two transverse pieces from the lumbar region (specifically targeting the L5 region) and both halves are embedded in paraffin into one block. Kidney samples are trimmed in half longitudinally and one half of each sample and are embedded in paraffin 1 per block. A representative piece from the left lateral and right medial lobe of each liver sample is trimmed. Both representative pieces are embedded in paraffin together in a single block. Gastrocnemius samples are embedded in OCT longitudinally 1 per block after being flash frozen. All FFPE blocks are sectioned (4 μm) and stained for H&E. In addition, all spinal column blocks are sectioned (4 μm) for an additional 3 slides for IHC staining of TMEM119, GFAP, and ChAT. All gastrocnemius samples are stained for VAChT+alpha-Bungarotoxin. In addition to FFPE blocks, OCT blocks are prepared for H&E and IHC.

The slides are subsequently imaged for IHC (or IF) expression using microscopy, and H&E-stained slides are assessed for safety/tolerability in a toxicological pathology evaluation by a pathologist. Changes specific to the disease model (ALS) are evaluated by IHC and image analysis parameters.

### Example 7: Trehalose in a Non-Human Primate (Macaques) Model of Alzheimer's Disease

### Methods

### AAV Trehaholose Treatment

In the following example, the ability of a viral vector to prevent neurodegeneration in a nonhuman primate (NHP) model of Alzheimer's disease (AD) is tested. Animals first receive an injection into the brain of a viral vector causing them to express a protein, tau. Then the animals receive an injection of a second vector causing them to express a naturally occurring sugar to prevent the misfolding of tau protein which typically leads to neurodegeneration.

Eight macaques (M/F, ≥22 years old) are used for the present experiment. All animals have baseline plasma, serum, and cerebrospinal fluid (CSF) collected. AAV-double mutant tau (2×20 μL) are injected into the left entorhinal cortex (ERC), and injections of AAV-empty vector control are injected into the right ERC (2×20 μL). Two months later, AAV-trehalose (pBK1928 (AAV9-CAG-otsA-P2A-otsB-bGHpA)) or AAV-empty vector control (2×20 μL) are delivered into the left hippocampus and AAV-empty vector control into the right hippocampus (2×20 μL). Three months following the second surgery, plasma, serum, and CSF are collected. Blood is collected from the femoral vein. CSF is collected using a lumbar or cisternal puncture.

The animals then are sacrificed, the brains removed and punched for neurochemistry, then fixed and sectioned for immunohistochemistry (IHC). During each injection (40 Ml total), a dose of 1E10¹³ vg/Ml is used.

### SLS005 Infusion

Before the surgery, baseline blood and CSF are collected from the animals. All animals received bilateral stereotaxic injections of AAV expressing a double tau mutant in the left ERC, and an AAV-empty vector control in the right ERC. One week after the surgery, animals are administered weekly infusion of SLS-005 (IV/PO) or saline for 23 weeks. Post surgery, blood is collected from the animals on weeks 11, 15, 19, and 23. Immediately prior to necropsy, blood and CSF are collected. After the animals are sacrificed, brains were removed and sectioned for immunohistochemistry.

### MRI Guided Neurosurgery

During the experiment, MRI scanning is performed. Stereotaxic intracranial injections are performed under intraoperative MRI guidance. Animals are prepared and are anesthetized with ketamine (3-10 mg/kg, IM) and either dexmedetomidine (0.03 mg/kg, IM) or midazolam (0.05-0.5 mg/kg) and maintained with gas anesthesia (e.g., isoflurane, sevoflurane) or booster injections of ketamine (1.5 mg/kg, IM) and dexmedetomidine (0.015 mg/kg, IM). Animals are intubated to maintain a stable airway. If necessary, cetacaine spray (200 mg, topical) may be applied to the throat to assist with intubation. Animals are placed in an MRI compatible stereotaxic frame and MRI-opaque fiducial markers are placed around the skull for neuronavigation registration. T1 and/or T2-weighted images are obtained. The locations of the fiducial markers are permanently marked with a tattoo dot on the skin using a commercial tattoo marker (Spaulding & Rogers Mfg., Voorheesville, NY) with sterile needle and ink. The MRI scan time is approximately 20 minutes, sedation lasts ≤1 hour. Following the procedure, dexmedetomidine/midazolam may be reversed with atipamezole (0.15-0.3 mg/kg, IM) or flumazenil (0.025 mg/kg, IV), respectively. A new MRI is performed prior to the second surgery as the skin healing following the first surgery affects the fiducial locations for neuronavigation registration. If necessary, up to one repeat MRI can be performed.

### Tissue Processing

The number of neurons in the entorhinal cortex and hippocampus by stereological counting of NeuN positive neurons is determined.

At time of sacrifice, animals are anesthetized with ketamine (10 mg/kg, IM), xylazine (2 mg/kg, IM) or midazolam (0.05-0.5 mg/kg), and either hydromorphone (0.2 mg/kg, IM) or morphine (1 mg/kg, IM), followed by gas anesthesia (e.g., isoflurane, sevoflurane) or additional drug delivered IV [ketamine (≤20 mg/kg, IV), xylazine (≤4 mg/kg, IV), and either hydromorphone (≤0.4 mg/kg, IV) or morphine (≤2 mg/kg, IV)], if needed to achieve a surgical plane of anesthesia. Once a surgical plane of anesthesia is achieved as verified by lack of response to toe/finger pinch, palpebral reflex, and corneal reflex, the thoracic cavity is opened, heparin (5,000 IU, IC) is injected into the left ventricle of the heart and the animal is euthanized via transcardial perfusion of 0.9% saline (1-2 L) followed by 4% buffered formaldehyde (1-2 L). If perfusion cannot be performed for any reason, animals is anesthetized with ketamine (10 mg/kg, IM) followed by a pentobarbital-containing euthanasia solution (86-120 mg/kg, IV).

### Results

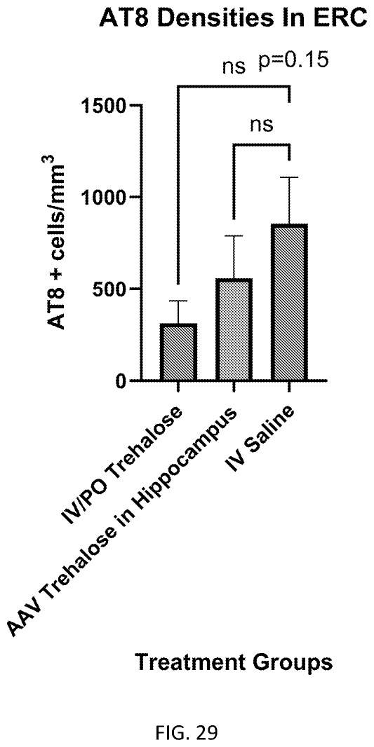

To determine the effect of AAV Trehalose and SLS-005 IV/PO Trehalose treatments on AT8 expression, animals are treated with AAV Trehalose (pBK1928; i.e., (AAV9-CAG-otsA-P2A-otsB-bGHpA), SLS-005 Trehalose, or Saline (control) as described above. Brain samples are collected, and AT8 expression is measured in the entorhinal cortex using IHC analysis. AT8 is used as a Tau pathology (phosphorylated Tau) marker. Treatment with AAV Trehalose and SLS-005 IV/PO Trehalose as compared to the control resulted in lower levels of AT8 in the ERC (FIG. 29). The results show that both AAV Trehalose and SLS-005 IV/PO Trehalose can reduce the overexpression of Tau in these animals, thereby rescuing neurodegeneration and reversing the disease progression.

These results imply that treatment with AAV trehalose and SLS-005 IV Trehalose both reduce the final neurodegeneration, i.e. the death of neurons due to Tau overexpression in these animals. This is because the results indicate that the disease progression is being reversed and Tau mediated neuronal death has been averted and reversed in animals in this study. Thus, both methods are viable treatment options for tauopathies in general and in Alzheimer's disease progression. This can both halt the disease progression and likely reverse disease pathology manifesting as death of neurons.

What is claimed is:

1. A method of treating a subject having a neurodegenerative disorder comprising administering to the subject a therapeutically effective amount of a vector comprising a nucleic acid that encodes trehalose-6-phosphate synthase (OtsA), a nucleic acid that encodes trehalose 6-phosphate phosphatase (OtsB), a nucleic acid that encodes OtsA and OtsB, or a nucleic acid that encodes a trehalose biosynthesis fusion polypeptide.

2. A method of preventing a neurodegenerative disorder comprising administering to the subject a therapeutically effective amount of a vector comprising a nucleic acid that encodes trehalose-6-phosphate synthase (OtsA), a nucleic acid that encodes trehalose 6-phosphate phosphatase (OtsB), a nucleic acid that encodes OtsA and OtsB, or a nucleic acid that encodes a trehalose biosynthesis fusion polypeptide.

3. A method of treating a subject having a neurodegenerative disorder comprising administering to the subject a therapeutically effective amount of trehalose and a therapeutically effective amount of a vector comprising a nucleic acid that encodes trehalose-6-phosphate synthase (OtsA), a nucleic acid that encodes trehalose 6-phosphate phosphatase (OtsB), a nucleic acid that encodes OtsA and OtsB, or nucleic acid that encodes a trehalose biosynthesis fusion polypeptide.

4. A method of treating a subject in need thereof, the method comprising
(a) identifying the subject as having a neurodegenerative disorder; and
(b) administering to the subject a therapeutically effective amount of a vector comprising a nucleic acid that encodes trehalose-6-phosphate synthase (OtsA), a nucleic acid that encodes trehalose 6-phosphate phosphatase (OtsB), a nucleic acid that encodes OtsA and OtsB, or a nucleic acid that encodes a trehalose biosynthesis fusion polypeptide.

5. The method of any of the preceding claims, wherein the neurodegenerative disorder is associated with the formation of protein plaques.

6. The method of any of the preceding claims, wherein the neurodegenerative disorder is selected from the group consisting of Alzheimer's disease (AD), Huntington's disease (HD), amyotrophic lateral sclerosis (ALS), Parkinson's disease (PD), ataxia (e.g., Friedreich's ataxia), progressive supranuclear palsy (PSLP), spinocerebellar ataxia (SCA) (e.g., SCA3), and amyloidosis.

7. The method of any one of the preceding claims, wherein the vector comprises a nucleic acid that encodes OtsA and OtsB.

8. The method of claim 7, wherein the vector further comprises a peptide linker sequence located between a nucleic acid encoding OtsA and a nucleic acid encoding OtsB.

9. The method of any one of the preceding claims, wherein the vector further comprises an enhancer.

10. The method of any of the preceding claims, wherein the vector is an adeno-associated viral (AAV) vector.

11. The method of claim 10, wherein the AAV vector is any one of AAV1, AAV2, AAV3, AAV4, AAV5, AAV6, AAV7, AAV8, or AAV9.

12. The method of any one of claims 10-11, wherein the AAV vector is a recombinant adeno-associated virus (rAAV).

13. The method of any one of the preceding claims, wherein the vector comprises a nucleic acid that encodes a trehalose biosynthesis fusion polypeptide.

14. The method of claim 13, wherein the trehalose biosynthesis fusion polypeptide comprises OtsA and OtsB, or functional fragments thereof.

15. The method of any one of the preceding claims, wherein the nucleic acid encoding the trehalose biosynthesis fusion polypeptide comprises a nucleic acid encoding OtsA, or a functional fragment thereof and a nucleic acid encoding OtsB, or a functional fragment thereof.

16. The method of claim 15, wherein the nucleic acid encoding the trehalose biosynthesis fusion polypeptide further comprises one or more self-cleaving peptides.

17. The method of claim 16, wherein the self-cleaving peptides are selected from the group consisting of P2A and T2A.

18. The method of claim 16, wherein the self-cleaving peptides are individually selected from the group consisting of P2A and T2A.

19. The method of any of the preceding claims, wherein the nucleic acid that encodes trehalose-6-phosphate synthase (OtsA), the nucleic acid that encodes trehalose 6-phosphate phosphatase (OtsB), the nucleic acid that encodes OtsA and OtsB, or the nucleic acid encoding a trehalose biosynthesis fusion polypeptide is operably linked to a promoter.

20. The method of any of the preceding claims, wherein the administering is intrathecal, intraparenchymal, intracisterna (ICM), or intracerebroventricular injection (ICV).

21. The method of any one of the preceding claims, wherein the subject is a mammal.

22. The method of claim 16, wherein the mammal is a human.

23. A method of treating a subject having a disorder associated with excessive inflammation comprising administering to the subject a therapeutically effective amount of a vector comprising a nucleic acid that encodes trehalose-6-phosphate synthase (OtsA), a nucleic acid that encodes trehalose 6-phosphate phosphatase (OtsB), a nucleic acid that encodes OtsA and OtsB, or a nucleic acid encoding a trehalose biosynthesis fusion polypeptide.

24. A method of preventing symptoms of a subject having a disorder associated with excessive inflammation comprising administering to the subject a therapeutically effective amount of a vector comprising a nucleic acid that encodes trehalose-6-phosphate synthase (OtsA), a nucleic acid that encodes trehalose 6-phosphate phosphatase (OtsB), a nucleic acid that encodes OtsA and OtsB, or a nucleic acid encoding a trehalose biosynthesis fusion polypeptide.

25. The method of claim 23 or 24, wherein the administering comprises intravenous administration.

26. The method of any one of claims 23-25, wherein the disorder associated with excessive inflammation is atopic dermatitis

27. The method of any one of claims 24-26, wherein the subject is a mammal.

28. The method of claim 27, wherein the mammal is a human.

29. A vector comprising a nucleic acid that encodes trehalose-6-phosphate synthase (OtsA), a nucleic acid that encodes trehalose 6-phosphate phosphatase (OtsB), a nucleic acid that encodes OtsA and OtsB, or a nucleic acid that encodes a trehalose biosynthesis fusion polypeptide.

30. The vector of claim 24, wherein the vector comprises a nucleic acid that encodes OtsA and OtsB.

31. The vector of any one of claim 29 or 30, wherein the vector further comprises a peptide linker sequence located between a nucleic acid encoding OtsA and a nucleic acid encoding OtsB.

32. The vector of any one of claims 29-31, wherein the vector further comprises an enhancer.

33. The vector of any one of claims 29-32, wherein the vector is an adeno-associated viral (AAV) vector.

34. The vector of claim 33, wherein the AAV vector is any one of AAV1, AAV2, AAV3, AAV4, AAV5, AAV6, AAV7, AAV8, or AAV9.

35. The vector of any one of claim 33 or 34, wherein the AAV vector is a recombinant adeno-associated virus (rAAV).

36. The vector of any one of claims 33-35, wherein the vector comprises a nucleic acid that encodes a trehalose biosynthesis fusion polypeptide.

37. The vector of claim 36, wherein the trehalose biosynthesis fusion polypeptide comprises OtsA and OtsB, or functional fragments thereof.

38. The vector of any one of claims 29-37, wherein the nucleic acid encoding the trehalose biosynthesis fusion polypeptide comprises a nucleic acid encoding OtsA, or a functional fragment thereof and a nucleic acid encoding OtsB, or a functional fragment thereof.

39. The vector of claim 38, wherein the nucleic acid encoding the trehalose biosynthesis fusion polypeptide further comprises one or more self-cleaving peptides.

40. The vector of claim 39, wherein the self-cleaving peptides are selected from the group consisting of P2A and T2A.

41. The vector of claim 39, wherein the self-cleaving peptides are individually selected from the group consisting of P2A and T2A.

42. The vector of any one of claims 29-41, wherein the nucleic acid that encodes trehalose-6-phosphate synthase (OtsA), the nucleic acid that encodes trehalose 6-phosphate phosphatase (OtsB), the nucleic acid that encodes OtsA and OtsB, or the nucleic acid encoding a trehalose biosynthesis fusion polypeptide is operably linked to a promoter.

43. The vector of any one of the preceding claims, wherein the nucleic acid that encodes OtsA is SEQ ID NO: 1.

44. The vector of any one of the preceding claims, wherein the OtsA comprises SEQ ID NO: 2.

45. The vector of any one of the preceding claims, wherein the nucleic acid that encodes OtsB is SEQ ID NO: 3.

46. The vector of any one of the preceding claims, wherein the OtsB comprises SEQ ID NO: 4.

47. The vector of any one of the preceding claims, wherein the nucleic acid that encodes OtsA and OtsB, or the nucleic acid encoding a trehalose biosynthesis fusion polypeptide comprises SEQ ID NO: 5.

48. The vector of any one of the preceding claims, wherein the trehalose biosynthesis fusion polypeptide comprises SEQ ID NO: 6.

49. The vector of any one of the preceding claims, wherein the nucleic acid that encodes P2A is SEQ ID NO: 7.

50. The vector of any one of the preceding claims, wherein the P2A comprises SEQ ID NO: 8.

51. The vector of any one of the preceding claims, wherein the nucleic acid that encodes T2A is SEQ ID NO: 9.

52. The vector of any one of the preceding claims, wherein the T2A comprises SEQ ID NO: 10.

53. A method of producing the vector of claim 29 from a host cell, the method comprising culturing the host cell comprising the vector of claim 29 in a medium in which the host cell can express the vector.

54. The method of any one of the preceding claims, wherein the nucleic acid that encodes OtsA is SEQ ID NO: 1.

55. The method of any one of the preceding claims, wherein the OtsA comprises SEQ ID NO: 2.

56. The method of any one of the preceding claims, wherein the nucleic acid that encodes OtsB is SEQ ID NO: 3.

57. The method of any one of the preceding claims, wherein the OtsB comprises SEQ ID NO: 4.

58. The method of any one of the preceding claims, wherein the nucleic acid that encodes OtsA and OtsB, or the nucleic acid encoding a trehalose biosynthesis fusion polypeptide comprises SEQ ID NO: 5.

59. The method of any one of the preceding claims, wherein the trehalose biosynthesis fusion polypeptide comprises SEQ ID NO: 6.

60. The method of any one of the preceding claims, wherein the nucleic acid that encodes P2A is SEQ ID NO: 7.

61. The method of any one of the preceding claims, wherein the P2A comprises SEQ ID NO: 8.

62. The method of any one of the preceding claims, wherein the nucleic acid that encodes T2A is SEQ ID NO: 9.

63. The method of any one of the preceding claims, wherein the T2A comprises SEQ ID NO: 10.


SEQUENCE LISTING (10 sequences)

SEQ ID NO: 1 - DNA, 1425 bp, Escherichia coli
  source 1..1425: mol_type = other DNA
atgagtcgtttagtcgtagtatctaaccggattgcaccaccagacgagcacgccgccagtgccggtggccttgccgttggcatactgggggcactgaaagccgcaggcggactgtggtttggctggagtggtgaaacagggaatgaggatcagccgctaaaaaaggtgaaaaaaggtaacattacgtgggcctcttttaacctcagcgaacaggaccttgacgaatactacaaccaattctccaatgccgttctctggcccgcttttcattatcggctcgatctggtgcaatttcagcgtcctgcctgggacggctatctacgcgtaaatgcgttgctggcagataaattactgccgctgttgcaagacgatgacattatctggatccacgattatcacctgttgccatttgcgcatgaattacgcaaacggggagtgaataatcgcattggtttctttctgcatattcctttcccgacaccggaaatcttcaacgcgctgccgacatatgacaccttgcttgaacagctttgtgattatgatttgctgggtttccagacagaaaacgatcgtctggcgttcctggattgtctttctaacctgacccgcgtcacgacacgtagcgcaaaaagccatacagcctggggcaaagcatttcgaacagaagtctacccgatcggcattgaaccgaaagaaatagccaaacaggctgccgggccactgccgccaaaactggcgcaacttaaagcggaactgaaaaacgtacaaaatatcttttctgtcgaacggctggattattccaaaggtttgccagagcgttttctcgcctatgaagcgttgctggaaaaatatccgcagcatcatggtaaaattcgttatacccagattgcaccaacgtcgcgtggtgatgtgcaagcctatcaggatattcgtcatcagctcgaaaatgaagctggacgaattaatggtaaatacgggcaattaggctggacgccgctttattatttgaatcagcattttgaccgtaaattactgatgaaaatattccgctactctgacgtgggcttagtgacgccactgcgtgacgggatgaacctggtagcaaaagagtatgttgctgctcaggacccagccaatccgggcgttcttgttctttcgcaatttgcgggagcggcaaacgagttaacgtcggcgttaattgttaacccctacgatcgtgacgaagttgcagctgcgctggatcgtgcattgactatgtcgctggcggaacgtatttcccgtcatgcagaaatgctggacgttatcgtgaaaaacgatattaaccactggcaggagtgcttcattagcgacctaaagcagatagttccgcgaagcgcggaaagccagcagcgcgataaagttgctacctttccaaagcttgcgtag

SEQ ID NO: 2 - protein, 474 aa, Escherichia coli
MSRLVVVSNRIAPPDEHAASAGGLAVGILGALKAAGGLWFGWSGETGNEDQPLKKVKKGNITWASFNLSEQDLDEYYNQFSNAVLWPAFHYRLDLVQFQRPAWDGYLRVNALLADKLLPLLQDDDIIWIHDYHLLPFAHELRKRGVNNRIGFFLHIPFPTPEIFNALPTYDTLLEQLCDYDLLGFQTENDRLAFLDCLSNLTRVTTRSAKSHTAWGKAFRTEVYPIGIEPKEIAKQAAGPLPPKLAQLKAELKNVQNIFSVERLDYSKGLPERFLAYEALLEKYPQHHGKIRYTQIAPTSRGDVQAYQDIRHQLENEAGRINGKYGQLGWTPLYYLNQHFDRKLLMKIFRYSDVGLVTPLRDGMNLVAKEYVAAQDPANPGVLVLSQFAGAANELTSALIVNPYDRDEVAAALDRALTMSLAERISRHAEMLDVIVKNDINHWQECFISDLKQIVPRSAESQQRDKVATFPKLA

SEQ ID NO: 3 - DNA, 798 bp, Escherichia coli
  source 1..798: mol_type = other DNA
gtgacagaaccgttaaccgaaacccctgaactatccgcgaaatatgcctggttttttgatcttgatggaacgctggcggaaatcaaaccgcatcccgatcaggtcgtcgtgcctgacaatattctgcaaggactacagctactggcaaccgcaagtgatggtgcattggcattgatatcagggcgctcaatggtggagcttgacgcactggcaaaaccttatcgcttcccgttagcgggcgtgcatggggcggagcgccgtgacatcaatggtaaaacacatatcgttcatctgccggatgcgattgcgcgtgatattagcgtgcaactgcatacagtcatcgctcagtatcccggcgcggagctggaggcgaaagggatggcttttgcgctgcattatcgtcaggctccgcagcatgaagacgcattaatgacattagcgcaacgtattactcagatctggccacaaatggcgttacagcagggaaagtgtgttgtcgagatcaaaccgagaggtaccagtaaaggtgaggcaattgcagcttttatgcaggaagctccctttatcgggcgaacgcccgtatttctgggcgatgatttaaccgatgaatctggcttcgcagtcgttaaccgactgggcggaatgtcagtaaaaattggcacaggtgcaactcaggcatcatggcgactggcgggtgtgccggatgtctggagctggcttgaaatgataaccaccgcattacaacaaaaaagagaaaataacaggagtgatgactatgagtcgtttagtcgtagtatc

SEQ ID NO: 4 - protein, 266 aa, Escherichia coli
MTEPLTETPELSAKYAWFFDLDGTLAEIKPHPDQVVVPDNILQGLQLLATASDGALALISGRSMVELDALAKPYRFPLAGVHGAERRDINGKTHIVHLPDAIARDISVQLHTVIAQYPGAELEAKGMAFALHYRQAPQHEDALMTLAQRITQIWPQMALQQGKCVVEIKPRGTSKGEAIAAFMQEAPFIGRTPVFLGDDLTDESGFAVVNRLGGMSVKIGTGATQASWRLAGVPDVWSWLEMITTALQQKRENNRSDDYESFSRSI

SEQ ID NO: 5 - DNA, 2331 bp, synthetic construct
  source 1..2331: mol_type = other DNA
atgagtcgtttagtcgtagtatctaaccggattgcaccaccagacgagcacgccgccagtgccggtggccttgccgttggcatactgggggcactgaaagccgcaggcggactgtggtttggctggagtggtgaaacagggaatgaggatcagccgctaaaaaaggtgaaaaaaggtaacattacgtgggcctcttttaacctcagcgaacaggaccttgacgaatactacaaccaattctccaatgccgttctctggcccgcttttcattatcggctcgatctggtgcaatttcagcgtcctgcctgggacggctatctacgcgtaaatgcgttgctggcagataaattactgccgctgttgcaagacgatgacattatctggatccacgattatcacctgttgccatttgcgcatgaattacgcaaacggggagtgaataatcgcattggtttctttctgcatattcctttcccgacaccggaaatcttcaacgcgctgccgacatatgacaccttgcttgaacagctttgtgattatgatttgctgggtttccagacagaaaacgatcgtctggcgttcctggattgtctttctaacctgacccgcgtcacgacacgtagcgcaaaaagccatacagcctggggcaaagcatttcgaacagaagtctacccgatcggcattgaaccgaaagaaatagccaaacaggctgccgggccactgccgccaaaactggcgcaacttaaagcggaactgaaaaacgtacaaaatatcttttctgtcgaacggctggattattccaaaggtttgccagagcgttttctcgcctatgaagcgttgctggaaaaatatccgcagcatcatggtaaaattcgttatacccagattgcaccaacgtcgcgtggtgatgtgcaagcctatcaggatattcgtcatcagctcgaaaatgaagctggacgaattaatggtaaatacgggcaattaggctggacgccgctttattatttgaatcagcattttgaccgtaaattactgatgaaaatattccgctactctgacgtgggcttagtgacgccactgcgtgacgggatgaacctggtagcaaaagagtatgttgctgctcaggacccagccaatccgggcgttcttgttctttcgcaatttgcgggagcggcaaacgagttaacgtcggcgttaattgttaacccctacgatcgtgacgaagttgcagctgcgctggatcgtgcattgactatgtcgctggcggaacgtatttcccgtcatgcagaaatgctggacgttatcgtgaaaaacgatattaaccactggcaggagtgcttcattagcgacctaaagcagatagttccgcgaagcgcggaaagccagcagcgcgataaagttgctacctttccaaagcttgcggctactaacttcagcctgctgaagcaggctggcgacgtggaggagaaccctggacctgtgacagaaccgttaaccgaaacccctgaactatccgcgaaatatgcctggttttttgatcttgatggaacgctggcggaaatcaaaccgcatcccgatcaggtcgtcgtgcctgacaatattctgcaaggactacagctactggcaaccgcaagtgatggtgcattggcattgatatcagggcgctcaatggtggagcttgacgcactggcaaaaccttatcgcttcccgttagcgggcgtgcatggggcggagcgccgtgacatcaatggtaaaacacatatcgttcatctgccggatgcgattgcgcgtgatattagcgtgcaactgcatacagtcatcgctcagtatcccggcgcggagctggaggcgaaagggatggcttttgcgctgcattatcgtcaggctccgcagcatgaagacgcattaatgacattagcgcaacgtattactcagatctggccacaaatggcgttacagcagggaaagtgtgttgtcgagatcaaaccgagaggtaccagtaaaggtgaggcaattgcagcttttatgcaggaagctccctttatcgggcgaacgcccgtatttctgggcgatgatttaaccgatgaatctggcttcgcagtcgttaaccgactgggcggaatgtcagtaaaaattggcacaggtgcaactcaggcatcatggcgactggcgggtgtgccggatgtctggagctggcttgaaatgataaccaccgcattacaacaaaaaagagaaaataacaggagtgatgactatgagtcgtttagtcgtagtatcgagggcagaggaagtcttctaacatgcggtgacgtggaggagaatcccggccct

SEQ ID NO: 6 - protein, 777 aa, synthetic construct
MSRLVVVSNRIAPPDEHAASAGGLAVGILGALKAAGGLWFGWSGETGNEDQPLKKVKKGNITWASFNLSEQDLDEYYNQFSNAVLWPAFHYRLDLVQFQRPAWDGYLRVNALLADKLLPLLQDDDIIWIHDYHLLPFAHELRKRGVNNRIGFFLHIPFPTPEIFNALPTYDTLLEQLCDYDLLGFQTENDRLAFLDCLSNLTRVTTRSAKSHTAWGKAFRTEVYPIGIEPKEIAKQAAGPLPPKLAQLKAELKNVQNIFSVERLDYSKGLPERFLAYEALLEKYPQHHGKIRYTQIAPTSRGDVQAYQDIRHQLENEAGRINGKYGQLGWTPLYYLNQHFDRKLLMKIFRYSDVGLVTPLRDGMNLVAKEYVAAQDPANPGVLVLSQFAGAANELTSALIVNPYDRDEVAAALDRALTMSLAERISRHAEMLDVIVKNDINHWQECFISDLKQIVPRSAESQQRDKVATFPKLAATNFSLLKQAGDVEENPGPMTEPLTETPELSAKYAWFFDLDGTLAEIKPHPDQVVVPDNILQGLQLLATASDGALALISGRSMVELDALAKPYRFPLAGVHGAERRDINGKTHIVHLPDAIARDISVQLHTVIAQYPGAELEAKGMAFALHYRQAPQHEDALMTLAQRITQIWPQMALQQGKCVVEIKPRGTSKGEAIAAFMQEAPFIGRTPVFLGDDLTDESGFAVVNRLGGMSVKIGTGATQASWRLAGVPDVWSWLEMITTALQQKRENNRSDDYESFSRSIEGRGSLLTCGDVEENPGP

SEQ ID NO: 7 - DNA, 57 bp, Porcine Teschovirus-1
  source 1..57: mol_type = other DNA
gctactaacttcagcctgctgaagcaggctggcgacgtggaggagaaccctggacct

SEQ ID NO: 8 - protein, 19 aa, Porcine Teschovirus-1
ATNFSLLKQAGDVEENPGP

SEQ ID NO: 9 - DNA, 54 bp, Thosea asigna virus
  source 1..54: mol_type = other DNA
gagggcagaggaagtcttctaacatgcggtgacgtggaggagaatcccggccct

SEQ ID NO: 10 - protein, 18 aa, Thosea asigna virus
EGRGSLLTCGDVEENPGP